(19) United States
(12) Patent Application Publication
(10) Pub. No.: US 2026/0216356 A1
(43) Pub. Date: Jul. 30, 2026

(54) POLYOXAZOLINE ANTIBODY-DRUG CONJUGATES AND METHODS OF USE

(71) Applicant: Serina Therapeutics (AL), Inc., Hunstville, AL (US)

(72) Inventors: Joshua D. Thomas, Natick, MA (US); Randall Moreadith, Huntsville, AL (US); Zhihao Fang, Madison, AL (US)

(73) Assignee: Serina Therapeutics (AL), Inc., Huntsville, AL (US)

(21) Appl. No.: 19/463,708

(22) Filed: Jan. 29, 2026

Related U.S. Application Data
(60) Provisional application No. 63/750,944, filed on Jan. 29, 2025.

(51) Int. Cl. A61K 47/68; A61K 47/59

(52) U.S. Cl. CPC A61K 47/68037; A61K 47/59; A61K 47/6809; A61K 47/6855; A61K 47/6889

(57) ABSTRACT

Polymer antibody drug conjugates including peptide linkers and methods of using such conjugates are disclosed. Polymer drug linker moieties useful to conjugate with a recognition moiety (i.e. antibody) and drug linker moieties useful to conjugate with a recognition moiety and a polymer moiety to produce the polymer antibody drug conjugates are also disclosed.

### CROSS-REFERENCE TO RELATED APPLICATIONS

This application claims priority to U.S. Provisional Patent Application No. 63/750,944, filed on Jan. 29, 2025, the entire disclosure of which is incorporated by reference herein.

### FIELD OF THE INVENTION

The present disclosure relates to polymer antibody drug conjugates including peptide linkers and methods of using such conjugates. In addition, polymer drug linker moieties useful to conjugate with a recognition moiety and drug linker moieties useful to conjugate with a recognition moiety and a polymer moiety to produce the polymer antibody drug conjugates are also disclosed.

### BACKGROUND OF THE INVENTION

Small molecule pharmaceuticals have long been the standard in treatment for a variety of diseases, including, but not limited to, cancer. Such small molecule pharmaceuticals are normally administered orally in the form of liquids, pills, capsule and the like or parenterally in the form of injectable or intravenous formulations. While effective in treating a great many conditions, challenges remain. These include, but are not limited to, controlling the rate of drug delivery, targeting the delivery of the compounds to the desired site of action and maximizing the half-life of the compounds in the circulation. For example, many small molecule compounds exhibit decreased efficacy and therapeutic benefit due to metabolism prior to reaching the site of action. In addition, small molecule compounds can be relatively insoluble in aqueous solvents, requiring the use of complicated formulations for administration. In some instances, this may limit the clinical usefulness of such compounds even though they are effective in preclinical studies.

One objective in the field of drug delivery is to preferentially deliver a small molecule compound to a desired site of action. Furthermore, an additional objective is to control, at least partially, the delivery of the compound once the compound reaches the site of action. Still further, an additional objective is to accomplish one or more of the foregoing objectives with a delivery system that stabilizes the compound when delivered in vivo, extends the half-life of the compound in vivo, and aids in solubilizing compounds, particularly those compounds that are insoluble in aqueous solvents.

In order to address the shortcomings in the art, antibody-drug conjugates (ADCs) have been explored. A great deal of research and development has shown that ADCs are effective at providing targeted killing of cancer cells in animal models as well as in humans (P. D. Senter and E. L. Sievers, Nature Biotechnology, 30, 631-637 (2012); C&EN, Antibody Drug Conjugates, Jun. 18, 2012, pages 12-18; Tsuchikama, K; et. al., Nature Reviews Clinical Oncology, 2024, 21, 203-223). The first “armed antibody” was approved in 2011 (P. D. Senter and E. L. Sievers, Nature Biotechnology, 30, 631-637 (2012)).

It is common for ADCs to have one to four molecules of a drug or other compound coupled to an antibody using linker chemistry designed to release the agent into the tumor mass or into a tumor cell. One limitation of this approach is that insufficient toxin may be attached to the antibody to kill certain tumors, especially when less powerful toxins are used or when there is reduced antigen density present on the tumor cell surface. Also, increasing the number of points of toxin attachment on the antibody may compromise antibody binding capacity and decrease its ability to kill targeted cancer cells. Additionally, this approach can be. common with conventional small molecule compounds. Finally, some toxins, when attached to antibodies, are so hydrophobic that the toxins are taken up by off-target tissues independent of the binding region of the antibody, thus limiting their effective therapeutic potential.

Another approach with ADCs is to use a biodegradable polymer such as the polyacetal derived from oxidized dextran (Yurokovetskiy et al, U.S. Pat. No. 8,685,383). In such an approach, the polymer-ADC may contain multiple copies of the agent linked to the polymer backbone with the polymer itself being linked to the antibody. A drug-antibody ratio (DAR) of greater than one allows for improved cytotoxicity results when compared to non-targeted polymer drug conjugates alone. Biodegradable polymers, such as the polyacetal above, have the advantage of degrading and, thus, avoiding accumulation in the body. However, they also have the disadvantage of degrading more quickly than desired, either during preparation or in the body after administration. This approach may lead to premature release of the cytotoxic agent during circulation, thus limiting its effectiveness before it reaches the antigen on the target cell. Secondly, many such polymers, including the polyacetal mentioned above, have a biological origin that can lead to manufacturing challenges related to removing dangerous impurities and it also may increase the risk of adverse reactions when administered to a subject.

An additional approach to ADCs has included attaching an antibody to a biocompatible polymer to form a polymer-ADC. To date, this approach involves attaching a linear polymer (such as polyethylene glycol (PEG)) between the antibody and the drug. More specifically, U.S. Patent Publication No. 2014/0088021 to Riggs-Sauthier et al., demonstrates that one linear heterobifunctional PEG of either 2 kDa or 20 kDa may be first attached to one thiol on the HER2 antibody by maleimide chemistry. The PEG antibody complex is then coupled with one cytotoxic small molecule through an ester linkage. However, this approach has significant disadvantages because the ester link is not stable in-vivo and will hydrolyze to release the cytotoxic agent in blood before it reaches the antigen on the target cell. Also, the in vivo efficacy study of this polymer-ADC did not demonstrate active targeting of the polymer-ADC when either a 2 kDa or a 20 kDa polymer was used. The efficacy that was demonstrated was likely due to a pharmacokinetic half-life extension of the cytotoxic agent due to the PEG moiety. In addition, the PEG polymer-ADCs lost bioactivity in the cytotoxicity assay when compared to the drug alone. Moreover, the drug to antibody ratio (DAR) in this scenario is one that offers no advantage over the non-polymer-ADC approach. Indeed, the arming of multiple copies of either the drug or the antibody on the linear PEG polymer cannot be achieved with this approach.

In consideration of the foregoing, there is a need in the art for an improved ADC, in particular an improved polymer-ADC, that provides a solution to the problems and/or limitations described above. The present application provides such a solution by providing a synthetically manufactured polymer-ADC that delivers for (1) increased loading (higher DAR values) of the agent onto the targeting antibody and (2) increased half-life of the conjugate in vivo, while also not interfering substantially with the binding activity of the targeting agent (i.e., an antibody). Furthermore, the polymer-ADC conjugates of the present disclosure are readily constructed, and they can provide enhanced solubility of the compound to be delivered. Finally, the polymer-ADC conjugates of the present disclosure do not release drug until they reach the antigen on the target cell and are internalized, where the agent is released following cleavage from the polymer.

### SUMMARY

The present disclosure relates to polyoxazoline antibody drug conjugates, drug linker compounds, methods of preparing and using them, and intermediates thereof. The polyoxazoline antibody drug conjugates of the present disclosure are stable in circulation, yet capable of inflicting cell death once the free drug is released from a conjugate in the vicinity or within tumor cells.

In some embodiments, the present disclosure provides a polymer antibody drug conjugate of Formula (I), or a pharmaceutically acceptable salt or solvate thereof:

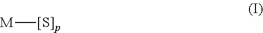

wherein:
M is a recognition moiety; and
S is a scaffold of Formula (II):

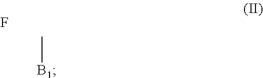

wherein:
F has Formula (III):

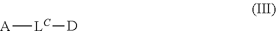

wherein:
A is A¹ after conjugation to M and A² prior to conjugation to M, wherein A¹ is a divalent linker moiety connecting M to L^{C};
L^{C} is a linker moiety connecting A¹ to D;
D is a drug moiety;
B¹ is a polymer moiety; and
p is an integer ranging from about 1 to about 8.

In some aspects, the present disclosure provides for A² being a monovalent linker moiety including a functional group capable of forming a covalent bond with a functional group of the recognition moiety.

In some embodiments, the scaffold S is of Formula (IIₐ), or a pharmaceutically acceptable salt or solvate thereof:

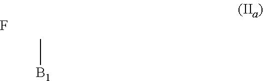

wherein
F is a drug linker moiety having Formula (IIIa), or a pharmaceutically acceptable salt or solvate thereof:

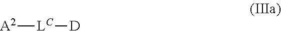

wherein:
A² is a monovalent linker moiety including a functional group capable of forming a covalent bond with a functional group of the recognition moiety.
L^{C} is a linker moiety connecting A² to D;
D is a drug moiety;
B¹ is a polymer moiety.

In some aspects, the present disclosure provides a drug linker moiety useful for conjugating with a recognition moiety and/or a polymer moiety, wherein the drug linker moiety is of Formula (IIIb), or a pharmaceutically acceptable salt or solvate thereof:

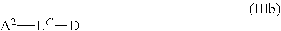

wherein:
A² is a monovalent linker moiety including a functional group capable of forming a covalent bond with a functional group of the recognition moiety;
L^{C} is a linker moiety connecting A² to D; and
D is a drug moiety;

In some aspects, the polymer moiety is a polyoxazoline polymer, wherein the polymer moiety is co-polymer including 50% to 99.5%, by weight of the total polymer components, of a polyoxazoline polymer.

In some aspects, the linker moiety L^{C} has the Formula (IV):

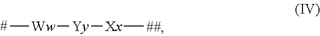

wherein:
each W independently is a peptide unit including at least two amino acids;
Y is a self-immolative moiety, a non-self-immolative releasable moiety or a non-cleavable moiety;
X is a spacer moiety;
w is an integer from about 0 to about 12;
y is an integer from 0 to 1;
x is an integer from 0 to 1;
# donates attachment to A¹ or A²; and

## donates attachment to D. In some embodiments, each A¹ independently is a divalent linker moiety connecting the recognition moiety to variable W, if present, or to variable Y, if W is absent; or connecting the recognition moiety to variable X, if both variables W and Y are absent; or connecting the recognition moiety to the drug moiety D, if variables W, Y and X are absent. In other embodiments, each A¹ independently is:

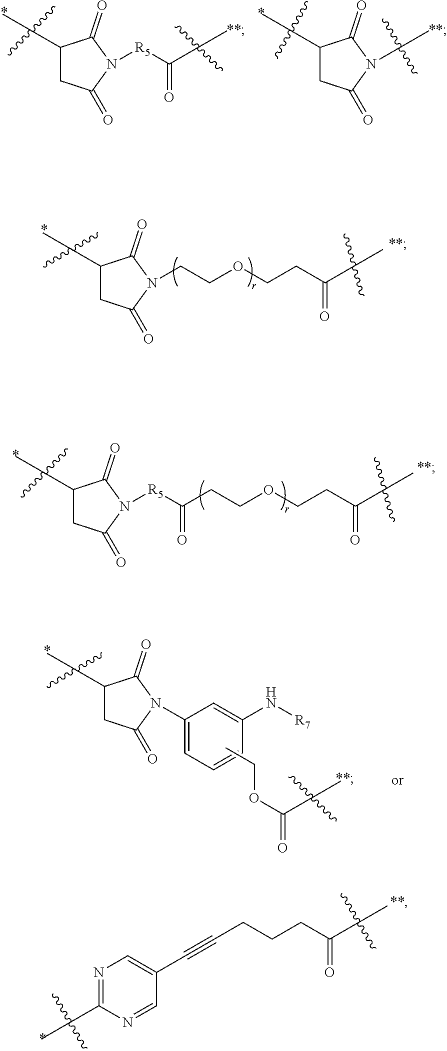

wherein:
R₅ is —O—, —NR₆—, —(C₁-C₁₀)-alkyl-, —(C₁-C₁₀)-alkenyl-, —(C₁-C₁₀)-alkynyl-, —(C₃-C₈)-cycloalkyl-, -aryl-, —O—(C₁-C₈)-alkyl-, —O—(C₁-C₁₀)-alkenyl-, —O—(C₁-C₁₀)-alkynyl-, -aryl-(C₁-C₁₀)-alkyl-C(O), —(C₁-C₁₀)-alkyl-aryl-, —O—C(O)—(CH₂CH₂O)₂—(CH₂)₂—, —(CH₂CH₂O)r-, or —(CH₂CH₂O)r-(CH₂)₂—,
R₆ is H, hydroxy, or (C₁-C₄)-alkyl;
R₇ is one or more amino acids;
r is an integer ranging from about 1 to about 12;
* denotes attachment to the recognition moiety;
** denotes attachment to L^{C}; and
*** donates attachment to the polymer moiety B¹.

In some embodiments, R₅ is —O—, —NH—, —N(CH₃)—, —(CH₂)₁₋₆—, —C₆H₅—CH₂—C(O), —C₆H₅—(CH₂)₂—C(O), —(C₁-C₁₀)-alkyl)-, —O—C(O)—(CH₂CH₂O)₆—(CH₂)₂—, —(CH₂CH₂O)₂—(CH₂)₂—, —(CH₂CH₂O)₄—(CH₂)₂ or —(CH₂CH₂O)₆—(CH₂)₂.

In some aspects, each W independently is a peptide unit connecting variable A¹ or A² to variable Y, if present, or to variable X if Y is absent; or connecting variable A¹ or A² to the drug moiety D if both variables Y and X are absent. In other aspects, Ww is a dipeptide, a tripeptide, a tetrapeptide, a pentapeptide, a hexapeptide, a heptapeptide, an octapeptide, a nonapeptide, a decapeptide, an undecapeptide, or a dodecapeptide unit. In still other aspects, Ww is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, citrulline, and derivatives thereof. In yet other aspects, Ww is -Lys-Val-Ala-, -Val-Ala-Lys-, Ala-Lys-Val-, Lys-Ala-Ala-, -Ala-Ala-Lys-, -Ala-Lys-Ala-, -Lys-Val-Cit-, Val-Cit-Lys-, Cit-Lys-Val-, -Val-Ala-, or -Ala-Val-. In still other aspects. Ww is:

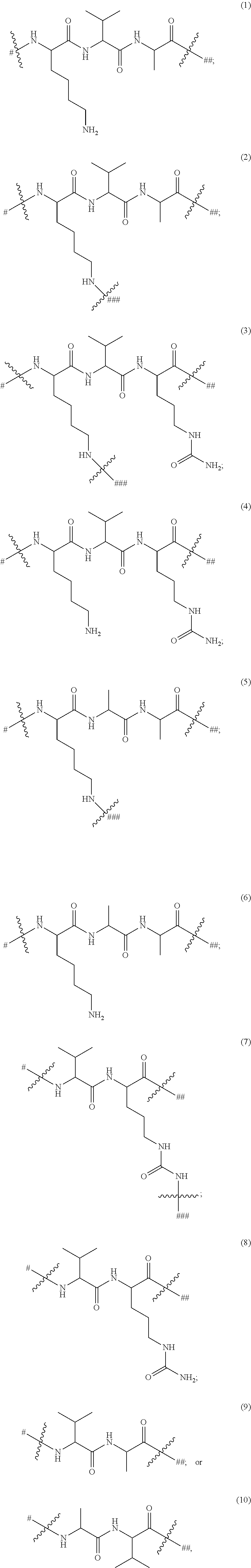

wherein:
# donates attachment to variable A¹ or A²;
## donates attachment to variable Y, if present, or to variable X if Y is absent, or to the drug moiety D, if both variables Y and X are absent; and
### donates attachment to the polymer moiety B¹.

In some embodiments, Y is a self-immolative moiety, a non-self-immolative releasable moiety, or a non-cleavable moiety connecting variable W to variable X, if present, or to the drug moiety D, if variable X is absent; or connecting variable A¹ or A² to variable X, if variable W is absent, or connecting variable A¹ or A² to the drug moiety D, if both variables W and X are absent. In some aspects, subscript y is 0 or 1.

In some embodiments, Y is

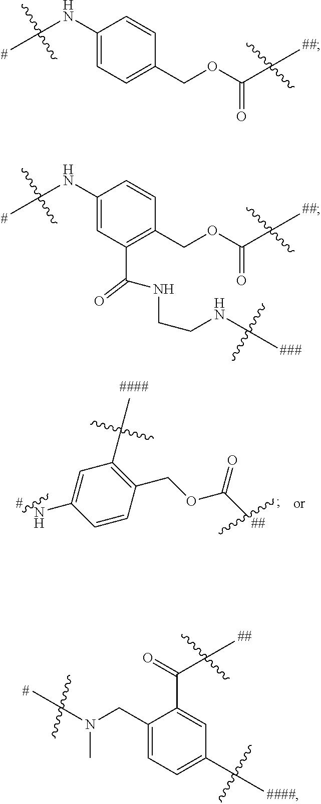

wherein:
# donates attachment to Ww or to variable A¹ or A² if Ww is absent;
## donates attachment to variable X, or to the drug moiety D, if X is absent;
### donates attachment to the polymer moiety B¹; and
#### donates attachment to variable A¹ or A². In other embodiments, X is a spacer moiety connecting variable Y to the drug moiety D; or connecting variable W to the drug moiety D, if variable Y is absent; or connecting variable A¹ or A² to the drug moiety D, if both variables W and X are absent. In some aspects, subscript x is 0 or 1. In other aspects, X is selected from the group consisting of:

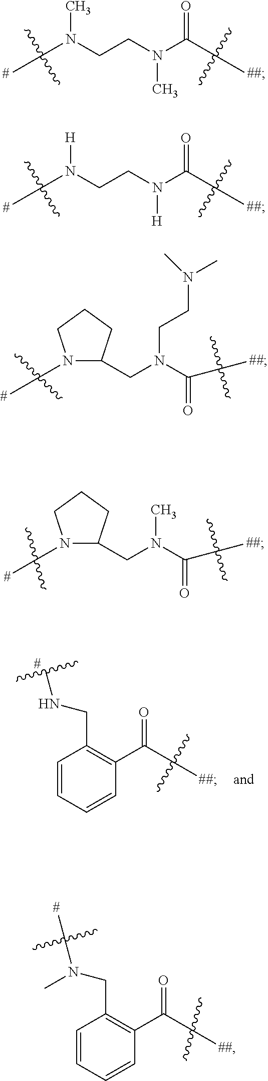

wherein:
# donates attachment to variable Y, if present; or attachment to variable W if Y is absent; or attachment to variable A₁ or A₂ if both variables W and Y are absent; and
## donates attachment to the drug moiety D.

In some embodiments, L^{C} is:

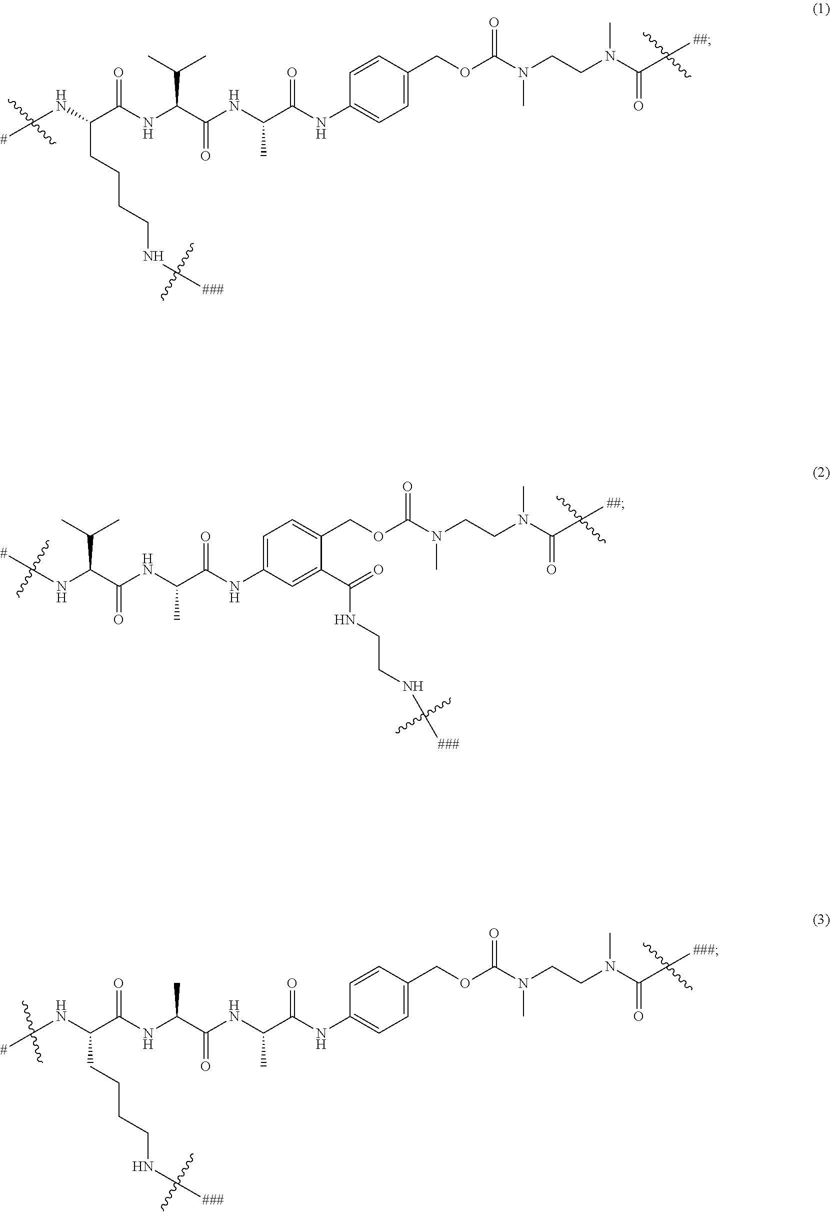

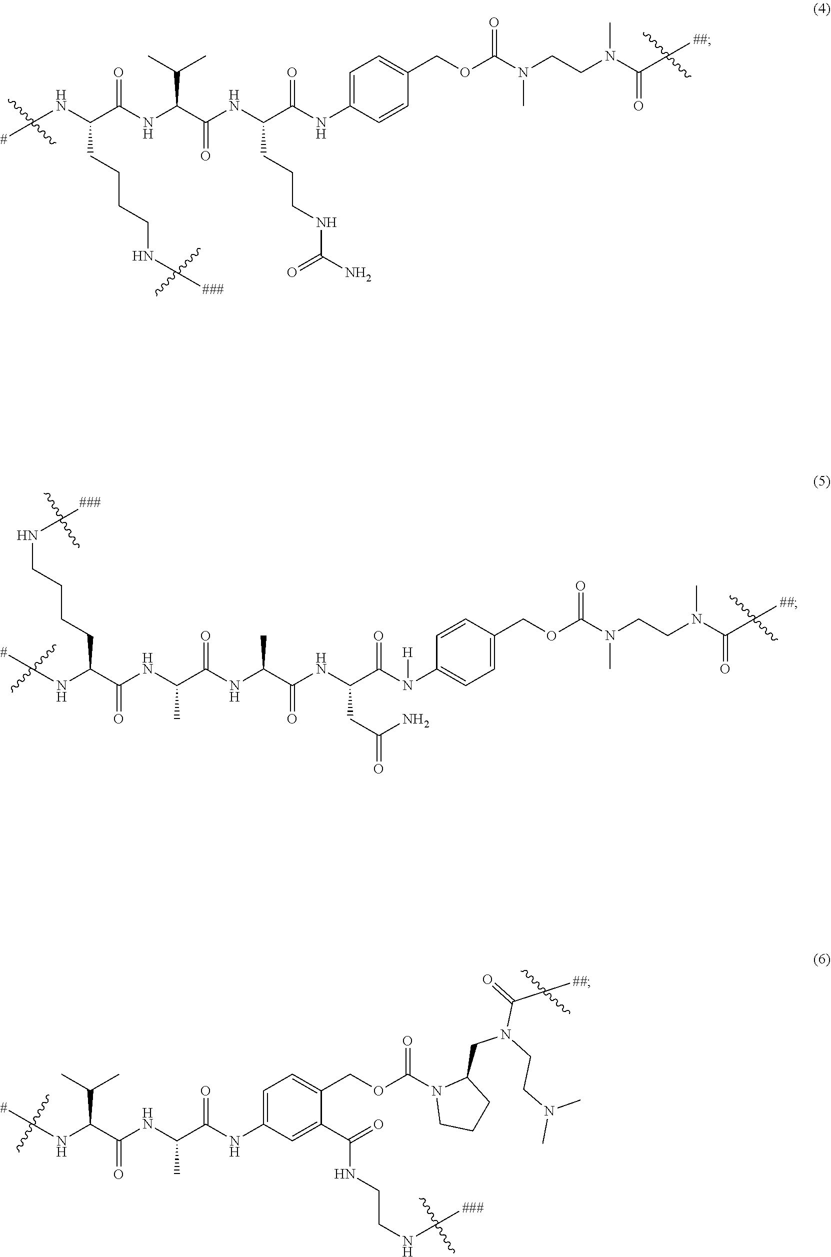

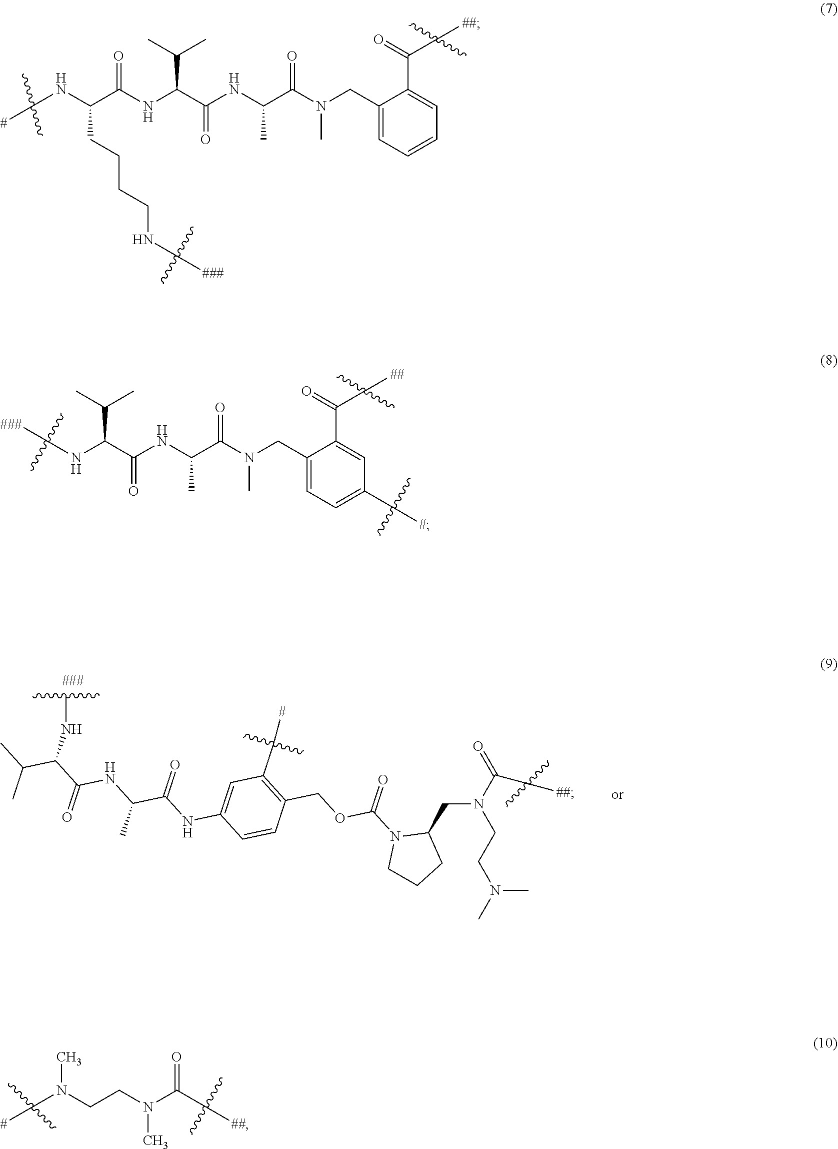

wherein:
# donates attachment to A¹ or A²;
## donates attachment to the drug moiety D; and
### donates attachment to the polymer moiety B¹.

In other embodiments, the drug moiety D is a camptothecin drug or a topoisomerase II inhibitor drug moiety, or a prodrug, solvate, pharmaceutically acceptable salt thereof. In yet other embodiments, the camptothecin drug moiety (D) is a compound of Formula (VI), or a prodrug, solvate, pharmaceutically acceptable salt thereof:

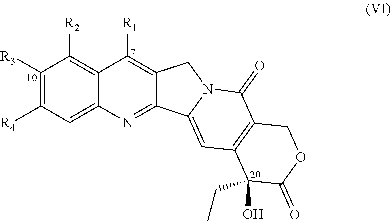

wherein:
R₁ is H,

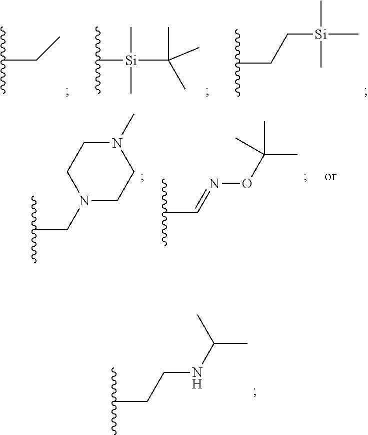

R₂ is H or

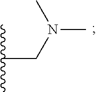 [SMILES: *CN(C)C]

or R₁ and R₂ together are

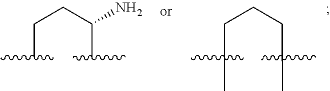 [SMILES: *CC[C@@H](*)N ; *CCC*]

R₃ is H, —OH, or —CH₃; or
R₄ is H or F; or R₃ and R₄ together are

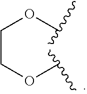 [SMILES: *OCCO*]

In still other embodiments, the camptothecin drug moiety of Formula (VI) is selected from the group consisting of SN38, camptothecin, 10-hydroxy-camptothecin, 7-ethyl camptothecin, exatecan, topotecan, irinotecan, belotecan, lurtotecan, rubitecan, silatecan, cositecan, gimatecan and topoisomerase I inhibitor 8. In yet other embodiments, the camptothecin drug moiety of Formula (VI) is covalently attached to L^{c} via a hydroxyl group wherein a hydrogen atom of the hydroxyl group is replaced with a bond to form a bond to L^{C}.

In some embodiments, the covalent attachment to L^{C} in the camptothecin drug moiety of Formula (VI) is via the hydroxyl group at position C-10 or at position C-20, wherein the hydrogen atom of the hydroxyl group at position C-10 or at position C-20 is replaced with a bond to form a bond to L^{C}. In some aspects, the camptothecin drug moiety of Formula (VI) for covalent attachment to L^{C} is selected from:

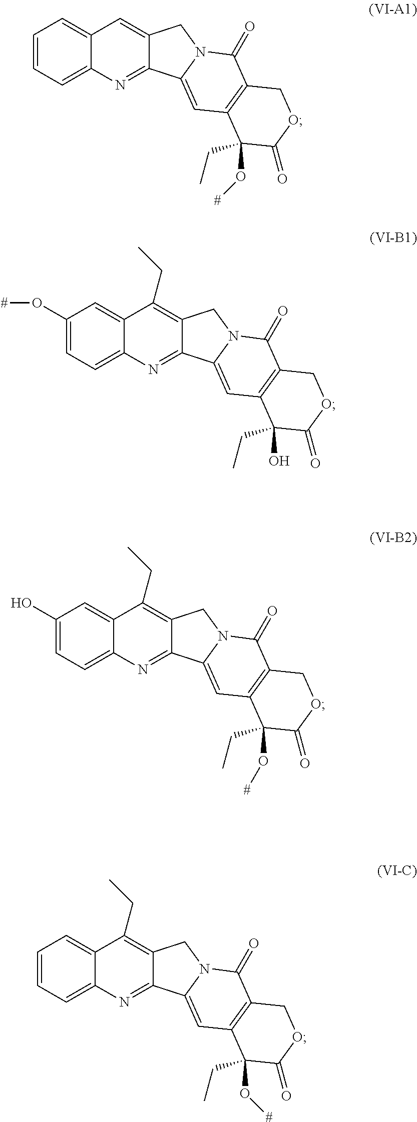

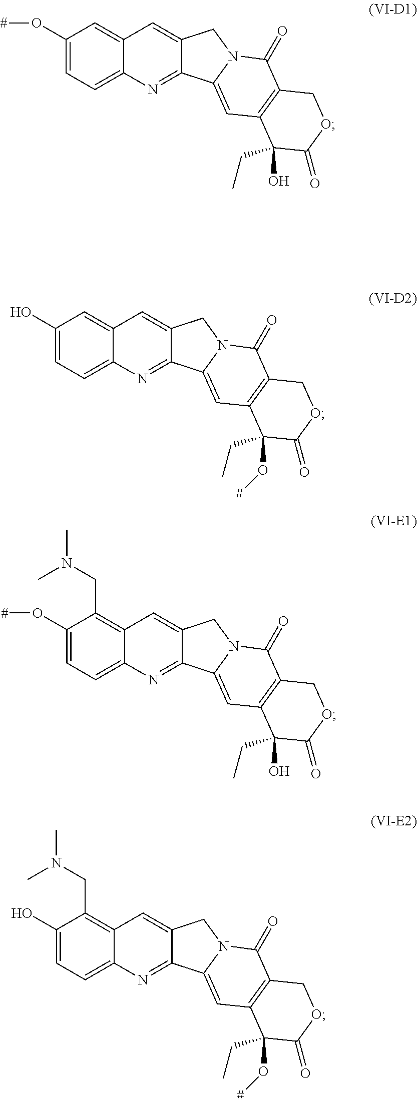

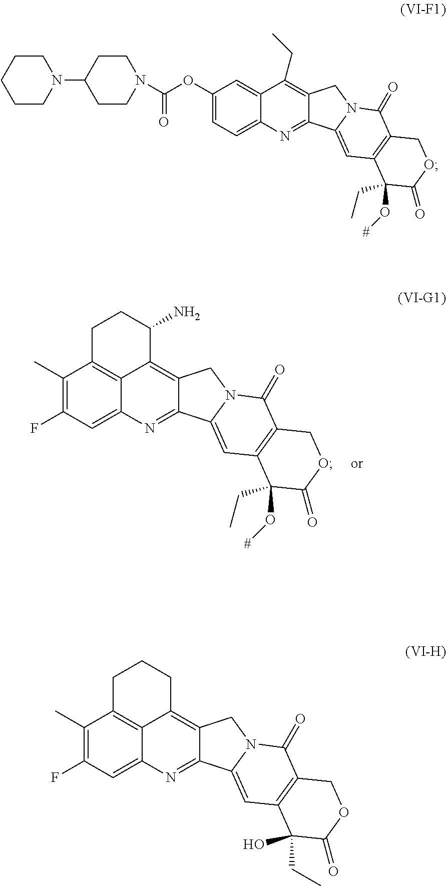

or a prodrug, solvate, a pharmaceutically acceptable salt; and
# donates attachment to L^{C}.

In some embodiments, the topoisomerase II inhibitor drug moiety is an anthracycline derivative of Formula (VII), or a prodrug, solvate, pharmaceutically acceptable salt thereof:

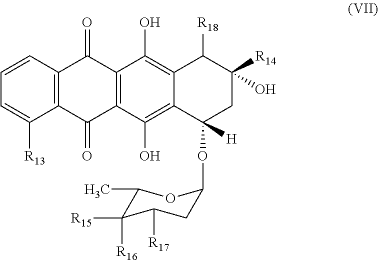

wherein:
R₁₃ is H, hydroxy or —OCH₃;
R₁₄ is —C₂H₅, —C(O)CH₃, —C(O)CH₂OH, —C(O)CH₂O—C(O)CH(OC₂H₅)₂, C(O)CH₂O—C(O)C₄H₉;
one of R₁₅ and R₁₆ is H; and the other is H, hydroxy or a tetrahydropyran-2-yloxy (OTHP) group;
R₁₇ is —NH₂, —N(CH₃)₂,

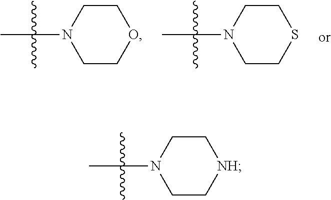 [SMILES: *N1CCOCC1 ; *N1CCSCC1 ; *N1CCNCC1]

and
R₁₈ is H or —COOCH₃.

In some embodiments, in the compound of Formula (VII):

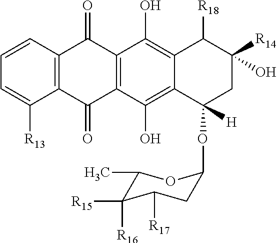

R₁₃ is H, hydroxy or —OCH₃;
R₁₄ is ethyl or —C(O)—R₂₀;
R₂₀ is methyl, hydroxymethyl, diethoxyacetoxymethyl or butyryloxymethyl;
R₁₅ is H;
R₁₆ is hydroxy, or a tetrahydropyran-2-yloxy (OTHP) group;
R₁₇ is NH₂, N(CH₃)₂, N(CH₂—CH₃)₂ or a morpholine group; and
R₁₈ is H or —COOCH₃.

In some embodiments, the topoisomerase II inhibitor drug moiety of Formula (VII) is selected from the group consisting of doxorubicin, dimethyldoxorubicin, diethyldoxorubicin, daunorubicin, dimthyldaunorubicin, epirubicin, dimethylepirubicin, diethylepirubicin, Idarubicin, dimethylidarubicin, diethylidarubicin and aclarubicin. In some aspects, the topoisomerase II inhibitor drug moiety of Formula (VII) is covalently attached to L^{c} via a hydroxyl group wherein a hydrogen atom of the hydroxyl group is replaced with a bond to form a bond to L^{C}. In other aspects, the topoisomerase II inhibitor drug moiety of Formula (VII) for covalent attachment to L^{C} is selected from:

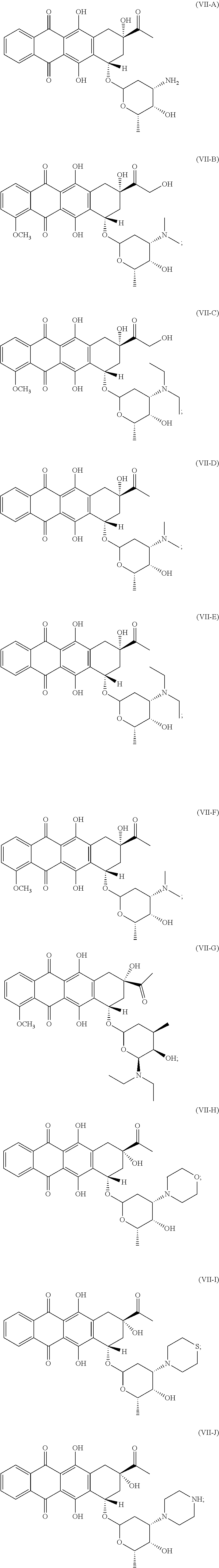

or a prodrug, solvate, pharmaceutically acceptable salt thereof, and
# donates attachment to L^{C}.

In some embodiments, F prior to linking to M and B₁ is:

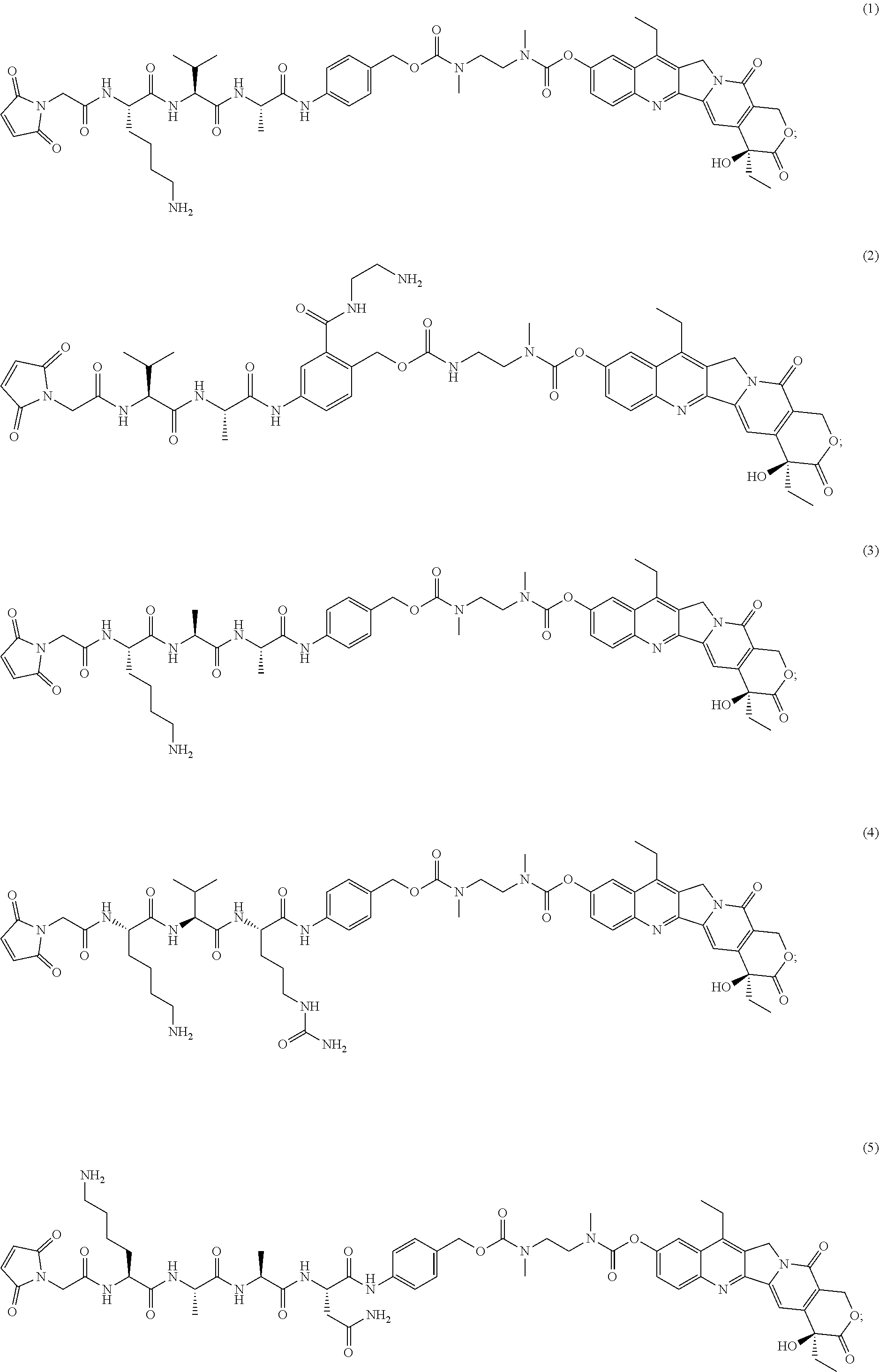

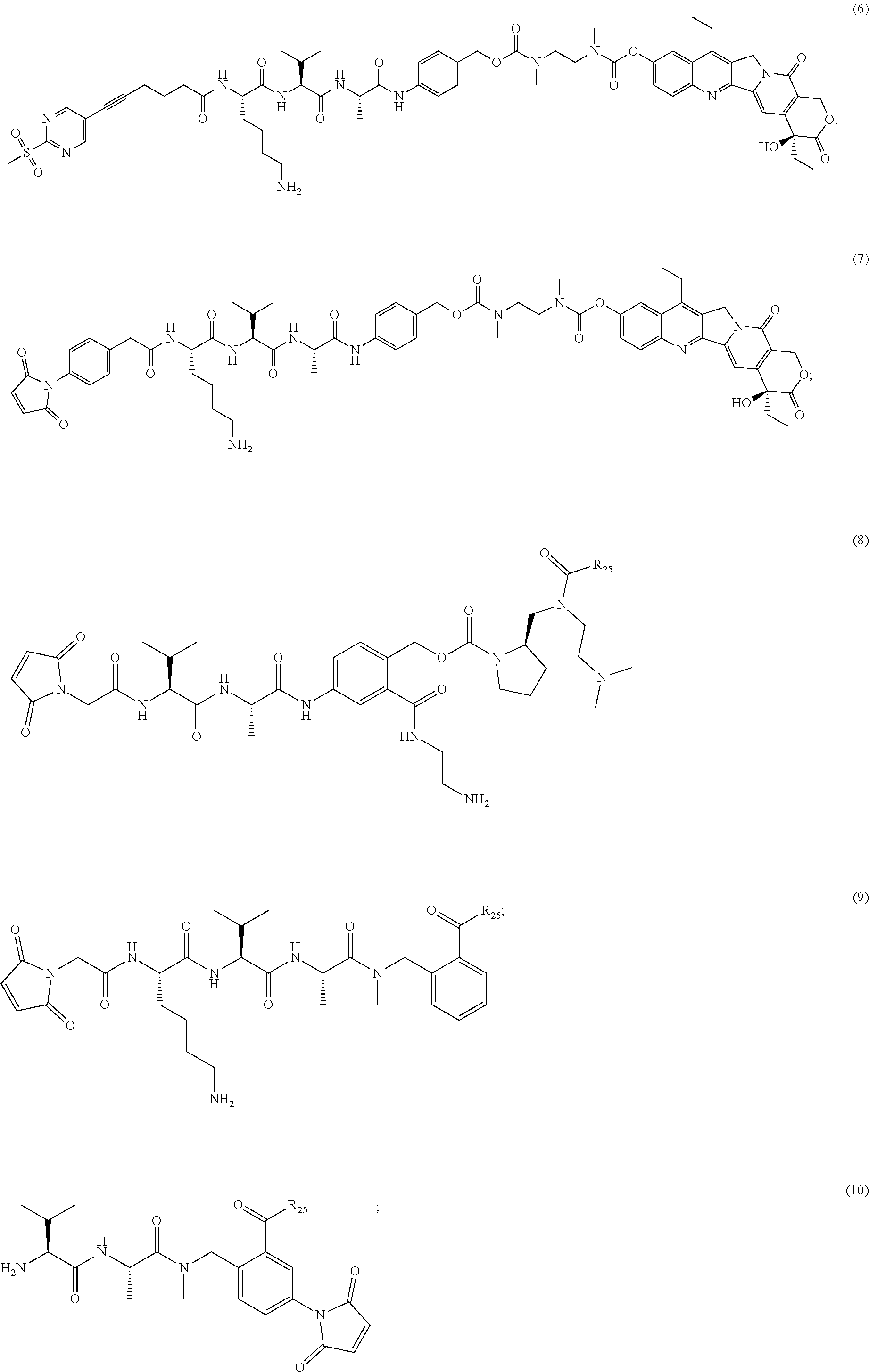

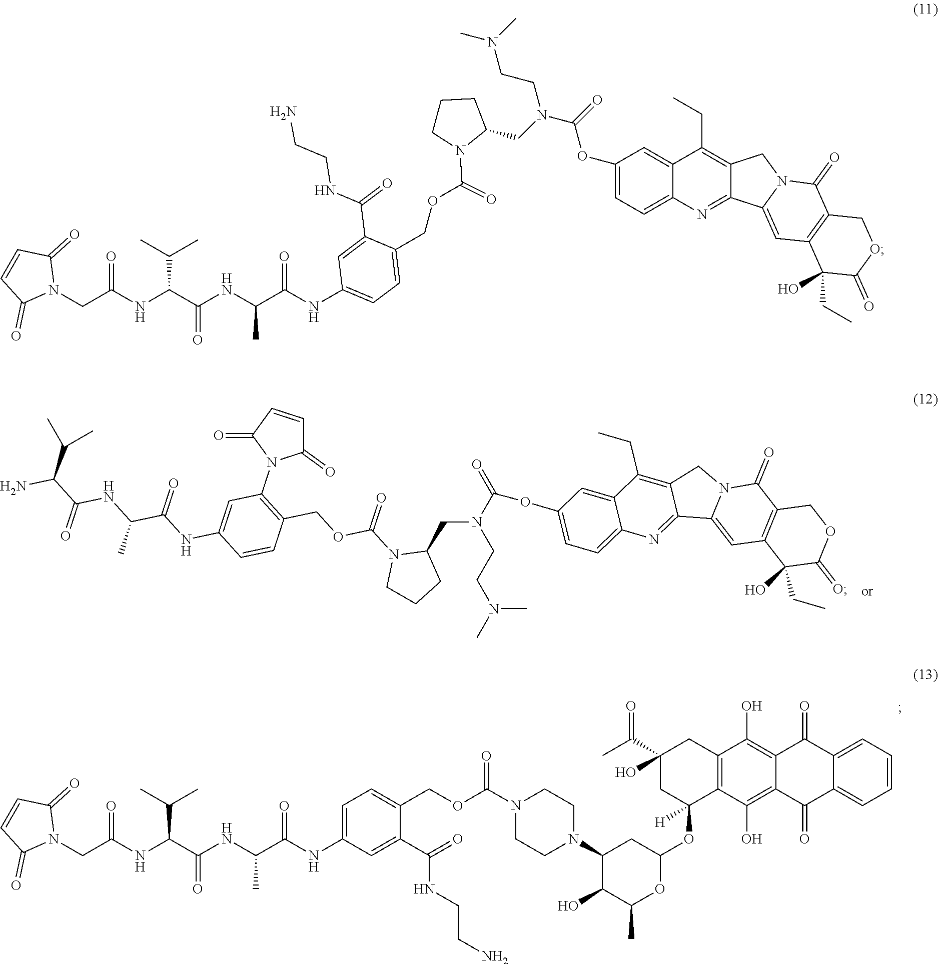 [SMILES: CCc1c2c(nc3ccc(OC(=O)N(CCN(C)C)C[C@H]4CCCN4C(=O)OCc4ccc(NC(=O)[C@@H](C)NC(=O)[C@H](NC(=O)CN5C(=O)C=CC5=O)C(C)C)cc4C(=O)NCCN)cc13)-c1cc3c(c(=O)n1C2)COC(=O)[C@]3(O)CC ; CCc1c2c(nc3ccc(OC(=O)N(CCN(C)C)C[C@H]4CCCN4C(=O)OCc4ccc(NC(=O)[C@H](C)NC(=O)[C@@H](N)C(C)C)cc4N4C(=O)C=CC4=O)cc13)-c1cc3c(c(=O)n1C2)COC(=O)[C@]3(O)CC ; CC(=O)[C@]1(O)Cc2c(O)c3c(c(O)c2[C@@H](OC2C[C@H](N4CCN(C(=O)OCc5ccc(NC(=O)[C@H](C)NC(=O)[C@@H](NC(=O)CN6C(=O)C=CC6=O)C(C)C)cc5C(=O)NCCN)CC4)[C@H](O)[C@H](C)O2)C1)C(=O)c1ccccc1C3=O]

wherein:
R₂₅ is:

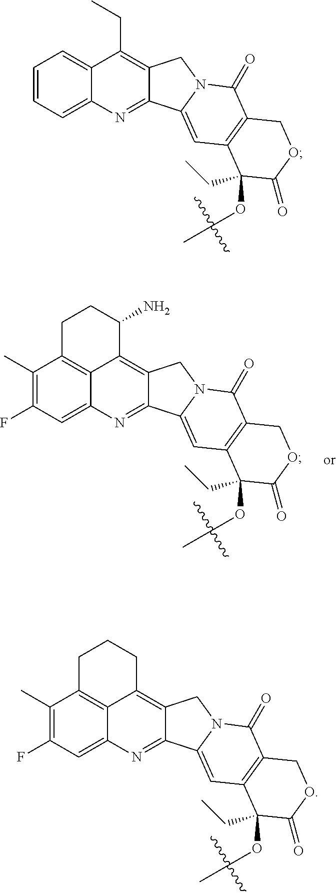 [SMILES: *O[C@]1(CC)C(=O)OCc2c1cc1n(c2=O)Cc2c-1nc1ccccc1c2CC ; *O[C@]1(CC)C(=O)OCc2c1cc1n(c2=O)Cc2c-1nc1cc(F)c(C)c3c1c2[C@@H](N)CC3 ; *O[C@]1(CC)C(=O)OCc2c1cc1n(c2=O)Cc2c-1nc1cc(F)c(C)c3c1c2CCC3]

In some embodiments, B₁ is a polymer of Formula (VIII-A), Formula (VIII-B), or a pharmaceutically acceptable salt thereof,

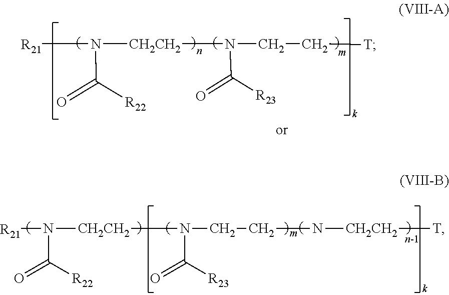

wherein
R₂₁ is an initiating group;
R₂₂ is independently selected for each repeating unit and is a pendent moiety containing an active functional group;
R₂₃ is a non-reactive pendent moiety and is independently selected for each repeating unit;
k indicates that the polymer units m and n are connected to each other in a random order;
n is an integer from 0 to 3;
m is an integer from about 1 to about 50, provided that the sum of n and m is less than or equal to 50; and
Tm is a terminating group.

In some aspects, R₂₁ is a hydrogen, an alkyl or a substituted alkyl. In other aspects, R₂₁ is an alkyl group. In yet other aspects, R₂₁ is a hydrogen.

In other aspects, R₂₃ for each repeating unit is a non-reactive pendent moiety independently selected from an unsubstituted alkyl, a substituted alkyl, an unsubstituted alkenyl, a substituted alkenyl, an unsubstituted aralkyl, a substituted aralkyl, an unsubstituted heterocyclylalkyl and a substituted heterocyclylalkyl group. In still other aspects, R₂₃ is an unsubstituted alkyl or a substituted alkyl. In yet other aspects, R₂₃ is methyl, hydroxy methyl, ethyl, propyl or butyl. In still other aspects, R₂₃ is ethyl, methyl or hydroxy methyl.

In other aspects, R₂₂ is a pendent moiety. In yet other aspects, R₂₂ for each repeating unit is independently selected from an unsubstituted alkyl, a substituted alkyl, an unsubstituted alkenyl, a substituted alkenyl, an unsubstituted aralkyl, a substituted aralkyl, an unsubstituted heterocyclylalkyl and a substituted heterocyclylalkyl group. In still other aspects, R₂₂ is a pendent moiety comprising an active functional group. In other aspects, R₂₂ is a pendent moiety comprising an active functional group selected from an alkyne, an amine, an oxyamine, an aldehyde, a ketone, an acetal, a ketal, a maleimide, an ester, a carboxylic acid, an activated carboxylic acid, an active carbonate, a chloroformate, an alcohol, an azide, a vinyl sulfone, and an orthopyridyl disulfide (OPSS). In some embodiments, the active functional group of R₂₂ is chemically orthogonal to one or more or all the other functional group on the conjugate. In other embodiments, the active functional group of R₂₂ is not chemically orthogonal to one or more or all the other functional group on the conjugate. In still other embodiments, the active functional group of R₂₂ is an unsubstituted alkyl, a substituted alkyl, an unsubstituted alkenyl, a substituted alkenyl, an unsubstituted aralkyl, a substituted aralkyl, an unsubstituted heterocyclylalkyl, a substituted heterocyclylalkyl or an alkyne group. In yet other embodiments, the active functional group of R₂₂ is an alkyne group. In other embodiments, the active functional group of R₂₂ is an acetylene.

In some embodiments, T is Z-B-Q. In other embodiments, Z is S, O or N; B is an optional linking group; and Q is a terminating group or a portion of a terminating group. In yet other embodiments, B is —(CH₂)₁₋₁₀—, —(CH₂)₁₋₈—, —(CH₂)₁₋₆—, —(CH₂)₁₋₄— or —(CH₂)₁₋₂—. In some aspects, B is —(CH₂)₂—. In other aspects, Q is —COOH, —COOCH₃, —NH₂ or NH-tBoc. In other aspects, T is —S—CH₂CH₂—CO₂H.

In some embodiments, B₁ is a polymer of Formula (VIII-J), Formula (VIII-K), Formula (VIII-L), Formula (VIII-M), Formula (VIII-N), Formula (VIII-O), Formula (VIII-P), Formula (VIII-Q) or Formula (VIII-R):

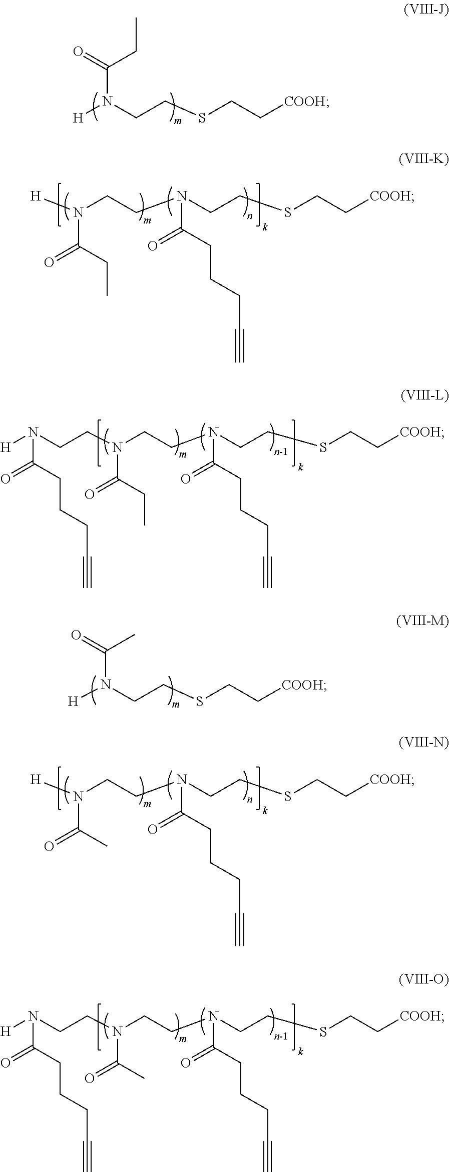

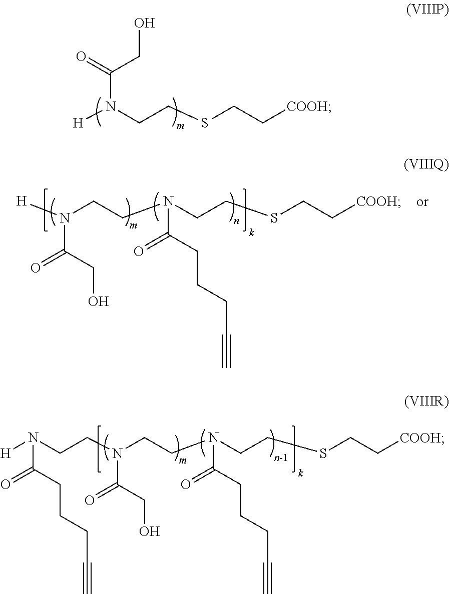

wherein
k indicates that the polymer units m and n and polymer units m and (n−1) are connected to each other in a random order;
m is an integer from about 10 to about 50; and
n is an integer from 1 to 3. In some aspects, B₁ has a molecular weight between about 1000 Da and about 5000 Da. In other aspects, B₁ has a molecular weight between about 1000 Da and about 2500 Da. In still other aspects, B₁ has a molecular weight of about 2000 Da.

In some embodiments, the acetylene group in the polymer of Formula (VIII-K), Formula (VIII-L), Formula (VIII-N), Formula (VIII-O), Formula (VIII-Q) or Formula (VIII-R) is reacted with an azido moiety via copper(I) catalyzed azide-acetylene click chemistry. In other embodiments, the azido moiety that reacts via click chemistry with the acetylene group at a pendent position of Formula (VIII-K), Formula (VIII-L), Formula (VIII-N), Formula (VIII-O), Formula (VIII-Q) or Formula (VIII-R) is:

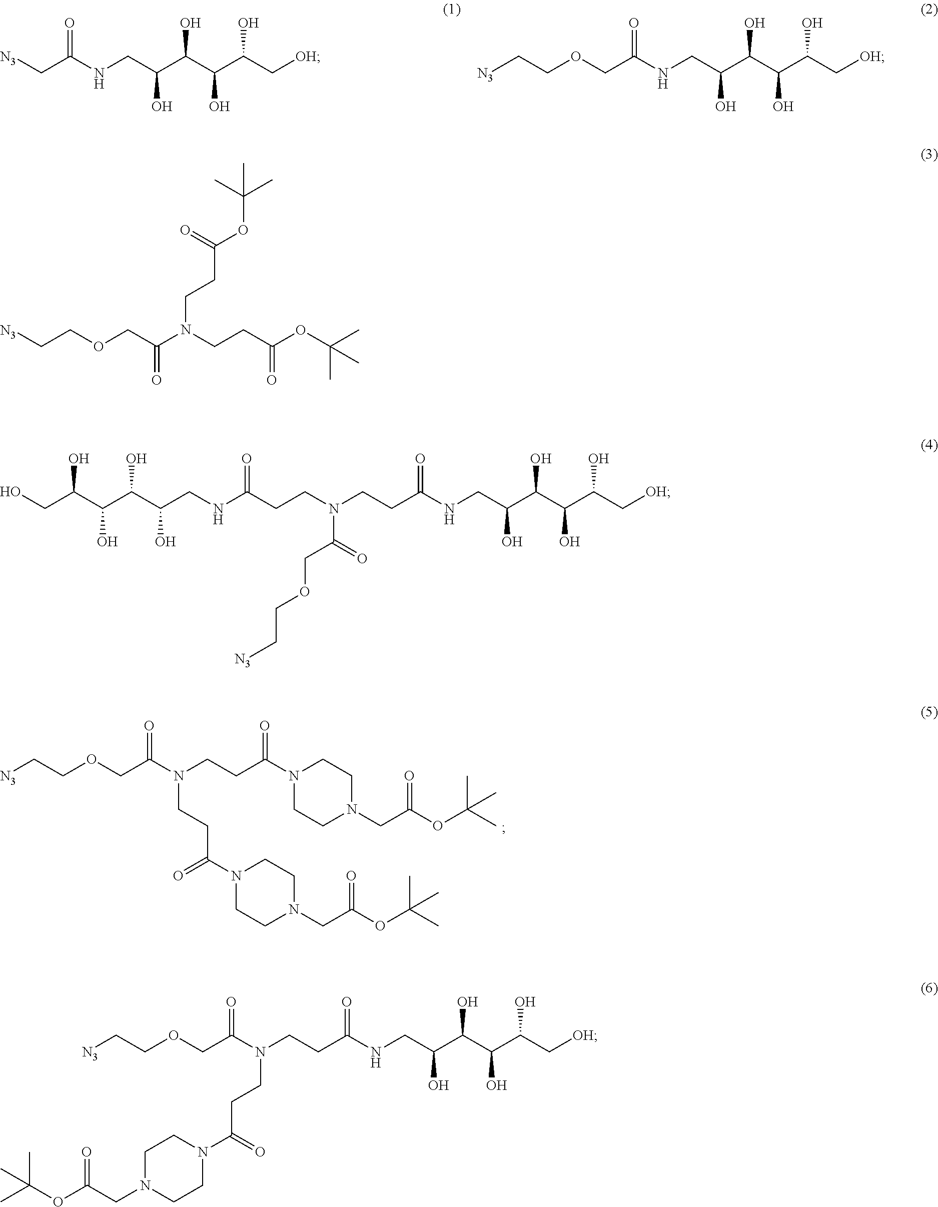 [SMILES: [N-]=[N+]=NCC(=O)NC[C@H](O)[C@@H](O)[C@H](O)[C@H](O)CO ; [N-]=[N+]=NCCOCC(=O)NC[C@H](O)[C@@H](O)[C@H](O)[C@H](O)CO ; CC(C)(C)OC(=O)CCN(CCC(=O)OC(C)(C)C)C(=O)COCCN=[N+]=[N-] ; [N-]=[N+]=NCCOCC(=O)N(CCC(=O)NC[C@H](O)[C@@H](O)[C@H](O)[C@H](O)CO)CCC(=O)NC[C@H](O)[C@@H](O)[C@H](O)[C@H](O)CO ; CC(C)(C)OC(=O)CN1CCN(C(=O)CCN(CCC(=O)N2CCN(CC(=O)OC(C)(C)C)CC2)C(=O)COCCN=[N+]=[N-])CC1 ; CC(C)(C)OC(=O)CN1CCN(C(=O)CCN(CCC(=O)NC[C@H](O)[C@@H](O)[C@H](O)[C@H](O)CO)C(=O)COCCN=[N+]=[N-])CC1]

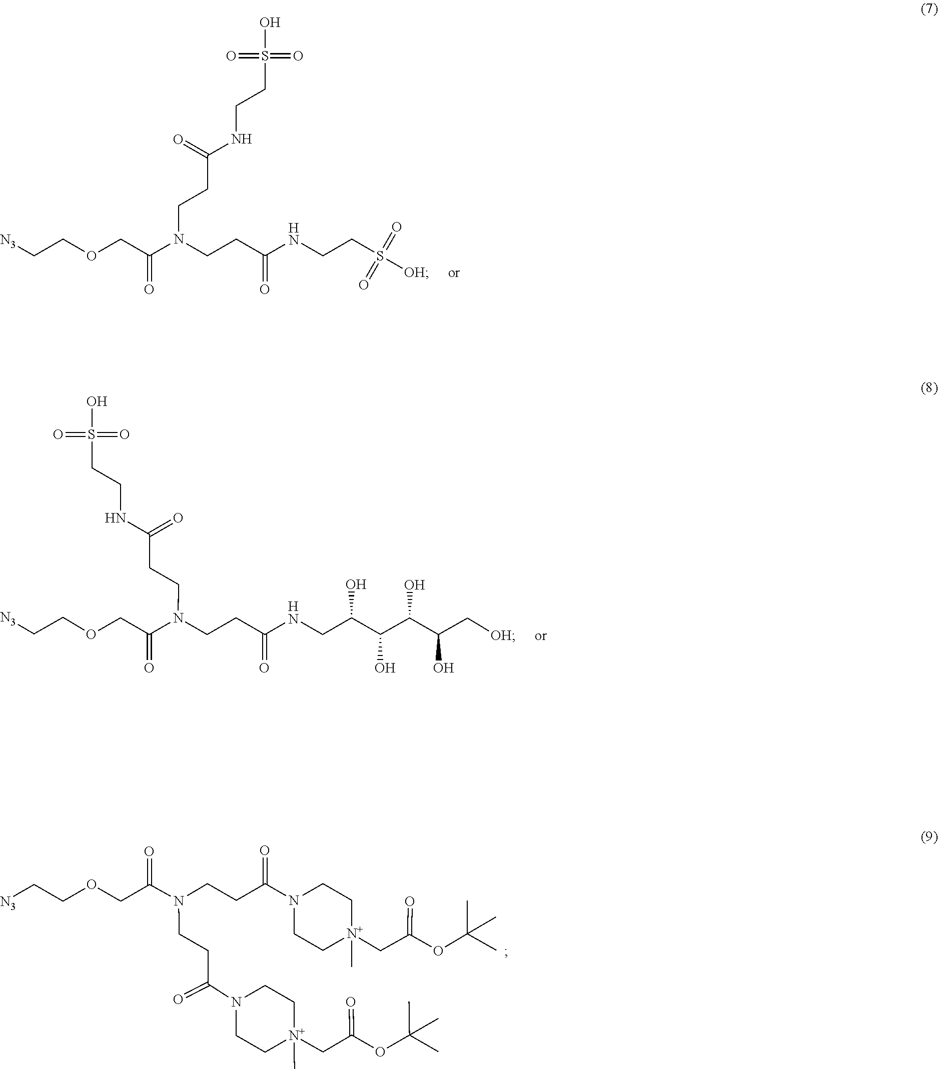 [SMILES: [N-]=[N+]=NCCOCC(=O)N(CCC(=O)NCCS(=O)(=O)O)CCC(=O)NCCS(=O)(=O)O ; [N-]=[N+]=NCCOCC(=O)N(CCC(=O)NCCS(=O)(=O)O)CCC(=O)NC[C@H](O)[C@@H](O)[C@H](O)[C@H](O)CO ; CC(C)(C)OC(=O)C[N+]1(C)CCN(C(=O)CCN(CCC(=O)N2CC[N+](C)(CC(=O)OC(C)(C)C)CC2)C(=O)COCCN=[N+]=[N-])CC1]

or an azido moiety comprising a carboxylic acid, zwitterionic base/carboxylic acid, polyhydroxy, or sulfonic acid group or a combination thereof. In some aspects, both arms of the azido moiety are identical. In other aspects, both arms of the azido moiety are not identical.

In some embodiments, the acetylene group of Formula (VIII-K), Formula (VIII-L), Formula (VIII-N), Formula (VIII-O), Formula (VIII-Q) or Formula (VIII-R) is reacted with an azido moiety via copper(I) catalyzed azide-acetylene click chemistry, to form a POZ azido acetylene click polymer. In some aspects, the POZ azido acetylene click polymer is:

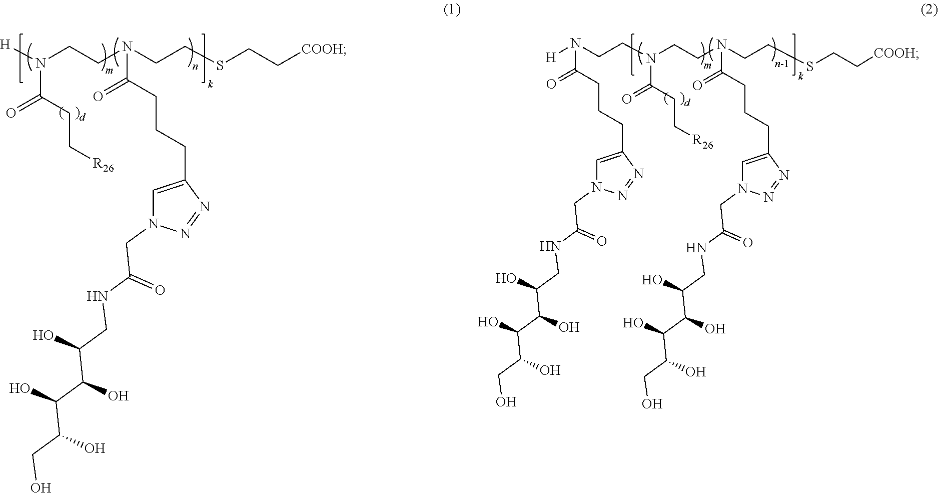

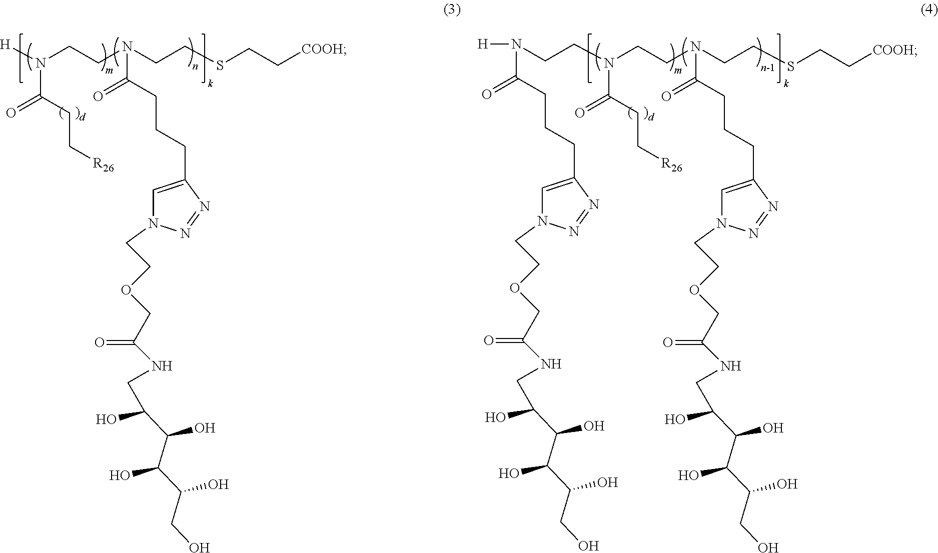

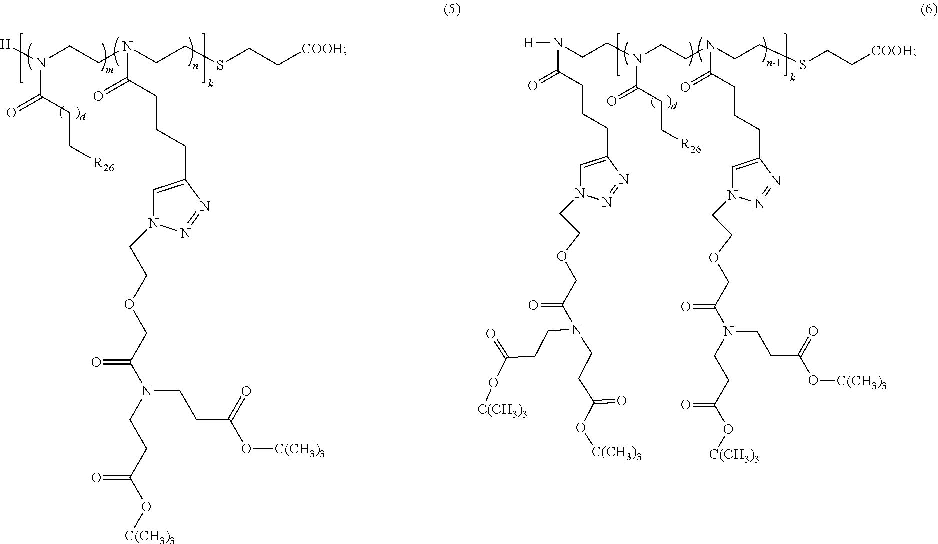

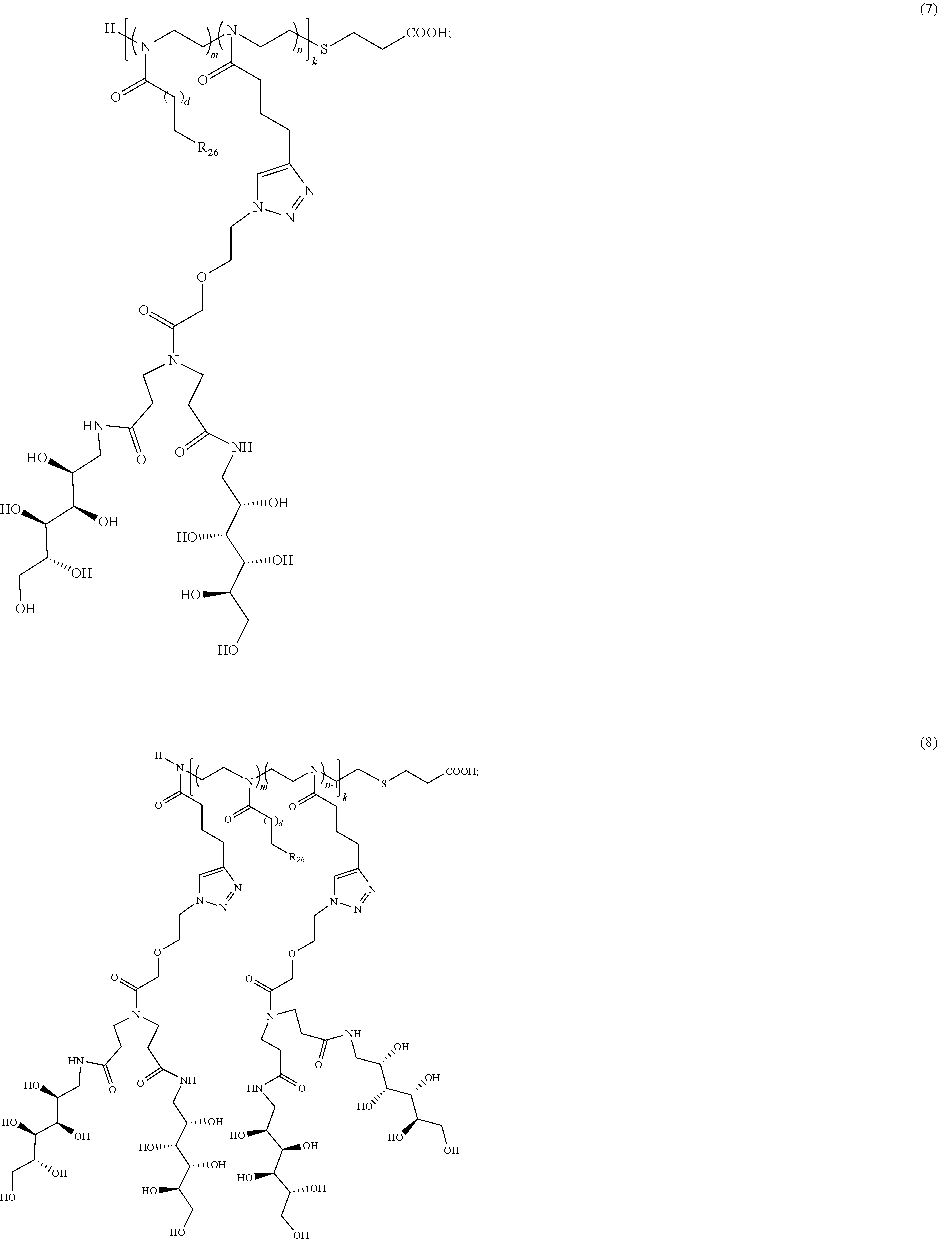

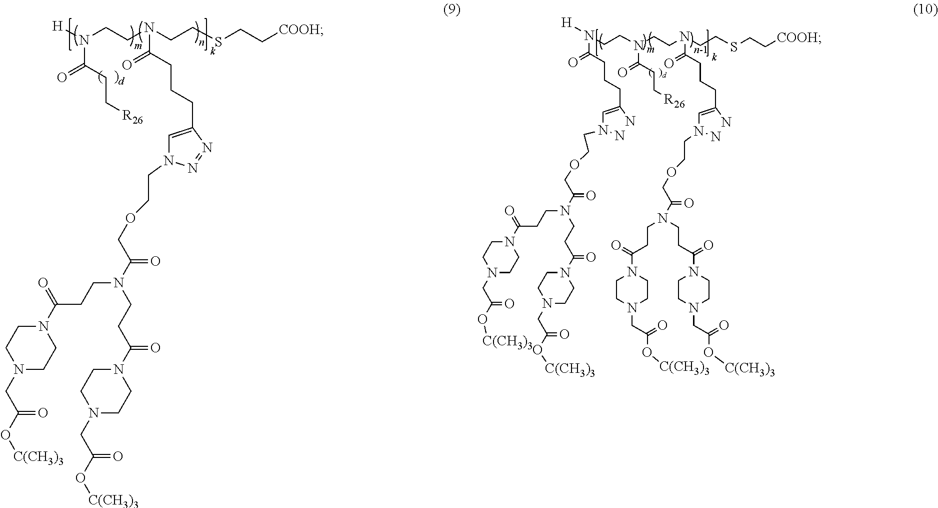

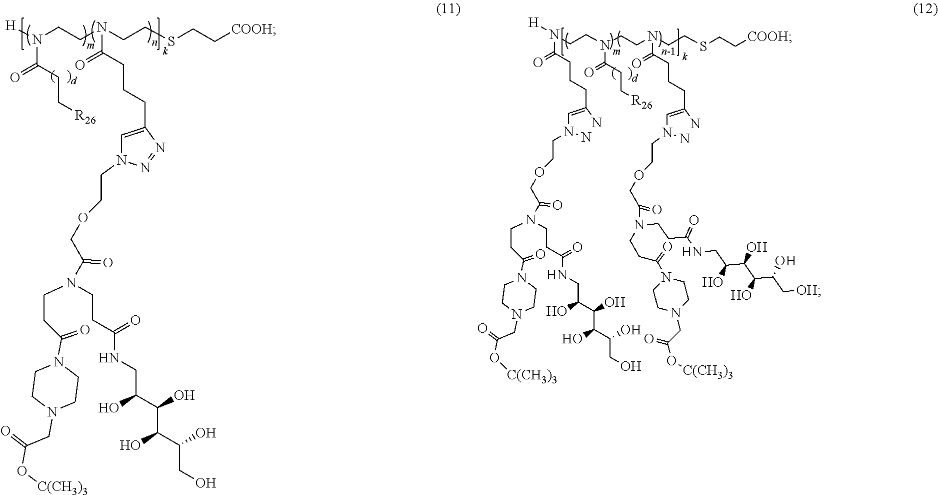

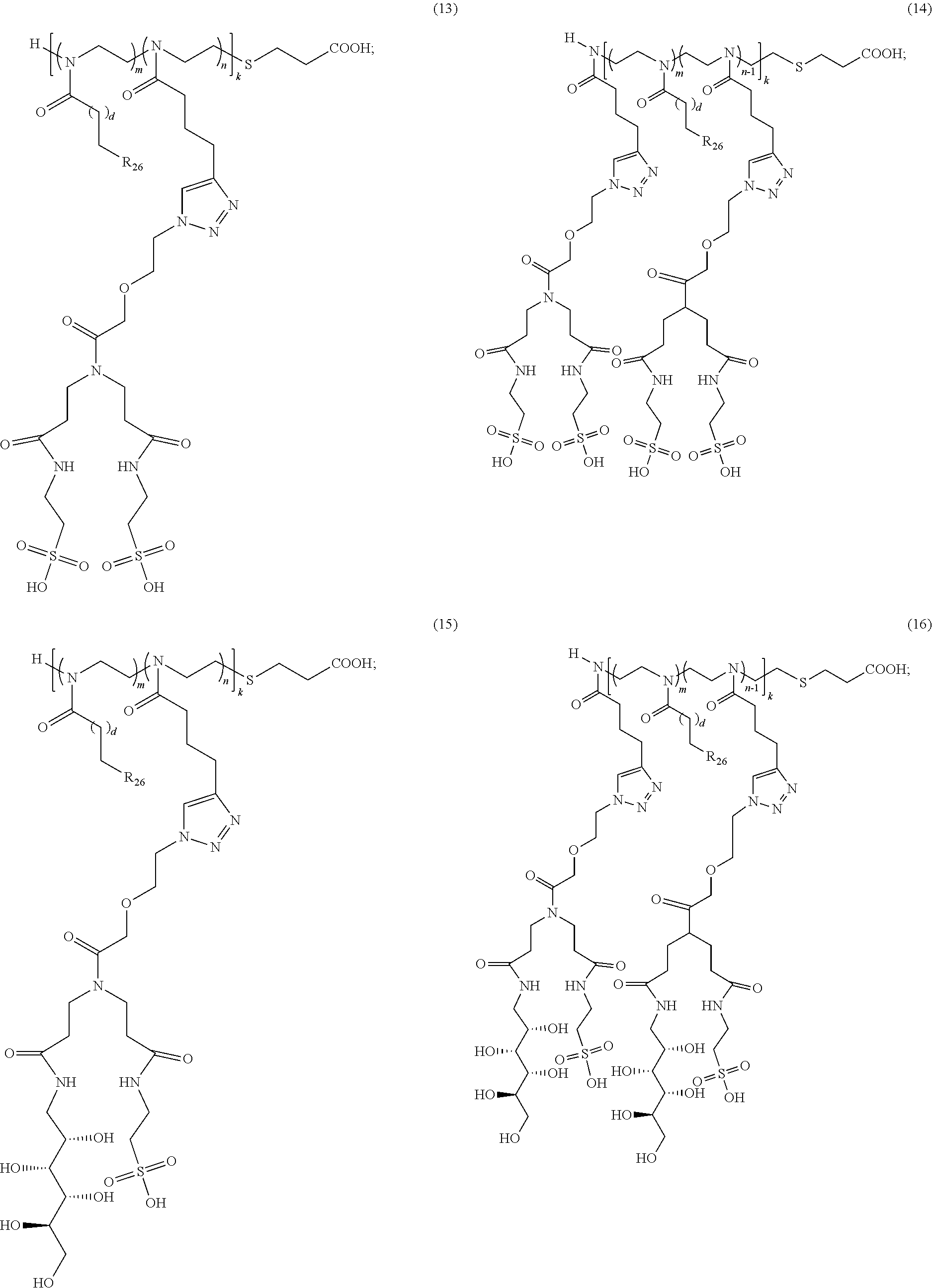

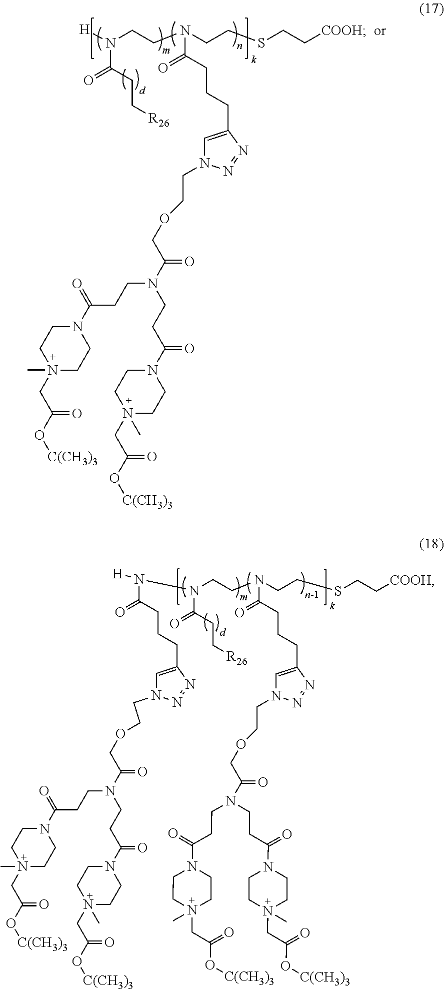

wherein
R₂₆ is H or OH;
d is 0 or 1;
k indicates that the polymer units m and n and polymer units m and (n−1) are connected to each other in a random order;
m is an integer from about 10 to about 50; and
n is an integer from 1 to 3.

In some aspects, the POZ azido acetylene click polymer of Formula (VIII-M) is:

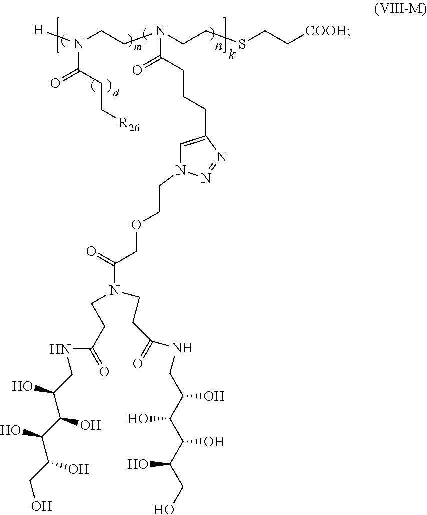

wherein R₂₆, d, m, n and k are as defined herein.

In some embodiments, B₁ is connected to the drug linker moiety via a terminal —COOH group of B₁.

In other embodiments, the scaffold is:

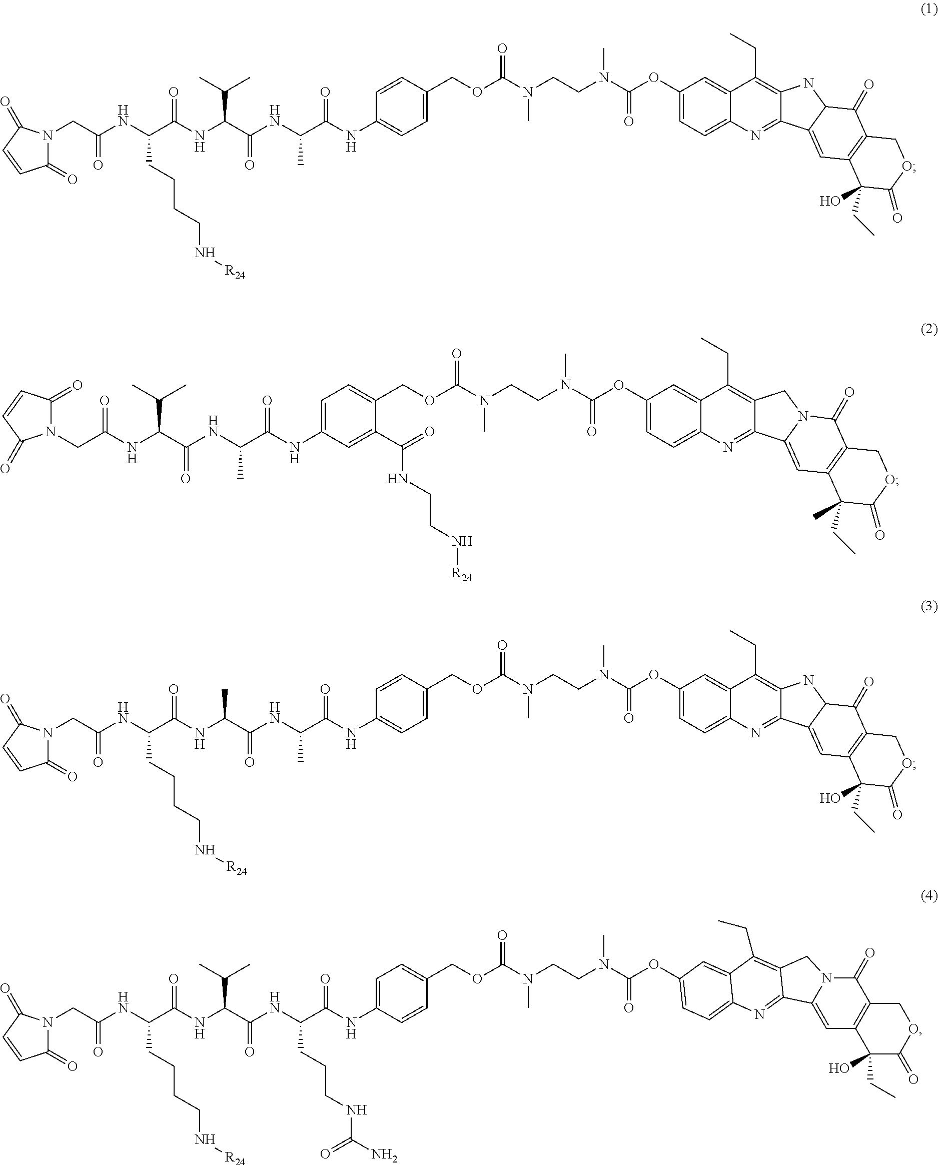

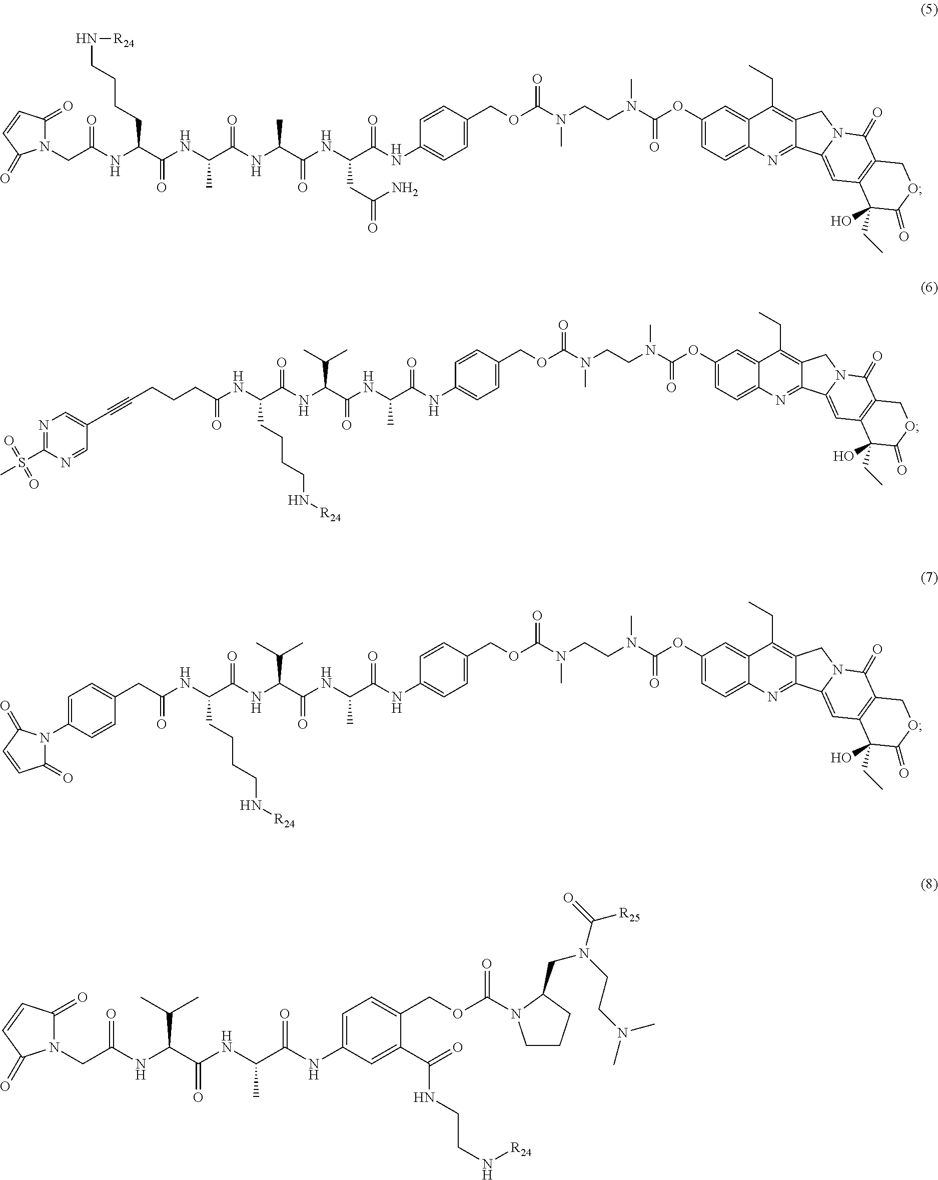

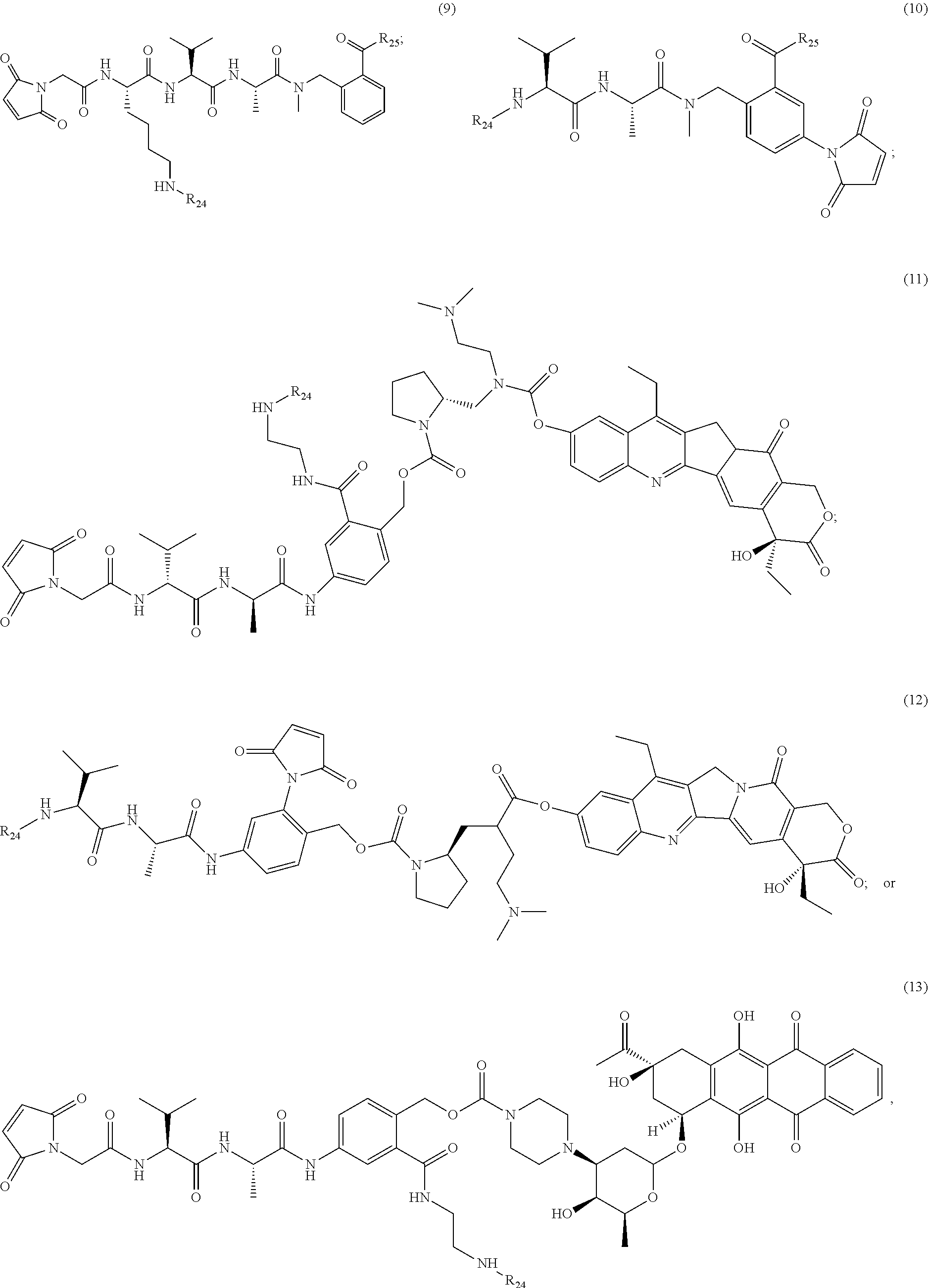

where
R₂₄ is

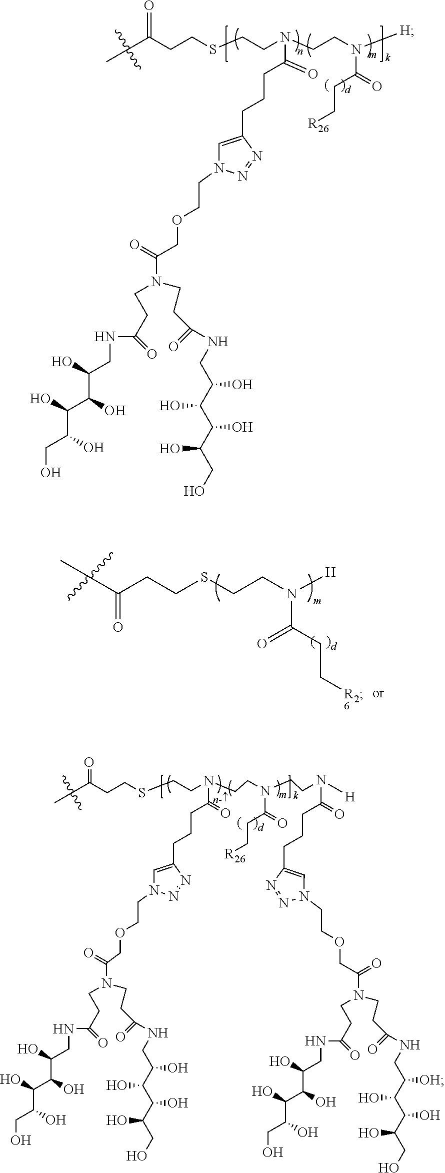

R₂₅ is:

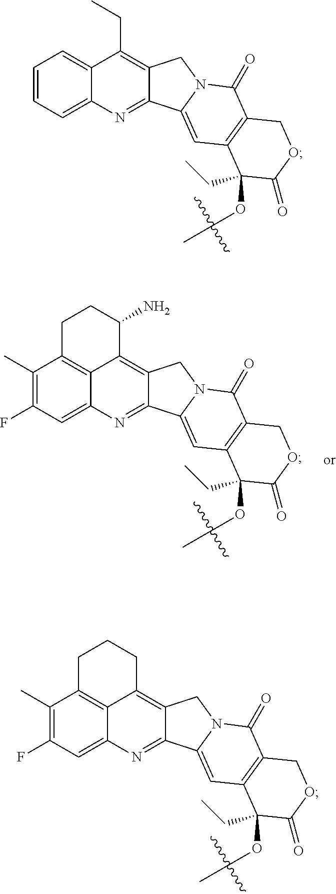 [SMILES: *O[C@]1(CC)C(=O)OCc2c1cc1n(c2=O)Cc2c-1nc1ccccc1c2CC ; *O[C@]1(CC)C(=O)OCc2c1cc1n(c2=O)Cc2c-1nc1cc(F)c(C)c3c1c2[C@@H](N)CC3 ; *O[C@]1(CC)C(=O)OCc2c1cc1n(c2=O)Cc2c-1nc1cc(F)c(C)c3c1c2CCC3]

R₂₆ is H or OH;
d is an integer 0 or 1;
m is an integer from about 10, 20 or 50;
n is an integer ranging from 1 to 3; and
k indicates that the polymer units m and n and polymer units m and (n−1) are connected to each other in a random order.

In some aspects, R₂₄ is:

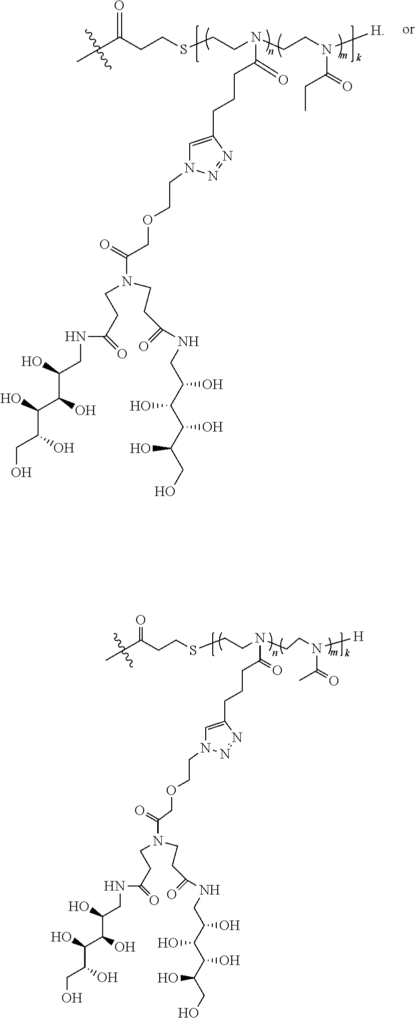

In other aspects, R₂₄ is:

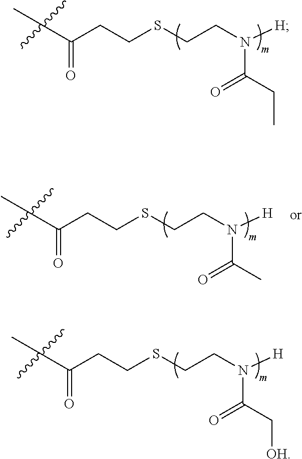

In some embodiments, the recognition moiety (M) includes or may be engineered to include at least one chemically reactive group for linking to the variable A¹. In other embodiments, the reactive group of the recognition moiety is a cysteine moiety or a lysine moiety. In yet other embodiments, the reactive group of the recognition moiety is a cysteine moiety. In still other embodiments, the reactive group of the recognition moiety is an engineered cysteine moiety. In other embodiments, the recognition moiety is an antibody or an antibody fragment.

In some embodiments, the -polymer antibody-drug conjugate is:

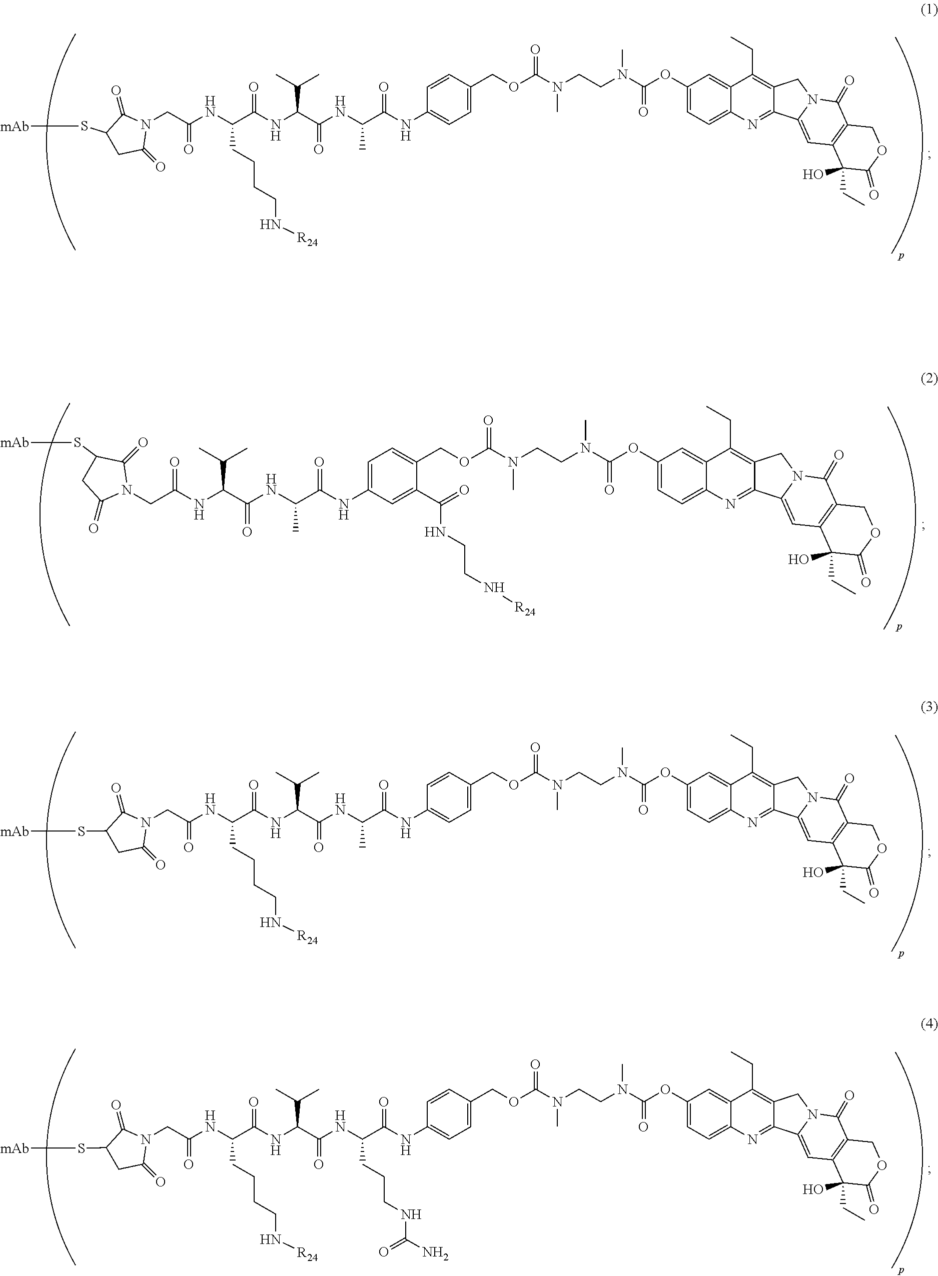

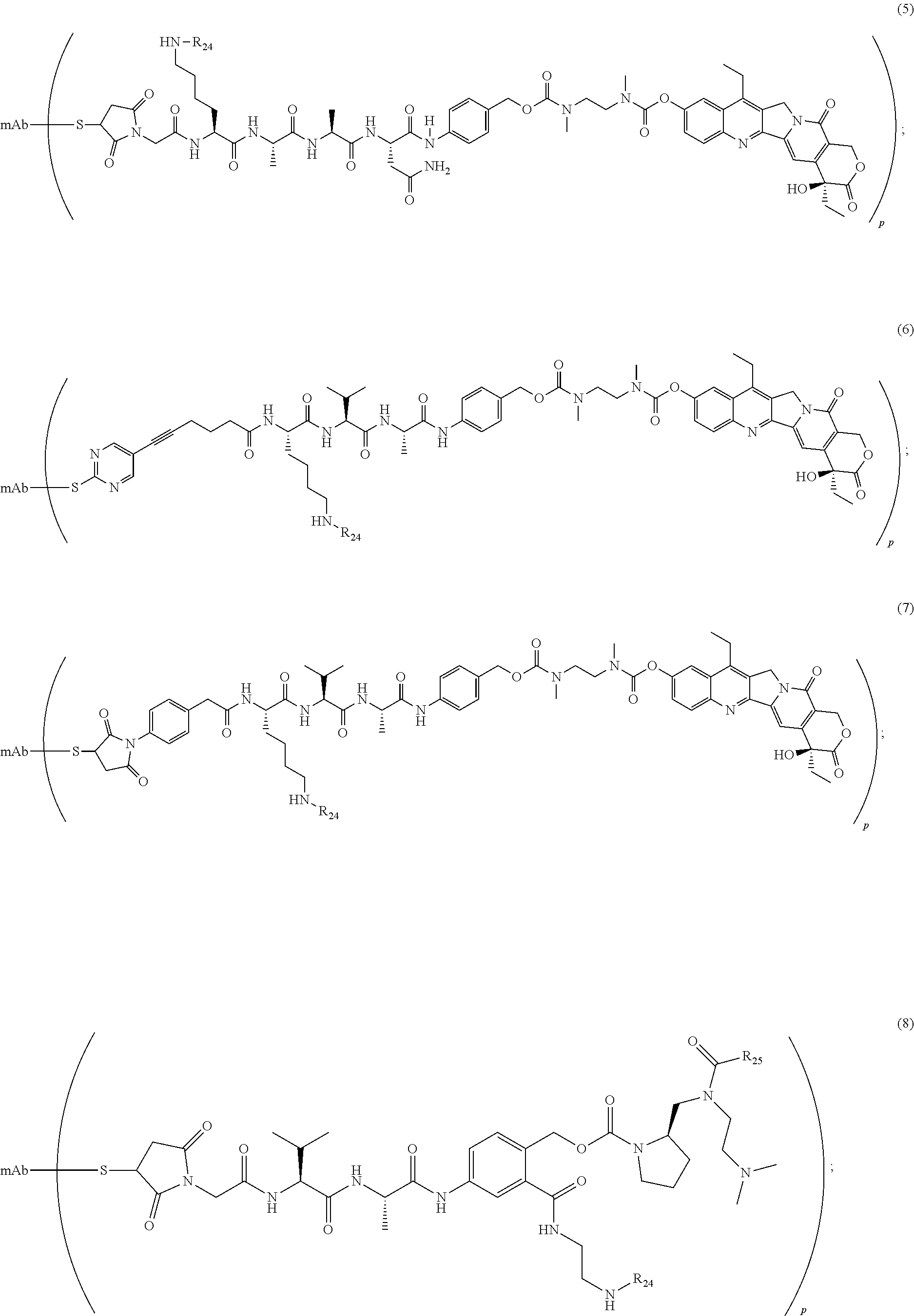

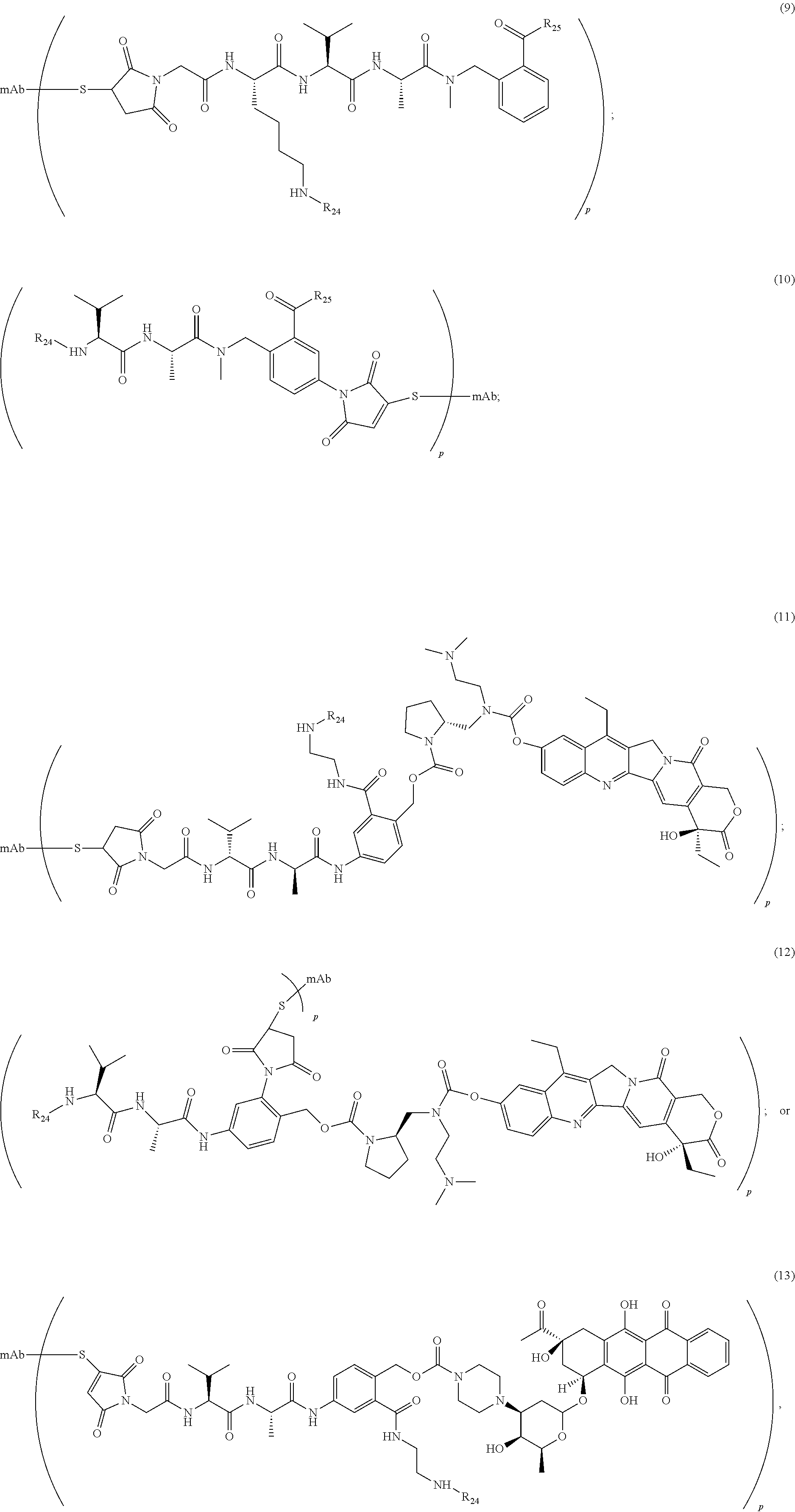

wherein:
R₂₄ is

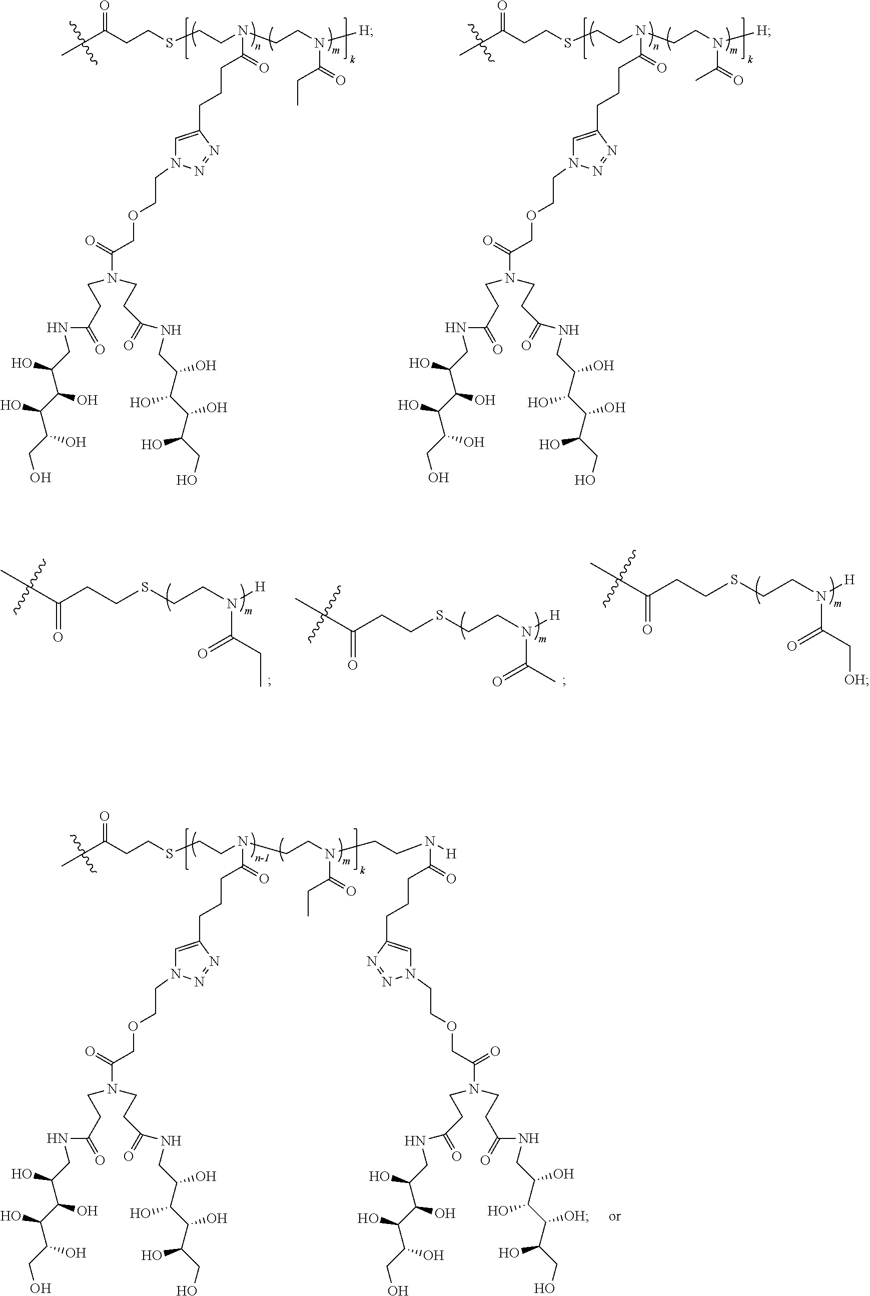

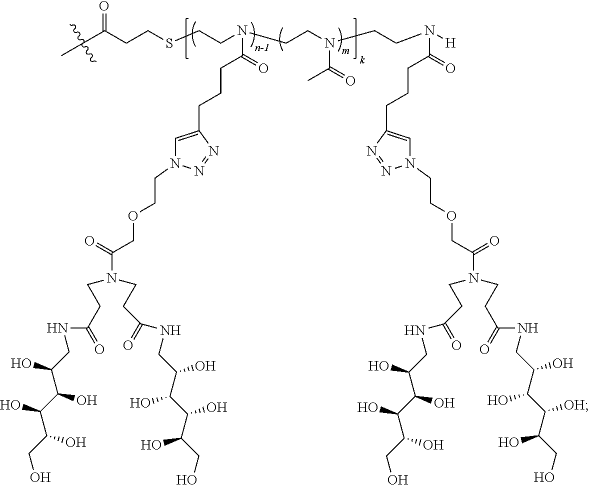

R₂₅ is:

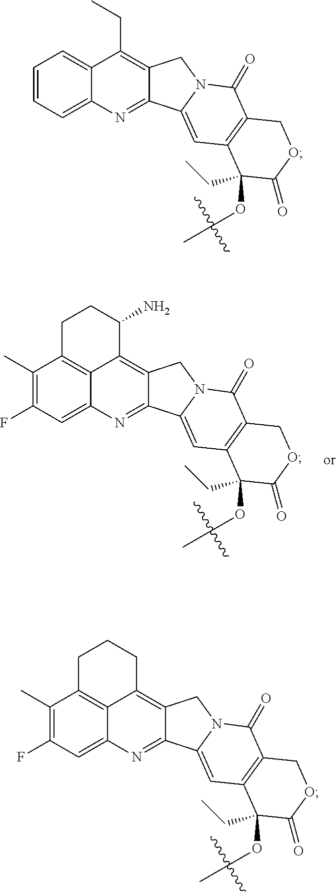 [SMILES: *O[C@]1(CC)C(=O)OCc2c1cc1n(c2=O)Cc2c-1nc1ccccc1c2CC ; *O[C@]1(CC)C(=O)OCc2c1cc1n(c2=O)Cc2c-1nc1cc(F)c(C)c3c1c2[C@@H](N)CC3 ; *O[C@]1(CC)C(=O)OCc2c1cc1n(c2=O)Cc2c-1nc1cc(F)c(C)c3c1c2CCC3]

S is the sulfur atom of an antibody (mAb) cysteine residue;
m is an integer from about 10, 20 or 50;
n ranges from 1 to 3;
k indicates that the polymer units m and n and polymer units m and (n−1) are connected to each other in a random order; and
p is 6 or 8. In some aspects, m is an integer of about 10. In other aspects, m is about 20. In still other aspects, m is about 50. In some aspects, n is 1. In other aspects, n is 2. In yet other aspects, n is 3.

In some embodiments, R₂₄ is

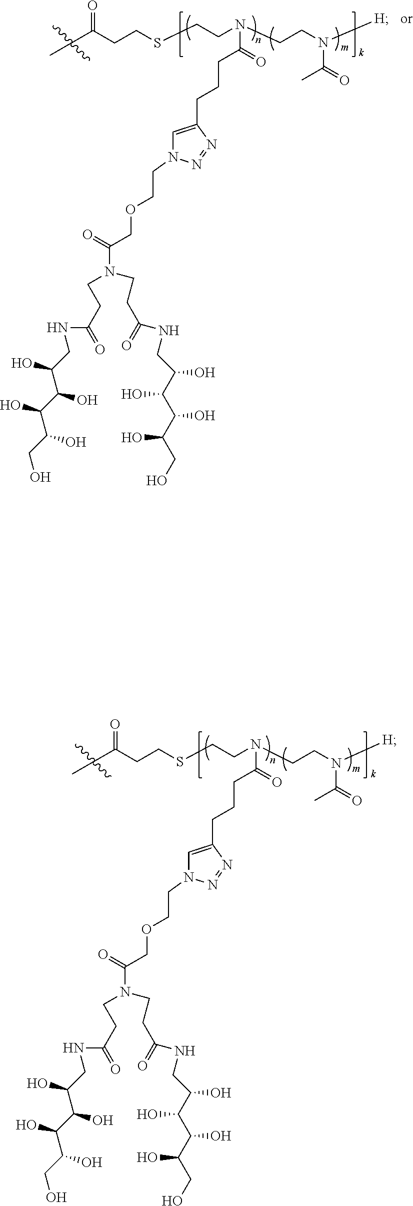

wherein n, m and k are as defined herein. In other embodiments, R₂₄ is:

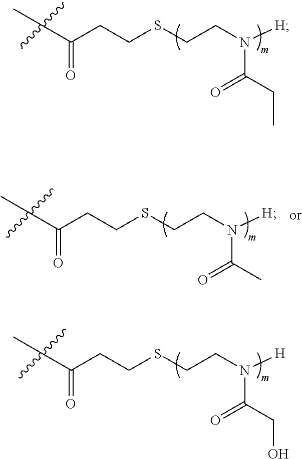

wherein m is as defined herein. In some aspects, p is 6. In other aspects, p is 8. In still other aspects, p is 8 and m is an integer of about 20.

In some embodiments, the antibody targets HER2, TROP-2, HER3 or B7-H3. In some aspects, the antibody that targets HER2 is trastuzumab, bevacizumab, pertuzumab, or margetuximab. In other aspects, the antibody that targets TROP-2 is datopotamab, sacituzumab or hRS7. In yet other aspects, the antibody that targets HER3 is patritumab, seribantumab, lumretuzumab, GSK2849330, CDX-3379, barecetamab, AV-203, elgemtumab, HMBD-001, U3P1287/01 or SIBP-03. In still other aspects, the antibody that targets B7-H3 is enoblituzumab, ifinatamab or omburtamab.

In some aspects, the present disclosure provides a method of preventing or treating a disease or disorder in a subject, including administering to the subject a therapeutically effective amount of a conjugate described herein or a pharmaceutically acceptable salt thereof. In some aspects, the disease or disorder is cancer. For example, the cancer may be breast cancer, cervical cancer, colon cancer, colorectal cancer, endometrioid endometrial cancer, esophageal cancer, gastric cancer, glioma, non-small cell lung cancer (NSCLC), ovarian cancer, prostate cancer, stomach cancer, bladder cancer, or uterine cancer.

In some aspects, the present disclosure provides a conjugate described herein, or a pharmaceutically acceptable salt thereof, for preventing or treating a disease or disorder in a subject.

In some aspects, the present disclosure provides a use of a conjugate described herein, or a pharmaceutically acceptable salt thereof, in the manufacture of a medicament for preventing or treating a disease or disorder in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure provides novel antibody-drug conjugates, synthetic methods for making the conjugates or scaffolds, pharmaceutical compositions containing them, and various uses of the conjugates.

### Definition/Terminology

The use of the articles “a”, “an”, and “the” in both the following description and claims are to be construed to cover both the singular and the plural, unless otherwise indicated herein or clearly contradicted by context. The terms “comprising”, “having”, “including”, and “containing” are to be construed as open terms (i.e., meaning “including but not limited to”) unless otherwise noted. Additionally whenever “comprising” or another open-ended term is used in an embodiment, it is to be understood that the same embodiment can be more narrowly claimed using the intermediate term “consisting essentially of” or the closed term “consisting of”.

As used herein, the term “about”, “approximately” or “approximate”, when used in connection with a numerical value, means that an acceptable degree of error or variation for the quantity measured given the nature or precision of the measurements. Numerical quantities given in this description are approximate unless stated otherwise, meaning that the term “about” or “approximately” can be inferred when not expressly stated.

Recitation of ranges of values are merely intended to serve as a shorthand method of referring individually to each separate value falling within the range, unless otherwise indicated herein, and each separate value is incorporated into the specification as if it were individually recited herein. A range used herein, unless otherwise specified, includes the two limits of the range. For example, the expressions “x being an integer between 1 and 6” and “x being an integer of 1 to 6” both mean “x being 1, 2, 3, 4, 5, or 6”.

As used herein, the term “antibody” is used in the broadest sense and specifically covers intact monoclonal antibodies, polyclonal antibodies, monospecific antibodies, multispecific antibodies (e.g., bispecific antibodies), and antibody fragments that exhibit the desired biological activity. The native form of an antibody is a tetramer and consists of two identical pairs of immunoglobulin chains, each pair having one light chain and one heavy chain. In each pair, the light and heavy chain variable regions (Vl and Vh) are together primarily responsible for binding to an antigen. The light chain and heavy chain variable domains consist of a framework region interrupted by three hypervariable regions, also called “complementarity determining regions” or “CDRs.” The constant regions may be recognized by and interact with the immune system. (see, e.g., Janeway el ah, 2001, Immunol. Biology, 5th Ed., Garland Publishing, New York). An antibody can be of any type (e.g., IgG, IgE, IgM, IgD, and IgA), class (e.g., IgG1, IgG2, IgG3, IgG4, IgA1 and IgA2) or subclass thereof. The antibody can be derived from any suitable species. In some embodiments, the antibody is of human or murine origin. An antibody can be, for example, human, humanized, or chimeric. The term antibody also includes functional equivalents.

As used herein, the term “functional equivalents” includes antibodies with homologous sequences, chimeric antibodies, artificial antibodies and modified antibodies. One of ordinary skill in the art will understand that there is an overlap in the group of molecules termed “antibody fragments” and the group termed “functional equivalents.”

As used herein, the term “monoclonal antibody” refers to an antibody obtained from a population of substantially homogeneous antibodies, Le., the individual antibodies including the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

As used herein, the term “intact antibody” refers to an antibody that includes an antigen-binding variable region as well as a light chain constant domain (C_{L}) and heavy chain constant domains, C_{H}1, C_{H}2, C_{H}3, and C_{H}4, as appropriate for the antibody class. The constant domains may be native sequence constant domains (e.g., human native sequence constant domains) or amino acid sequence variant thereof.

As used herein, the term “antibody fragment” includes a portion of an intact antibody, including the antigen-binding or variable region thereof. Examples of antibody fragments include Fab, Fab′, F(ab′)₂, and Fv fragments, diabodies, triabodies, tetrabodies, linear antibodies, single chain antibody molecules, scFv, scFv-Fc, multispecific antibody fragments formed from antibody fragment(s), a fragment(s) produced by a Fab expression library, or an epitope binding fragments of any of the above which immunospecifically bind to a target antigen (e.g., a cancer cell antigen, a viral antigen or a microbial antigen). Antibody fragments of the present disclosure include at least one cysteine residue (natural or engineered) that provides a site for attachment of a linker and/or drug-linker moiety. In some embodiments, an antibody fragment includes Fab, Fab′, or F(ab′)2.

As used herein, the term “engineered cysteine residue” or “eCys residue” refers to a cysteine amino acid or a derivative thereof that is incorporated into an antibody. In those embodiments one or more eCys residues can be incorporated into an antibody, and typically, the eCys residues are incorporated into either the heavy chain or the light chain of an antibody. Generally, incorporation of an eCys residue into an antibody is performed by mutagenizing a nucleic acid sequence of a parent antibody to encode for one or more amino acid residues with a cysteine or a derivative thereof. Suitable mutations include replacement of a desired residue in the light or heavy chain of an antibody with a cysteine or a derivative thereof, incorporation of an additional cysteine or a derivative thereof at a desired location in the light or heavy chain of an antibody, as well as adding an additional cysteine or a derivative thereof to the N- and/or C-terminus of a desired heavy or light chain of an amino acid. Derivatives of cysteine (Cys) include but are not limited to beta-2-Cys, beta-3-Cys, homocysteine, and N-methyl cysteine.

As used herein, the term “antigen” refers to an entity to which an antibody specifically binds.

As used herein, the terms “specific binding” and “specifically binds” mean that the antibody or antibody derivative will bind, in a highly selective manner, with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

As used herein, the term “alkyl”, whether used alone or as part of another term refers to a substituted or unsubstituted straight chain or branched, saturated or unsaturated hydrocarbon having the indicated number of carbon atoms. (e.g., “—(C₁-C₈)-alkyl” or “—(C₁-C₁₀)-alkyl refer to an alkyl group having from 1 to 8 or 1 to 10 carbon atoms, respectively). When the number of carbon atoms is not indicated, the alkyl group has from 1 to 8 carbon atoms. Representative straight chain “—C₁-C₈-alkyl” groups include, but are not limited to, -methyl, -ethyl, -n-propyl, -n-butyl, -n-pentyl, -n-hexyl, -n-heptyl and -n-octyl; while branched —C3-C8 alkyls include, but are not limited to, -isopropyl, -sec-butyl, -isobutyl, -tert-butyl, -isopentyl, and -2-methylbutyl; unsaturated —C₂-C₈ alkyls include, but are not limited to, -vinyl, -allyl, 1-butenyl, -2-butenyl, -isobutylenyl, -1 pentenyl, -2 pentenyl, -3-methyl-1-butenyl, -2 methyl-2-butenyl, -2,3 dimethyl-2-butenyl, -1-hexyl, 2-hexyl, -3-hexyl, -acetylenyl, propynyl, -1 butynyl,-2 butynyl, -1 pentynyl, -2 pentynyl and -3 methyl-1-butynyl. The term alkyl also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The term alkyl also includes polycyclic alkyl groups such as, but not limited to, adamantyl norbornyl, and bicyclo[2.2.2]octyl and such rings substituted with straight and branched chain alkyl groups as defined above. Sometimes an alkyl group is unsubstituted. An alkyl group can be substituted with one or more groups. In other aspects, an alkyl group will be saturated.

As used herein, the term “alkylene,” by itself of as part of another term, refers to a substituted or unsubstituted saturated, branched or straight chain or cyclic hydrocarbon radical of the stated number of carbon atoms, typically 1-10 carbon atoms, and having two monovalent radical centers derived by the removal of two hydrogen atoms from the same or two different carbon atoms of a parent alkane. Typical alkylene radicals include, but are not limited to methylene (—CH₂—), 1,2-ethylene (—CH₂CH₂—), 1,3-propylene (—CH₂CH₂CH₂—), 1,4butylene (—CH₂CH₂CH₂CH₂—), and the like. In preferred aspects, an alkylene is a branched or straight chain hydrocarbon (i.e., it is not a cyclic hydrocarbon).

As used herein, the term “alkenyl”, by itself or as part of another term, unless otherwise stated or implied by context, refers to an organic moiety, substituent, or group that includes one or more double bond functional groups (e.g., a —CH═CH-moiety) or 1, 2, 3, 4, 5, or 6 or more, typically 1, 2, or 3 of such functional groups, more typically one such functional group, and in some aspects may be substituted (i.e., is optionally substituted) with an aryl moiety or group such as phenyl, or may contain non-aromatic linked normal, secondary, tertiary or cyclic carbon atoms, i.e., linear, branched, cyclic or any combination thereof as part of the base moiety unless the alkenyl substituent, moiety or group is a vinyl moiety (e.g., a —CH═CH moiety). An alkenyl moiety, group or substituent having multiple double bonds may have the double bonds arranged contiguously (i.e., a 1,3-butadienyl moiety) or non-contiguously with one or more intervening saturated carbon atoms or a combination thereof, provided that a cyclic, contiguous arrangement of double bonds do not form a cyclic conjugated system of 4n+2 electrons (i.e., is not aromatic). Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

As used herein, the term “alkenyl”, by itself or as part of another term, unless otherwise stated or implied by context, refers to an organic moiety, substituent, or group that includes one or more double bond functional groups (e.g., a —CH═CH-moiety) or 1, 2, 3, 4, 5, or 6 or more, typically 1, 2, or 3 of such functional groups, more typically one such functional group, and in some aspects may be substituted (i.e., is optionally substituted) with an aryl moiety or group such as phenyl, or may contain non-aromatic linked normal, secondary, tertiary or cyclic carbon atoms, i.e., linear, branched, cyclic or any combination thereof as part of the base moiety unless the alkenyl substituent, moiety or group is a vinyl moiety (e.g., a —CH═CH moiety). An alkenyl moiety, group or substituent having multiple double bonds may have the double bonds arranged contiguously (i.e., a 1,3-butadienyl moiety) or non-contiguously with one or more intervening saturated carbon atoms or a combination thereof, provided that a cyclic, contiguous arrangement of double bonds do not form a cyclic conjugated system of 4n+2 electrons (i.e., is not aromatic). Representative examples of alkynyl include, but are not limited, to acetylenyl, 1-propynyl, 2-propynyl, 3-butynyl, 2-pentynyl, 4-pentynyl, and 1-butynyl.

As used herein, the term “unsubstituted alkyl”, “unsubstituted alkenyl” and “unsubstituted alkynyl” refers to alkyl, alkenyl and alkynyl groups that do not contain heteroatoms.

As used herein, the term “substituted alkyl”, “substituted alkenyl”, and “substituted alkynyl” refers to alkyl, alkenyl and alkynyl groups as defined above in which one or more bonds to a carbon(s) or hydrogen(s) are replaced by a bond to non-hydrogen or non-carbon atoms such as, but not limited to, a halogen atom in halides such as F, Cl, Br, and I; and oxygen atom in groups such as carbonyl, carboxyl, hydroxyl groups, alkoxy groups, aryloxy groups, heterocyclyloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, enamines imines, oximes, hydrazones, heterocyclylamine, (alkyl)(heterocyclyl)-amine, (aryl)(heterocyclyl)amine, diheterocyclylamine, triazoles, and nitriles; a silicon atom in groups such as in trialkyl silyl groups, dialkylarylsilyl groups, alkyldiaryl silyl groups, and triaryl silyl groups; and other heteroatoms in various other groups. In a specific embodiment, a “polar alkyl”, “polar alkenyl”, and “polar alkynyl”, refers to alkyl, alkenyl, and alkynyl groups substituted with an atom that results in a polar covalent bond. In another specific embodiment, a “polar alkyl”, “polar alkenyl”, and “polar alkynyl” refers to C₁ to C₅ alkyl, alkenyl, and alkynyl, groups substituted with an atom that results in a polar covalent bond. In a specific embodiment, a “polar alkyl”, “polar alkenyl”, and “polar alkynyl”, refers to alkyl, alkenyl, alkynyl groups, such as C₁-C₅ alkyl, alkenyl, and alkynyl groups, substituted with an —OH group and/or a —C(O)—OH group.

As used herein, the term “aralkyl” or “arylalkyl”, by itself or as part of another term refers to an alkyl group substituted with an aryl group, wherein the moiety is appended to the parent molecule through the alkyl group. An arylalkyl group may be optionally substituted. A “substituted aralkyl” has the same meaning with respect to unsubstituted aralkyl groups that substituted aryl groups had with respect to unsubstituted aryl groups. However, a substituted aralkyl group also includes groups in which a carbon or hydrogen bond of the alkyl part of the group is replaced by a bond to a non-carbon or a non-hydrogen atom.

As used herein, the term “aryl,” by itself or as part of another term, means a substituted or unsubstituted monovalent carbocyclic aromatic hydrocarbon radical of the stated number of carbon atoms, typically 6-20 carbon atoms, derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

As used herein, the term “unsubstituted aryl” refers to monocyclic or bicyclic aromatic hydrocarbon groups having 6 to 12 carbon atoms in the ring portion, such as, but not limited to, phenyl, naphthyl, anthracenyl, biphenyl and diphenyl groups, that do not contain heteroatoms. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. Unsubstituted aryl groups may be bonded to one or more carbon atom(s), oxygen atom(s), nitrogen atom(s), and/or sulfur atom(s) in the parent compound, however.

As used herein, the term “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms, such as, but not limited to, those atoms described above with respect to a substituted alkyl, and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl or alkenyl, group to define a fused ring system (e.g. dihydronaphthyl or tetrahydronaphthyl). Thus, the phrase “substituted aryl” includes, but is not limited to tolyl, and hydroxyphenyl among others.

As used herein, the term “heterocyclyl”, by itself or as part of another term refers to a radical of a non-aromatic ring system, including, but not limited to, monocyclic, bicyclic, tricyclic and polycyclic rings, which can be completely saturated or which can contain one or more units of unsaturation, for the avoidance of doubt, the degree of unsaturation does not result in an aromatic ring system, and having 3 to 15 atoms including at least one heteroatom, such as nitrogen, oxygen, or sulfur. For purposes of exemplification, which should not be construed as limiting the scope of this invention, the following are examples of heterocyclic rings: aziridinyl, azirinyl, oxiranyl, thiiranyl, thiirenyl, dioxiranyl, diazirinyl, diazepanyl, 1,3-dioxanyl, 1,3-dioxolanyl, 1,3-dithiolanyl, 1,3-dithianyl, imidazolidinyl, isothiazolinyl, isothiazolidinyl, isoxazolinyl, isoxazolidinyl, azetyl, oxetanyl, oxetyl, thietanyl, thietyl, diazetidinyl, dioxetanyl, dioxetenyl, dithietanyl, dithietyl, dioxalanyl, oxazolyl, thiazolyl, triazinyl, isothiazolyl, isoxazolyl, azepines, azetidinyl, morpholinyl, oxadiazolinyl, oxadiazolidinyl, oxazolinyl, oxazolidinyl, oxopiperidinyl, oxopyrrolidinyl, piperazinyl, piperidinyl, pyranyl, pyrazolinyl, pyrazolidinyl, pyrrolinyl, pyrrolidinyl, quinuclidinyl, thiomorpholinyl, tetrahydropyranyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. A heterocyclyl group may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like.

As used herein, the term “unsubstituted heterocyclyl” refers to both aromatic and nonaromatic ring compounds including monocyclic, bicyclic, and polycyclic ring compounds containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members, as compounds such as 2-methylbenzimidazolyl are “substituted heterocyclyl” groups as defined below. Examples of heterocyclyl groups include, but are not limited to: unsaturated 3 to 8 membered rings containing 1 to 4 nitrogen atoms, condensed unsaturated heterocyclic groups containing 1 to 4 nitrogen atoms, unsaturated 3 to 8 membered rings containing 1 to 2 oxygen atoms and 1 to 3 nitrogen atoms, saturated 3 to 8 membered rings containing 1 to 2 oxygen atoms and 1 to 3 nitrogen atoms such, unsaturated condensed heterocyclic groups containing 1 to 2 oxygen atoms and 1 to 3 nitrogen atoms, unsaturated 3 to 8 membered rings containing 1 to 3 sulfur atoms and 1 to 3 nitrogen atoms, saturated 3 to 8 membered rings containing 1 to 2 sulfur atoms and 1 to 3 nitrogen atoms, saturated and unsaturated 3 to 8 membered rings containing 1 to 2 sulfur atoms, unsaturated condensed heterocyclic rings containing 1 to 2 sulfur atoms and 1 to 3 nitrogen atoms, unsaturated 3 to 8 membered rings containing oxygen atoms, unsaturated condensed heterocyclic rings containing 1 to 2 oxygen atoms, unsaturated 3 to 8 membered rings containing an oxygen atom and 1 to 2 sulfur atoms, saturated 3 to 8 membered rings containing 1 to 2 oxygen atoms and 1 to 2 sulfur atoms, unsaturated condensed rings containing 1 to 2 sulfur atoms, and unsaturated condensed heterocyclic rings containing an oxygen atom and 1 to 2 oxygen atoms. Heterocyclyl group also include those described above in which one or more S atoms in the ring is double-bonded to one or two oxygen atoms (sulfoxides and sulfones).

As used herein, the term “substituted heterocyclyl” has the same meaning with respect to unsubstituted heterocyclyl groups that substituted alkyl groups had with respect to unsubstituted alkyl groups. However, a substituted heterocyclyl group also includes heterocyclyl groups in which one of the carbons is bonded to one of the non-carbon or non-hydrogen atom, such as, but not limited to, those atoms described above with respect to a substituted alky and substituted aryl groups and also includes heterocyclyl groups in which one or more carbons of the heterocyclyl group is bonded to a substituted and/or unsubstituted alkyl, alkenyl or aryl group as defined herein. This includes bonding arrangements in which two carbon atoms of an heterocyclyl group are bonded to two atoms of an alkyl, alkenyl, or alkynyl group to define a fused ring system. Examples, include, but are not limited to, 2-methylbenzimidazolyl, 5-methylbenzimidazolyl, 5-chlorobenzthiazolyl, 1-methyl piperazinyl, and 2-chloropyridyl among others.

As used herein, the term “halogen” by itself or in combination with another term, unless otherwise stated or implied by context, refers to fluorine, chlorine, bromine, or iodine and is typically —F or —Cl.

As used herein, the term “POZ”, “POZ derivative” or “polyoxazoline derivative” refers to a structure including a poly(oxazoline) moiety. The POZ polymer has a single active functional group on the terminal end of the POZ polymer, wherein the functional group is capable of forming a linkage, directly or indirectly, with a chemical group on drug-linker moiety. In one embodiment the POZ polymer is a monofunctional POZ derivative.

As used herein, the term “pendent” refers to a side chain group of the POZ polymer.

As used herein, the term “ran” indicates that the POZ polymer is a random copolymer.

As used herein, the term “bond”, “link”, “linked” or“linkage” when used with respect to a polymer or compound described herein, or components thereof, refers to bonds that normally are formed as the result of a chemical reaction and typically are covalent bonds. In certain embodiments, specific linkages are covalent linkages. In certain embodiments, specific linkages are hydrolyzable.

As used herein, the term “cleavable linkage”, “cleavable linker”, “hydrolysable linker”, “hydrolysable functionality”, “releasable linker” or “releasable functionality” refers to a chemical linkage containing a cleavable moiety. The terms hydrolysable and releasable do not imply any particular mechanism by which the linker is cleaved.

As used herein, the term “cleavable moiety” refers to a group (such as in a cleavable linkage) that is cleavable in a subject in-vivo under physiological conditions in the subject after a conjugate of the present disclosure has been administered to the subject. In one embodiment, the cleavable moiety is cleaved by a chemical reaction. In one aspect of this embodiment, the cleavage is by hydrolysis of an ester group or reduction, such as, but not limited to, reduction of a disulfide. In one embodiment, the cleavable moiety is cleaved by a substance that is naturally present or induced to be present in the subject. In one aspect of this embodiment, such a substance is an enzyme or polypeptide. Therefore, in one embodiment, the cleavable moiety is cleaved by an enzymatic reaction. In one embodiment, the cleavable moiety is cleaved by a combination of the foregoing.

As used herein, the term “inert” or “non-reactive” or “non-cleavable” when used in conjunction with a particular functional group refers to a functional group that does not reacts readily with an electrophile or a nucleophile on another molecule and require catalysts or impractical reaction conditions in order to react.

As used herein, the term “hydrolyzable linker” or “releasable, hydrolyzable linker” refers to a chemical linkage that is cleavable in a subject in vivo under specific physiological conditions in the subject after a conjugate of the present disclosure containing the hydrolyzable linker has been administered to the subject. In a further embodiment, the hydrolyzable linker may be biostable. A hydrolyzable linker may contain a hydrolyzable moiety as discussed herein; in certain circumstances a hydrolyzable linker may contain more than one hydrolyzable linker. In one embodiment, the hydrolyzable linker is cleavable on delivery to the site of action, such as, but not limited to, a tumor cell. In one embodiment, the hydrolyzable linker is cleavable only when taken up (for example by endocytosis) by a cell, such as, but not limited to, a tumor cell. In one embodiment, the hydrolyzable linker is cleavable in a lysosome or endosome. In one embodiment, the hydrolyzable linker is cleaved by a chemical reaction. In aspect of this embodiment, the cleavage is by reduction of an easily reduced group, such as, but not limited to, a disulfide or a self-immolative reaction. In one embodiment, the hydrolyzable linker is cleaved by a substance that is naturally present or induced to be present in the subject. In an aspect of this embodiment, such a substance is an enzyme or polypeptide. Therefore, in one embodiment, the hydrolyzable linker is cleaved by an enzymatic reaction. In one embodiment, the hydrolyzable linker is cleaved by a combination of the foregoing. In one embodiment, the hydrolyzable moiety contained within the hydrolyzable linker is cleaved under the same conditions and by the same mechanisms.

As used herein, the term “biostable” refers to a polymer or polymer conjugate (including any linkers or linkages, such as but not limited to a hydrolyzable linker, contained therein) that is resistant to cleavage when administered to a subject, including a human subject, until the polymer or polymer conjugate reaches the site of action (such as, but not limited to, a tumor cell). By “resistant to cleavage” it is meant that at least 80%, 90%, 95%, 98% or 99% or more of the polymer or polymer conjugate is not cleaved until the polymer reaches the site of action. In one embodiment, a biostable polymer or polymer conjugate is resistant to cleavage in the bloodstream of the subject, such that over 80%, 90%, 95%, 98%, 99% or more of the polymer or polymer conjugate is not cleaved in the bloodstream. The term biostable” may also refer to a linker or linkage such as, but not limited to a hydrolyzable linker, such that the linker or linkage is resistant to cleavage when administered to a subject, including a human subject, until the linker or linkage is placed at the site of action (such as, but not limited to, a tumor cell). Therefore, in one embodiment, a biostable linker or linkage is resistant to cleavage in the bloodstream of the subject, such that over 80%, 90%, 95%, 98%, 99% or more of the linker or linkage is not cleaved in the bloodstream.

As used herein, the term “therapeutically effective amount” refers to an amount of a compound or conjugate, either alone or as a part of a pharmaceutical composition, effective to treat a disease or disorder in a subject. By way of non-limiting example, in the case of cancer, the therapeutically effective amount of a conjugate of the present disclosure may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

As used herein terms “treat” or “treatment,” unless otherwise indicated by context, refer to therapeutic treatment and prophylactic where the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of: killing tumor cells; inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of: inhibiting replication of cells associated with an autoimmune disease state including, but not limited to, cells that produce an autoimmune antibody, lessening the autoimmune-antibody burden and ameliorating one or more symptoms of an autoimmune disease.

As used herein, the term “inhibits” or “inhibition of” means to reduce by a measurable amount, or

As used herein, the term “individual”, “subject” or “patient” refers to any animal, including mammals, such as mice, rats, other rodents, rabbits, dogs, cats, swine, cattle, sheep, horses, or primates, and humans. The term may specify male or female or both, or exclude male or female.

As used herein, the term “patient” refers to a subject to whom is administered a conjugate of the present invention. Patient includes, but are not limited to, a human, rat, mouse, guinea pig, non-human primate, pig, goat, cow, horse, dog, cat, bird, and fowl. Typically, the patient is a rat, mouse, dog, human, or non-human primate, more typically a human.

As used herein, the term “compound” refers to and encompasses the chemical compound itself, either named or represented by structure, and salt form(s) thereof, whether explicitly stated or not, unless context makes clear that such salt forms are to be excluded. The term “compound” further encompasses solvate forms of the compound, in which solvent is noncovalently associated with the compound or is reversibly associated covalently with the compound, as when a carbonyl group of the compound is hydrated to form a gem-diol. Solvate forms include those of the compound itself and its salt form(s) and are inclusive of hemisolvates, monosolvates, disolvates, including hydrates; and when a compound can be associated with two or more solvent molecules, the two or more solvent molecules may be the same or different.

In some instances, a compound of the invention will include an explicit reference to one or more of the above forms, e.g., salts and solvates, which does not imply any solid-state form of the compound; however, this reference is for emphasis only, and is not to be construed as excluding any other of the forms as identified above. Furthermore, when explicit reference to a salt and/or solvate form of a compound or a drug linker moiety composition is not made, that omission is not to be construed as excluding the salt and/or solvate form(s) of the compound or conjugate unless context make clear that such salt and/or solvate forms are to be excluded.

As used herein, the term “salt thereof” refers to a salt form of a compound (e.g., a drug, a drug linker moiety or a polymer drug linker moiety or conjugate). A salt form of a compound is of one or more internal salt forms and/or involves the inclusion of another molecule such as an acetate ion, a succinate ion or other counterion. The counterion in a salt form of a compound is typically an organic or inorganic moiety that stabilizes the charge on the parent compound. A salt form of a compound has one or more than one charged atom in its structure. In instances where multiple charged atoms are part of the salt form, multiple counter ions and/or multiple charged counter ions are present. Hence, a salt form of a compound typically has one or more charged atoms corresponding to those of the non-salt form of the compound and one or more counterions. In some aspects, the non-salt form of a compound contains at least one amino group or other basic moiety, and accordingly in the presence of an acid, an acid addition salt with the basic moiety is obtained. In other aspects, the non-salt form of a compound contains at least one carboxylic acid group or other acidic moiety, and accordingly in the presence of a base, a carboxylate or other anionic moiety is obtained. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., I,Γ-methylene-bis(2-hydroxy-3-naphthoate)) salts.

As used herein the term “pharmaceutically acceptable salt” includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt can include forms wherein the ratio of molecules including the salt is not 1:1. For example, the salt may include more than one inorganic or organic acid molecule per molecule of base, such as two hydrochloric acid molecules per molecule of conjugate. As another example, the salt may include less than one inorganic or organic acid molecule per molecule of base, such as two molecules of conjugate per inorganic or organic acid molecule.

As used herein the terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, incorporated herein by reference in its entirety.

Certain compounds contained in the conjugates and/or compositions of the present disclosure may exist in particular geometric or stereoisomeric forms. In addition, compounds o contained in the conjugates and/or compositions of the present disclosure may also be optically active. The present disclosure contemplates all such compounds, including cis- and trans-isomers, (R)- and (S)-enantiomers. diastereoisomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction. It will be understood that when a group is specified as a part of a compound, the substitution of the group may be adjusted to accommodate the particular bonds. For example, when an alkyl group is joined to two other groups, the alkyl group is considered an alkylene group.

The term “substituted” is also contemplated to include all permissible substituents of organic compounds. In a broad aspect, the permissible substituents include acyclic and cyclic, branched and unbranched substituents, carbocyclic and heterocyclyl, aromatic and nonaromatic substituents of organic compounds. Illustrative substituents include, for example, those described herein above. For purposes of this disclosure, the heteroatoms, such as oxygen or nitrogen, may have hydrogen substituents and/or any permissible substituents of organic compounds described herein which satisfy the valences of the heteroatoms. Exemplary substitutions include, but are not limited to, hydroxy, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. This invention is not intended to be limited in any manner by the permissible substituents of organic compounds.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference in its entirety. Unless otherwise defined, all technical and scientific terms used herein have the same meaning as commonly understood by one of ordinary skill in the art to which this invention pertains.

A number of embodiments of the invention are described below, which are not meant to limit the invention in any way, are followed by a more detailed discussion of the components that make up the conjugates. One of skill in the art will understand that each of the conjugates identified and any of the selected embodiments thereof is meant to include the full scope of each component and linker.

### Conjugates and Scaffolds of the Present Disclosure

In some embodiments, the present disclosure provides a polymer antibody drug conjugate of Formula (I), or a pharmaceutically acceptable salt or solvate thereof:

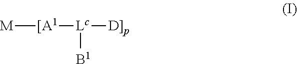

wherein:
M is a recognition moiety;
A¹ is a divalent linker moiety connecting the recognition moiety to the linker moiety L^{C};
L^{C} is a linker moiety;
B¹ is a polymer moiety
D is a drug moiety; and
p is an integer ranging from about 1 to 8.

In some aspects, the present disclosure provides a polymer drug linker moiety useful for conjugating with a recognition moiety, wherein the polymer drug linker moiety is of Formula (II), or a pharmaceutically acceptable salt or solvate thereof:

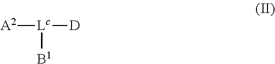

wherein:
A² is a monovalent linker moiety including a functional group capable of forming a covalent bond with a functional group of the recognition moiety;
L^{C} is a linker moiety;
B¹ is a POZ polymer moiety; and
D is a drug moiety.

In some aspects, the present disclosure provides a drug linker moiety useful for conjugating with a recognition moiety and/or a polymer moiety, wherein the drug linker moiety is of Formula (III), or a pharmaceutically acceptable salt or solvate thereof:

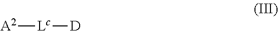

wherein:
A² is a monovalent linker moiety including a functional group capable of forming a covalent bond with a functional group of the recognition moiety;
L^{C} is a linker moiety; and
D is a drug moiety.

In some aspects, the linker moiety L^{C} has the Formula (IV):

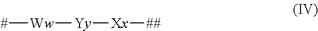

wherein:
each W independently is a peptide unit including at least two amino acids;
Y is a self-immolative moiety, a non-self-immolative releasable moiety or a non-cleavable moiety;
X is a spacer moiety;
w is an integer from about 0 to about 12;
y is an integer from 0 to 1;
x is an integer from 0 to 1;
# donates attachment to A¹ or A²; and
## donates attachment to D.

It is understood that, for a conjugate of any one of Formulae (I), (II), (III) or (IV), or a pharmaceutically acceptable salt or solvate thereof, variables M, L^{C}, A¹, A², D, B¹, D, Ww, Yy, Xx and p can each be, where applicable, selected from the groups described herein, and any group described herein for any of variables M, L^{C}, A¹, A², D, B¹, D, Ww, Yy, Xx and p can be combined, where applicable, with any group described herein for one or more of the remainder of variables M, L^{C}, A¹, A², D, B¹, D, Ww, Yy, Xx and p.

Variables A¹ and A²

In some embodiments, each A¹ independently is a divalent linker moiety connecting the recognition moiety to the linker moiety L^{C}.

In some embodiments, each A¹ independently is a divalent linker moiety connecting the recognition moiety to variable W, if present, or to variable Y, if W is absent; or connecting the recognition moiety to variable X, if both variables W and Y are absent; or connecting the recognition moiety to the drug moiety D, if variables W, Y and X are absent.

In some embodiments, each A¹ independently is:

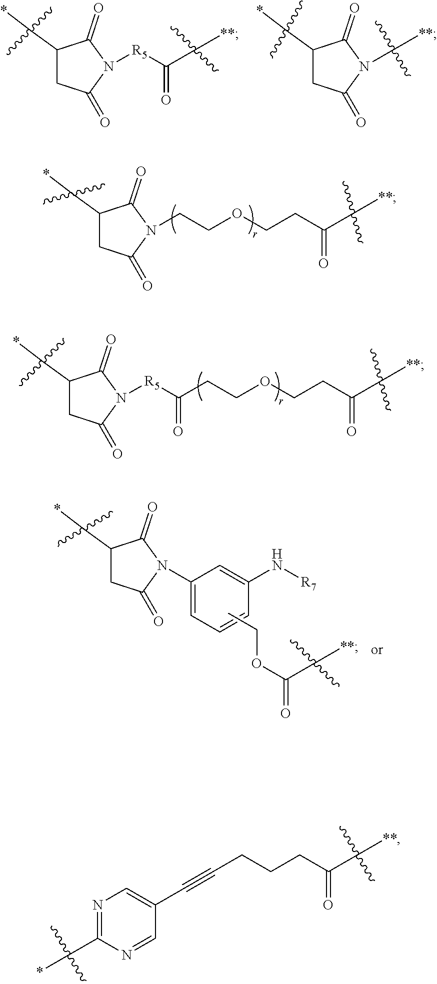

wherein:
R₅ is —O—, —NR₆—, —(C₁-C₁₀)-alkyl-, —(C₁-C₁₀)-alkenyl-, —(C₁-C₁₀)-alkynyl-, —(C₃-C₈)-cycloalkyl-, -aryl-, —O—(C₁-C₈)-alkyl-, —O—(C₁-C₁₀)-alkenyl-, —O—(C₁-C₁₀)-alkynyl-, -aryl-(C₁-C₁₀)-alkyl-C(O), —(C₁-C₁₀)-alkyl-aryl-, —O—C(O)—(CH₂CH₂O)r-(CH₂)₂—, —(CH₂CH₂O)r-, or —(CH₂CH₂O)r-(CH₂)₂—,
R₆ is H, hydroxy, or (C₁-C₄)-alkyl;
R₇ is one or more amino acids;
r is an integer ranging from about 1 to about 12;
* denotes attachment to the recognition moiety;
** denotes attachment to L^{C}; and
*** donates attachment to the polymer moiety B¹.

In some embodiments, R₅ is —O—, —NH—, —N(CH₃)—, —(CH₂)₁₋₆—, —C₆H₅—CH₂—C(O), —C₆H₅—(CH₂)₂—C(O), —(C₁-C₁₀)-alkyl)-, —O—C(O)—(CH₂CH₂O)₆—(CH₂)₂—, —(CH₂CH₂O)₂—(CH₂)₂—, —(CH₂CH₂O)₄—(CH₂)₂ or —(CH₂CH₂O)₆—(CH₂)₂.

In some embodiments, each A¹ independently is:

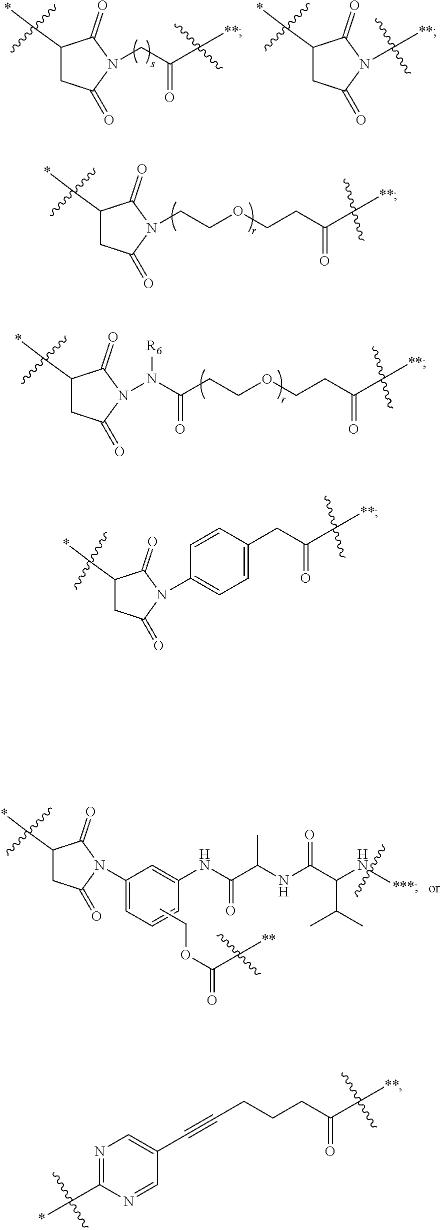

wherein
R₆ is H, hydroxy, or (C₁-C₄)-alkyl;
r is an integer ranging from about 4 to about 6;
s is an integer ranging from about 1 to about 6;
* denotes attachment to the recognition moiety;
** denotes attachment to L^{C}; and
*** donates attachment to the polymer moiety B¹.

In some embodiments, each A¹ independently is:

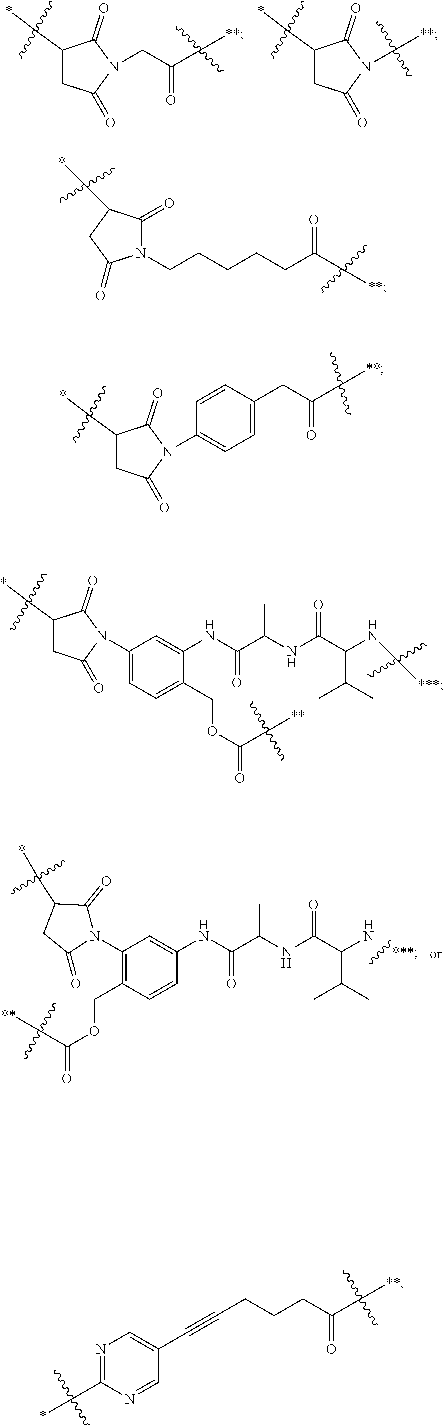

wherein:
* denotes attachment to the recognition moiety;
** denotes attachment to L^{C}; and
*** donates attachment to the polymer moiety B¹.

In some embodiments, each A¹ independently is:

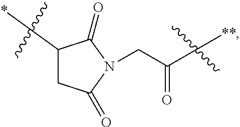

wherein:
* denotes attachment to the recognition moiety; and
** denotes attachment to L^{C}.

In some embodiments, each A¹ independently is:

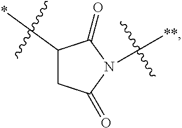

wherein:
* denotes attachment to the recognition moiety; and
** denotes attachment to L^{C}.

In some embodiments, each A¹ independently is:

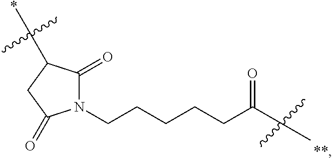

wherein:
* denotes attachment to the recognition moiety; and
   ** denotes attachment to L^{C}.

In some embodiments, each A¹ independently is:

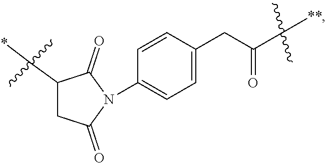

wherein:
* denotes attachment to the recognition moiety; and
** denotes attachment to L^{C}.

In some embodiments, each A¹ independently is:

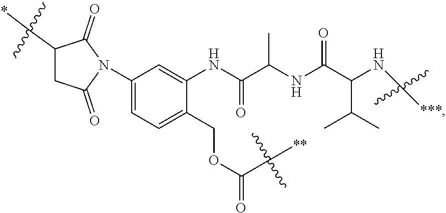

wherein:
* denotes attachment to the recognition moiety;
** denotes attachment to L^{C}; and
*** donates attachment to the polymer moiety B¹.

In some embodiments, each A¹ independently is:

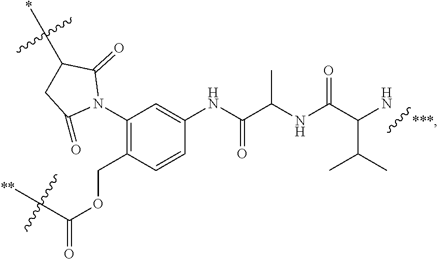

wherein:
* denotes attachment to the recognition moiety;
   ** denotes attachment to L^{C}; and
*** donates attachment to the polymer moiety B¹.

In some embodiments, each A¹ independently is:

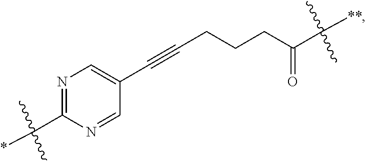

wherein:
* denotes attachment to the recognition moiety; and
   ** denotes attachment to L^{C}.

It is understood that each A¹, prior to being connected to the recognition moiety independently corresponds to a monovalent moiety A².

In some embodiments, each A² independently is:

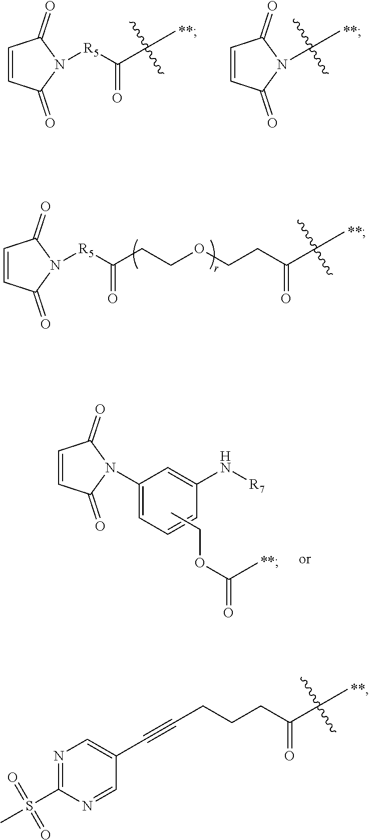

wherein
R₅ is —O—, —NR₆—, —(C₁-C₁₀)-alkyl)-, —(C₁-C₁₀)-alkenyl-, —(C₁-C₁₀)-alkynyl-, —(C₃-C₈)-cycloalkyl-, -aryl-, —O—(C₁-C₈)-alkyl-, —O—(C₁-C₁₀ alkenyl)-, —O—(C₁-C₁₀)-alkynyl-, -aryl-(C₁-C₁₀)-alkyl-C(O), —(C₁-C₁₀)-alkyl-aryl-, —O—C(O)—(CH₂CH₂O)r-(CH₂)₂—, —(CH₂CH₂O)r-, or —(CH₂CH₂O)r-(CH₂)₂—,
R₆ is H, hydroxy, or C₁-C₄ alkyl;
R₇ is one or more amino acids;
r is an integer ranging from about 1 to about 12;
** denotes attachment to L^{C}; and
*** donates attachment to the polymer moiety B¹.

In some embodiments, R₅ is —O—, —NH—, —N(CH₃)—, —(CH₂)₁₋₆—, —C₆H₅—CH₂—C(O), —C₆H₅—(CH₂)₂—C(O), —(C₁-C₁₀)-alkyl-, —O—C(O)—(CH₂CH₂O)₆—(CH₂)₂—, —(CH₂CH₂O)₂—(CH₂)₂—, —(CH₂CH₂O)₄—(CH₂)₂ or —(CH₂CH₂O)₆—(CH₂)₂.

In some embodiments, each A² independently is:

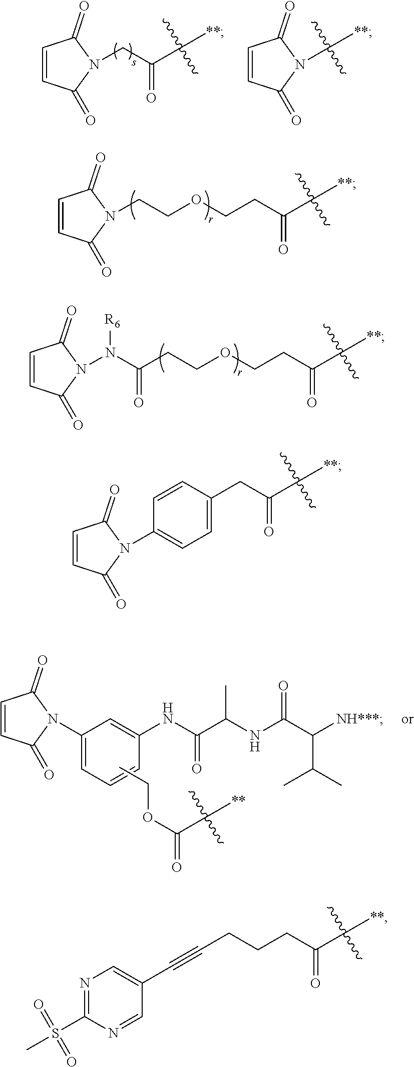

wherein:
R₆ is H, hydroxy, or (C₁-C₄)-alkyl;
r is an integer ranging from about 4 to about 6;
s is an integer ranging from about 1 to about 6;
** denotes attachment to L^{C}; and
*** donates attachment to the polymer moiety B¹.

In some embodiments, each A² independently is:

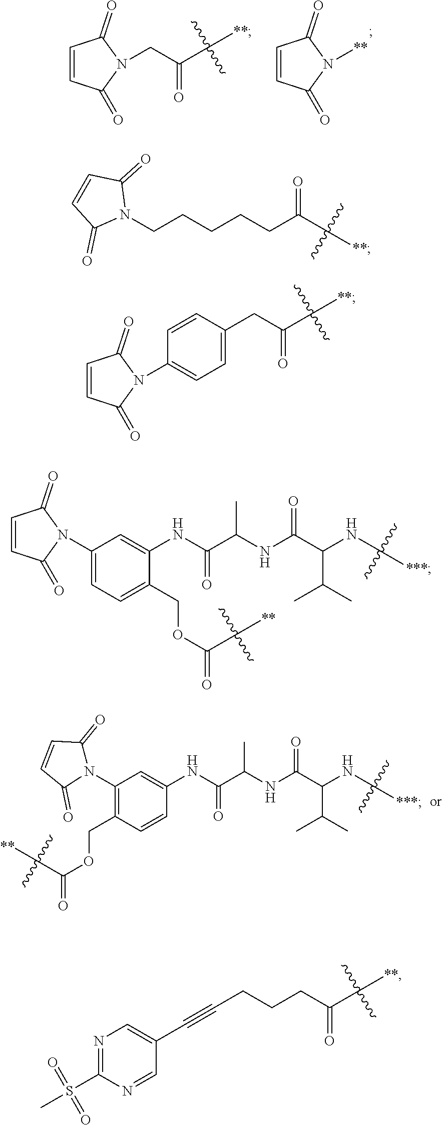

wherein:
** denotes attachment to L^{C}; and
*** donates attachment to the polymer moiety B¹.

In some embodiments, each A² independently is:

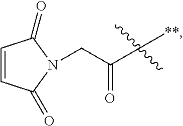

wherein:
** denotes attachment to L^{C}.

In some embodiments, each A² independently is:

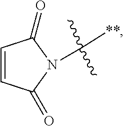

wherein:
** denotes attachment to L^{C}.

In some embodiments, each A² independently is:

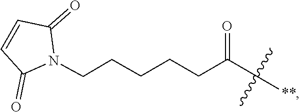

wherein:
** denotes attachment to L^{C}.

In some embodiments, each A² independently is:

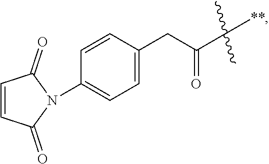

wherein:
** denotes attachment to L^{C}.

In some embodiments, each A² independently is:

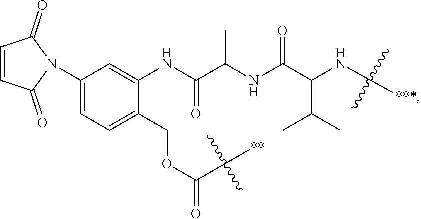

wherein:
** denotes attachment to L^{C}; and
*** donates attachment to the polymer moiety B¹.

In some embodiments, each A² independently is:

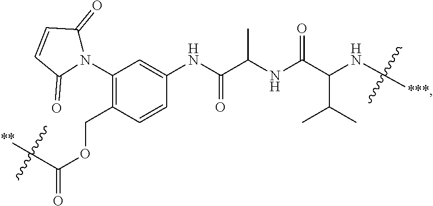

wherein:
** denotes attachment to L^{C}; and
*** donates attachment to the polymer moiety B¹.

In some embodiments, each A² independently is:

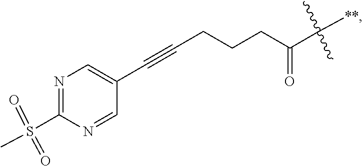

wherein:
** denotes attachment to L^{C}.

### Variable L^{C} (Linker Moiety)

In some embodiments, the linker moiety L^{C} has the Formula (IV):

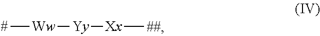

wherein:
each W independently is a peptide unit;
Y is a self-immolative moiety, a non-self-immolative releasable moiety or a non-cleavable moiety;
X is a spacer moiety;
w is an integer from about 0 to about 12;
y is an integer from 0 to 1;
x is an integer from 0 to 1;
# donates attachment to A¹ or A²; and
## donates attachment to the drug moiety D.

### Variable Ww

In some embodiments, each W independently is a peptide unit connecting variable A¹ or A² to variable Y, if present, or to variable X if Y is absent; or connecting variable A¹ or A² to the drug moiety D if both variables Y and X are absent.

In some embodiments, amino acid is referred to herein as “AA” and amino acids as “AA's”.

In some embodiments, W includes an amino acid that is capable of forming a covalent bond with a polymer moiety B¹.

In some embodiments, W includes one or more AA units (e.g., from 0 to 12, from 2 to 10, 2 to 6, or 2, 3, 4, 5 or 6) wherein the AA units are each independently a natural or non-natural amino acid, an amino alcohol, an amino aldehyde, a diamine, a polyamine, or a combination thereof.

In some embodiments, each W independently is a natural or unnatural amino acid and/or a D or L isomer.

In some embodiments, each W independently is an alpha, beta, or gamma amino acid that is natural or non-natural.

In some embodiments, each W independently is a natural amino acid.

In some embodiments, at least one W is a natural amino acid. In some embodiments, at least one W is a non-natural amino acid.

In some embodiments, each W independently is a L isomer.

In some embodiments, each W independently is a D isomer.

In some embodiments, Ww does not include a natural amino acid. In some embodiments, Ww does not include a non-natural amino acid.

In some embodiments, Ww includes a natural amino acid linked to a non-natural amino acid. In some embodiments, Ww includes a natural amino acid linked to a D-isomer of a natural amino acid.

In some embodiments, W is selective for enzymatic cleavage (e.g., by a particular enzyme). In some embodiments, the particular enzyme is a tumor-associated protease.

In some embodiments, Ww includes a bond whose cleavage is catalyzed by the tumor-associated protease cathepsin B, cathepsin C, cathepsin D, or a plasmin protease.

In some embodiments, the bond between W and Y or between W and X is enzymatically cleavable by a tumor-associated protease.

In some embodiments, Ww is a dipeptide, a tripeptide, a tetrapeptide, a pentapeptide, a hexapeptide, a heptapeptide, an octapeptide, a nonapeptide, a decapeptide, an undecapeptide, or a dodecapeptide unit.

In some embodiments, Ww is a dipeptide. In some embodiments, Ww is a tripeptide. In some embodiments, Ww is a tetrapeptide. In some embodiments, Ww is a pentapeptide. In some embodiments, Ww is a hexapeptide.

In some embodiments, each amino acid in Ww is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In some embodiments, each amino acid in Ww is independently selected from alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, citrulline, and derivatives thereof.

In some embodiments, each amino acid in Ww is independently selected from the proteinogenic and the non-proteinogenic amino acids.

In some embodiments, each amino acid in Ww is independently selected from L or D isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, proline, tryptophan, valine, cysteine, methionine, selenocysteine, ornithine, penicillamine, aminoalkanoic acid, aminoalkynoic acid, aminoalkanedioic acid, aminobenzoic acid, amino-heterocyclo-alkanoic acid, heterocyclo-carboxylic acid, citrulline, statine, diaminoalkanoic acid, and derivatives thereof.

In some embodiments, each amino acid in Ww is independently cysteine, homocysteine, penicillamine, ornithine, lysine, serine, threonine, glycine, glutamine, alanine, aspartic acid, glutamic acid, selenocysteine, proline, glycine, isoleucine, leucine, methionine, valine, citrulline, or alanine.

In some embodiments, each amino acid in Ww is independently selected from the L-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in Ww is independently selected from the D-isomers of the following amino acids: alanine, β-alanine, arginine, aspartic acid, asparagine, histidine, glycine, glutamic acid, glutamine, phenylalanine, lysine, leucine, serine, tyrosine, threonine, isoleucine, tryptophan, citrulline, and valine.

In some embodiments, each amino acid in Ww is alanine, β-alanine, glycine, glutamic acid, isoglutamic acid, isoaspartic acid, citrulline, lysine, valine or aspartic acid.

In some embodiments, Ww includes alanine, β-alanine, glycine, glutamic acid, isoglutamic acid, lysine or valine.

In some embodiments, Ww includes alanine, lysine and valine.

In some embodiments, Ww includes lysine, valine and citrulline.

In some embodiments, Ww includes alanine and valine.

In some embodiments, Ww includes alanine and lysine.

In some embodiments, Ww is -Lys-Val-Ala-, -Val-Ala-Lys-, Ala-Lys-Val-, Lys-Ala-Ala-, -Ala-Ala-Lys-, -Ala-Lys-Ala-, -Lys-Val-Cit-, Val-Cit-Lys-, Cit-Lys-Val-, -Val-Ala-, or -Ala-Val-.

In some embodiments, Ww is not -Val-Cit- or -Cit-Val-.

In some embodiments, w is an integer ranging from about 0 to about 12 (e.g., 2 to 10, or 2 to 6, or 2 to 4, or 2 to 3, or 0, 1, 2, 3, 4, 5, 6, 7, 8, 9, 10, 11 or 12).

In some embodiments, w is 2, 3, 4, 5 or 6.

In some embodiments, w is 2.

In some embodiments, w is 3.

In some embodiments, w is 4.

In some embodiments, w is 5.

In some embodiments, w is 6.

In some embodiments, w is 2. In some embodiments, w is 3. In some embodiments, w is 4.

In some embodiments, Ww is conjugated to the polymer moiety B¹.

In some embodiments, Ww is conjugated to the polymer moiety B¹ via the amino amino acid lysine.

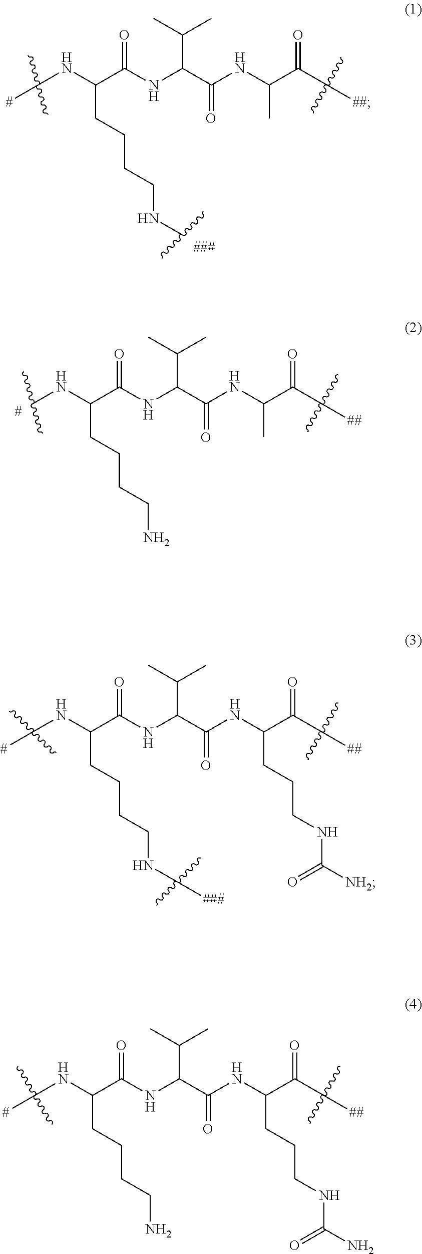

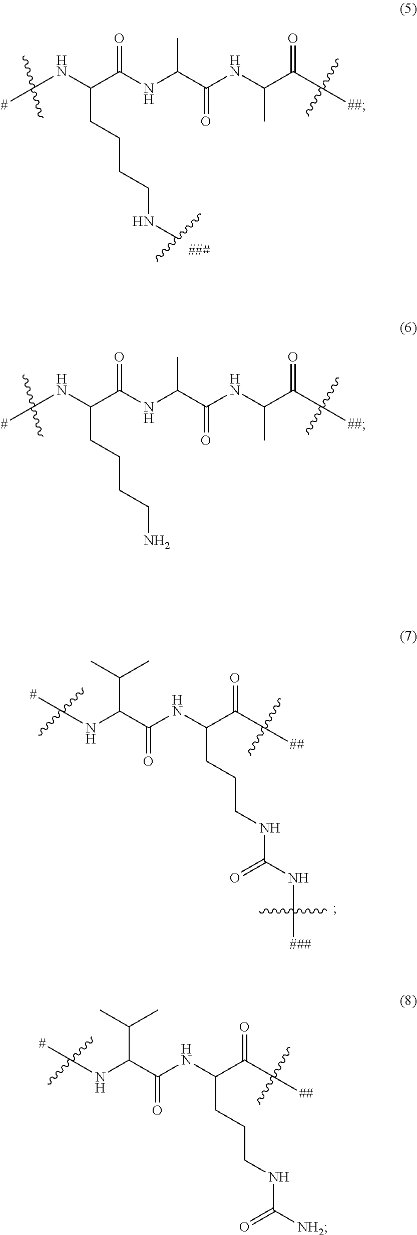

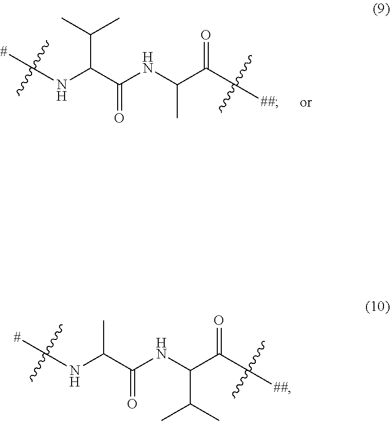

wherein:
# donates attachment to variable A¹ or A²;
## donates attachment to variable Y, if present, or to variable X if Y is absent, or to the drug moiety D, if both variables Y and X are absent; and
### donates attachment to the polymer moiety B¹.

### Variable Yy

In some embodiments, Y is a self-immolative moiety, a non-self-immolative releasable moiety, or a non-cleavable moiety connecting variable W to variable X, if present, or to the drug moiety D, if variable X is absent; or connecting variable A¹ or A² to variable X, if variable W is absent, or connecting variable A¹ or A² to the drug moiety D, if both variables W and X are absent.

In some embodiments, subscript y is an integer 0. In some embodiments subscript y is an integer 1.

In some embodiments, Y is a self-immolative moiety, a non-self-immolative releasable moiety, or a non-cleavable moiety. In some embodiments, Y is a self-immolative moiety or a non-self-immolative releasable moiety. In some embodiments, Y is a self-immolative moiety. In some embodiments, Y is a non-self-immolative moiety.

A non-self-immolative moiety is one which requires enzymatic cleavage, and in which part or all of the group remains bound to the drug moiety after cleavage from the ADC, thereby forming free drug. Examples of a non-self-immolative moiety include, but are not limited to: -glycine-; -glycine-glycine-; a p-aminobenzyl alcohol (PAB) optionally substituted with one to four substituents independently selected from halogen, (C₁-C₆)-alkoxy, —N((C₁-C₆)alkyl)₂, —NHC(═O)(C₁-C₆)-alkyl, —NHC(═O)(C₁-C₆)-alkyl)-NH₂, —C(═O)NH(C₁-C₆)-alkyl, —C(═O)NH(C₁-C₆)-alkyl-NH₂, —CN, —CF₃, acyl, carboxamido, (C₁-C₆)-alkyl, or —NO₂; and a para-aminobenzyloxy-carbonyl (PABC) group optionally substituted with one to four substituents independently selected from halogen, (C₁-C₆)-alkoxy, —N((C₁-C₆)-alkyl)₂, —NHC(═O)(C₁-C₆)-alkyl, —NHC(═O)(C₁-C₆)-alkyl-NH₂, —C(═O)NH(C₁-C₆)-alkyl, —C(═O)NH(C₁-C₆)-alkyl-NH₂, —CN, —CF₃, acyl, carboxamido, C₁-C₆ alkyl, or —NO₂.

In some embodiments, when an ADC having Y as -glycine- or -glycine-glycine-undergoes enzymatic cleavage (for example, via a cancer-cell-associated protease or a lymphocyte-associated protease), the drug moiety is cleaved from the ADC such that the free drug includes the glycine or glycine-glycine group from Y. In some embodiments, an independent hydrolysis reaction takes place within, or in proximity to, the target cell, further cleaving the glycine or glycine-glycine group from the free drug. For example, an ADC with a non-self-immolative linker with a PAB optionally substituted with one to four substituents independently selected from halogen, cyano, and nitro, can undergo enzymatic cleavage of the linker (for example, via a cancer-cell-associated protease or a lymphocyte-associated protease), releasing a free drug which includes the optionally substituted PAB. This compound may further undergo 1,6-elimination of the PAB, removing any portion of Y from the free drug. See, e.g., Told et ah, 2002, J. Org. Chem. 67:1866-1872. In some embodiments, enzymatic cleavage of the non-self-immolative moiety, as described herein, does not result in any further hydrolysis step(s).

Other examples of self-immolative groups include, but are not limited to, aromatic compounds that are electronically similar to the PAB group such as 2-aminoimidazol-5-methanol derivatives (see, e.g., Hay et ah, 1999, Bioorg. Med. Chem. Lett. 9:2237), ortho or para-aminobenzylacetals, substituted and unsubstituted 4-aminobutyric acid amides (see, e.g., Rodrigues et ah, 1995, Chemistry Biology 2:223), appropriately substituted bicyclo[2.2.1] and bicyclo[2.2.2] ring systems (see, e.g., Storm et ah, 1972, J Amer. Chem. Soc. 94:5815), 2-aminophenylpropionic acid amides (see, e.g., Amsberry et ah, 1990, J. Org. Chem. 55:5867), and elimination of amine-containing drugs that are substituted at the α-position of glycine (see, e.g., Kingsbury et ah, 1984, J Med. Chem. 27:1447).

In some embodiments, Y is a p-aminobenzyl alcohol (PAB) optionally substituted with one to four substituents independently selected from halogen, (C₁-C₆)-alkoxy, —N((C₁-C₆)-alkyl)₂, —NHC(═O)(C₁-C₆)-alkyl, —NHC(═O)(C₁-C₆)-alkyl-NH₂, —C(═O)NH(C₁-C₆)-alkyl, —C(═O)NH(C₁-C₆)-alkyl-NH₂, —CN, —CF₃, acyl, carboxamido, (C₁-C₆)-alkyl, or —NO₂.

In some embodiments, Y is an unsubstituted p-aminobenzyl alcohol (PAB).

In some embodiments, Y is a p-aminobenzyl alcohol (PAB substituted with —NHC(═O)(C₁-C₆)-alkyl-NH₂.

In some embodiments, Y is a p-aminobenzyl alcohol (PAB) substituted with —NHC(═O)(CH₂)₂—NH₂.

In some embodiments, Y is a p-aminobenzyl alcohol (PAB) group substituted with —C(═O)NH(C₁-C₆)-alkyl-NH₂.

In some embodiments, Y is a p-aminobenzyl alcohol (PAB) group substituted with —C(═O)NH(CH₂)₂—NH₂.

In some embodiments, Y is a para-aminobenzyloxy-carbonyl (PABC) group optionally substituted with one to four substituents independently selected from halogen, (C₁-C₆)-alkoxy, —N((C₁-C₆)-alkyl)₂, —NHC(═O)(C₁-C₆)-alkyl, —NHC(═O)(C₁-C₆)-alkyl-NH₂, —C(═O)NH(C₁-C₆)-alkyl, —C(═O)NH(C₁-C₆)-alkyl-NH₂, —CN, —CF₃, acyl, carboxamido, (C₁-C₆)-alkyl, or —NO₂.

In some embodiments, Y is an unsubstituted para-aminobenzyloxy-carbonyl (PABC) group

In some embodiments, Y is a para-aminobenzyloxy-carbonyl (PABC) group substituted with —NHC(═O)(C₁-C₆)-alkyl-NH₂.

In some embodiments, Y is a para-aminobenzyloxy-carbonyl (PABC) group substituted with NHC(═O)(CH₂)₂—NH₂.

In some embodiments, Y is a para-aminobenzyloxy-carbonyl (PABC) group substituted with —C(═O)NH(C₁-C₆)-alkyl-NH₂.

In some embodiments, Y is a para-aminobenzyloxy-carbonyl (PABC) group substituted with —C(═O)NH(CH₂)₂—NH₂.

In some embodiments, Y is a para-aminobenzyloxy-carbonyl (PABC) group optionally substituted with a sugar moiety. In some embodiments, Y is -glycine- or -glycine-glycine-In some embodiments, Y is a branched bis(hydroxymethyl)styrene (BHMS) unit, which is capable of incorporating (and releasing) multiple drug moieties.

In some embodiments, Y is

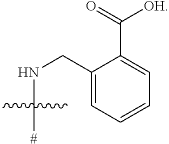

In some embodiments Y is:

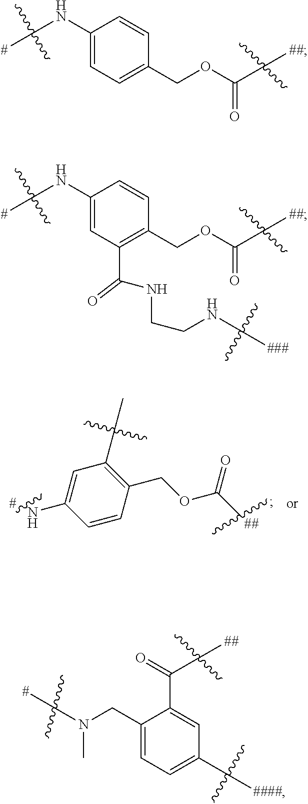

wherein:
# donates attachment to Ww or to variable A¹ or A² if Ww is absent;
## donates attachment to variable X, or to the drug moiety D, if X is absent;
### donates attachment to the polymer moiety B¹; and
#### donates attachment to variable A¹ or A².

In some embodiments Y is:

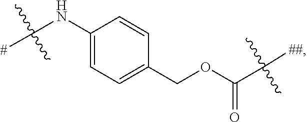

wherein:
# donates attachment to Ww or to variable A¹ or A² if Ww is absent; and
## donates attachment to variable X, or to the drug moiety D, if X is absent.

In some embodiments Y is:

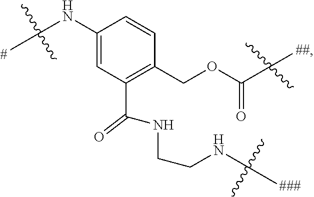

wherein:
# donates attachment to Ww or to variable A¹ or A², if Ww is absent;
## donates attachment to variable X, or to the drug moiety D, if X is absent; and
### donates attachment to the polymer moiety B¹.

In some embodiments Y is:

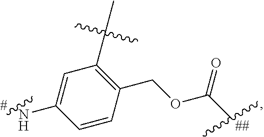

wherein:
# donates attachment to Ww or to variable A¹ or A² if Ww is absent;
## donates attachment to variable X, or to the drug moiety D, if X is absent; and
#### donates attachment to variable A¹ or A².

In some embodiments Y is:

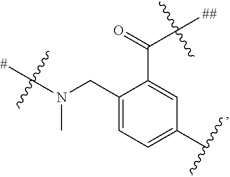

wherein:
# donates attachment to Ww or to variable A¹ or A² if Ww is absent; and
## donates attachment to variable X, or to the drug moiety D, if X is absent.

In some embodiments Y is:

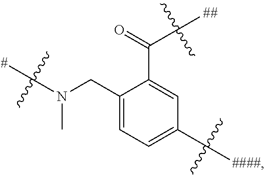

wherein:
# donates attachment to Ww or to variable A¹ or A² if Ww is absent;
## donates attachment to variable X, or to the drug moiety D, if X is absent; and
#### donates attachment to variable A¹ or A².

### Variable Xx

In some embodiments, X is spacer moiety connecting variable Y to the drug moiety D; or connecting variable W to the drug moiety D, if variable Y is absent; or connecting variable A¹ or A² to the drug moiety D, if both variables W and X are absent.

In some embodiments, subscript x is an integer 0. In some embodiments subscript x is an integer 1.

In some embodiments, X is selected from the group consisting of:

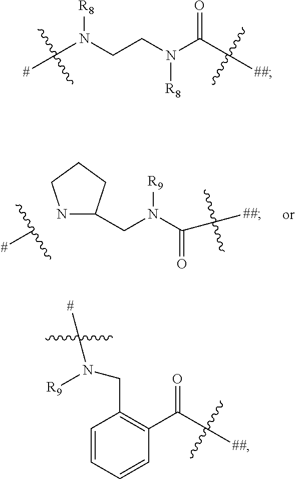

wherein
each R₈ independently is H or (C₁-C₄)-alkyl;
R₉ is H, (C₁-C₄)-alkyl or (C₁-C₄)-alkylene-N—((C₁-C₄)-alkyl)₂;
# donates attachment to variable Y, if present; or attachment to variable W if Y is absent; or attachment to variable A₁ or A₂ if both variables W and Y are absent; and
## donates attachment to the drug moiety D.

In some embodiments each R₈ is independently H or —CH₃.

In some embodiments, each R₈ is H.

In some embodiments, each R₈ is —CH₃.

In some embodiments, one R₈ is H and the other is —CH₃.

In some embodiments, R₉ is H, —CH₃ or (C₁-C₂)-alkylene-N—((C₁-C₂)-alkyl)₂.

In some embodiments, R₉ is H, —CH, or —(CH₂)₂—N—(CH₃)₂.

In some embodiments, X is selected from the group consisting of

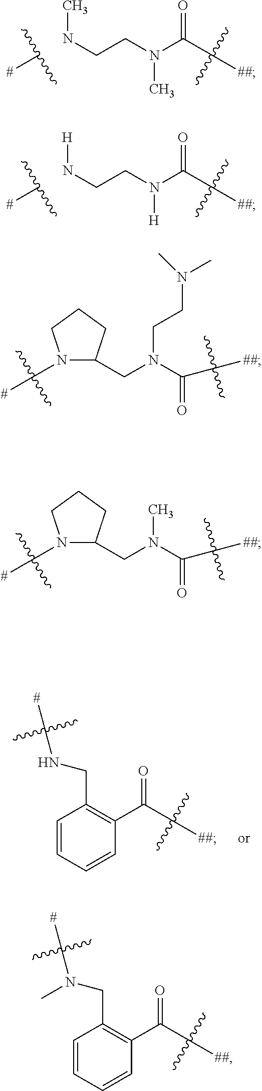

wherein:
# donates attachment to variable Y, if present; or attachment to variable W if Y is absent; or attachment to variable A₁ or A₂ if both variables W and Y are absent; and
## donates attachment to the drug moiety D.

In some embodiments, the linker moiety L^{c} is:

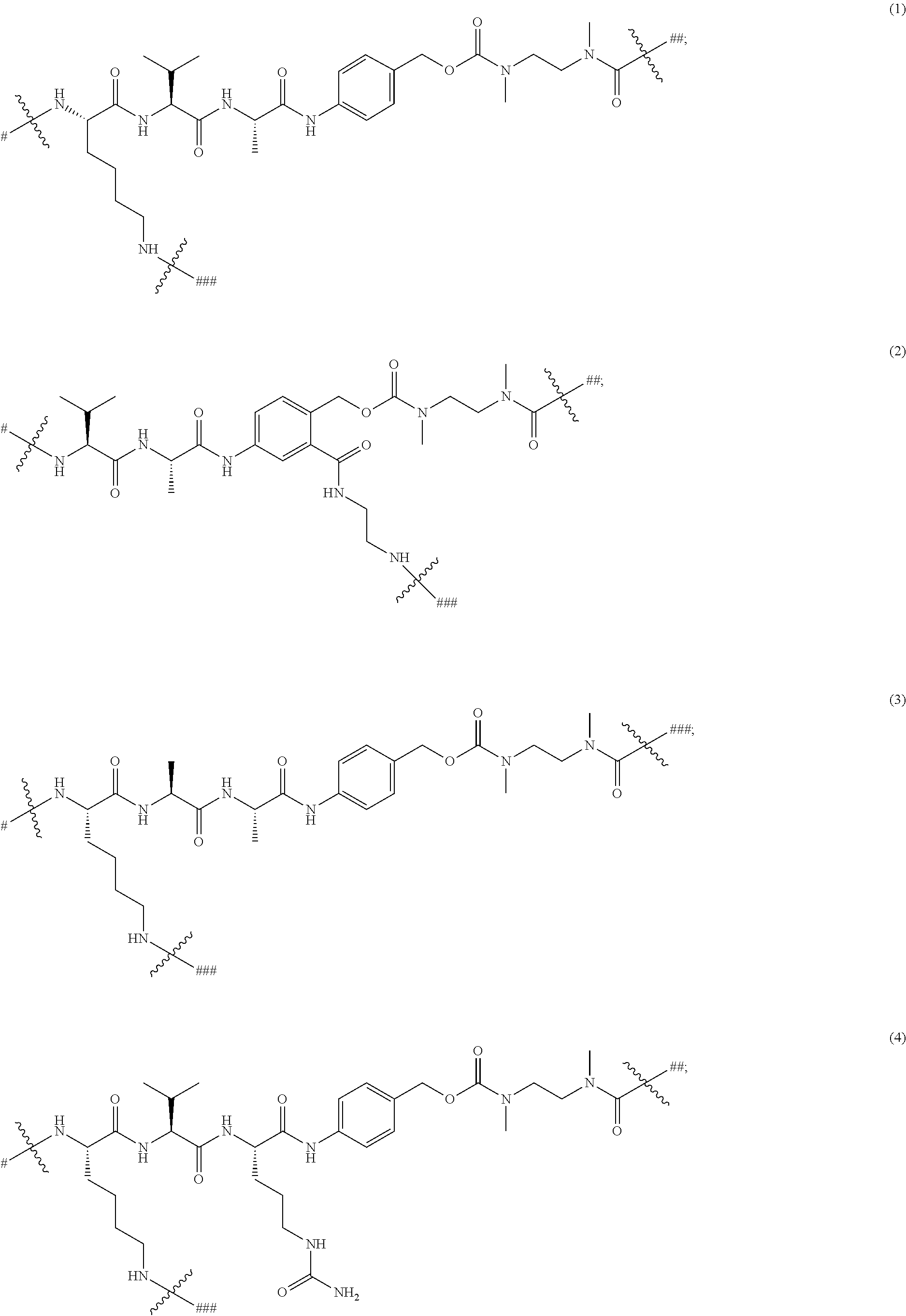

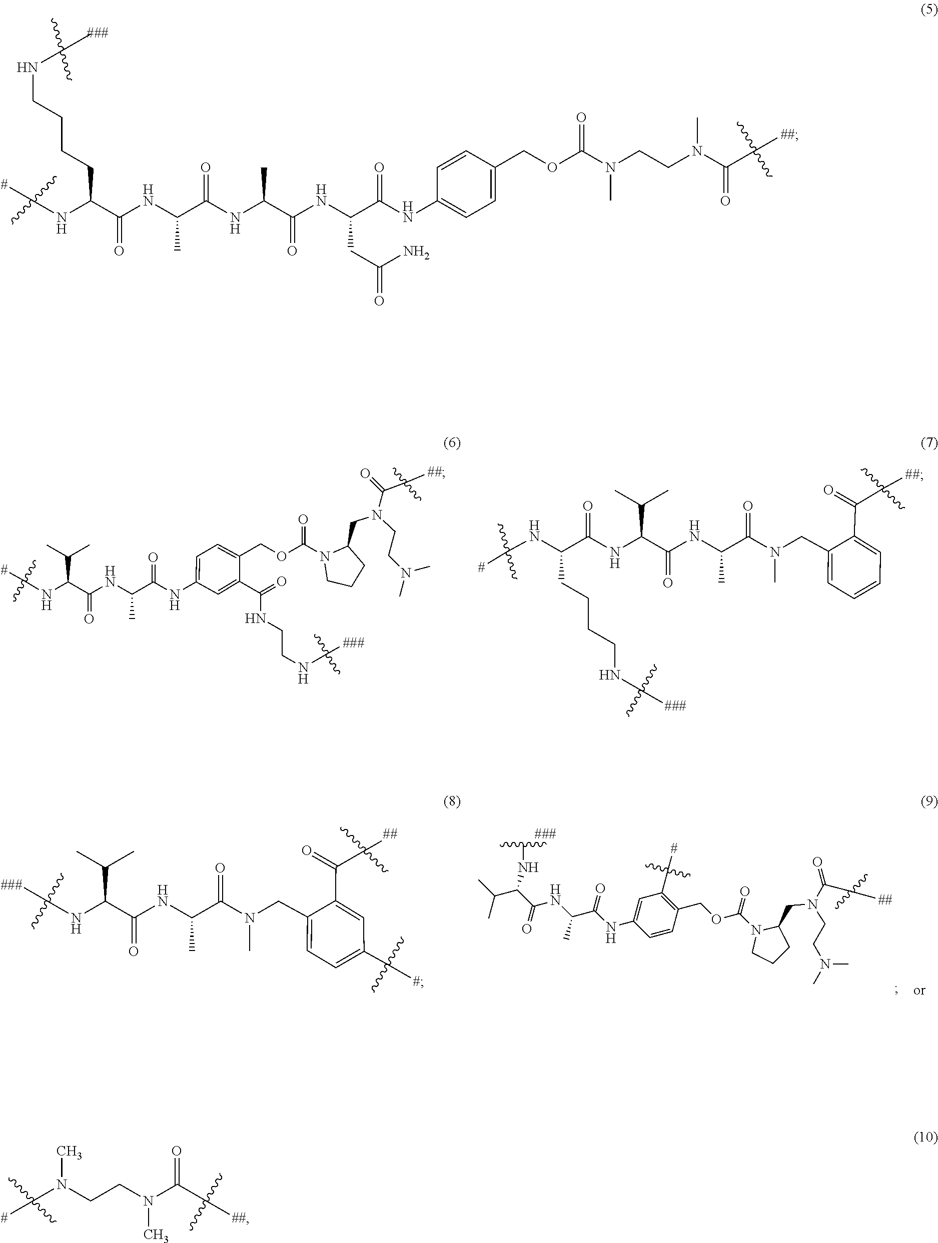

wherein:
# donates attachment to A¹ or A²;
## donates attachment to the drug moiety D; and
### donates attachment to the polymer moiety B¹.

### Variable D (Drug Moiety)

In some embodiments, the drug moiety, variable D, is a small molecule having a molecular weight preferably <about 5 kDa, more preferably <about 4 kDa, more preferably <about 3 kDa, most preferably <about 1.5 kDa or <about 1 kDa.

In one embodiment, the drug moiety has an IC₅₀ of about less than 1 nM.

In another embodiment, the drug moiety has an IC₅₀ of about greater than 1 nM, for example, the drug moiety has an IC₅₀ of about 1 to 50 nM.

Some drug moieties having an IC₅₀ of greater than about 1 nM (e.g., “less potent drugs”) are unsuitable for conjugation with an antibody using art-recognized conjugation techniques. Without wishing to be bound by any particular theory, such drug moieties have a potency that is insufficient for use in targeted antibody-drug conjugates using conventional techniques as sufficient copies of the drug (i.e., more than 8) cannot be conjugated using art-recognized techniques without resulting in diminished pharmacokinetic and physiochemical properties of the conjugate. Thus, the disclosure also relates to an antibody-drug conjugate that includes an antibody, a scaffold and one to eight drug moiety compounds, wherein the drug moiety has an IC₅₀ of greater than about 1 nM.

In some embodiment the drug moiety is a camptothecin drug moiety or a topoisomerase II inhibitor drug moiety.

### Camptothecin Drug Moiety

The term “camptothecin drug moiety” includes camptothecin itself and analogues of camptothecin. Camptothecin is a quinolone alkaloid and a topoisomerase 1 poison, which was discovered in 1966 by M. E. Wall and M. C. Wani in systematic screening of natural products for anticancer drugs. Camptothecin was isolated from the bark and stem of Camptotheca acuminata (Camptotheca, Happy tree), a tree native to China used as a cancer treatment in Traditional Chinese Medicine. It inhibits type I DNA topoisomerase, leading to DNA damage and cell death. Camptothecin has the following structure:

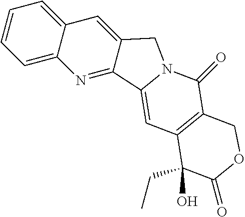 [SMILES: CC[C@@]1(O)C(=O)OCc2c1cc1n(c2=O)Cc2cc3ccccc3nc2-1]

The term “campthothecin drug moiety” also includes camptothecin analogues. In this regard, the term “camptothecin moiety” denotes any moiety that includes the structure of camptothecin:

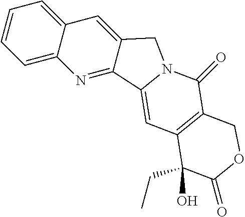 [SMILES: CC[C@@]1(O)C(=O)OCc2c1cc1n(c2=O)Cc2cc3ccccc3nc2-1]

and which may be optionally substituted. The optional substituents may include, as illustrative non-limiting examples (C₁-C₁₀)-alkyl, (C₃-C₈)-carbocyclo, (C₃-C₈)-heterocyclo, aryl, an amino group, a hydroxy group, a carbonyl group, an amide group, an ester group, a carbamate group, a carbonate group and/or a silyl group. The camptothecin drug moiety may have one or more functional group(s) which are capable of forming a bond to the linker L^{c}. A person skilled in the art will readily select a suitable camptothecin drug moiety having a desired biological activity. Camptothecin analogues have been approved and are used in cancer chemotherapy today, such as, for example, topotecan, irinotecan, belotecan or exatecan,

In some embodiments, the following camptothecin analogs are envisioned by the term camptothecin drug moiety of Formula (VI), or a prodrug, solvate, pharmaceutically acceptable salt thereof, wherein the compound of Formula (VI) is:

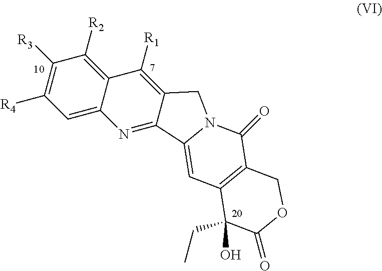

| Analogue | R₁ | R₂ | R₃ | R₄ |
|---|---|---|---|---|
| Camptothecin | —H | —H | —H | —H |
| 10-hydroxy- | —H | —H | —OH | —H |
| camptothecin | | | | |
| 7-ethyl camptothecin | 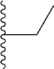 [SMILES: *CC] | —H | —H | —H |
| SN-38 | 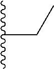 [SMILES: *CC] | —H | —OH | —H |
| Topotecan | —H | 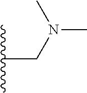 [SMILES: *CN(C)C] | —OH | —H |
| Irinotecan (CPT-11) | 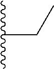 [SMILES: *CC] | —H | 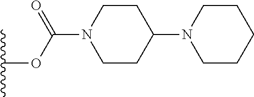 [SMILES: *OC(=O)N1CCC(N2CCCCC2)CC1] | —H |
| Silatecan (DB-67, AR-67) | 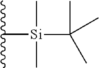 [SMILES: *[Si](C)(C)C(C)(C)C] | —H | —OH | —H |
| Cositecan (BNP-1350) | 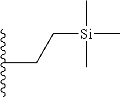 [SMILES: *CC[Si](C)(C)C] | —H | —H | —H |
| Exatecan | 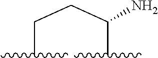 [SMILES: *CC[C@@H](*)N] | | —CH₃ | —F |
| Lurotecan | 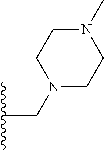 [SMILES: *CN1CCN(C)CC1] | —H | 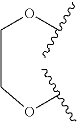 [SMILES: *OCCO*] | |
| Gimatecan (ST1481) | 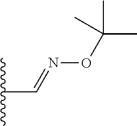 [SMILES: */C=N/OC(C)(C)C] | —H | —H | —H |
| Belotecan (CKD-602) | 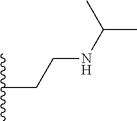 [SMILES: *CCNC(C)C] | —H | —H | —H |
| Rubitecan | —H | 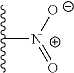 [SMILES: *[N+](=O)[O-]] | —H | —H |

| | | | |
|---|---|---|---|
| Topoisomerase I inhibitor 8 | 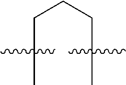 [SMILES: *CCC*] | —CH₃ | —F |

Additional camptothecin analogues that may be used in the polymer-ADCs of the present disclosure are described in International Publication No. WO2019236954A1 and European Patent No. 0495432, each of which is incorporated herein by reference in its entirety.

In some embodiments, the compound of Formula (VI) may be SN38, camptothecin, 10-hydroxy-camptothecin, 7-ethyl camptothecin, exatecan, topotecan, irinotecan, belotecan, lurtotecan, rubitecan, silatecan, cositecan, gimatecan and topoisomerase I inhibitor 8.

In other embodiments, the compound of Formula (VI) may be camptothecin, SN38, 7-ethyl camptothecin, 10-hydroxy-camptothecin, topotecan, irinotecan, exatecan, belotecan and topoisomerase I inhibitor 8.

In still other embodiments, the compound of Formula (VI) may be camptothecin, SN38, 7-ethyl camptothecin, 10-hydroxy-camptothecin, exatecan and topoisomerase I inhibitor 8.

In some embodiments, the compound of Formula (VI) is selected from those described in Table A1.

TABLE A1

| | |
|---|---|
| 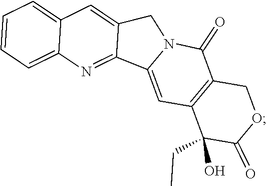 [SMILES: CC[C@@]1(O)C(=O)OCc2c1cc1n(c2=O)Cc2cc3ccccc3nc2-1] | (VI-A) |
| 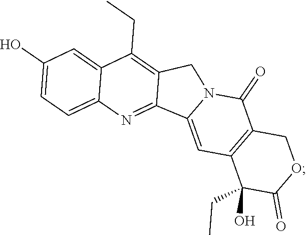 [SMILES: CCc1c2c(nc3ccc(O)cc13)-c1cc3c(c(=O)n1C2)COC(=O)[C@]3(O)CC] | (VI-B) |
| 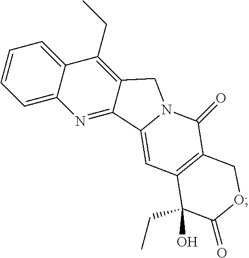 [SMILES: CCc1c2c(nc3ccccc13)-c1cc3c(c(=O)n1C2)COC(=O)[C@]3(O)CC] | (VI-C) |
| 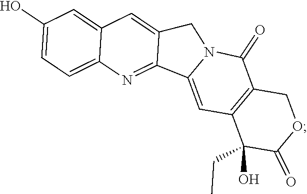 [SMILES: CC[C@@]1(O)C(=O)OCc2c1cc1n(c2=O)Cc2cc3cc(O)ccc3nc2-1] | (VI-D) |
| 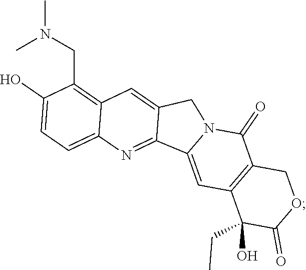 [SMILES: CC[C@@]1(O)C(=O)OCc2c1cc1n(c2=O)Cc2cc3c(CN(C)C)c(O)ccc3nc2-1] | (VI-E) |
| 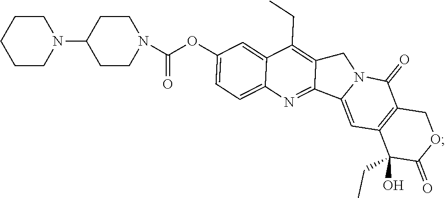 [SMILES: CCc1c2c(nc3ccc(OC(=O)N4CCC(N5CCCCC5)CC4)cc13)-c1cc3c(c(=O)n1C2)COC(=O)[C@]3(O)CC] | (VI-F) |
| 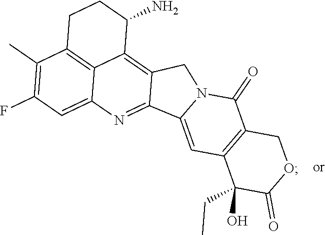 [SMILES: CC[C@@]1(O)C(=O)OCc2c1cc1n(c2=O)Cc2c-1nc1cc(F)c(C)c3c1c2[C@@H](N)CC3] | (VI-G) |
| 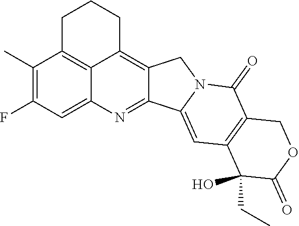 [SMILES: CC[C@@]1(O)C(=O)OCc2c1cc1n(c2=O)Cc2c-1nc1cc(F)c(C)c3c1c2CCC3] | (VI-H) |

or a prodrug, solvate, or a pharmaceutically acceptable salt thereof.

In some embodiments, the compound of Formula (VI) is covalently attached to the linker moiety (L^{c}) via any suitable attachment site on the compound.

In some embodiments, the compound of Formula (VI) is covalently attached to L^{c} via any suitable attachment site on the compound via a hydroxyl group wherein a hydrogen atom of a hydroxyl group is replaced with a bond to form a bond to L^{c}.

In some embodiments, the compound of Formula (VI) is covalently attached to L^{c} via the hydroxyl group at position C-10 of the compound, wherein the hydrogen atom of the hydroxyl group at position C-10 is replaced with a bond to form a bond to L^{c}.

In some embodiments, the compound of Formula (VI) is covalently attached to L^{c} via the hydroxyl group at position C-20 of the compound, wherein the hydrogen atom of the hydroxyl group at position C-20 is replaced with a bond to form a bond to Lc.

In some embodiments, the compound of Formula (VI) for covalent attachment to linker moiety (L^{C}) is via the hydroxyl group at position C-10 or at position C-20 of the compound, wherein the hydrogen atom of the hydroxyl group at position C-10 or at position C-20 is replaced with a bond to form a bond to linker moiety (L^{C}). is selected from those described in Table A2.

TABLE A2

| | |
|---|---|
| 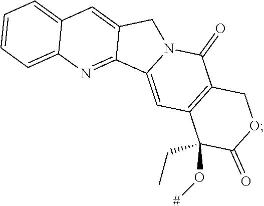 | (VI-A1) |
| 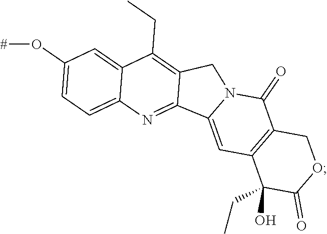 | (VI-B1) |
| 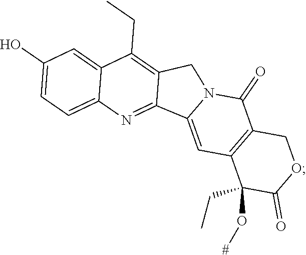 | (VI-B2) |
| 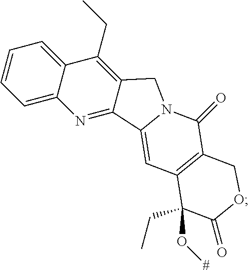 | (VI-C) |
| 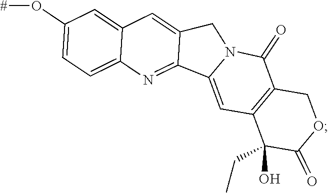 | (VI-D1) |
| 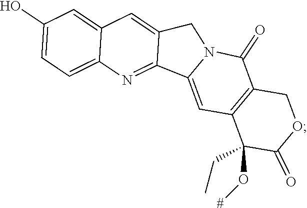 | (VI-D2) |
| 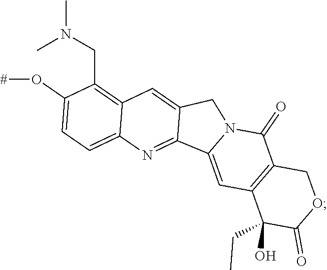 | (VI-E1) |
| 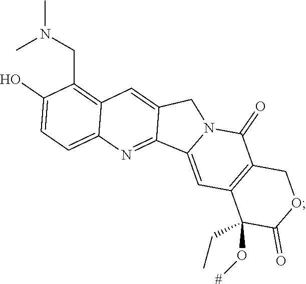 | (VI-E2) |
| 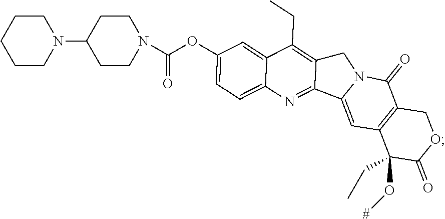 | (VI-F1) |
| 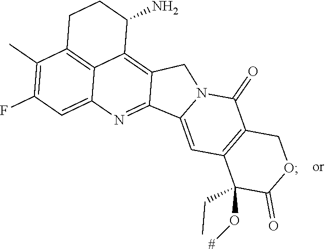 | (VI-G1) |
| 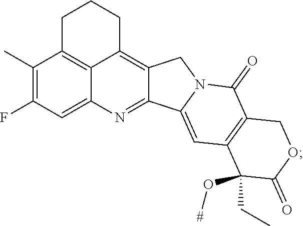 | (VI-H1) |

or a prodrug, solvate, a pharmaceutically acceptable salt; and
# donates attachment to the linker moiety L^{c}.

### Topoisomerase II Inhibitor Drug Moiety

In some embodiments, the drug moiety is a topoisomerase II inhibitor drug moiety. In other embodiments, the topoisomerase II inhibitor drug moiety is an anthracycline derivative of Formula (VII), or a prodrug, solvate, pharmaceutically acceptable salt thereof, wherein the compound of Formula (VII) is:

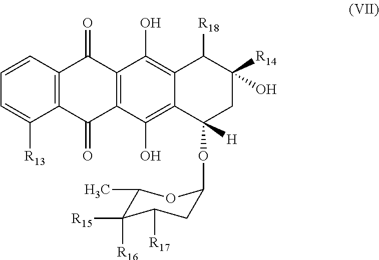

wherein:
R₁₃ is H, hydroxy or (C₁-C₆)-alkoxy;
R₁₄ is (C₁-C₆)-alkyl or —C(O)—R₂₀;
R₂₀ is (C₁-C₆)-alkyl, (C₁-C₆)-hydroxyalkyl or (C₁-C₆)-alkanoyloxy (C₁-C₆)-alkyl; one of R₁₅ and R₁₆ is H; and the other is H, hydroxy or a tetrahydropyran-2-yloxy (OTHP) group;
R₁₇ is —N(R₁₉)₂,

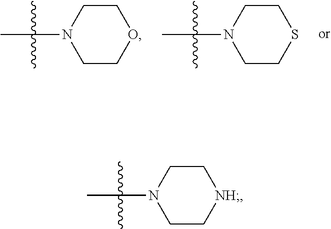 [SMILES: *N1CCOCC1 ; *N1CCSCC1 ; *N1CCNCC1]

each R₁₉ is independently H, or (C₁-C₄)-alkyl; and
   R₁₈ is H or —COOCH₃

In some embodiments, R₁₃ is H, hydroxy or —OCH₃.

In other embodiments, R₁₄ is ethyl.

In still other embodiments, R₁₄ is —C(O)—R₂₀, wherein R₂₀ is methyl, hydroxymethyl, diethoxyacetoxymethyl or butyryloxymethyl.

In some embodiments, R₁₅ is H and R₁₆ is hydroxy, or a tetrahydropyran-2-yloxy (OTHP) group;

In other embodiments, R₁₇ is NH₂, N(CH₃)₂, N(CH₂—CH₃)₂ or a morpholine group.

In still other embodiments, R₁₈ is H or —COOCH₃

In some embodiments, the compound of topoisomerase II inhibitor drug moiety of Formula (VII), or a prodrug, solvate, pharmaceutically acceptable salt thereof,

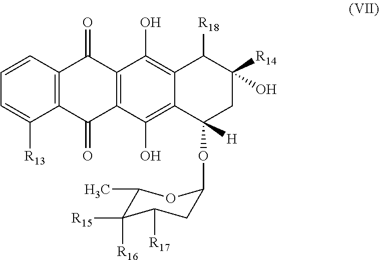

| Analogue | R₁₃ | R₁₄ | R₁₅ | R₁₆ | R₁₇ | R₁₈ |
|---|---|---|---|---|---|---|
| Doxorubicin | —OCH₃ | —C(O)CH₂OH | —H | —OH | —NH₂ | —H |
| Dimethyldoxorubicin | —OCH₃ | —C(O)CH₂OH | —H | —OH | —N(CH₃)₂ | —H |
| Diethyldoxorubicin | —OCH₃ | —C(O)CH₂OH | —H | —OH | —N(C₂H₅)₂ | —H |
| Daunorubicin | —OCH₃ | —C(O)CH₃ | —H | —OH | —NH₂ | —H |
| Dimethyldaunorubicin | —OCH₃ | —C(O)CH₃ | —H | —OH | —N(CH₃)₂ | —H |
| Epirubicin | —OCH₃ | —C(O)CH₂OH | —OH | —H | —NH₂ | —H |
| Dimethylepirubicin | —OCH₃ | —C(O)CH₂OH | —OH | —H | —N(CH₃)₂ | —H |
| Diethylepirubicin | —OCH₃ | —C(O)CH₂OH | —OH | —H | —N(C₂H₅)₂ | —H |
| Idarubicin | —H | —C(O)CH₃ | —H | —OH | —NH₂ | —H |
| Dimethylidarubicin | —H | —C(O)CH₃ | —H | —OH | —N(CH₃)₂ | —H |
| Diethylidarubicin | —H | —C(O)CH₃ | —H | —OH | —N(C₂H₅)₂ | —H |
| THP | —OCH₃ | —C(O)CH₂OH | —H | —OTHP | —NH₂ | —H |
| Esorubicin | —OCH₃ | —C(O)CH₂OH | —H | H | —NH₂ | —H |
| Detorubicin | —OCH₃ | —C(O)CH₂O— | —H | —OH | —NH₂ | —H |
| | | C(O)CH(OC₂H₅)₂ | | | | |
| Carminorubicin | —H | —C(O)CH₂OH | —H | —OH | —NH₂ | —H |
| Aclarubicin | —OH | —C₂H₅ | —H | —(OTHP)₂ | —N(CH₃)₂ | —COOCH₃ |
| | —OCH₃ | —C(O)CH₂O— | —H | —OH | —NH₂ | —H |
| | | C(O)C₄H₉ | | | | |
| | —OCH₃ | —C(O)CH₂OH | —H | —OH | 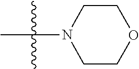 [SMILES: *N1CCOCC1] | —H |
| | —OCH₃ | —C(O)CH₂OH | —H | —OH | 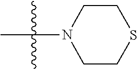 [SMILES: *N1CCSCC1] | —H |
| | —OCH₃ | —C(O)CH₂OH | —H | —OH | 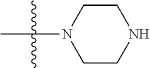 [SMILES: *N1CCNCC1] | —H |

In some embodiments, the compound of Formula (VII) may be doxorubicin, dimethyldoxorubicin, di ethyl doxorubicin, daunorubicin, dimthyldaunorubicin, epirubicin, dimethylepirubicin, di ethyl epirubicin, Idarubicin, dimethylidarubicin diethylidarubicin, or aclarubicin,

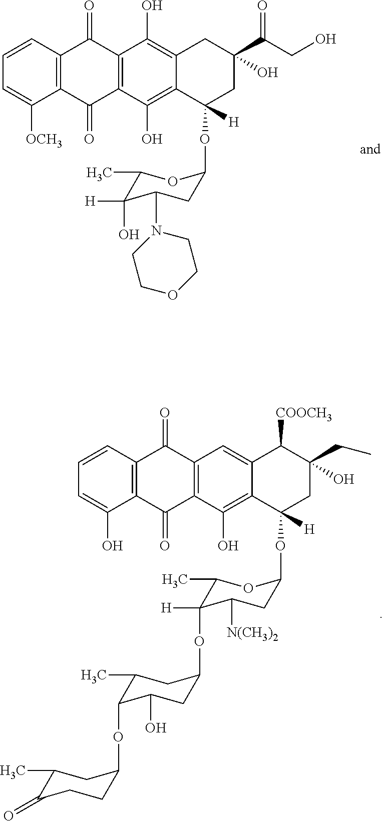 [SMILES: COc1cccc2c1C(=O)c1c(O)c3c(c(O)c1C2=O)C[C@@](O)(C(=O)CO)C[C@@H]3OC1CC(N2CCOCC2)C(O)C(C)O1 ; CC[C@@]1(O)C[C@H](OC2CC(N(C)C)C(OC3CC(C)C(OC4CCC(=O)C(C)C4)C(O)C3)C(C)O2)c2c(cc3c(c2O)C(=O)c2c(O)cccc2C3=O)[C@H]1C(=O)OC]

In some embodiments, the compound of Formula (VII) is selected from those described in Table A3.

TABLE A3

| | |
|---|---|
| 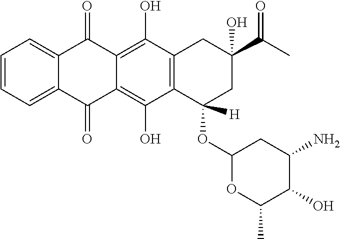 [SMILES: CC(=O)[C@]1(O)Cc2c(O)c3c(c(O)c2[C@@H](OC2C[C@H](N)[C@H](O)[C@H](C)O2)C1)C(=O)c1ccccc1C3=O] | (VII-A) |
| 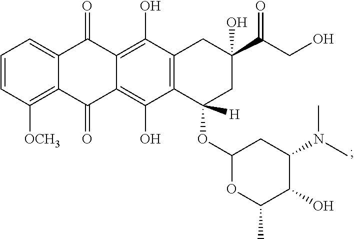 [SMILES: COc1cccc2c1C(=O)c1c(O)c3c(c(O)c1C2=O)C[C@@](O)(C(=O)CO)C[C@@H]3OC1C[C@H](N(C)C)[C@H](O)[C@H](C)O1] | (VII-B) |
| 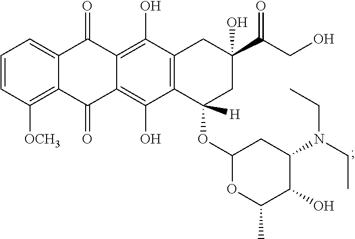 [SMILES: CCN(CC)[C@H]1CC(O[C@H]2C[C@](O)(C(=O)CO)Cc3c(O)c4c(c(O)c32)C(=O)c2c(OC)cccc2C4=O)O[C@@H](C)[C@H]1O] | (VII-C) |
| 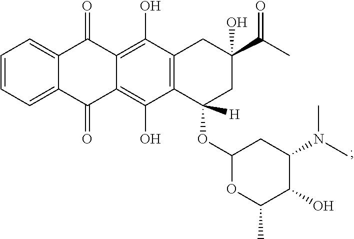 [SMILES: CC(=O)[C@]1(O)Cc2c(O)c3c(c(O)c2[C@@H](OC2C[C@H](N(C)C)[C@H](O)[C@H](C)O2)C1)C(=O)c1ccccc1C3=O] | (VII-D) |
| 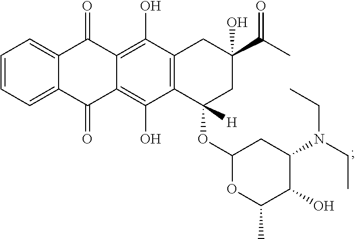 [SMILES: CCN(CC)[C@H]1CC(O[C@H]2C[C@](O)(C(C)=O)Cc3c(O)c4c(c(O)c32)C(=O)c2ccccc2C4=O)O[C@@H](C)[C@H]1O] | (VII-E) |
| 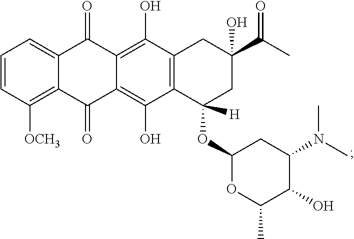 [SMILES: COc1cccc2c1C(=O)c1c(O)c3c(c(O)c1C2=O)C[C@@](O)(C(C)=O)C[C@@H]3O[C@H]1C[C@H](N(C)C)[C@H](O)[C@H](C)O1] | (VII-F) |
| 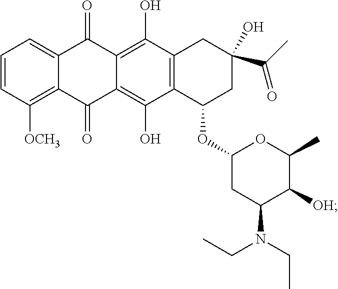 [SMILES: CCN(CC)[C@H]1C[C@H](O[C@H]2C[C@](O)(C(C)=O)Cc3c(O)c4c(c(O)c32)C(=O)c2c(OC)cccc2C4=O)O[C@@H](C)[C@H]1O] | (VII-G) |
| 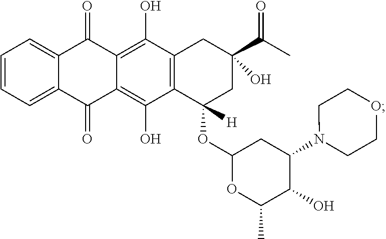 [SMILES: CC(=O)[C@]1(O)Cc2c(O)c3c(c(O)c2[C@@H](OC2C[C@H](N4CCOCC4)[C@H](O)[C@H](C)O2)C1)C(=O)c1ccccc1C3=O] | (VII-H) |
| 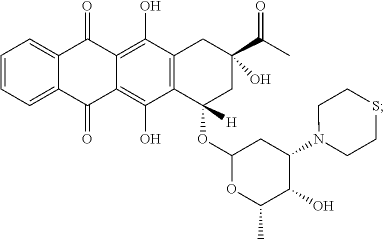 [SMILES: CC(=O)[C@]1(O)Cc2c(O)c3c(c(O)c2[C@@H](OC2C[C@H](N4CCSCC4)[C@H](O)[C@H](C)O2)C1)C(=O)c1ccccc1C3=O] | (VII-I) |
| 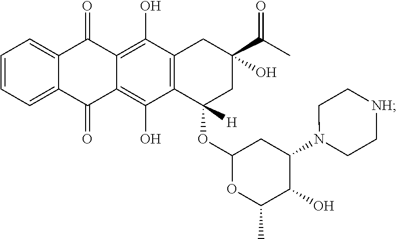 [SMILES: CC(=O)[C@]1(O)Cc2c(O)c3c(c(O)c2[C@@H](OC2C[C@H](N4CCNCC4)[C@H](O)[C@H](C)O2)C1)C(=O)c1ccccc1C3=O] | (VII-J) |

or a prodrug, solvate, a pharmaceutically acceptable salt;

In some embodiments, the compound of Formula (VII) is covalently attached to linker moiety (L^{c}) via any suitable attachment site on the compound.

In some embodiments, the compound of Formula (VII) is covalently attached to linker moiety (L^{c}) via any suitable attachment site on the compound. via a hydroxyl group wherein a hydrogen atom of a hydroxyl group is replaced with a bond to form a bond to L^{c}.

In some embodiments, the compound of Formula (VII) for covalent attachment to linker moiety (L^{c}) via a hydroxyl group, wherein the hydrogen atom of the hydroxyl group is replaced with a bond to form a bond to linker moiety (L^{c}), is selected from those described in Table A4.

TABLE A4

| | |
|---|---|
| 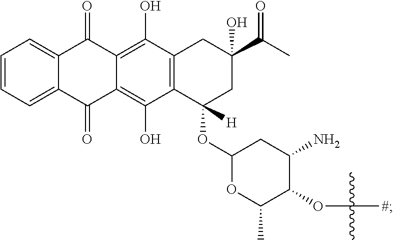 | (VII-A1) |
| 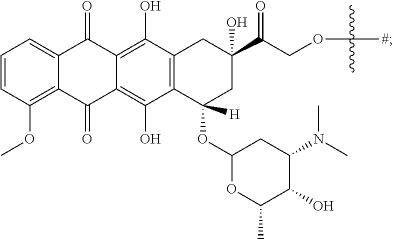 | (VII-B1) |
| 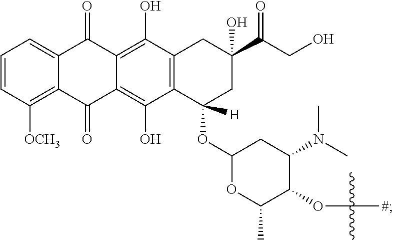 | (VII-B2) |
| 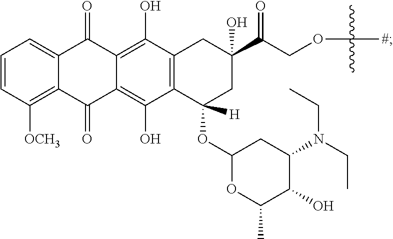 | (VII-C1) |
| 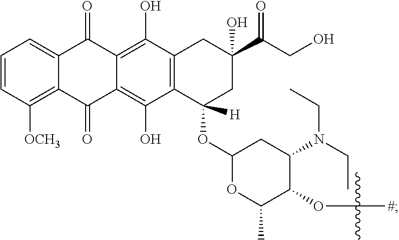 | (VII-C2) |
| 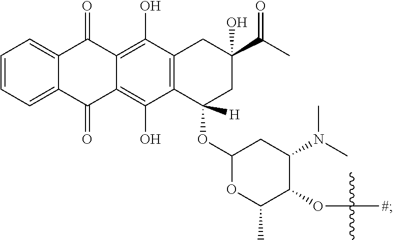 | (VII-D1) |
| 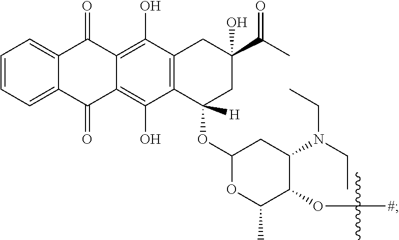 | (VII-E1) |
| 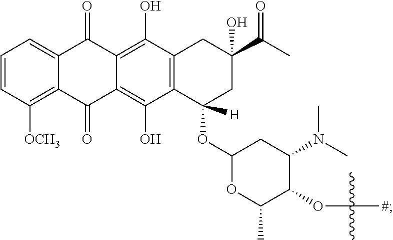 | (VII-F1) |
| 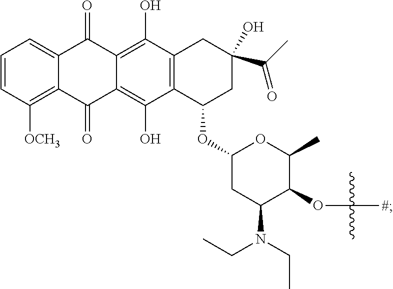 | (VII-G1) |
| 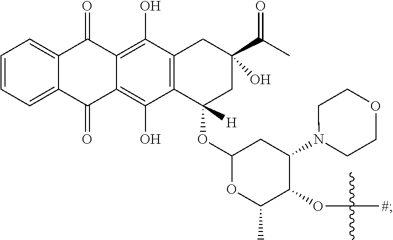 | (VII-H1) |
| 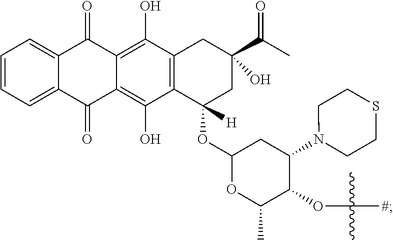 | (VII-I1) |
| 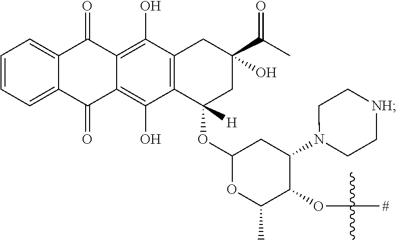 | (VII-J1) |

or a prodrug, solvate, pharmaceutically acceptable salt thereof, and
# donates attachment to the linker moiety L^{c}.

In some embodiments, the drug-linker moiety prior to linking to a recognition moiety and the POZ polymer are selected from those described in Table A5.

TABLE A5

| | |
|---|---|
| 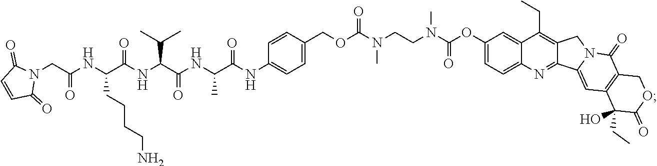 [SMILES: CCc1c2c(nc3ccc(OC(=O)N(C)CCN(C)C(=O)OCc4ccc(NC(=O)[C@H](C)NC(=O)[C@@H](NC(=O)[C@H](CCCCN)NC(=O)CN5C(=O)C=CC5=O)C(C)C)cc4)cc13)-c1cc3c(c(=O)n1C2)COC(=O)[C@]3(O)CC] | (1) |
| 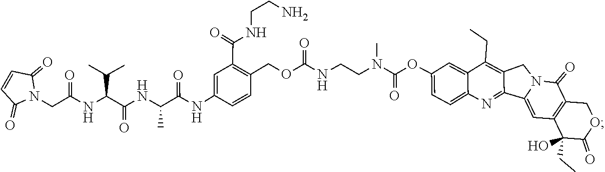 [SMILES: CCc1c2c(nc3ccc(OC(=O)N(C)CCNC(=O)OCc4ccc(NC(=O)[C@H](C)NC(=O)[C@@H](NC(=O)CN5C(=O)C=CC5=O)C(C)C)cc4C(=O)NCCN)cc13)-c1cc3c(c(=O)n1C2)COC(=O)[C@]3(O)CC] | (2) |
| 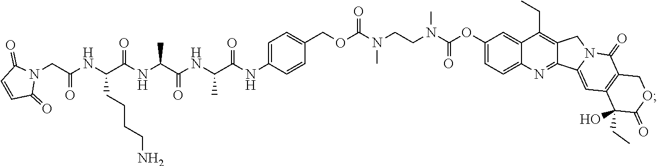 [SMILES: CCc1c2c(nc3ccc(OC(=O)N(C)CCN(C)C(=O)OCc4ccc(NC(=O)[C@H](C)NC(=O)[C@H](C)NC(=O)[C@H](CCCCN)NC(=O)CN5C(=O)C=CC5=O)cc4)cc13)-c1cc3c(c(=O)n1C2)COC(=O)[C@]3(O)CC] | (3) |
| 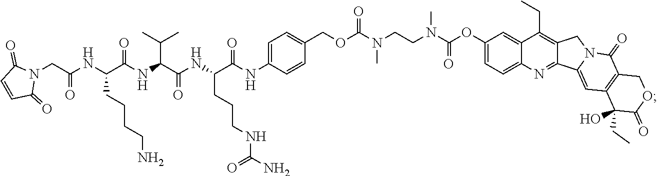 [SMILES: CCc1c2c(nc3ccc(OC(=O)N(C)CCN(C)C(=O)OCc4ccc(NC(=O)[C@H](CCCNC(N)=O)NC(=O)[C@@H](NC(=O)[C@H](CCCCN)NC(=O)CN5C(=O)C=CC5=O)C(C)C)cc4)cc13)-c1cc3c(c(=O)n1C2)COC(=O)[C@]3(O)CC] | (4) |
| 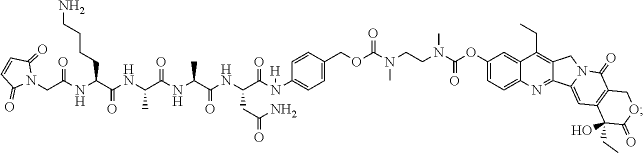 [SMILES: CCc1c2c(nc3ccc(OC(=O)N(C)CCN(C)C(=O)OCc4ccc(NC(=O)[C@H](CC(N)=O)NC(=O)[C@H](C)NC(=O)[C@H](C)NC(=O)[C@H](CCCCN)NC(=O)CN5C(=O)C=CC5=O)cc4)cc13)-c1cc3c(c(=O)n1C2)COC(=O)[C@]3(O)CC] | (5) |
| 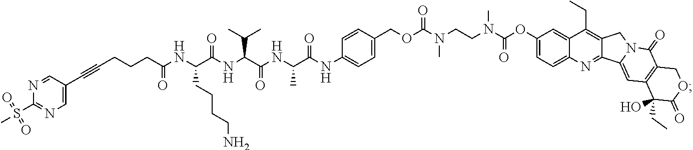 [SMILES: CCc1c2c(nc3ccc(OC(=O)N(C)CCN(C)C(=O)OCc4ccc(NC(=O)[C@H](C)NC(=O)[C@@H](NC(=O)[C@H](CCCCN)NC(=O)CCCC#Cc5cnc(S(C)(=O)=O)nc5)C(C)C)cc4)cc13)-c1cc3c(c(=O)n1C2)COC(=O)[C@]3(O)CC] | (6) |
| 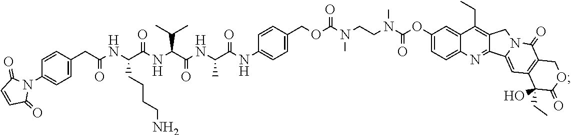 [SMILES: CCc1c2c(nc3ccc(OC(=O)N(C)CCN(C)C(=O)OCc4ccc(NC(=O)[C@H](C)NC(=O)[C@@H](NC(=O)[C@H](CCCCN)NC(=O)Cc5ccc(N6C(=O)C=CC6=O)cc5)C(C)C)cc4)cc13)-c1cc3c(c(=O)n1C2)COC(=O)[C@]3(O)CC] | (7) |
| 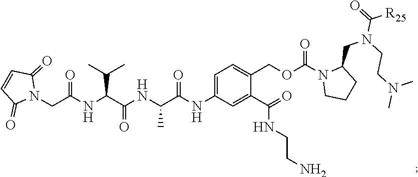 | (8) |
| 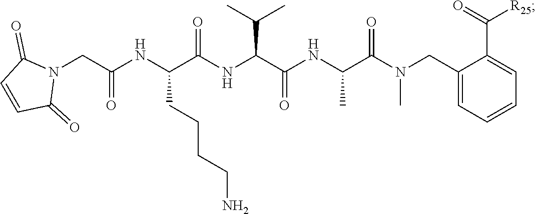 | (9) |
| 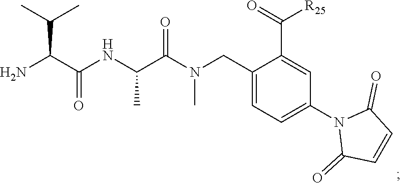 | (10) |
| 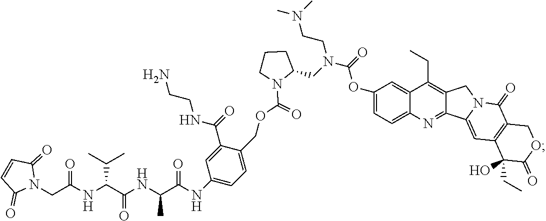 [SMILES: CCc1c2c(nc3ccc(OC(=O)N(CCN(C)C)C[C@H]4CCCN4C(=O)OCc4ccc(NC(=O)[C@@H](C)NC(=O)[C@H](NC(=O)CN5C(=O)C=CC5=O)C(C)C)cc4C(=O)NCCN)cc13)-c1cc3c(c(=O)n1C2)COC(=O)[C@]3(O)CC] | (11) |
| 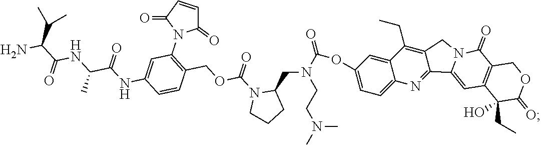 [SMILES: CCc1c2c(nc3ccc(OC(=O)N(CCN(C)C)C[C@H]4CCCN4C(=O)OCc4ccc(NC(=O)[C@H](C)NC(=O)[C@@H](N)C(C)C)cc4N4C(=O)C=CC4=O)cc13)-c1cc3c(c(=O)n1C2)COC(=O)[C@@]3(O)CC] | (12) |
| 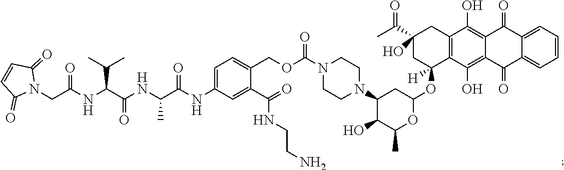 [SMILES: CC(=O)[C@]1(O)Cc2c(O)c3c(c(O)c2[C@@H](OC2C[C@H](N4CCN(C(=O)OCc5ccc(NC(=O)[C@H](C)NC(=O)[C@@H](NC(=O)CN6C(=O)C=CC6=O)C(C)C)cc5C(=O)NCCN)CC4)[C@H](O)[C@H](C)O2)C1)C(=O)c1ccccc1C3=O] | (13) |
| wherein: | |
| R₂₅ is: | |
| 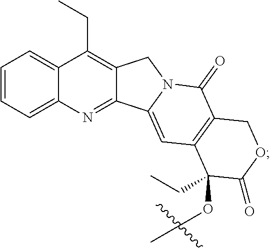 [SMILES: *O[C@]1(CC)C(=O)OCc2c1cc1n(c2=O)Cc2c-1nc1ccccc1c2CC] | |
| 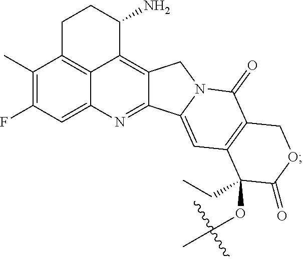 [SMILES: *O[C@]1(CC)C(=O)OCc2c1cc1n(c2=O)Cc2c-1nc1cc(F)c(C)c3c1c2[C@@H](N)CC3] | |
| or | |
| 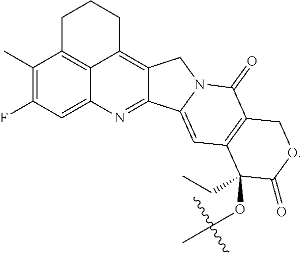 [SMILES: *O[C@]1(CC)C(=O)OCc2c1cc1n(c2=O)Cc2c-1nc1cc(F)c(C)c3c1c2CCC3] | |

### Polymer Moiety

In some embodiments, the polymer moiety of the polymer antibody drug conjugates of the present invention may be in a variety of forms. In certain embodiments, the polymer is a poly(oxazoline) (POZ), poly(5,6-dihydro-4 h-1,3-oxazine), a dextran, a dextran modified by oxidation, a polyethylene glycol (PEG), a poly(hydroxypropylmethacrylate), a polyglutamic acid, a polylactic-polyglutamic acid mixture, a polysialic acid, a polycaprolactone, a polyvinylpyrrolidone, a glycosaminoglycans, a polyglycerol, a poly(acryloyloxyethylphosphorylcholine), or a methacrylate-based co polymer with synthetic forms of phosphorylcholine; or a combination thereof.

In one embodiment, the polymer is a PEG polymer. In another embodiment, the polymer is a dextran polymer. In still another embodiment, the polymer is a dextran polymer modified by oxidation. In still yet another embodiment, the polymer is a POZ polymer. In one embodiment, the polymer is a POZ polymer.

In one embodiment, the polymer is a co-polymer. These co-polymers may be manufactured by reacting one of more monomer units of a first water-soluble polymer and one or more monomer units of at least a second polymer (which may optionally be a water-soluble polymer). Such a co-polymer includes both block co-polymers and random co-polymers. In a particular aspect, the co-polymer includes a POZ polymer and at least a second polymer. In a particular aspect, the co-polymer includes a POZ polymer and at least a second polymer, wherein the POZ polymer portion includes greater than 25%, 50%, 75%, 85%, 95%, 98%, 99% or 99.5% of the polymer (on a weight-to-weight basis with regard to the total polymer components). In a particular aspect, the co-polymer includes a POZ polymer and at least a second polymer, wherein the POZ polymer includes greater than 25%, 50%, 75%, 85%, 95%, 98%, 99% or 99.5% of the polymer (on a weight-to-weight basis with regard to the total polymer components) and at least one of the additional polymers is a water-soluble polymer. In another aspect, the co-polymer comprises a POZ polymer and at least a second polymer, wherein the POZ polymer includes greater than 50% to 99.5% of the polymer (on a weight to weight basis with regard to the total polymer components) and at least one of the additional polymers is a water-soluble polymer. In any of the foregoing, the additional water-soluble polymer(s) may be any water-soluble polymer described above. In any of the foregoing, the additional polymer(s) may be PEG, dextran, and/or dextran modified by oxidation. In a particular aspect, the co-polymer includes a POZ polymer and at least a second polymer, wherein the POZ polymer includes greater than 25%, 50%, 75%, 85%, 95%, 98%, 99% or 99.5% of the polymer (on a weight-to-weight basis) and the additional polymer(s) is not a water-soluble polymer. In another aspect, the co-polymer includes a POZ polymer and at least a second polymer, wherein the POZ polymer includes greater than 50% to 99.5% of the polymer (on a weight-to-weight basis) and the additional polymer(s) is not a water-soluble polymer.

A variety of POZ polymers may be used in the present invention. The POZ polymer may contain a single type or class of functional groups or may contain more than one type or class of functional groups. The POZ polymer may be a linear POZ polymer, a branched POZ polymer, or a multi-armed POZ polymer, wherein any of the foregoing may contain pendent groups. Various representative POZ polymers are described herein. The POZ polymer may be prepared by living cation polymerization or by other methods as is known in the art. Polymers and their methods of preparation are described in U.S. Pat. Nos. 7,943,141, 8,088,884, 8,110,651; 8,101,706, 8,383,093, 9,284,411, 10,071,168 and 11,065,340, each of which is incorporated herein by reference in its entirety. The polyoxazoline polymer may be a homopolymer; likewise, the polyoxazoline polymer may be a random or block copolymer containing one or more units of a first polyoxazoline polymer separated by one or more units of a second polyoxazoline polymer. In some embodiments, the POZ may be a homopolymer, such as, for example, poly(methyloxazoline) (PMOZ), which is quite hydrophilic, poly(ethyloxazoline) (PEOZ), which is less hydrophilic or poly(propyloxazoline), which is even less hydrophilic.

In one embodiment, the POZ polymer is prepared by living cation polymerization. Other methods known in the art may also be used to prepare the POZ polymer. As discussed in more detail below, the POZ can be conjugated directly to the drug linker moiety or may be linked to the drug linker moiety via a linker moiety. Any linker moiety suitable for coupling the POZ to a drug linker moiety can be used including, but not limited to, non-ester-containing linker moieties and ester-containing linker moieties. The POZ polymer may be conjugated to the drug linker moiety via an appropriate chemical group on the initiator or the terminal end of the polymer or via an appropriate chemical group at a pendant position on the polymer. In one embodiment, the POZ polymer may be conjugated to the drug linker moiety via an appropriate chemical group on the terminal end of the polymer.

The POZ polymers of the present invention are of increased purity and with low PD values suitable for use in pharmaceutical applications. As is known in the art, PD values will vary with MW; in general, as the molecular weight increases the PD value also increases. Using the methods described in U.S. Pat. Nos. 7,943,141, 8,088,884, 8,110,651; 8,101,706, 8,383,093, 10,071,168 and 11,065,340, each of which is incorporated herein by reference in its entirety. POZ polymers of various MWs can be produced on commercial scale with lower PD values at a given MW. In one embodiment, the POZ polymer portion has a molecular weight of about 500 to about 5,000 Daltons. In another embodiment, the POZ polymer portion has a molecular weight of about 1,000 Daltons about 2,500 Daltons. In yet another embodiment, the POZ polymer portion has a molecular weight of about 2,000 Daltons to about 5,000 Daltons. In yet another embodiment, the POZ polymer portion has a molecular weight of about 1,000 Daltons. In yet another embodiment, the POZ polymer portion has a molecular weight of about 2,000 Daltons. In yet another embodiment, the POZ polymer portion has a molecular weight of about 5,000 Daltons. In such embodiments, the POZ polymer chain has a polydispersity value of less than or equal to 1.2, less than or equal to 1.1, or less than or equal to 1.0. In one embodiment, the POZ polymer chain has a polydispersity value of 1.0. Methods of synthesizing polyoxazoline polymers and derivatives thereof with low PD values are discussed in International Application Nos. PCT/US2008/002626 and PCT/US2008/078159, each of which is incorporated herein by reference in its entirety.

In one embodiment, the POZ polymer contains at least one reactive group capable of forming a linkage to the drug linker moiety. Any linkage moiety suitable for coupling the POZ polymer to a drug linker moiety can be used including, but not limited to, non-ester-containing linker moieties and ester-containing linker moieties. In one embodiment, the linkage is a direct linkage and the drug linker moiety is linked to the POZ polymer via a direct linkage through a reactive group on the drug linker moiety and a reactive group on the polymer. In one embodiment, the POZ polymer may be linked to the drug linker moiety via an appropriate chemical group on the initiator or the terminal end of the polymer or via an appropriate chemical group at a pendant position on the polymer. In another embodiment, the POZ polymer is linked to the drug linker moiety via an appropriate chemical group on the terminal end of the polymer. The linkage (whether a direct linkage or a linkage utilizing a linking group) is physiologically degradable. In this aspect, the linkage contains a cleavable moiety. Suitable linking groups include, but are not limited to ethers, esters, carbonate esters amines, amides, and a combination thereof. In one embodiment, the direct linkage is a cleavable moiety such that in-vivo under physiological conditions in the body of a subject, such as, but not limited to, a human, the drug linker moiety is released from the polymer after administration of the polymer antibody drug conjugate to the subject. The direct linkage forms the cleavable moiety in the reaction linking the polymer to the drug linker moiety. In yet another embodiment, the linkage between the POZ polymer and the drug linker moiety is a non-cleavable moiety. In yet another embodiment, the linkage between the POZ polymer and the drug linker moiety is an amide moiety.

In one embodiment, the POZ polymer, prior to linking to the drug-linker moiety, is a polymer of Formula (VIII-A), or a pharmaceutically acceptable salt thereof,

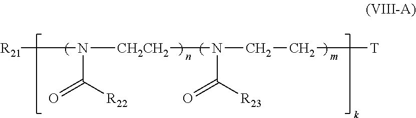

wherein
R₂₁ is an initiating group;
R₂₂ is independently selected for each repeating unit and is a pendent moiety containing an active functional group;
R₂₃ is a non-reactive pendent moiety and is independently selected for each repeating unit;
k indicates that the polymer units m and n are connected to each other in a random order;
n is an integer from 0 to 3;
m is an integer from about 1 to about 50, provided that the sum of n and m is less than or equal to 50; and
Tm is a terminating group.

In one embodiment, the POZ polymer, prior to linking to the drug-linker moiety, is a polymer of Formula (VIII-B), or a pharmaceutically acceptable salt thereof,

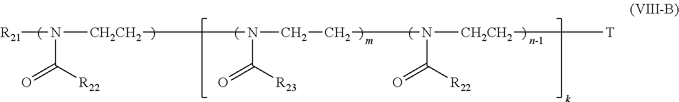

wherein:
R₂₁ is an initiating group;
R₂₂ is independently selected for each repeating unit and is a pendent moiety containing an active functional group;
R₂₃ is a non-reactive pendent moiety and is independently selected for each repeating unit;
k indicates that the polymer units m and (n−1) are connected to each other in a random order;
n is an integer from 0 to 3;
m is an integer from about 1 to about 50, provided that the sum of n and m is less than or equal to 50; and
Tm is a terminating group.

Unless otherwise specified, the following descriptions apply to each of the polymers the Formula (VIII-A), and (VIII-B).

In one embodiment, R₂₁ is H, an alkyl and a substituted alkyl. In another embodiment, R₂₁ is an alkyl group, such as, for example, a C₁-C₄ alkyl group. In a specific embodiment, R₂₁ is a methyl group. In another embodiment, R₂₁ is H. In yet another embodiment, R₂₁ is selected to lack a functional group. Additional exemplary initiating groups are disclosed in U.S. Pat. Nos. 7,943,141, 8,088,884, 8,110,651, 8,101,706, 8,883,211, and 9,284,411, and U.S. patent application Ser. Nos. 13/003,306, 13/549,312 and 13/524,994, each of which is incorporated herein by reference in its entirety.

In one embodiment of the polymers of Formula (VIII-A) and (VIII-B), R₂₃ is a non-reactive pendent moiety independently selected for each repeating unit from an unsubstituted alkyl, a substituted alkyl, an unsubstituted alkenyl, a substituted alkenyl, an unsubstituted aralkyl, a substituted aralkyl, an unsubstituted heterocyclylalkyl and a substituted heterocyclylalkyl group. In one embodiment, R₂₃ is an unsubstituted alkyl or a substituted alkyl, such as a C₁-C₄ unsubstituted alkyl or a C₁-C₄ substituted alkyl. In a particular embodiment, R₂₃ is methyl, ethyl, propyl or butyl. In one embodiment, R₂₃ is ethyl. In another embodiment, R₂₃ is methyl. In another embodiment, R₂₃ is hydroxy methyl. Exemplary R₂₃ groups are described in U.S. Pat. Nos. 7,943,141, 8,088,884, 8,110,651, 8,101,706, 8,883,211, and 9,284,411, and U.S. patent application Ser. Nos. 13/003,306, 13/549,312 and 13/524,994, each of which is incorporated herein by reference in its entirety.

In one embodiment of the polymers of Formula (VIII-A) and Formula (VIII-B), Tm is a functional group capable of forming a linkage with a drug-linker moiety. Exemplary functional groups include, but are not limited to, alkyne, amine, oxyamine, aldehyde, ketone, acetal, ketal, maleimide, ester, carboxylic acid, activated carboxylic acid (such as, but not limited to, N-hydroxysuccinimidyl (NHS) and 1-benzotriazineyl active ester), an active carbonate, a chloroformate, alcohol, azide, vinyl sulfone, isocyanate, or orthopyridyl disulfide (OPSS).

In one embodiment of the polymers of Formula (VIII-A) and Formula (VIII-B), Tm is a thioalkyl carboxylic acid, a thiocarboxylic ester, or a hydroxyl. In one embodiment, of the polymers of Formula (VIII-A) and Formula (VIII-B), Tm is Z—B₂-Q, wherein Z is S, O, or N; B₂ is an optional linking group; and Q is a functional group or a portion of a terminating group. In certain embodiments, of the polymers of Formula (VIII-A) and Formula (VIII-B), Q is non-reactive (i.e., does not contain a functional group); in other embodiments, Q contains a functional group.

Exemplary B₂ groups include, but are not limited to, alkylene groups. In a particular embodiment, B₂ is —(CH₂)₁₋₁₆. In certain embodiments, B₂ is —(CH₂)₁₋₁₀, —(CH₂)₁₋₈, —(CH₂)₁₋₆. —(CH₂)₁₋₄ or —(CH₂)₁₋₂.

In one embodiment, Q is a non-reactive functional group. In another embodiment, Q is a functional group. When Q contains a functional group, exemplary functional groups include, but are not limited to, alkyne, alkene, amine, oxyamine, aldehyde, ketone, acetal, thiol, ketal, maleimide, ester, carboxylic acid, activated carboxylic acid (such as, but not limited to, N-hydroxysuccinimidyl (NHS) and 1-benzotriazine active ester), an active carbonate, a chloroformate, alcohol, azide, vinyl sulfone, isocyanate, or orthopyridyl disulfide (OPSS). When Q contains a functional group, the functional group may be chemically orthogonal to one or more or all other functional groups present on the conjugate. When Q is a non-reactive group, any non-reactive group may be used, including, but not limited to unsubstituted alkyl and —C₆H₅. In one embodiment of the polymers of Formula (VIII-A) and Formula (VIII-B), Q is a functional group selected from —COOH, —COOCH₃, —NH₂ and NH-tBoc.

In one embodiment of the polymers of Formula (VIII-A) and (VIII-B), Z is S, B is —(CH₂)y₁- and Q is —COOH. In another specific embodiment, Z is O, B is —(CH₂)y₁- and Q is —COOH. In still another specific embodiment, Z is N, B is —(CH₂)y₁- and Q is —COOH. In any of the foregoing, y₁ is 2

In one embodiment of the polymers of Formula (VIII-A) and (VIII-B), Z is S, B is —(CH₂)y₁ and Q is —COOCH₃. In another specific embodiment, Z is O, B is —(CH₂)y₁- and Q is —COOCH₃. In still another specific embodiment, Z is N, B is —(CH₂)y₁- and Q is —COOCH₃. In any of the foregoing, y₁ is 2.

In one embodiment of the polymers of Formula (VIII-A) and (VIII-B), Z is S, B is —(CH₂)y₁- and Q is —NH₂. In another specific embodiment, Z is O, B is —(CH₂)y₁- and Q is —NH₂. In still another specific embodiment, Z is N, B is —(CH₂)y₁- and Q is —NH₂. In any of the foregoing, y1 is 2.

In one embodiment of the polymers of Formula (VIII-A) and (VIIB), Z is S, B is —(CH₂)y₁- and Q is NH-tBoc. In another specific embodiment, Z is O, B is —(CH₂)y₁ and Q is NH-tBoc. In still another specific embodiment, Z is N, B is —(CH₂)y₁- and Q is —NH-tBoc. In any of the foregoing, y₁ is 2

In a particular embodiment, Z is S. The polyoxazoline polymers containing a sulfur group as described herein may be prepared by terminating the cation at the end of the polyoxazoline polymer with a mercaptide reagent, such as, but not limited to, a mercapto-ester (for example, —S—CH₂—CH₂—CO₂CH₃ or —S—CH₂—CH₂—CO₂H), an amine (for example, —S—CH₂—CH₂—NH₂) or mercapto-protected amine (for example, —S—CH₂—CH₂—NH-tBoc). Such POZ conjugates provide for effective, large-scale purification by ion-exchange chromatography (to remove secondary amines), as well as allowing for control of polydispersity values (with polydispersity values of 1.10 or less) and for the creating of conjugates with higher molecular weight POZ polymers. In another embodiment, Z is N. In a further embodiment, Z is O.

In one embodiment of the polymers of Formula (VIII-A) and (VIIB), each R₂₂ independently is a pendent moiety. In one embodiment, R₂₂ is a pendent moiety containing an active functional group. In one embodiment when R₂₂ is a pendent moiety, wherein R₂₂ for each repeating unit is independently selected from an unsubstituted alkyl, a substituted alkyl, an unsubstituted alkenyl, a substituted alkenyl, an unsubstituted aralkyl, a substituted aralkyl, an unsubstituted heterocyclylalkyl and a substituted heterocyclylalkyl group.

In one embodiment, R₂₂ is an unsubstituted alkyl or a substituted alkyl. In a particular embodiment, R₂₂ is a pendent moiety including an active functional group. Suitable reactive functional groups include, but are not limited to, alkyne, amine, oxyamine, aldehyde, ketone, acetal, ketal, maleimide, ester, carboxylic acid, activated carboxylic acid (such as, but not limited to, N-hydroxysuccinimidyl (NHS) and 1-benzotriazineyl active ester), an active carbonate, a chloroformate, alcohol, azide, vinyl sulfone, isocyanate, and orthopyridyl disulfide (OPSS).

In certain embodiments, the active functional group of R₂₂ is chemically orthogonal to one or more or all other functional group on the conjugate. In certain embodiments, the active functional group of R₂₂ is not chemically orthogonal to one or more or all other functional group on the conjugate. In one embodiment, the active functional group of R₂₂ is an unsubstituted alkyl, a substituted alkyl, an unsubstituted alkenyl, a substituted alkenyl, an unsubstituted aralkyl, a substituted aralkyl, an unsubstituted heterocyclylalkyl, a substituted heterocyclylalkyl or an alkyne group.

In one embodiment, the active functional group of R₂₂ is an alkyne group, such as an acetylene.

In one embodiment, R₂₂ is an alkyne group i.e. an acetylene group at a pendent position of the POZ polymer. In one embodiment the acetylene group is reacted with an azido moiety via copper(I) catalyzed azide-acetylene click chemistry. In such a reaction, the azido moiety is linked to an alkyne group and a copper(I) catalyzed click reaction is executed with an alkyne group on the polymer. In one embodiment of the foregoing, the alkyne group is in a pendent position on the POZ polymer.

In the foregoing embodiments where the polymer is a POZ polymer, the POZ polymer has a molecular weight between 1000 Da and 5000 Da. In one embodiment, the POZ polymer has a molecular weight between 1000 Da and 2500 Da. In one embodiment, the POZ polymer has a molecular weight of 1000 Da. In one embodiment, the POZ polymer has a molecular weight of 2000 Da. In one embodiment, the POZ polymer has a molecular weight of 5000 Da. In the current specification, when a molecular weight is provided for a POZ polymer, unless specifically stated otherwise the molecular weight is a number average molecular weight.

Embodiments of the POZ polymer of Formula (VIII-A) and Formula (VIII-B), include, but are not limited to:

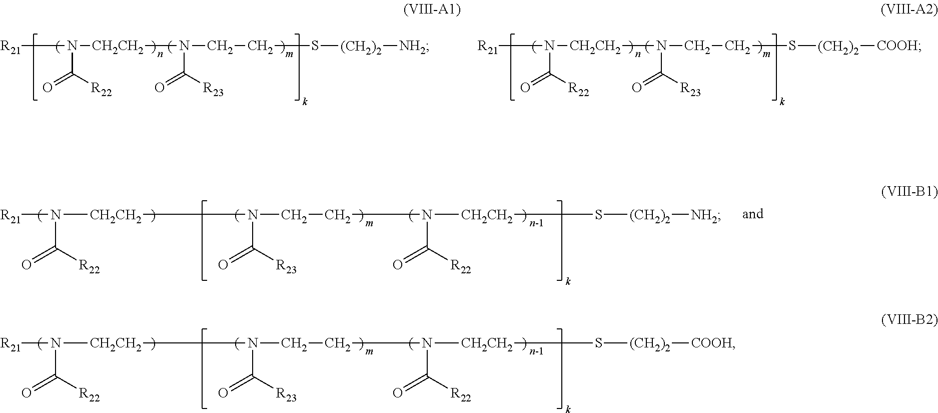

wherein R₂₁, R₂₂, R₂₃, n, m and k are as defined herein.

In one embodiment, n is an integer 0, 1, 2 or 3.

In one embodiment, n is 0

In one embodiment, n is 1.

In one embodiment, n is 2.

In one embodiment, n is 3.

In one embodiment, m is an integer from 1 to less than or equal to 50; with the proviso that n+m is 50.

In one embodiment, m is an integer from 1 to less than or equal to 40; with the proviso that n+m is 40.

In one embodiment, m is an integer from 1 to less than or equal to 30; with the proviso that n+m is 30.

In one embodiment, m is from an integer 1 to less than or equal to 20; with the proviso that n+m is 20.

In one embodiment, m is an integer from 1 to less than or equal to 10; with the proviso that n+m is 10.

In some embodiments the POZ polymer of Formula (VIII-A), is a POZ polymer of Formula (VIII-C) or Formula (VIII-D):

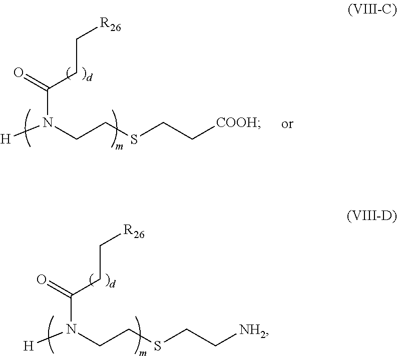

wherein
R₂₆ is H or —OH;
d is an integer 0 or 1;
m is an integer from about 10 to about 50; and
d is an integer from 0 to 3.

In some embodiments the POZ polymer of Formula (VIII-B), is a POZ polymer of Formula (VIII-E), Formula VIII-F), Formula (VIII-G) or Formula (VIII-H):

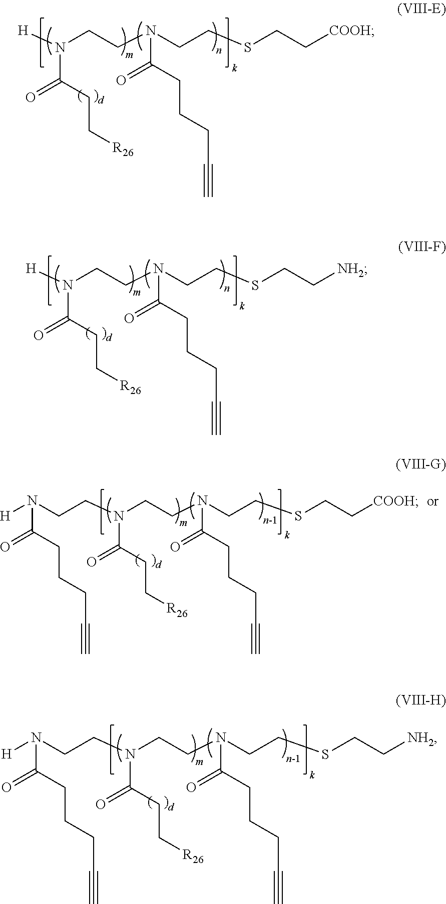

wherein
R₂ is H or OH;
k indicates that the polymer units m and n and polymer units m and (n−1) are connected to each other in a random order;
n is an integer from 1 to 3;
m is an integer from about 10 to about 50; and
d is an integer from 0 to 3.

In some embodiments, d is 0.

In some embodiments, d is 1.

In some embodiments, R₂₆ is H.

In some embodiments, R₂₆ is OH.

In some embodiments the POZ polymer is a polymer of Formula (VIII-J), Formula (VIII-K), Formula (VIII-L), Formula (VIII-M), Formula (VIII-N), Formula (VIII-O), Formula (VIII-P), Formula (VIII-Q) or Formula (VIII-R):

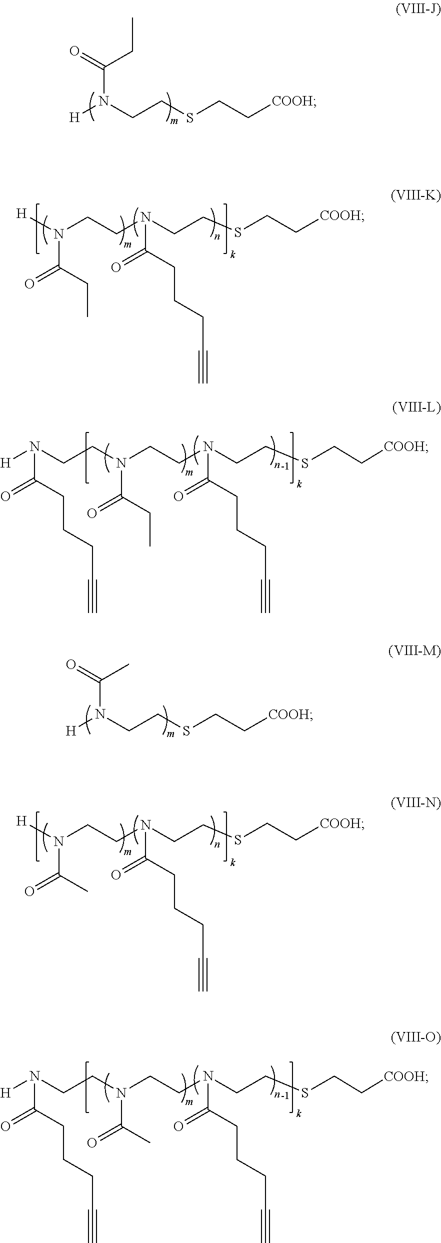

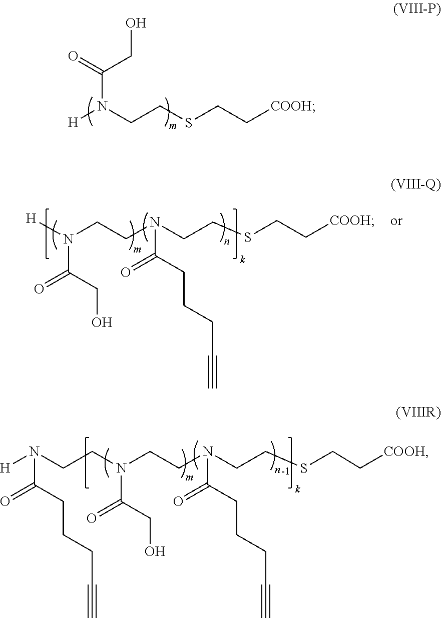

wherein:
k indicates that the polymer units m and n and polymer units m and (n−1) are connected to each other in a random order;
m is an integer from about 10 to about 50; and
n is an integer from 1 to 3.

In one embodiment, the acetylene group in the polymer of Formula (VIII-K), Formula (VIII-L), Formula (VIII-N), Formula (VIII-O), Formula (VIII-Q) or Formula (VIII-R) can be reacted with an azido moiety via copper(I) catalyzed azide-acetylene click chemistry. In such a reaction, the azido moiety is linked to the acetylene group (i.e., alkyne group) and a copper(I) catalyzed click reaction is executed with an alkyne group on the polymer. In one embodiment, the azido moiety that reacts via click chemistry with the acetylene group at a pendent position of the POZ polymer of Formula (VIII-K), Formula (VIII-L), Formula (VIII-N), Formula (VIII-O), Formula (VIII-Q) or Formula (VIII-R) is selected from those described in Table A6.

TABLE A6

| | |
|---|---|
| 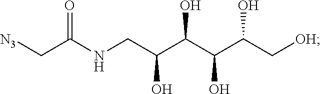 [SMILES: [N-]=[N+]=NCC(=O)NC[C@H](O)[C@@H](O)[C@H](O)[C@H](O)CO] | (1) |
| 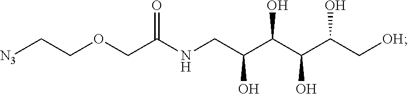 [SMILES: [N-]=[N+]=NCCOCC(=O)NC[C@H](O)[C@@H](O)[C@H](O)[C@H](O)CO] | (2) |
| 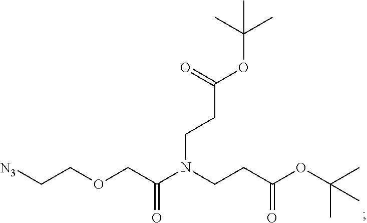 [SMILES: CC(C)(C)OC(=O)CCN(CCC(=O)OC(C)(C)C)C(=O)COCCN=[N+]=[N-]] | (3) |
| 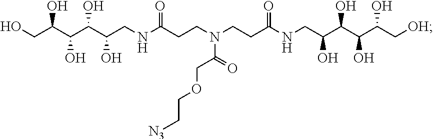 [SMILES: [N-]=[N+]=NCCOCC(=O)N(CCC(=O)NC[C@H](O)[C@@H](O)[C@H](O)[C@H](O)CO)CCC(=O)NC[C@H](O)[C@@H](O)[C@H](O)[C@H](O)CO] | (4) |
| 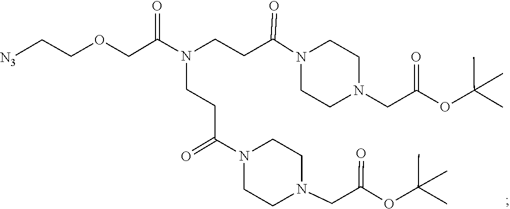 [SMILES: CC(C)(C)OC(=O)CN1CCN(C(=O)CCN(CCC(=O)N2CCN(CC(=O)OC(C)(C)C)CC2)C(=O)COCCN=[N+]=[N-])CC1] | (5) |
| 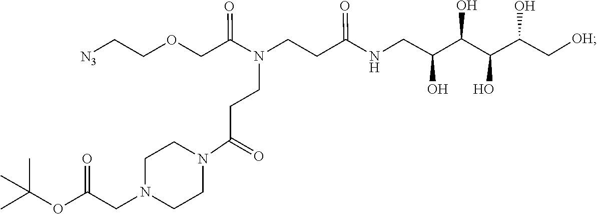 [SMILES: CC(C)(C)OC(=O)CN1CCN(C(=O)CCN(CCC(=O)NC[C@H](O)[C@@H](O)[C@H](O)[C@H](O)CO)C(=O)COCCN=[N+]=[N-])CC1] | (6) |
| 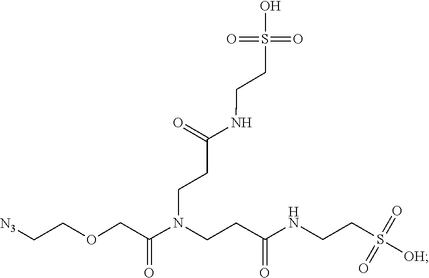 [SMILES: [N-]=[N+]=NCCOCC(=O)N(CCC(=O)NCCS(=O)(=O)O)CCC(=O)NCCS(=O)(=O)O] | (7) |
| 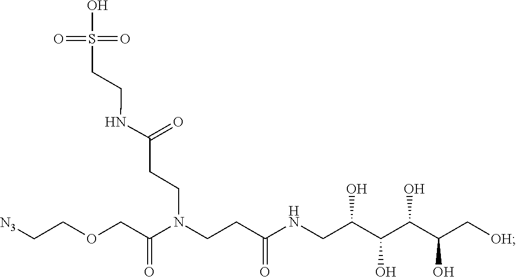 [SMILES: [N-]=[N+]=NCCOCC(=O)N(CCC(=O)NCCS(=O)(=O)O)CCC(=O)NC[C@H](O)[C@@H](O)[C@H](O)[C@H](O)CO] | (8) |
| or | |
| 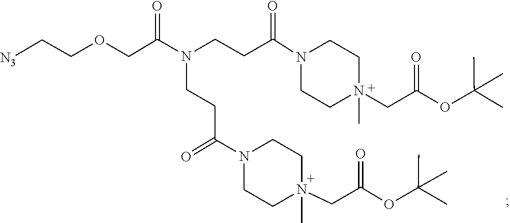 | (9) |

or an azido moiety including a carboxylic acid, zwitterionic base/carboxylic acid, polyhydroxy, or sulfonic acid group or a combination thereof. In some embodiments, both arms of the azido moiety are identical. In other embodiments, both arms of the azido moiety are not identical

In one embodiment, the azido moiety that reacts via click chemistry with the acetylene group at a pendent position of the POZ polymer of Formula (VIII-K), Formula (VIII-L), Formula (VIII-N), Formula (VIII-O), Formula (VIII-Q) or Formula (VIII-R) is:

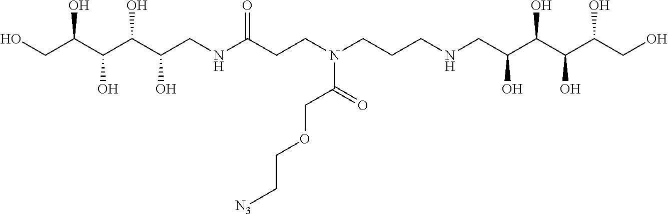 [SMILES: [N-]=[N+]=NCCOCC(=O)N(CCCNC[C@H](O)[C@@H](O)[C@H](O)[C@H](O)CO)CCC(=O)NC[C@H](O)[C@@H](O)[C@H](O)[C@H](O)CO]

In some embodiments, the acetylene group of the POZ polymer of Formula (VIII-K), Formula (VIII-L), Formula (VIII-N), Formula (VIII-O), Formula (VIII-Q) or Formula (VIII-R) is reacted with an azido moiety via copper(I) catalyzed azide-acetylene click chemistry, to form a POZ azido acetylene click polymer. In some embodiments, the POZ azido acetylene click polymer is selected from those described in Table A7.

TABLE A7

| | |
|---|---|
| 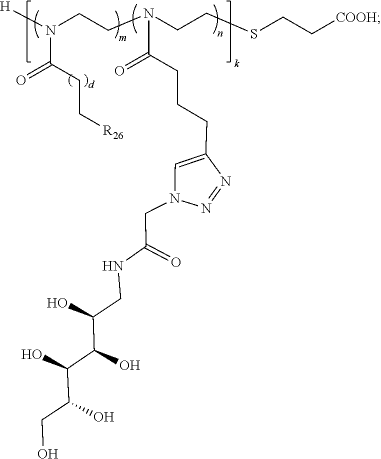 | (1) |
| 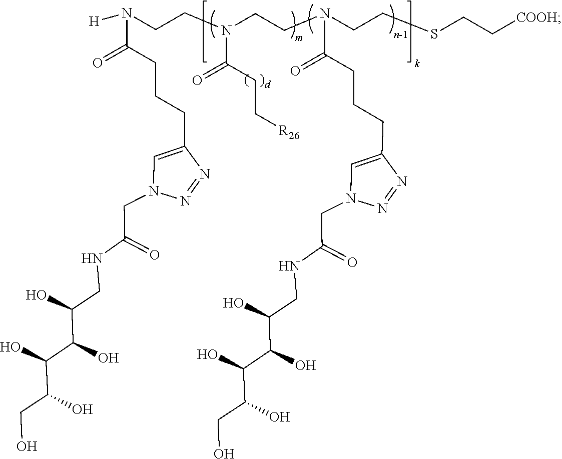 | (2) |
| 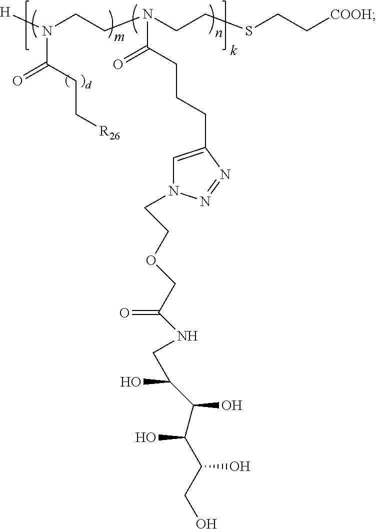 | (3) |
| 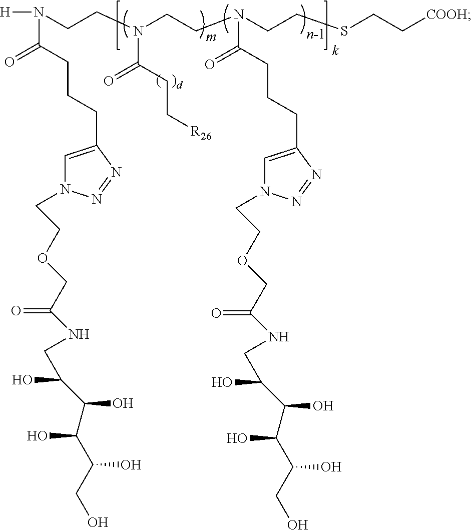 | (4) |
| 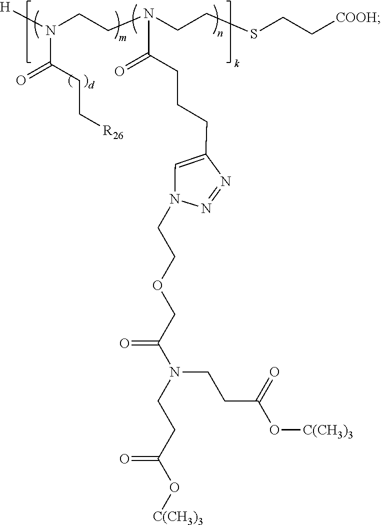 | (5) |
| 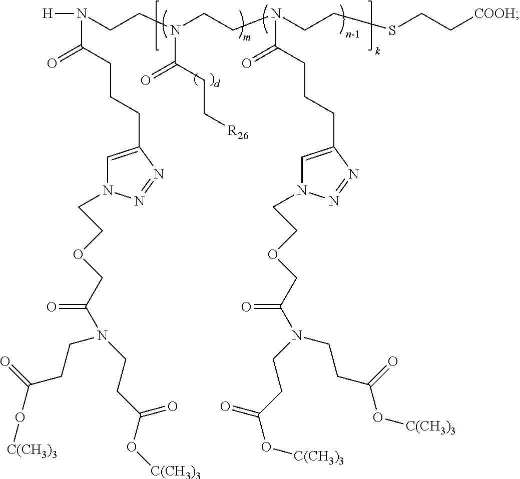 | (6) |
| 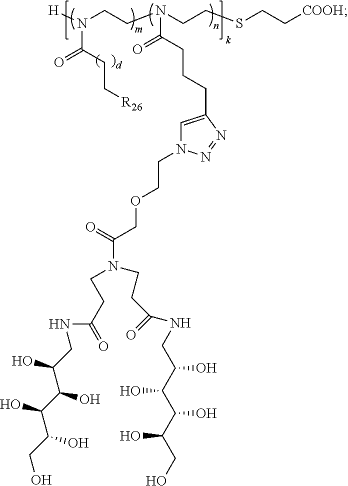 | (7) |
| 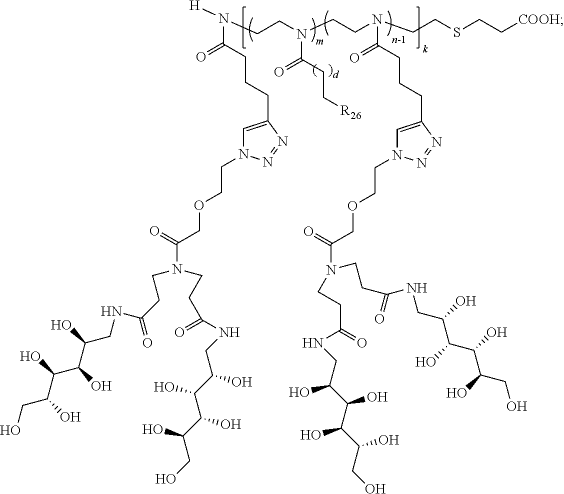 | (8) |
| 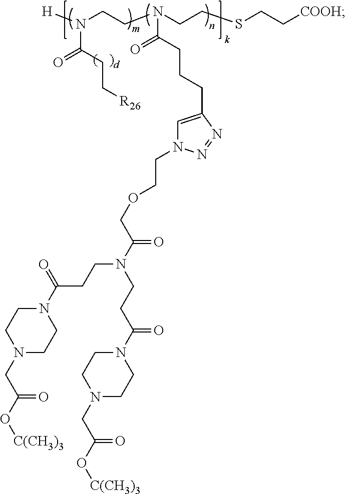 | (9) |
| 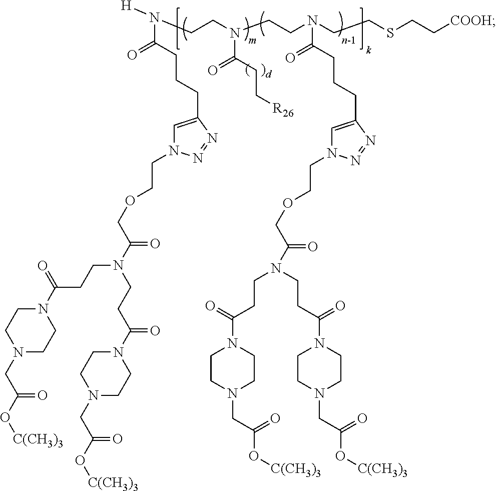 | (10) |
| 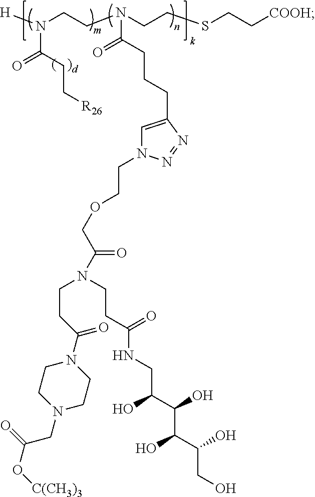 | (11) |
| 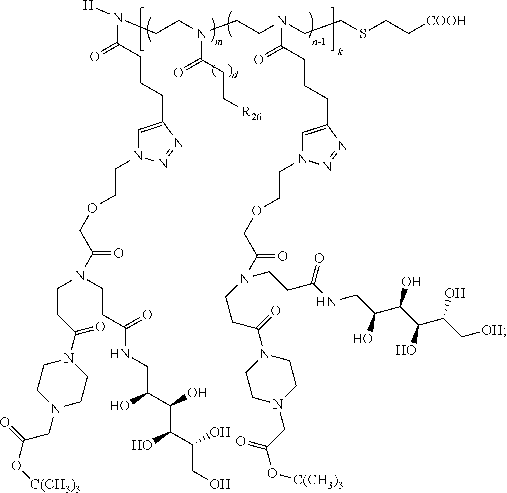 | (12) |
| 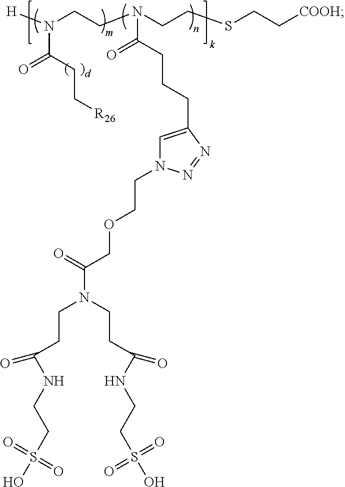 | (13) |
| 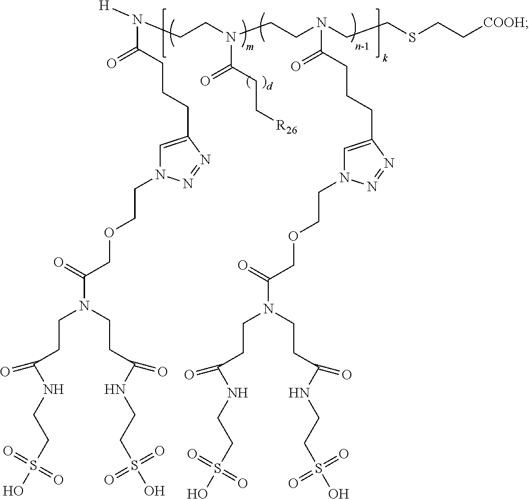 | (14) |
| 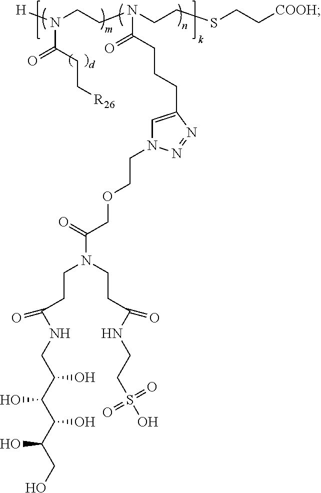 | (15) |
| 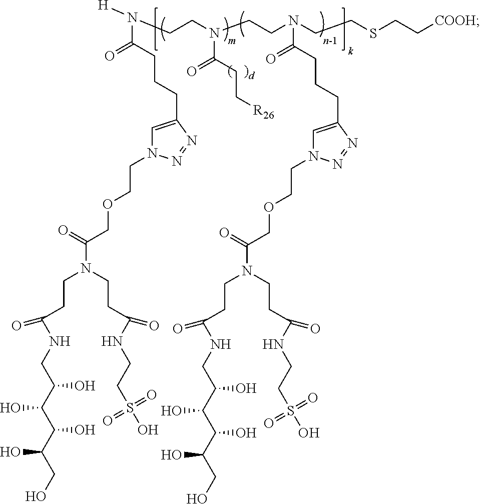 | (16) |
| 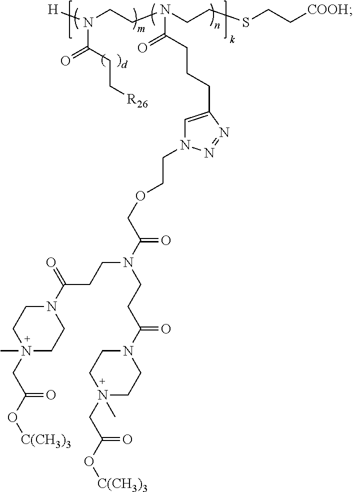 | (17) |
| 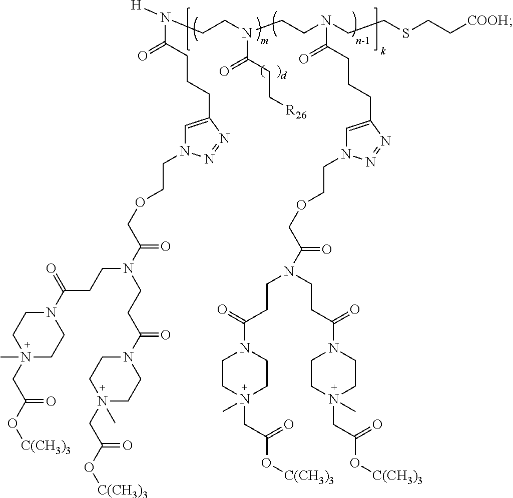 | (18) |

and

wherein:
R₂₆ is H or OH;
d is an integer 0 or 1;
k indicates that the polymer units m and n and polymer units m and (n−1) are connected to each other in a random order;
m is an integer from about 10 to about 50; and
n is an integer from 1 to 3.

In some embodiments, the POZ azido acetylene click polymer is of Formula (VIII-M):

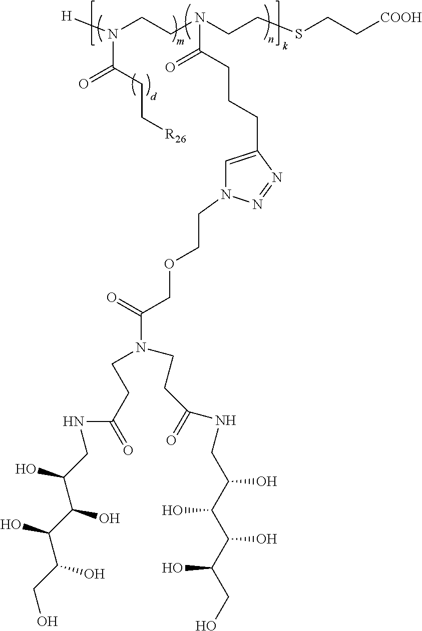

wherein R₂₆, d, m, n and k are as defined herein.

In some embodiments, the POZ polymer of Formula (VIII-J), Formula (VIII-K), Formula (VIII-L), Formula (VIII-M), Formula (VIII-N), Formula (VIII-O), Formula (VIII-P), Formula (VIII-Q) or Formula (VIII-R) can be connected to the drug linker moiety via the terminal —COOH group of the POZ polymer.

In some embodiments, the POZ polymer drug-linker scaffolds (i.e., without linking to a recognition moiety), described herein each typically have a polydispersity index (PDI) of 1.

In some embodiments, the POZ polymer drug-linker scaffold (i.e., prior to linking to a recognition moiety), is selected from those described in Table A8.

TABLE A8

| | |
|---|---|
| 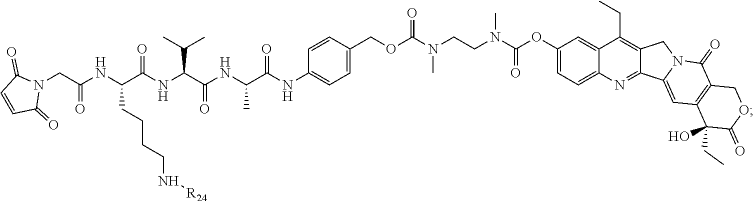 | (1) |
| 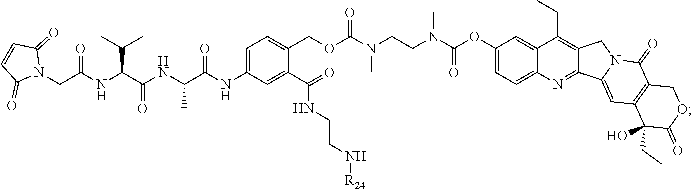 | (2) |
| 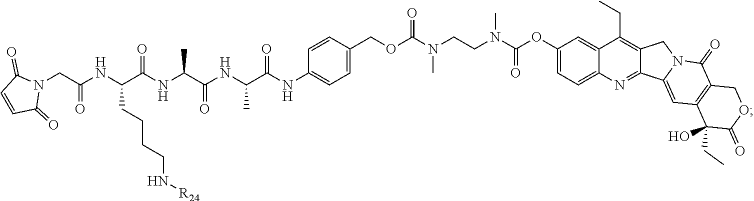 | (3) |
| 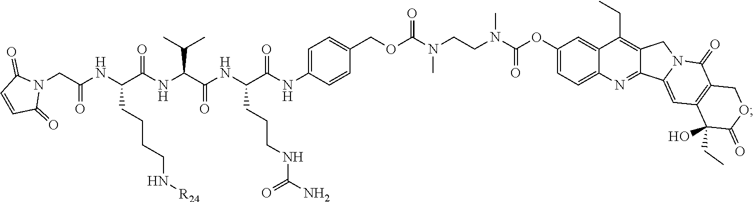 | (4) |
| 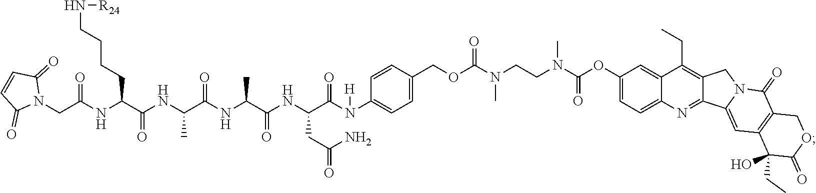 | (5) |
| 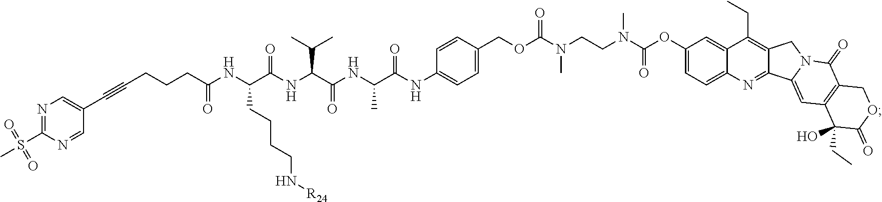 | (6) |
| 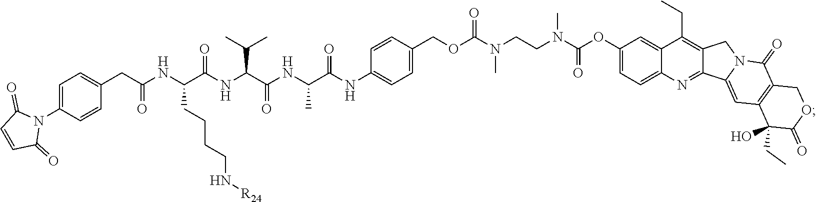 | (7) |
| 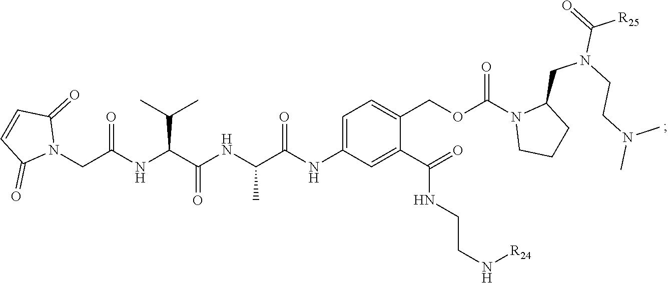 | (8) |
| 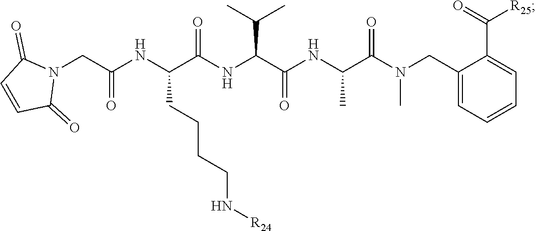 | (9) |
| 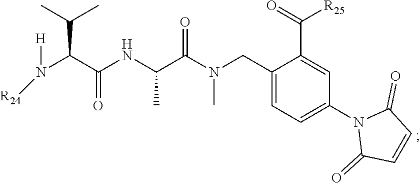 | (10) |
| 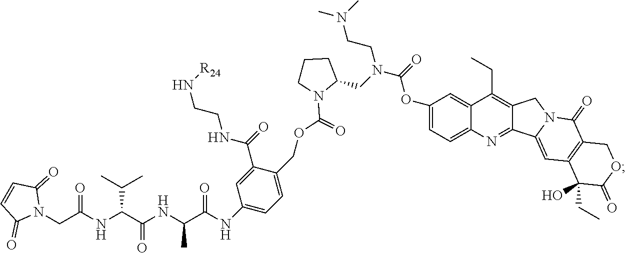 | (11) |
| 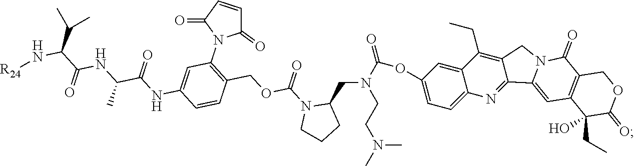 | (12) |
| 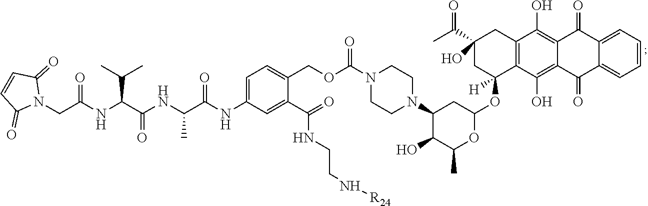 | (13) |
| wherein: | |
| R₂₄ is | |
| 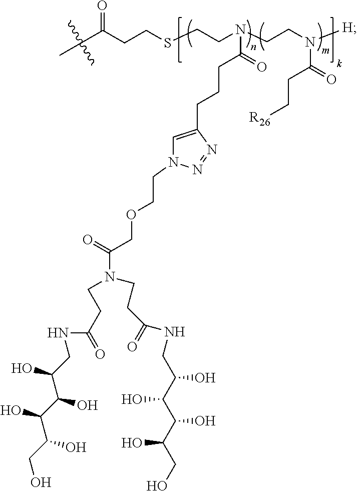 | |
| 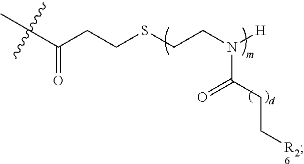 | |
| or | |
| 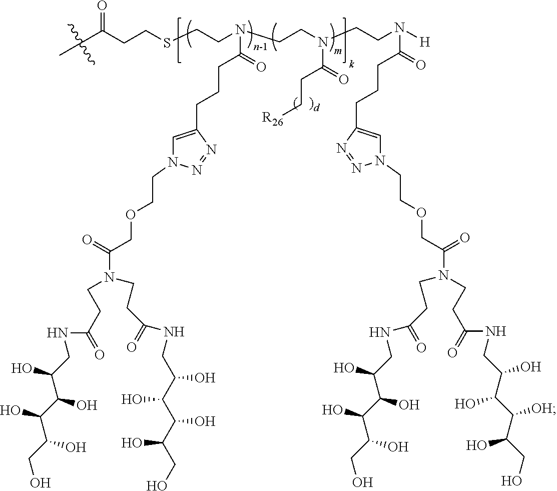 | |
| 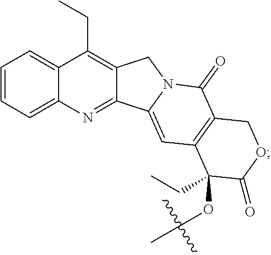 [SMILES: *O[C@]1(CC)C(=O)OCc2c1cc1n(c2=O)Cc2c-1nc1ccccc1c2CC] | |
| 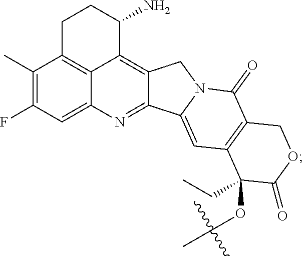 [SMILES: *O[C@]1(CC)C(=O)OCc2c1cc1n(c2=O)Cc2c-1nc1cc(F)c(C)c3c1c2[C@@H](N)CC3] | |
| or | |
| 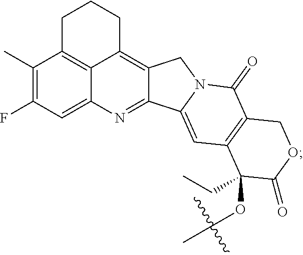 [SMILES: *O[C@]1(CC)C(=O)OCc2c1cc1n(c2=O)Cc2c-1nc1cc(F)c(C)c3c1c2CCC3] | |

R₂₆ is H or OH; d is an integer 0 or 1;
m is an integer from about 10, 20 or 50;
n is an integer 1 to 3; and
k indicates that the polymer units m and n and polymer units m and (n−1) are connected to each other in a random order.

In some embodiments, m is an integer of about 10.

In some embodiments, m is an integer of about 20.

In some embodiments, m is an integer of about 50.

In some embodiments, n is integer 1.

In some embodiments, n is integer 2.

In some embodiments, n is integer 3.

In some embodiments, R₂₄ is:

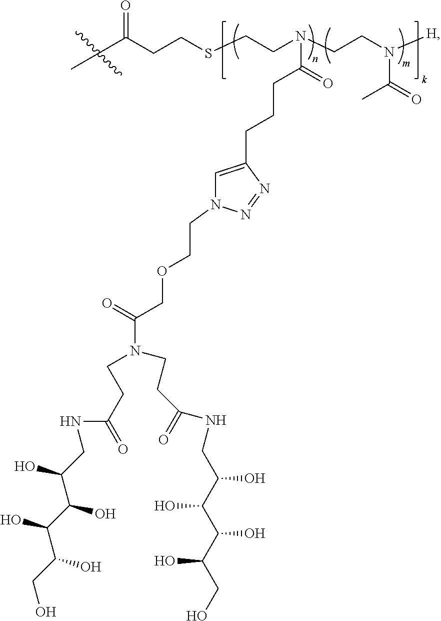

wherein n, m and k are as defined herein.

In some embodiments, R₂₄ is:

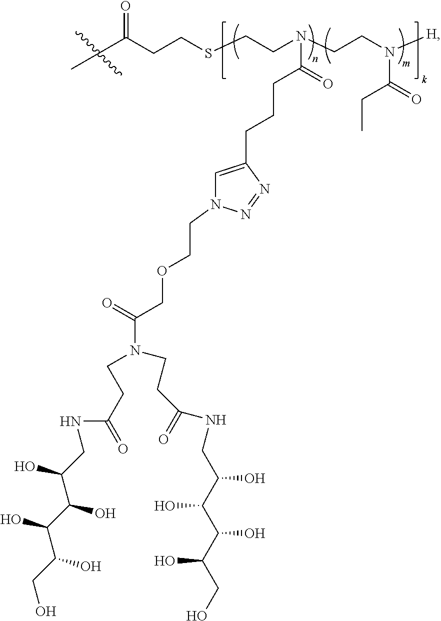

wherein n, m and k are as defined herein.

In some embodiments, R₂₄ is

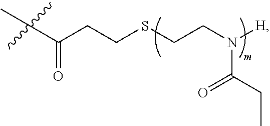

wherein m is as defined herein.

In some embodiments, R₂₄ is

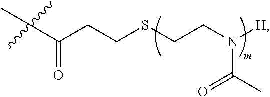

wherein m is as defined herein.

In some embodiments, R₂₄ is

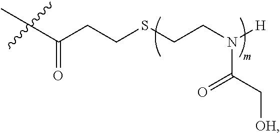

wherein m is as defined herein.

### Recognition Moiety

The conjugates of the present disclosure include a recognition moiety. As used in the present disclosure, the term “recognition moiety” refers to a molecule that recognizes and binds to a specific epitope/antigen/cell surface marker, including but not limited to, a protein that undergoes internalization into the cellular cytoplasm where it may fuse with endosomes that catalyze the release of the attached agent. Exemplary recognition moieties include, but are not limited to, antibodies, fragments of antibodies, single chain antibodies, polypeptides or peptide mimics, and the like. The recognition moiety, in addition to targeting the conjugate to a specific cell, tissue or location, may also have certain therapeutic effect (for example anti-proliferative (i.e., cytostatic and/or cytotoxic) activity against a target cell or pathway). The recognition moiety includes at least one chemically reactive group for linking to the variable A¹. In some embodiments, the recognition moiety may be engineered to include at least one chemically reactive group for linking to the variable A¹. In some embodiments the reactive group of the recognition moiety is a cysteine moiety or a lysine moiety. In some embodiments the reactive group of the recognition moiety is lysine moiety. In some embodiments the reactive group of the recognition moiety is a cysteine moiety. In other embodiments, the reactive group of the recognition moiety is an engineered cysteine moiety.

In some embodiments, the recognition moiety is an antibody or an antibody fragment. In one embodiment, the recognition moiety is an antibody. As used herein, the term antibody means any polypeptide chain-containing molecular structure that has a specific shape which binds to and recognizes an antigen/epitope on a target cell, where one or more non-covalent binding interactions stabilize the complex between the polypeptide chain-containing molecular structure and the antigen/epitope. In one embodiment, the antibody molecule is an immunoglobulin. The term antibody therefore includes all types of immunoglobulins (IgG, IgM, IgA, IgE, IgD, etc.), from all sources (e.g., human, rodent, rabbit, cow, sheep, pig, dog, llama, other mammal, etc.). Antibodies may be monoclonal antibodies or polyclonal antibodies. In one embodiment, the antibodies are monoclonal antibodies. Methods of raising antibodies and generating monoclonal antibodies are known to those of skill in the art. Antibodies or antigen binding fragments may also be produced by genetic engineering. In another embodiment, the recognition moiety is an antibody fragment, such as a single-chain antibody.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAh) to an antigen-of-interest in some embodiments is prepared by using any technique known in the art, which provides for production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies can be made by any of numerous techniques known in the art (e.g., Teng et al, 1983, Proc. Natl. Acad. Sci. USA. 80:73087312; Kozbor et ah, 1983, Immunology Today 4:72-79; and Olsson et ah, 1982, Meth. Enzymol. 92:3-16).

In some embodiments, antibodies useful for practicing the invention are intact antibodies or a functionally active fragments, derivatives or analogs of an antibody, wherein the antibody or fragment thereof is capable of immunospecifically binding to target cells (e.g., cancer cell antigens, viral antigens, or microbial antigens) or other antibodies that are bound to tumor cells or matrix. In this regard, “functionally active” means that the fragment, derivative or analog is able to immunospecifically bind to target cells. To determine which CDR sequences bind the antigen, synthetic peptides containing the CDR sequences in some embodiments are used in binding assays with the antigen by a binding assay method known in the art (e.g., the Biacore™ assay) (See, e.g., Kabat et al, 1991, Sequences of Proteins of Immunological Interest, Fifth Edition, National Institute of Health, Bethesda, Md; Kabat E et al, 1980, J. Immunology 125(3):961-969).

Other useful antibodies include fragments of antibodies such as, but not limited to, F(ab′)₂ fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, including both human and non-human portions, which in some embodiments are made using standard recombinant DNA techniques, are useful antibodies. A chimeric antibody is a molecule in which different portions are derived from different animal species, such as for example, those having a variable region derived from a murine monoclonal and human immunoglobulin constant regions. See, e.g., U.S. Pat. Nos. 4,816,567; and 4,816,397, each of which is incorporated herein by reference in its entirety. Humanized antibodies are antibody molecules from non-human species having one or more complementarity determining regions (CDRs) from the non-human species and a framework region from a human immunoglobulin molecule. See, e.g., U.S. Pat. No. 5,585,089, which is incorporated herein by reference in its entirety. Such chimeric and humanized monoclonal antibodies in some embodiments are produced by recombinant DNA techniques known in the art, for example using methods described in International Publication No. WO 87/02671; European Patent Publication No. 0 184 187; European Patent Publication No. 0 171 496; each of which is incorporated herein by reference in its entirety.

Completely human antibodies in some instances (e.g., when immunogenicity to a non-human or chimeric antibody may occur) are more desirable and in some embodiments are produced using transgenic mice that are incapable of expressing endogenous immunoglobulin heavy and light chains genes, but which are capable of expressing human heavy and light chain genes.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivitization by known protecting/blocking groups, proteolytic cleavage, linkage to a cellular antibody unit or other protein, etc. In some embodiments one or more of those numerous chemical modifications are carried out by known techniques including, but not limited to, specific chemical cleavage, acetylation, formylation, metabolic synthesis in the presence of tunicamycin, etc. In other embodiments, an analog or derivative of an antibody contains one or more unnatural amino acids, which is sometimes in combination with one or more of the above-described chemical modifications.

In some embodiments the antibody has one or more modifications (e.g., substitutions, deletions or additions) in amino acid residues that interact with Fc receptors. Those include modifications in amino acid residues identified as involved in the interaction between the anti-Fc domain and the FcRn receptor (see, e.g., International Publication No. WO 97/34631, which is incorporated herein by reference in its entirety.

In some embodiments, antibodies immunospecific for a cancer cell antigen are obtained commercially or produced by a method known to one of skill in the art such as, recombinant expression techniques. The nucleotide sequence encoding antibodies immunospecific for a cancer cell antigen is sometimes obtained, e.g., from the GenBank database or a database like it, the literature publications, or by routine cloning and sequencing.

In a specific embodiment, a known antibody for the treatment of cancer is used. In another specific embodiment, an antibody for the treatment of an autoimmune disease is used in accordance with the compositions and methods of the invention.

Exemplary antigens antibodies useful for practicing the invention are provided below. Exemplary antibodies that bind the indicated antigen are shown in parentheses.

In some embodiments, the antigen is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD 147 (exemplary antibodies include gavilimomab and metuzumab), CD 19 (exemplary antibodies include denintuzumab), CD20 (exemplary antibodies include rituximab, divozilimab, crelizumab and ibritumomab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR (exemplary antibodies include laprituximab, losatuxizumab, serclutamab, depatuxizumab and cetuximab), ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SITI, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab and KM4018), MFSD13A, Mincle, NOX1, SLC10A2, SLC12A2, SLC17A2, SLC38A1, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-I (exemplary antibodies include alemtuzumab), carcinoembryonic antigen (exemplary antibodies include arcitumomab, cergutuzumab, amunaleukin, and labetuzumab), CD112, CD155, CD24, CD247, CD37 (exemplary antibodies include lilotomab), CD38 (exemplary antibodies include felzartamab and daratumumab), CD3D, CD3E (exemplary antibodies include foralumab and teplizumab), CD3G, CD96, CDCP1 (exemplary antibodies include patritumab, elgemtumab, seribantumab and lumretuzumab), CDH17, CDH3, CDH6, CEACAM1, CEACAM5 (exemplary antibodies include labetuzumab), CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN1, GplOO, GPA33, gpNMB (exemplary antibodies include glembatumumab), ICAM1, L1CAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab and enapotamab), BMPR2, DCLK1, DDR1, EPHA receptors, EPHA2 (exemplary antibodies include hlC1 and IC1), ERBB2 also known as HER2 (exemplary antibodies include trastuzumab, bevacizumab, pertuzumab, and margetuximab), ERBB3 also known as HER3 (exemplary antibodies include patritumab, seribantumab, lumretuzumab, GSK2849330, CDX-3379, barecetamab, AV-203, elgemtumab, HM1BD-001, U3P1287/01 and SIBP-03), FLT3, PDGFR-B (exemplary antibodies include rinucumab), PTK7 (exemplary antibodies include cofetuzumab and ladiratuzumab,), RET, ROR1 (exemplary antibodies include cirmtuzumab and zilovertamab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab), IL13Ra2, ILIRAP (exemplary antibodies include nidanilimab), NT5E, 0X40, Ras mutant, RGS5, RhoC, SLAMF7 (exemplary antibodies include elotuzumab and azintuxizumab), and VSIR.

In some embodiments, the tumor-associated antigen is a transmembrane G-protein coupled receptor (GPCR). For example, the following antigens are GPCRs: CALCR, CD97, GPR87, and KISS IR.

In some embodiments, the tumor-associated antigen is cell-surface-associated or a cell-surface receptor. For example, the following antigens are cell-surface-associated and/or cell-surface receptors: 5T4 (exemplary antibodies include polatuzumab), B6A, B7-DC, BCMA (exemplary antibodies include belantamab), C4.4a (exemplary antibodies include lupartumab), CD137, CD138 (exemplary antibodies include indatuxumab), CD166 (exemplary antibodies include praluzatamab), CD 244, CD3 (exemplary antibodies include otelixizumab and visilizumab), CD48 (exemplary antibodies include hMEM102), CD5 (exemplary antibodies include zolimomab aritox), CD51 (exemplary antibodies include intetumumab), CD56 (exemplary antibodies include lorvotuzumab), CD70 (exemplary antibodies include cusatuzumab and vorsetuzumab), CD74 (exemplary antibodies include milatuzumab), CD79A, CD-262 (exemplary antibodies include tigatuzumab), claudin 18.2 (exemplary antibodies include is zolbetuximab and 163E12), DLL3 (exemplary antibodies include rovalpituzumab), DPEP-3 (exemplary antibodies include tamrintamab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), GCC (exemplary antibodies include indusatumab), GPC3 (exemplary antibodies include ragifilimab), HAVCR2, HLA-E, HLA-F, HLA-G, IGF-1R (exemplary antibodies include cixutumumab), LAG-3 (exemplary antibodies include encelimab), LY6G6D, LY9, c-Met (exemplary antibodies include telisotuzumab), mesothelin (exemplary antibodies include anetumab), MICA, MICB, MSLN, MUC1 (exemplary antibodies include gatipotuzumab), MUC5AC, MUC16 (exemplary antibodies include sofituzumab), NaPi2b (exemplary antibodies include lifastuzumab), NY-ESO-1, 0Y-TES1, Prolactin (exemplary antibodies include rolinsatamab), PVRIG, SLTRK6 (exemplary antibodies include sirtratumab), Sialyl-Thomsen-Nouveau Antigen, STN (exemplary antibodies include h2G12 and 2G12-2B2), Spermprotein 17, STEAP1 (exemplary antibodies include vandortuzumab), TROP-2 (exemplary antibodies include datopotamab, sacituzumab and hRS7), TNFRSF12, and uPAR.

In some embodiments, the tumor-associated antigen is a chemokine receptor or cytokine receptor. For example, the following antigens are chemokine receptors or cytokine receptors: CD 115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab, ifinatamab and omburtamab), B7-H4 (exemplary antibodies include mirzotamab), B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: αvβ6 (exemplary antibodies include hl5H3 and h2A2), ITGAV (exemplary antibodies include abituzumab), ITGB6, and ITGB8.

In some embodiments, the tumor-associated antigen is a glycolipid. For example, the following are glycolipid antigens: FucGM 1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormonereceptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM12, ADAM9, TMPRSS1 ID, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C5 complement, CCNB1, CSPG4, CYP1B 1, De2-7 EGFR, EGF, Fas-related antigen 1, FBP, G250, GAGE, HAS3, HPV E6 E7, hTERT, IDOl, LCK, Legumain, LYPD1, MAD-CT-1, MAD-CT-2, MAGEA3, MAGEA4, MAGEC2, MerTk, ML-IAP, NA17, NY-BR-1, p53, p53 mutant, PAP, PLAVI, polysialic acid, PR1, PSA, Sarcoma translocation breakpoints, SART3, sLe,SSX2, Survivin, Tn, TRAIL, TRAIL1, TRP-2, and XAGEL

In some embodiments, the antigen is an immune-cell-associated antigen. In some embodiments, the immune-cell-associated antigen is a transmembrane protein. For example, the following antigens are transmembrane proteins: BAFF-R, CD 163, CD 19 (exemplary antibodies include denintuzumab), CD20 (exemplary antibodies include rituximab, divozilimab, crelizumab and ibritumomab), CD25 (exemplary antibodies include basiliximab and daclizumab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-I (exemplary antibodies include ipilimumab, nivolumab, pembrolizumab, balstilimab, budigalimab, geptanolimab, toripalimab, and pidilizumabsf), SITI, and TLR2/4/1 (exemplary antibodies includetomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CDl 12, CD155, CD24, CD247, CD28, CD30L, CD37 (exemplary antibodies include lilotomab and SWAl 1), CD38 (exemplary antibodies include felzartamab and daratumumab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab and enapotamab) and FLT3.

In some embodiments, the immune-cell-associated antigen is transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48 (exemplary antibodies include hMEM102), CD5 (exemplary antibodies include zolimomab), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab and Clone 13 (also known as ADI-23674 or mAbl3)), Trem2, and uPAR.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

In some embodiments, the antibody targets HER2, TROP-2, HER3 or B7-H3.

In some embodiments, the antibody targets HER2. In some embodiments the antibodies include, but are not limited to, trastuzumab, bevacizumab, pertuzumab, and margetuximab. In some embodiments the antibody is trastuzumab.

In some embodiments, the antibody targets TROP-2. In some embodiments the antibodies include, but are not limited to, datopotamab, sacituzumab and hRS7. In some embodiments the antibody is datopotamab. In some embodiments the antibody is sacituzumab.

In some embodiments, the antibody targets HER3. In some embodiments the antibodies include, but are not limited to, patritumab, seribantumab, lumretuzumab, GSK2849330, CDX-3379, barecetamab, AV-203, elgemtumab, HMBD-001, U3P1287/01 and SIBP-03. In some embodiments the antibody is patritumab.

In some embodiments, the antibody targets B7-H3. In some embodiments the antibodies include, but are not limited to, enoblituzumab, ifinatamab and omburtamab. In some embodiments the antibody is ifinatamab.

It has been observed for bioconjugates that the site of conjugation can affect a number of parameters including ease of conjugation, drug-linker stability, effects on biophysical properties of the resulting bioconjugates, and in vitro cytotoxicity. Sites for conjugation on a recognition moiety include, for example, a reduced interchain disulfide, selected cysteine residues at engineered sites or a lysine site.

In some embodiments, the recognition moiety includes at least one chemically reactive group for linking to the variable A¹. In some embodiments, the recognition moiety may be engineered to include at least one chemically reactive group for linking to the variable A¹. In some embodiments the reactive group is a cysteine moiety or a lysine moiety. In some embodiments the reactive group is a lysine moiety. In some embodiments the reactive group is a cysteine moiety. In other embodiments, the reactive group is an engineered cysteine (eCys) moiety.

In some embodiments, the antibodies of the present disclosure include those having one or more cysteine residues, or one or more engineered cysteine (eCys) residues. In some embodiments, derivatives of cysteine (Cys) include, but are not limited to beta-2-Cys, beta-3-Cys, homocysteine, and N-methyl cysteine.

In some embodiments, the antibody is covalently linked to the variable A¹ via a sulfur atom of a cysteine or eCys residue in the antibody. In another embodiment, the antibody is covalently linked to the variable A¹ by a sulfhydryl group generated by reduction of an interchain disulfide of the antibody. Accordingly, in some embodiments, the variable A¹ is conjugated to a cysteine residue from reduced interchain disulfide(s).

### Variable p

In some embodiments, p is an integer ranging from about 2 to about 8, from about 2 to about 6, from about 2 to about 4, from about 4 to about 10, from about 4 to about 8, from about 4 to about 6.

In some embodiments, p is an integer ranging from about 2 to about 8.

In some embodiments, p is an integer 2, 4, 6, or 8. In some embodiments, p is an integer 6 or 8.

In some embodiments, p is an integer 8. In some embodiments, p is an integer 6.

### Conjugates

In some embodiments, conjugates of the disclosure include one or more occurrences of D, wherein D is camptothecin drug moiety or a topoisomerase II inhibitor drug moiety, wherein the one or more occurrences of D may be the same or different.

In some embodiments, one or more occurrences of recognition moiety is attached to the POZ polymer drug-linker moiety, wherein the one or more occurrences of recognition moiety may be the same or different. In some embodiments, one or more POZ polymer drug-linker moieties that includes one or more occurrences of D are connected to one recognition moiety (e.g., a antibody), wherein D is camptothecin drug moiety or a topoisomerase II inhibitor drug moiety.

In some embodiments, the conjugate of the disclosure include a recognition moiety that has a molecular weight of about 40 kDa or greater (e.g., about 60 kDa or greater; about 80 kDa or greater; about 100 kDa or greater; about 120 kDa or greater; about 140 kDa or greater; about 160 kDa or greater; about 180 kDa or greater; or about 200 kDa or greater, or about 40-200 kDa, about 40-180 kDa, about 40-140 kDa, about 60-200 kDa, about 60-180 kDa, about 60-140 kDa, about 80-200 kDa, about 80-180 kDa, about 80-140 kDa, about 100-200 kDa, about 100-180 kDa, or about 100-140 kDa) and has a sulfhydryl (i.e., —SH or thiol) group.

In some embodiments, the total number of sulfide bonds formed between the POZ polymer drug-linker moieties and the recognition moiety (or total number of attachment points) is 10 or less (e.g., 8, 6, 4, or 2).

In some embodiments, for conjugation with one or more POZ drug-linker moieties, the recognition moiety has a molecular weight of about 40 kDa or greater (e.g., about 60 kDa or greater, about 80 kDa or greater, about 100 kDa or greater, about 120 kDa or greater, about 140 kDa or greater, about 160 kDa or greater, or about 180 kDa or greater; or about 40-200 kDa, about 40-180 kDa, about 40-140 kDa, about 60-200 kDa, about 60-180 kDa, about 60-140 kDa, about 80-200 kDa, about 80-180 kDa, about 80-140 kDa, about 100-200 kDa, about 100-180 kDa, or about 100-140 kDa).

In some embodiments, for conjugation with one or more POZ polymer drug-linker moieties, the recognition moiety has a molecular weight of about 40 kDa to about 200 kDa. In some embodiments, for conjugation with one or more POZ polymer drug-linker moieties, the recognition moiety has a molecular weight of about 40 kDa to about 80 kDa.

In some embodiments, recognition moieties in this molecular weight range include, but are not limited to, for example, antibody fragments, such as, for example, Fabs.

In some embodiments, for conjugation with one or more POZ polymer drug-linker moieties, the recognition moiety has a molecular weight of about 60 kDa to about 120 kDa.

In some embodiments, recognition moieties in this molecular weight range include, but are not limited to, for example, camelids, Fab2, scFvFc, and the like.

In some embodiments, for conjugation with one or more POZ polymer drug-linker moieties, the recognition moiety has a molecular weight of about 140 kDa to about 180 kDa.

In some embodiments, recognition moieties in this molecular weight range include, but are not limited to, for example, full length antibodies, such as, IgG, IgM.

In some embodiments, the targeting ligands, the linkers and the drug or prodrug fragments described herein can be assembled into the conjugate or scaffold of the disclosure, for example according to the disclosed techniques and methods. Therapeutic and targeting conjugates of the disclosure, and methods for producing them, are described below by way of non-limiting example.

In some embodiments, the total number of sulfide bonds formed between the POZ polymer drug-linker moiety and the recognition moiety (or total number of attachment points) is 8 or less.

In some embodiments, the total number of sulfide bonds formed between the POZ polymer drug-linker moiety and the recognition moiety (or total number of attachment points) is 8. In some embodiments, the total number of sulfide bonds formed between the POZ polymer drug-linker moiety and the recognition moiety (or total number of attachment points) is 6. In some embodiments, the total number of sulfide bonds formed between the POZ polymer drug-linker moiety and the recognition moiety (or total number of attachment points) is 5. In some embodiments, the total number of sulfide bonds formed between the POZ polymer drug-linker moiety and the recognition moiety (or total number of attachment points) is 4. In some embodiments, the total number of sulfide bonds formed between the POZ polymer drug-linker moiety and the recognition moiety (or total number of attachment points) is 3. In some embodiments, the total number of sulfide bonds formed between the POZ polymer drug-linker moiety and the recognition moiety (or total number of attachment points) is 2.

In some embodiments, the ratio between POZ polymer drug-linker moiety and the recognition moiety is between about 1:1 and about 8:1. In some embodiments, the ratio between POZ polymer drug-linker moiety and the recognition moiety is between about 1:1 and about 6:1. In some embodiments, the ratio between POZ polymer drug-linker moiety and the recognition moiety is between about 1:1 and about 4:1. In some embodiments, the ratio between POZ polymer drug-linker moiety and the recognition moiety is between about 2:1 and about 2:1.

In some embodiments, the ratio between POZ polymer drug-linker moiety and the recognition moiety is between about 6:1 and about 8:1.

In some embodiments, the ratio between POZ polymer drug-linker moiety and the recognition moiety is about 8:1.

In some embodiments, the ratio between POZ polymer drug-linker moiety and the recognition moiety is about 6:1.

In some embodiments, the disclosure also relates to a POZ polymer drug-linker moiety including at least two moieties, wherein each moiety is capable of conjugation to a thiol group in a recognition moiety so as to form a recognition moiety-drug-linker conjugate.

In some embodiments, one or more thiol groups of a recognition moiety are produced by reducing a protein. The one or more thiol groups of the recognition moiety may then react with one or more POZ polymer drug-linker moieties that are capable of conjugation to a thiol group from the recognition moiety with the POZ polymer drug-linker moiety. In some embodiments, the at least two moieties connected to the recognition moiety are maleimide groups.

In some embodiments, the antibodies may be activated for conjugation with POZ polymer drug-linker moiety by treatment with a reducing agent such as DTT (Cleland's reagent, dithiothreitol) or TCEP (tris(2-carboxyethyl)phosphine hydrochloride). In some embodiments, full length, monoclonal antibodies can be reduced with an excess of TCEP to reduce disulfide bonds (e.g., between the cysteine present in the corresponding parent antibodies) to yield a reduced form of the antibody. The newly introduced and unpaired cysteine may remain available for reaction with POZ polymer drug-linker moiety to form the antibody conjugates of the present disclosure. In some embodiments, an excess of POZ polymer drug-linker moiety is added to effect conjugation and form the antibody-drug conjugate, and the conjugation mixture is purified to remove excess Drug-linker intermediate and other impurities.

In some embodiments, for conjugating of the POZ polymer drug-linker moiety, a recognition moiety has a molecular weight of 40 kDa or greater (e.g., 60 kDa or greater; 80 kDa or greater; or 100 kDa or greater; 120 kDa or greater; 140 kDa or greater; 160 kDa or greater or 180 kDa or greater). In some embodiments, the ratio of recognition moiety per POZ polymer drug-linker moiety is between about 1:1 and about 1:8; about 1:1 and about 1:6; between about 1:1 and about 1:5; between about 1:1 and about 1:4; between about 1:1 and about 1:3; or between about 1:1 and about 1:2.

Recognition moieties in this molecular weight range include, but are not limited to, for example, full length antibodies, such as, IgG, IgM.

In some embodiments, for conjugation with one or more POZ polymer drug-linker moieties a recognition moiety has a molecular weight of 60 kDa to 120 kDa. In some embodiments, the ratio of recognition moiety per POZ polymer drug-linker moiety is about 1:1 and about 1:8; between about 1:1 and about 1:6; between about 1:1 and about 1:5; between about 1:1 and about 1:4; between about 1:1 and about 1:3; or between about 1:1 and about 1:2.

Recognition moieties in this molecular weight range include, but are not limited to, for example, antibody fragments such as Fab2, scFcFv and camelids.

In some embodiments, for conjugation with one or more POZ polymer drug-linker moieties a recognition moiety has a molecular weight of 40 kDa to 80 kDa. In some embodiments, the ratio of recognition moiety per linker-drug moiety is about 1:1 and about 1:8; between about 1:1 and about 1:6; between about 1:1 and about 1:5; between 1:1 and about 1:4; between about 1:1 and about 1:3, or between about 1:1 and about 1:2.

In some embodiments, recognition moieties in this molecular weight range include, but are not limited to, for example, antibody fragments, such as, Fabs.

In some embodiments, the disclosure features a drug-linker moiety useful to conjugate with either or both of a protein-based recognition-molecule (recognition moiety) and POZ.

In some embodiments, the POZ polymer drug-linker moiety (i.e., without linking to a recognition moiety), described herein each typically have a polydispersity index (PDI) of 1.

Conjugates and scaffolds disclosed herein can be purified (i.e., removal of any starting materials) by extensive diafiltration. If necessary, additional purification by size exclusion chromatography can be conducted to remove any aggregated conjugates. In general, the conjugates as purified typically contain less than 5% (e.g., <2% w/w) aggregated conjugates as determined by SEC; less than 0.5% (e.g., <0.1% w/w) free (unconjugated) drug as determined by RP-HPLC; less than 1% drug carrying-peptide-containing scaffolds as determined by SEC and less than 2% (e.g., <1% w/w) unconjugated recognition moiety as determined by HIC-HPLC.

In some embodiments, the POZ polymer antibody-drug conjugate are selected from those described in Table A9.

TABLE A9

| | |
|---|---|
| 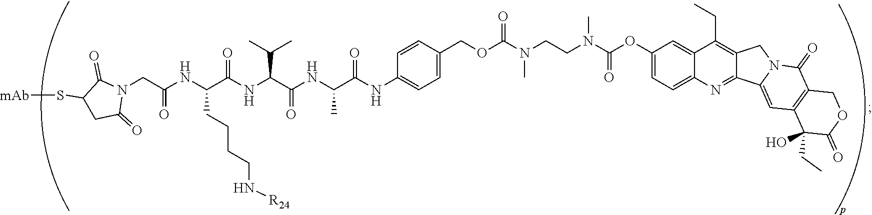 | (1) |
| 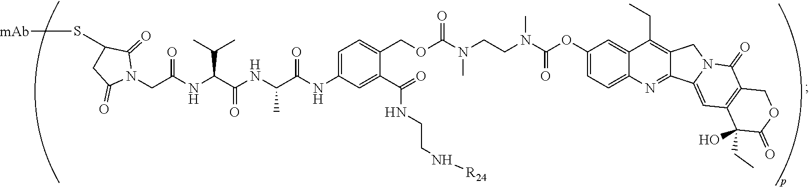 | (2) |
| 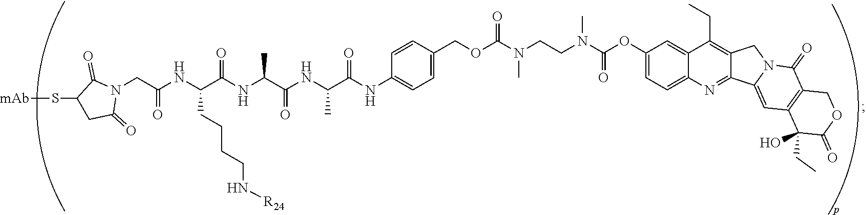 | (3) |
| 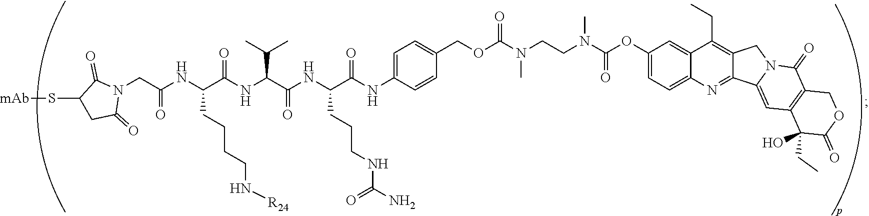 | (4) |
| 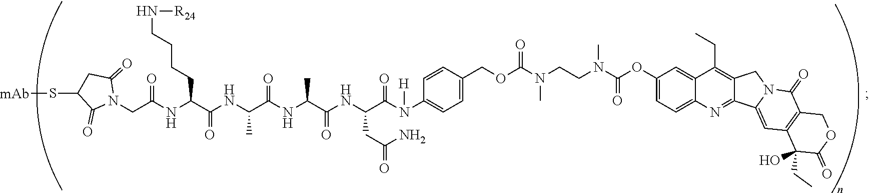 | (5) |
| 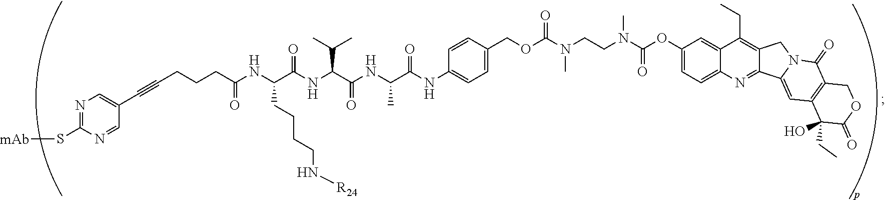 | (6) |
| 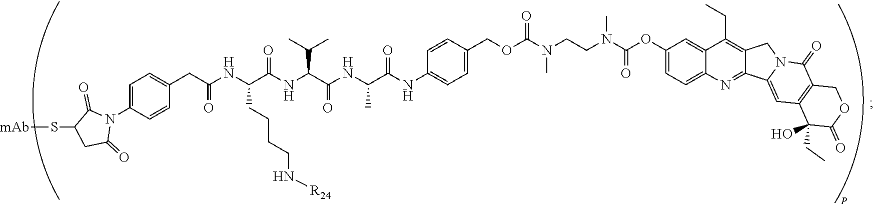 | (7) |
| 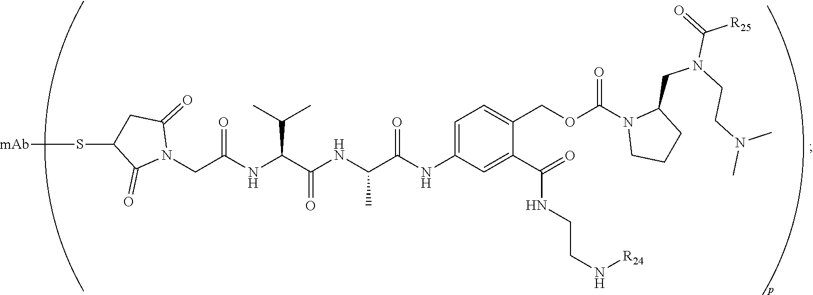 | (8) |
| 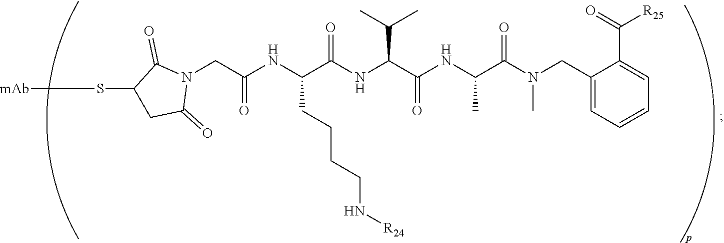 | (9) |
| 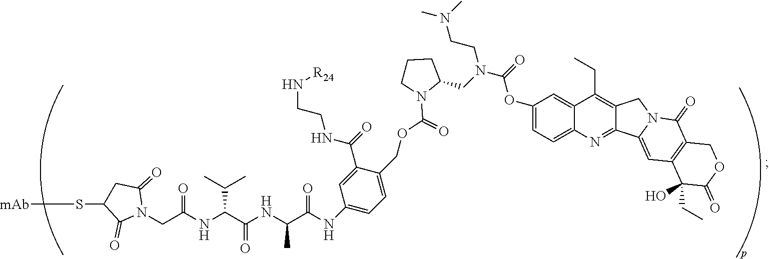 | (11) |
| 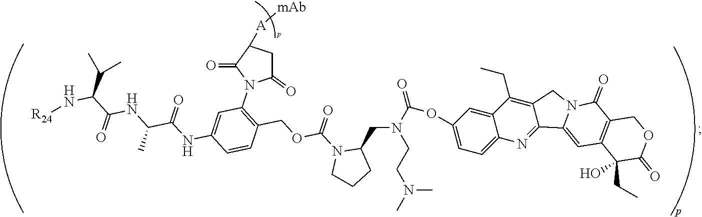 | (12) |
| 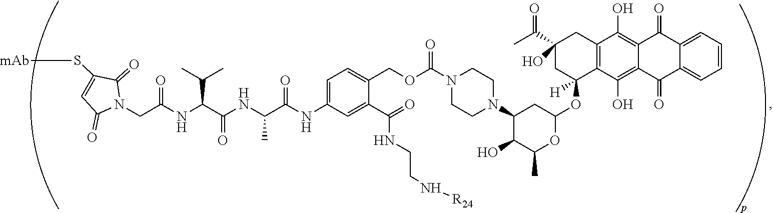 | (13) |
| wherein: | |
| R₂₄ is | |
| 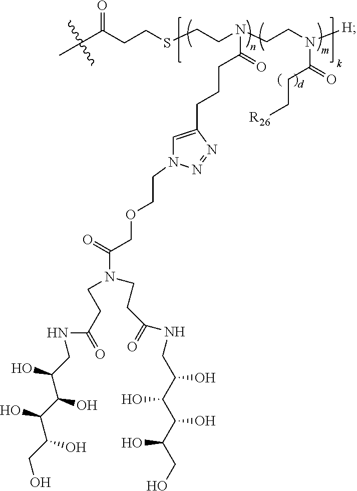 | |
| 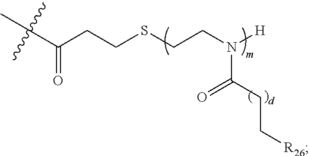 | |
| or | |
| 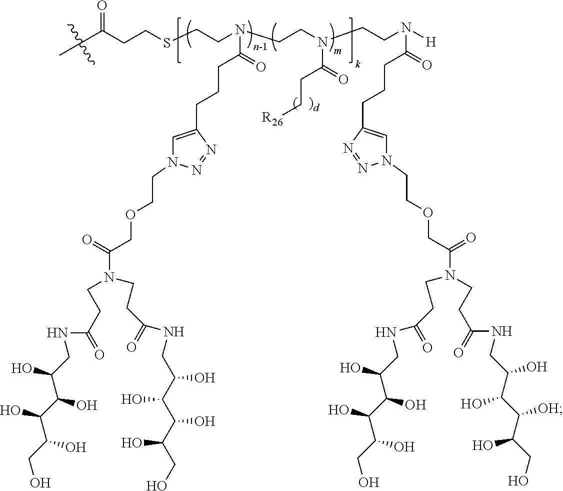 | |
| R₂₅ is: | |
| 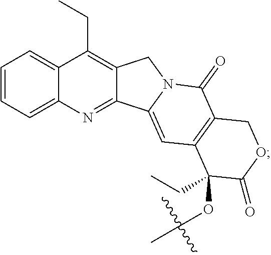 [SMILES: *O[C@]1(CC)C(=O)OCc2c1cc1n(c2=O)Cc2c-1nc1ccccc1c2CC] | |
| 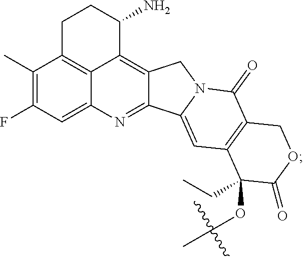 [SMILES: *O[C@]1(CC)C(=O)OCc2c1cc1n(c2=O)Cc2c-1nc1cc(F)c(C)c3c1c2[C@@H](N)CC3] | |
| or | |
| 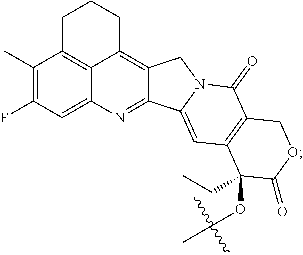 [SMILES: *O[C@]1(CC)C(=O)OCc2c1cc1n(c2=O)Cc2c-1nc1cc(F)c(C)c3c1c2CCC3] | |

R₂₆ is H or OH;
S is the sulfur atom of an antibody (mAb) cysteine residue;
d is an integer 0 or 1;
m is an integer from about 10, 20 or 50;
n is an integer 1 to 3;
k indicates that the polymer units m and n and polymer units m and (n−1) are connected to each other in a random order; and
p is an integer 6 or 8.

In some embodiments, m is an integer of about 10.

In some embodiments, m is an integer of about 20.

In some embodiments, m is an integer of about 50.

In some embodiments, n is an integer 1.

In some embodiments, n is an integer 2.

In some embodiments, n is an integer 3.

In some embodiments, p is an integer 6.

In some embodiments, p is an integer 8.

In some embodiments, p is an integer 8 and m is an integer of about 20.

In some embodiments, the antibody targets HER2

In some embodiments, the antibody targets TROP-2.

In some embodiments, the antibody targets HER3.

In some embodiments, the antibody targets B7-H3.

In some embodiments, R₂₄ is:

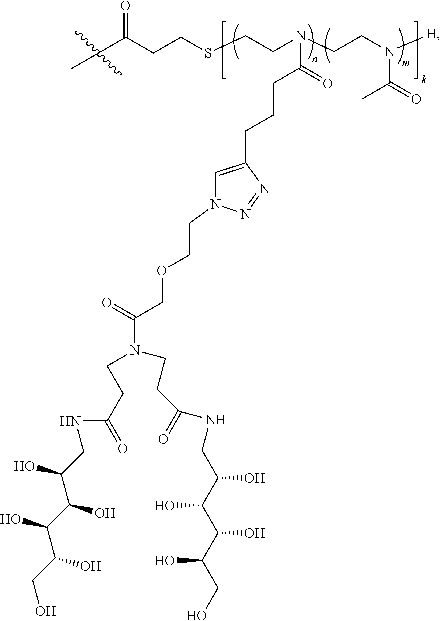

wherein n, m and k are as defined herein.

In some embodiments, R₂₄ is

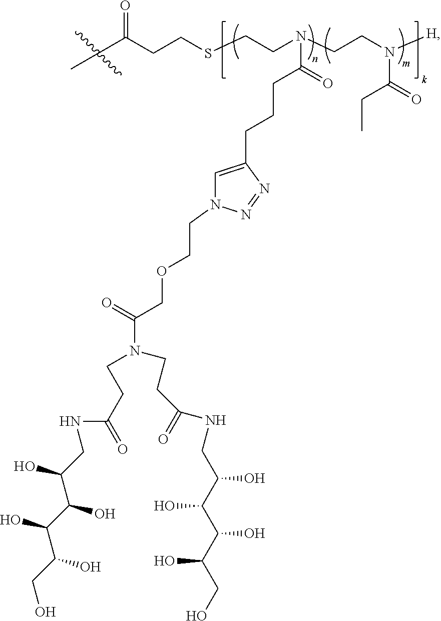

wherein n, m and k are as defined herein.

In some embodiments, R₂₄ is

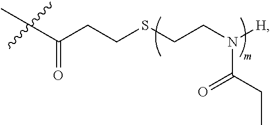

wherein m is as defined herein.

In some embodiments, R₂₄ is

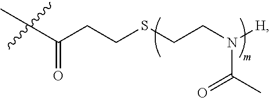

wherein m is as defined herein.

In some embodiments, R₂₄ is

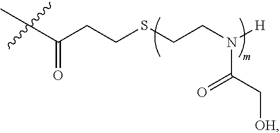

wherein m is as defined herein.

In some embodiments, R₂₄ is

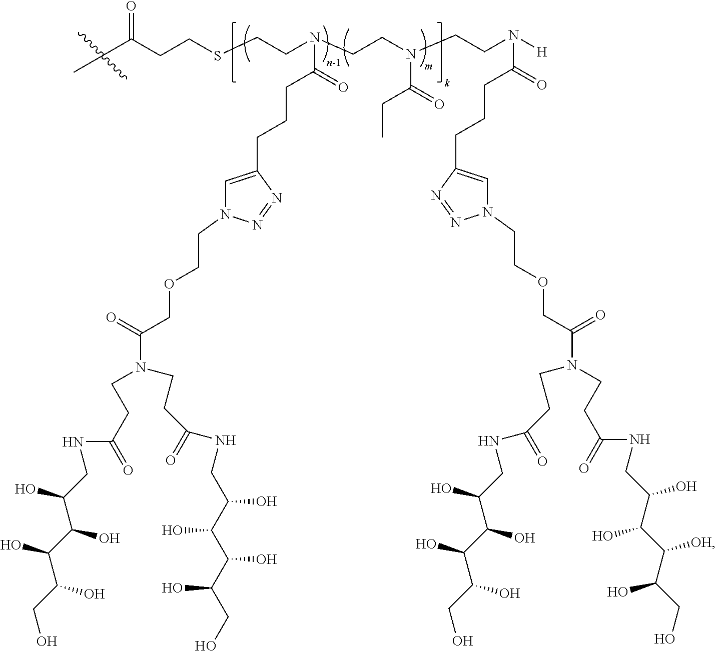

wherein m is as defined herein.

In some embodiments, R₂₄ is

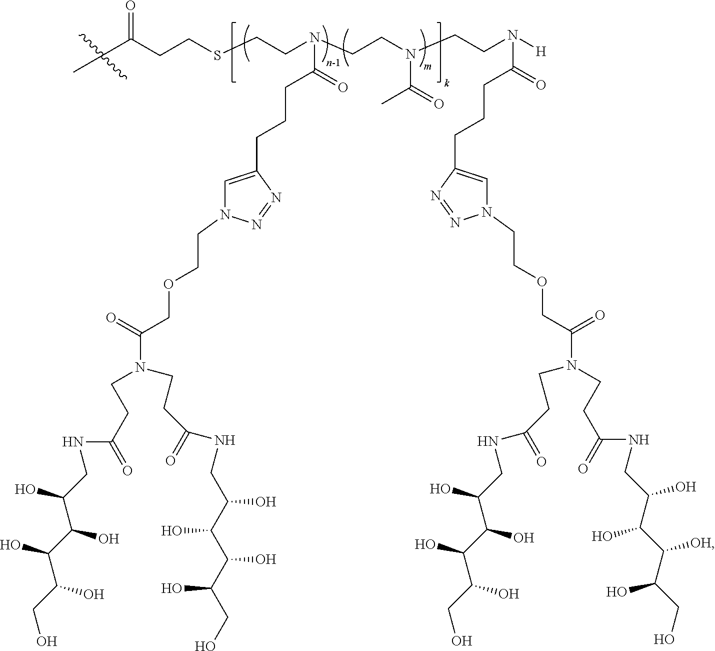

wherein m is as defined herein.

### Synthetic Methods

Any available techniques can be used to make the polymer conjugates or compositions including them, and intermediates and components useful for making them. For example, semi-synthetic and fully synthetic methods such as those illustrated below may be used.

Scheme I is an illustrative method for making a POZ polymer antibody-drug conjugate wherein the POZ polymer does not include a reactive pendent group, such as, for example a POZ polymer of Formula (VIII-J). The drug linker moiety (A) is reacted with a POZ polymer of Formula (VIII-J) in the presence of HATU and DMF to form the POZ polymer drug-linker moiety, (B). TCEP conjugation of the POZ polymer drug-linker moiety, (B), with an antibody having a cysteine group result in a POZ polymer antibody-drug conjugate (C).

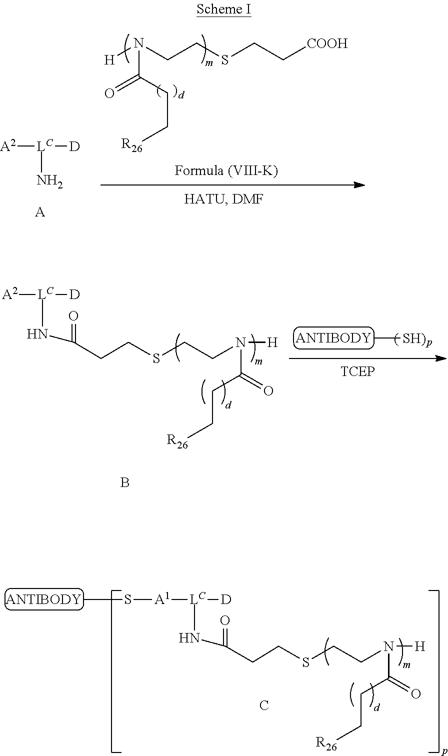

wherein:
A¹, A², L^{c}, D, R₂₆, d, m and p are as defined herein

The synthesis of a POZ polymer antibody-drug conjugate including a POZ azido acetylene click polymer can be prepared using the illustrative methods as depicted in Scheme II, Path A and Path B.

In Scheme II, Path A, a POZ azido acetylene click polymer (E) is formed by reacting a POZ polymer with a reactive acetylene pendent group such as, for example, a POZ polymer of Formula (VIII-K), with an azido moiety via copper(I) catalyzed azide-acetylene click chemistry. The POZ polymer (E) is then reacted with a drug linker moiety (A) in the presence of HATU and DMF to form the POZ polymer drug-linker moiety (F).

In Scheme II, Path B, a POZ polymer with a reactive acetylene pendent group such as, for example, a POZ polymer of Formula (VIII-K), is reacted with a drug linker moiety (A) in the presence of HATU and DMF to form the POZ polymer drug-linker moiety (G). The POZ polymer drug-linker moiety (G) is then reacted with an azido moiety via copper(I) catalyzed azide-acetylene click chemistry to form the POZ polymer drug-linker moiety, (F).

TCEP conjugation of the POZ polymer drug-linker moiety, (F), with an antibody having a cysteine group result in a POZ polymer antibody-drug conjugate (H).

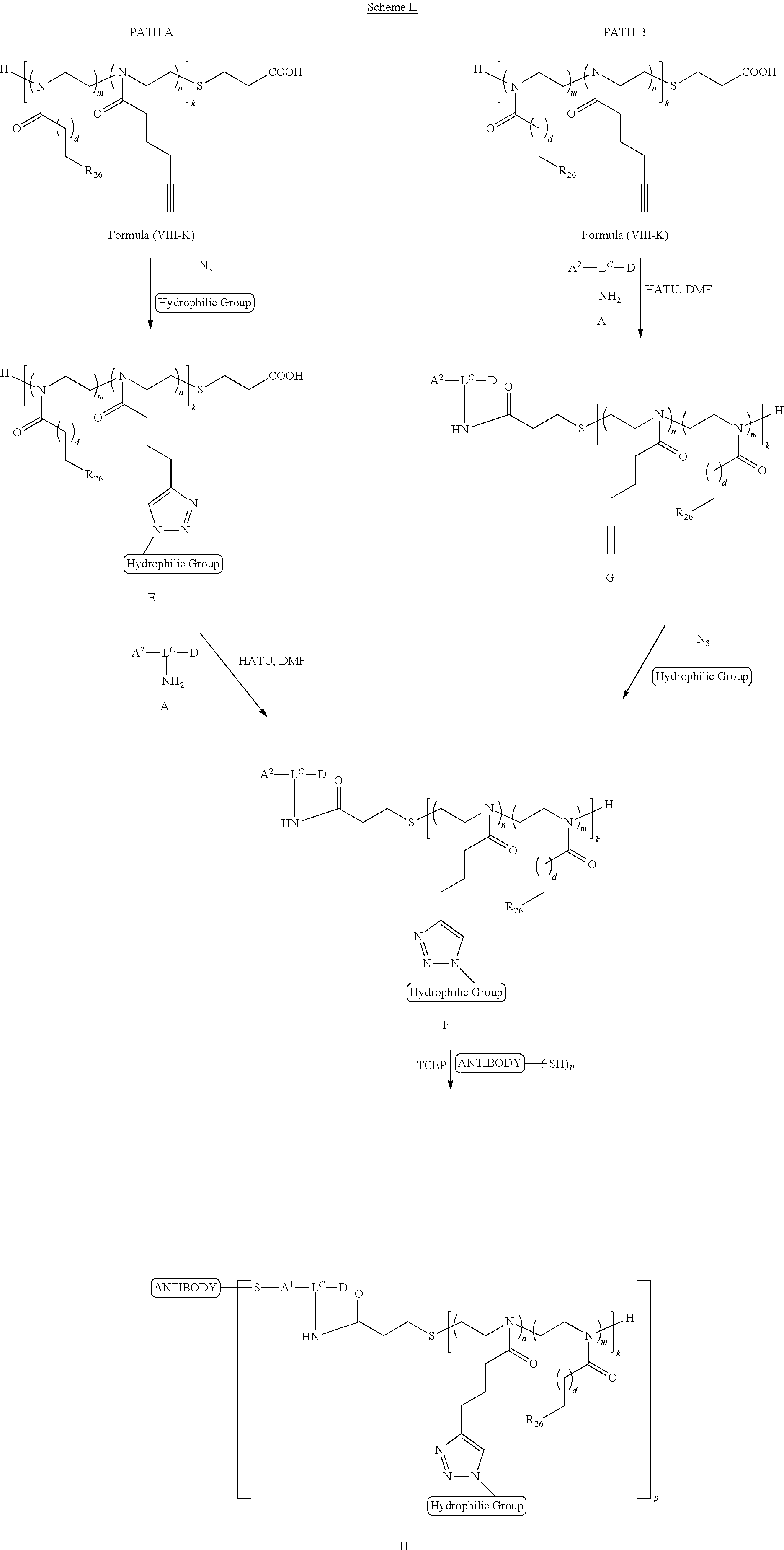

A¹, A², L^{c}, D, R₂₆, d, m, n and p are as defined herein.

### Pharmaceutical Compositions

The present invention provides pharmaceutical compositions including the conjugates described herein and at least one pharmaceutically acceptable carrier. The pharmaceutical composition is in any form that allows the compound to be administered to both humans and animals for treatment of a disorder associated with expression of the antigen to which the POZ antibody drug-linker moiety binds. For example, the conjugates are in the form of a liquid or solid.

The conjugates, including polymer-ADCs, of the present invention can be formulated for both human and veterinary use and may include the polymer-ADC and a pharmaceutically acceptable carrier. In general, a pharmaceutical composition will include a polymer-ADC of the present invention in addition to one or more inactive agents such as a sterile, biocompatible carrier including, but not limited to, sterile water, saline, buffered saline, or dextrose solution. The pharmaceutical compositions may be administered either alone or in combination with other therapeutic regimens including other chemotherapeutic compounds, hormones, vaccines, and/or radiation therapy. By “in combination with”, it is not intended to imply that the additional regimens must be administered at the same time or formulated for delivery together, although these methods of delivery are within the scope of the invention. In general, each will be administered at a dose and on a time schedule determined for that regimen. Additionally, the invention encompasses the delivery of the polymer-ADC of the present invention in combination with agents that may improve bioavailability, reduce or modify metabolism, inhibit excretion, or modify distribution within the body. Alternatively or additionally, the polymer-ADC of the present invention may be administered together with one or more other compounds that address a symptom or cause of the disease or disorder being treated, or of any other ailment from which the patient suffers. Although the pharmaceutical compositions of the present invention can be used for treatment of any subject (e.g., any animal) in need thereof, they are most preferably used in the treatment of humans.

The pharmaceutical compositions of the invention can be administered to humans and other animals by a variety of routes including oral, intravenous, intramuscular, intraarterial, subcutaneous, intraventricular, transdermal, rectal, intravaginal, intraperitoneal, topical (as by powders, ointments, or drops), buccal, or as an oral or nasal spray or aerosol. In general, the most appropriate route of administration will depend upon a variety of factors including the nature of the compound (e.g., its stability in the environment of the gastrointestinal tract), the condition of the patient (e.g., whether the patient is able to tolerate oral administration). The preferred route of administration is parenteral. Parenteral administration includes subcutaneous injections, intravenous, intramuscular, intrasternal injection or infusion techniques. In one embodiment, the pharmaceutical compositions is administered parenterally. In one embodiment, the conjugates are administered intravenously. Administration is by any convenient route, for example by infusion or bolus injection. In one embodiment, when administered to a patient, the conjugate or compositions thereof and pharmaceutically acceptable carriers are sterile.

Pharmaceutical compositions are formulated to allow a conjugate to be bioavailable upon administration of the composition to a patient. Compositions sometimes take the form of one or more dosage units.

Materials used in preparing the pharmaceutical compositions are preferably non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

The composition in some embodiments is in the form of a liquid. The liquid in some of those embodiments is useful for delivery by injection. In some embodiments a composition for administration by injection, in addition to the conjugate, contains one or more excipients selected from the group consisting of a surfactant, preservative, wetting agent, dispersing agent, suspending agent, buffer, stabilizer and isotonic agent.

The liquid compositions, whether they are solutions, suspensions or other like form, in some embodiments include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is sometimes enclosed in ampoule, a disposable syringe or a multiple-dose vial made of glass, plastic or other material. Physiological saline is an exemplary adjuvant. An injectable composition is preferably sterile.

The amount of the conjugate that is effective in the treatment of a particular disorder or condition will depend on the nature of the disorder or condition, which in some embodiments is determined by standard clinical techniques. In addition, in vitro or in vivo assays are optionally employed to help identify optimal dosage ranges. The precise dose to be employed in the compositions will also depend on the route of administration, and the seriousness of the disease or disorder, and should be decided according to the judgment of the practitioner and each patient's circumstances.

The compositions include an effective amount of a conjugate such that a suitable dosage amount will be obtained. Typically, that amount is at least about 0.01% of a compound by weight of the composition.

For intravenous administration, the pharmaceutical composition typically i from about 0.01 to about 100 mg of a conjugate per kg of the subject's body weight. In one embodiment, the composition can include from about 1 to about 100 mg of a conjugate per kg of the subject's body weight. In another aspect, the amount administered will be in the range from about 0.1 to about 25 mg/kg of body weight of a compound. Depending on the drug used, the dosage can be even lower, for example, 1.0 pg/kg to 5.0 mg/kg, 4.0 mg/kg, 3.0 mg/kg, 2.0 mg/kg or 1.0 mg/kg, or 1.0 pg/kg to 500.0 pg/kg of the subject's body weight.

Generally, the dosage of a conjugate administered to a patient is typically about 0.01 mg/kg to about 100 mg/kg of the subject's body weight or from 1.0 pg/kg to 5.0 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight. In some embodiments, the dosage administered is between about 0.1 to 4 mg/kg, even more preferably 0.1 to 3.2 mg/kg, or even more preferably 0.1 to 2.7 mg/kg of the subject's body weight over a treatment cycle.

In some embodiments, the conjugates are administered as an infusion every one week, every two weeks, every three weeks, every four weeks, every five weeks, every six weeks, every seven weeks, or every eight weeks.

In yet another embodiment, the conjugates are formulated in accordance with routine procedures as a pharmaceutical composition adapted for intravenous administration to animals, particularly humans. Typically, the carriers or vehicles for intravenous administration are sterile isotonic aqueous buffer solutions. Where necessary, the compositions include a solubilizing agent. Compositions for intravenous administration optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachets indicating the quantity of active agent. Where a conjugate is to be administered by infusion, it is typically dispensed, for example, with an infusion bottle containing sterile pharmaceutical grade water or saline. Where the conjugate is administered by injection, an ampoule of sterile water for injection or saline is sometimes provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

The frequency and timing of administration, and the dosage amounts, can be administered periodically over a cycle of administration to maintain a continuous and/or long-term effect of the active agents for a desired length of time. The conjugate can be administered hourly, daily, weekly, monthly, yearly or once. The length of time of the cycle of administration can be empirically determined, and is dependent on the disease to be treated, the severity of the disease, the particular patient, and other considerations within the level of skill of the treating physician. The length of time of treatment with a combination therapy provided herein can be one week, two weeks, one months, several months, one year, several years or more.

Dosage levels can be determined based on a variety of factors, such as body weight of the individual, general health, age, the activity of the specific compound employed, sex, diet, time of administration, rate of excretion, drug combination, the severity and course of the disease, and the patient's disposition resulting from the disease and the judgment of the treating physician.

It is understood that the amount to administer will be a function of the type of cancer being treated, the route of administration, and the tolerability of possible side effects. If necessary, dosage can be empirically determined.

### Methods of Use

The conjugates of the present invention are useful for inhibiting the multiplication of a tumor cell or cancer cell, causing apoptosis in a tumor or cancer cell, or for treating a cancer in a patient. The conjugates are used accordingly in a variety of settings for the treatment of cancers. The conjugates are intended to deliver a drug to a tumor cell or cancer cell. Without being bound by theory, in one embodiment, the recognition moiety of a conjugate binds to or associates with a cancer-cell or a tumor-cell associated antigen, and the Conjugate is taken up (internalized) inside the tumor cell or cancer cell through receptor-mediated endocytosis or other internalization mechanism. In some embodiments, the antigen is attached to a tumor cell or cancer cell or is an extracellular matrix protein associated with the tumor cell or cancer cell. Once inside the cell, the drug is released within the cell. In an alternative embodiment, the free drug is released from the conjugate outside the tumor cell or cancer cell, and the free drug subsequently penetrates the cell.

In one embodiment, the recognition moiety binds to the tumor cell or cancer cell.

In another embodiment, the recognition moiety binds to a tumor cell or cancer cell antigen which is on the surface of the tumor cell or cancer cell.

In another embodiment, the recognition moiety binds to a tumor cell or cancer cell antigen that is an extracellular matrix protein associated with the tumor cell or cancer cell.

The specificity of the recognition moiety for a particular tumor cell or cancer cell is an important consideration for determining the tumors or cancers that are most effectively treated. For example, conjugates that target a cancer cell antigen present on hematopoietic cancers are useful treating hematologic malignancies (e.g., anti-CD30, antiCD70, anti-CD19, anti-CD33 binding recognition moiety (e.g., antibody) are useful for treating hematologic malignancies). Conjugates that target a cancer cell antigen present on solid tumors in some embodiments are useful treating such solid tumors.

Cancers that are intended to be treated with a conjugate include, but are not limited to, hematopoietic cancers such as, for example, lymphomas (Hodgkin Lymphoma and Non-Hodgkin Lymphomas) and leukemias and solid tumors. Nonlimiting examples of hematopoietic cancers include follicular lymphoma, anaplastic large cell lymphoma, mantle cell lymphoma, acute myeloblastic leukemia, chronic myelocytic leukemia, chronic lymphocytic leukemia, diffuse large B cell lymphoma, and multiple myeloma. Examples of solid tumors include fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, endometrioid endometrial cancer (EEC)leiomyosarcoma, rhabdomyosarcoma, colon cancer, colorectal cancer, kidney cancer, pancreatic cancer, bone cancer, breast cancer, ovarian cancer, prostate cancer, esophageal cancer, gastric cancer, stomach cancer, oral cancer, nasal cancer, non-small cell lung cancer (NSCLC), throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, neuroblastoma, and retinoblastoma.

In one embodiment, the treated cancer is any one of the above-listed lymphomas and leukemias.

In another embodiment, the conjugates can be administered in vitro, in vivo and/or ex vivo to treat, prevent, reduce the risk of developing and/or delay onset of certain pathologies or disorders, for example, a cancer. For example, the conjugates of the disclosure are useful in treating, preventing, delaying the progression of or otherwise ameliorating a symptom of a cancer selected from the group consisting of anal cancer, astrocytoma, leukemia, lymphoma, head and neck cancer, liver cancer, testicular cancer, cervical cancer, colorectal cancer, sarcoma, stomach cancer, hemangioma, endometrioid endometrial cancer, esophageal cancer, eye cancer, glioma, laryngeal cancer, mouth cancer, mesothelioma, skin cancer, myeloma, oral cancer, rectal cancer, throat cancer, bladder cancer, breast cancer, uterine cancer, ovarian cancer, prostate cancer, lung cancer, non-small cell lung cancer (NSCLC), colon cancer, pancreatic cancer, renal cancer, and gastric cancer, pancreatic cancer,

In another embodiment, the conjugates of the disclosure are useful in treating, preventing, delaying the progression of or otherwise ameliorating a symptom of a cancer selected from the group consisting of breast cancer, cervical cancer, colon cancer, colorectal cancer, endometrioid endometrial cancer, esophageal cancer, gastric cancer, glioma, non-small cell lung cancer (NSCLC), ovarian cancer, prostate cancer, stomach cancer, bladder cancer and uterine cancer,

### Multi-Modality Therapy for Cancer

Cancers, including, but not limited to, a tumor, metastasis, or other disease or disorder characterized by uncontrolled cell growth are intended to be treated or inhibited by administration of an effective amount of a conjugate.

In one embodiment, methods for treating cancer are provided, including administering to a patient in need thereof an effective amount of a conjugate and a chemotherapeutic agent. In one embodiment the chemotherapeutic agent is one in which treatment of the cancer has not been found to be refractory to that agent. In another embodiment, the chemotherapeutic agent one in which the treatment of cancer has been found to be refractory to that agent.

In another embodiment, the conjugate is administered to a patient that has also undergone surgery as treatment for the cancer. In such embodiments a chemotherapeutic agent is typically administered over a series of sessions, or one or a combination of the chemotherapeutic agents, such a standard of care chemotherapeutic agent(s), is administered.

In either embodiment, the patient also receives an additional treatment, such as radiation therapy. In a specific embodiment, the conjugate is administered concurrently with the chemotherapeutic agent or with radiation therapy. In another specific embodiment, the chemotherapeutic agent or radiation therapy is administered prior or subsequent to administration of a conjugate.

Additionally, methods of treatment of cancer with a conjugate are provided as an alternative to chemotherapy or radiation therapy where the chemotherapy or the radiation therapy has proven or can prove too toxic, e.g., results in unacceptable or unbearable side effects, for the subject being treated. The patient being treated is optionally treated with another cancer treatment such as surgery, radiation therapy or chemotherapy, depending on which treatment is found to be acceptable or bearable.

### Treatment of Autoimmune Diseases

The conjugates are intended to be useful for killing or inhibiting the unwanted replication of cells that produce an autoimmune disease or for treating an autoimmune disease.

The conjugates are used accordingly in a variety of settings for the treatment of an autoimmune disease in a patient. The conjugates are typically used to deliver a drug to a target cell. Without being bound by theory, in one embodiment, the conjugate associates with an antigen on the surface of a pro-inflammatory or inappropriately stimulated immune cell, and the conjugate is then taken up inside the targeted cell through receptor-mediated endocytosis. Once inside the cell, the linker moiety is cleaved, resulting in release of the drug as a free drug. The free drug is then able to migrate within the cytosol and induce a cytotoxic or cytostatic activity. In an alternative embodiment, the drug moiety is cleaved from the conjugate outside the target cell, and the free drug resulting from that release subsequently penetrates the cell.

In one embodiment, the recognition moiety binds to an autoimmune antigen. In one such embodiment, the antigen is on the surface of a cell involved in an autoimmune condition.

In one embodiment, the recognition moiety binds to activated lymphocytes that are associated with the autoimmune disease state.

In a further embodiment, the conjugate kills or inhibits the multiplication of cells that produce an autoimmune antibody associated with a particular autoimmune disease.

Particular types of autoimmune diseases intended to be treated with the conjugates include, but are not limited to, Th2 lymphocyte related disorders (e.g., atopic dermatitis, atopic asthma, rhino conjunctivitis, allergic rhinitis, Omenn's syndrome, systemic sclerosis, and graft versus host disease); Thl lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

### Multi-Drug Therapy of Autoimmune Diseases

Methods for treating an autoimmune disease are also contemplated including administering to a patient in need thereof an effective amount of a conjugate and another therapeutic agent known for the treatment of an autoimmune disease.

### Kits

The present invention provides a pharmaceutical kit including, consisting essentially of or consisting of a conjugate of the present disclosure, packaging material, and instructions for administering the foregoing to a subject for the treatment of a disease or condition such a, but not limited to cancer.

In one embodiment, the pharmaceutical kits include one or more containers filled with one or more of the conjugates and/or compositions of the present disclosure, and optionally, one or more chemotherapeutic agents. Such kits can also include, for example, other compounds and/or compositions, a device(s) for administering the compounds and/or compositions, and written instructions in a form prescribed by a governmental agency regulating the manufacture, use or sale of pharmaceuticals or biological products. The compositions described herein can be packaged as a single dose or for continuous or periodic discontinuous administration. For continuous administration, a package or kit can include the conjugates in each dosage unit (e.g., solution or other unit described above or utilized in drug delivery), and optionally instructions for administering the doses daily, weekly, or monthly, for a predetermined length of time or as prescribed. If varying concentrations of a composition, of the components of the composition, or the relative ratios of the conjugates or agents within a composition over time is desired, a package or kit may contain a sequence of dosage units which provide the desired variability.

A number of packages or kits are known in the art for dispensing pharmaceutical agents for periodic oral use. In one embodiment, the package has indicators for each period. In another embodiment, the package is a labeled blister package, dial dispenser package, or bottle. The packaging means of a kit may itself be geared for administration, such as a syringe, pipette, eye dropper, or other such apparatus, from which the formulation may be applied to an affected area of the body, injected into a subject, or even applied to and mixed with the other components of the kit.

### EXAMPLES

Conjugates described herein can be prepared by the schemes generally outlined above and by methods described in the Examples below. The term “content” as used in certain examples below, unless otherwise specified, means the molar fraction of the polymer moieties that are substituted with the intended moiety, such as the linker, the drug molecule, or recognition moiety

The following working examples are illustrative of the linkers, drug molecules, antibodies or antibody drug conjugates, and methods for preparing same. These are not intended to be limiting and it will be readily understood by one of skill in the art that other reagents or methods may be utilized.

### Abbreviations

The following abbreviations are used in the reaction schemes and synthetic examples, which follow. This list is not meant to be an all-inclusive list of abbreviations used in the application as additional standard abbreviations, which are readily understood by those skilled in the art of organic synthesis, can also be used in the synthetic schemes and examples

| | |
|---|---|
| ACN | Acetonitrile |
| BOC | tert-butyloxycarbonyl |
| DCC | N,N′-Dicyclohexylcarbodiimide |
| DCM | Dichlolomethane |
| DIPEA | N,N-diisopropylethylamine |
| DMAP | 4-(dimethylamino)pyridine |
| DMF | Dimethylformamide |
| EDC | 1-Ethyl-3-(3-dimethylaminopropyl)carbodiimide |
| EEDQ | Ethyl 2-ethoxyquinoline-1(2H)-carboxylate |
| EtOAc | Ethyl acetate |
| FMOC | Fluorenylmethoxycarbonyl |
| HATU | Hexafluorophosphate azabenzotriazole tetramethyl |
| | uronium |
| HPLC | High pressure liquid chromatography |
| LiOH | Lithium hydroxide |
| MeOH | Methanol |
| NHS | 1-Hydroxypyrrolidine-2,5-dione (N- |
| | hydroxysuccinimide) |
| PBS | Phosphate buffered saline |
| PDI | Polydispersity index |
| PEOZ | POZ polymer with ethyl as the pendent group |
| RT | Room temperature |
| TCEP | (tris(2-carboxyethyl) phosphine hydrochloride). |
| TFA | Trifluoroacetic acid |

### General Information

All reagents were purchased from relevant providers unless otherwise stated.

POZ polymers were prepared as described in U.S. Pat. Nos. 7,943,141, 8,088,884, 8,110,651; 8,101,706, 8,383,093, 10,071,168 and 11,065,340 each of which is incorporated herein by reference in its entirety.

HPLC purification was performed on a YMC Triart C18 (250×20 mm, 5 μm) column, preparative column.

POZ polymer drug-linker moiety was purified by flash column purification using a SNAP Ultra 25 g column and Isolera System purchased from Biotage.

Purity of the POZ polymer drug-linker moiety was determined by reverse phase HPLC.

Molecular weights were determined by MALDI-TOF.

Proton NMR was conducted on a Varian, 500 MHZ, using DMSO-d6 or CDCl₃ as the solvent.

Antibody concentration was determined using the BCA Assay (Pierce™ BCA Protein Assay Kit, Thermo Fisher Scientific, Catalog No. 23225).

Drug to antibody ratio (DAR) was determined by analytical hydrophobic interaction chromatography, reverse phase HPLC and/or SDS page.

### Example 1: Synthesis of Target A, TFA Salt

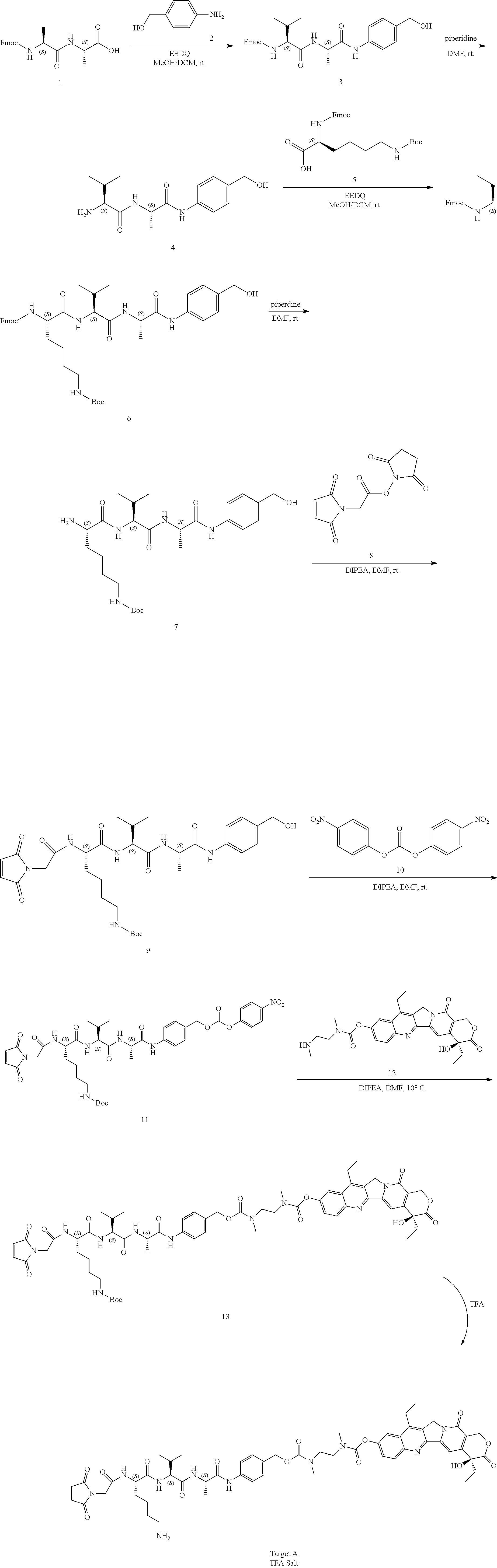

### Synthesis of (9H1-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate: Compound 3

To a stirred solution of (((9H-fluoren-9-yl)methoxy)carbonyl)-L-valyl-L-alanine, compound 1 (2.5 g, 6.09 mmol) and (4-aminophenyl)methanol, compound 2 (1.13 g, 9.14 mmol) in DCM (80 mL) and MeOH (20 mL) was added EEDQ (2.71 g, 10.96 mmol) at rt and the resulting mixture was stirred for 3 h. The reaction mixture was then diluted with ether (500 mL). The resulting precipitate was filtered and dried to provide (9H-fluoren-9-yl)methyl ((S)-1-(((S)-1-((4-(hydroxymethyl)phenyl)amino)-1-oxopropan-2-yl)amino)-3-methyl-I-oxobutan-2-yl) carbamate, compound 3 (2.1 g, 4.07 mmol, 66.9% yield) as a white solid. LC-MS: C₃₀H34N₃O₅ (M+H): calc. 516.25, found 516.20 (M+H).

### Synthesis of (S)-2-amino-N—((S)-1-((4-(hydroxymethyl)phenyl)amino)-1-oxopropan-2-yl)-3-methylbutanamide: Compound 4

To a stirred solution of (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate, compound 3 (2.1 g, 4.07 mmol) in DMF (20 mL) was added piperidine (1.21 mL, 12.22 mmol) at rt and the resulting mixture was stirred for 2 h. The reaction mixture was washed with excess n-pentane then washed with diethyl ether and finally dried under reduced pressure to afford (S)-2-amino-N—((S)-1-((4-(hydroxymethyl)phenyl)amino)-1-oxopropan-2-yl)-3-methylbutanamide, compound 4 (1.1 g, 3.75 mmol, 92% yield) as an off-white solid. LC-MS: C₁₅H₂₄N₃O₃ (M+H): calc. 294.18, found 294.10 (M+H).

### Synthesis of (9H-fluoren-9-yl)methyl tert-butyl ((S)-6-(((S)-1-(((S)-1-((4-(hydroxymethyl)phenyl)amino)-1-oxopropan-2-yl)amino)-3-methyl-1-oxobutan-2-yl)amino)-6-oxohexane-1,5-diyl)dicarbamate: Compound 6

To a stirred solution of (S)-2-amino-N—((S)-1-((4-(hydroxymethyl)phenyl)amino)-1-oxopropan-2-yl)-3-methylbutanamide, compound 4 (1.1 g, 3.75 mmol) and N2-(((9H-fluoren-9-yl)methoxy)carbonyl)-N6-(tert-butoxycarbonyl)-L-lysine, compound 5 (1.933 g, 4.12 mmol) in DCM (80 mL) and MeOH (20 mL) was added to ethyl 2-ethoxyquinoline-1(2H)-carboxylate (1.85 g, 7.50 mmol) at rt and the reaction mixture was stirred for 3 h. The resulting solid was diluted with ether (400 mL), filtered and dried to provide (9H-fluoren-9-yl)methyl tert-butyl ((S)-6-(((S)-1-(((S)-1-((4-(hydroxymethyl)phenyl)amino)-1-oxopropan-2-yl)amino)-3-methyl-1-oxobutan-2-yl)amino)-6-oxohexane-1,5-diyl)dicarbamate, compound 6 (2.4 g, 3.23 mmol, 86% yield) as an off-white solid. LC-MS: C₄₁H₅₄N₅O₈ (M+H): calc. 744.38, found 744.35 (M+H), 644.20 (M−Boc+H).

### Synthesis of tert-butyl ((S)-5-amino-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate: Compound 7

To a stirred solution of (9H-fluoren-9-yl)methyl tert-butyl ((S)-6-(((S)-1-(((S)-1-((4-(hydroxymethyl)phenyl)amino)-1-oxopropan-2-yl)amino)-3-methyl-1-oxobutan-2-yl)amino)-6-oxohexane-1,5-diyl)dicarbamate, compound 6 (9.8 g, 13.17 mmol) in DMF (25 mL) was added piperidine (3.91 mL, 39.5 mmol) at rt and the resulting mixture was stirred for about 3 h. The reaction mixture was washed with excess n-pentane, followed by washing with diethyl ether, then dried under reduced pressure to afford tert-butyl ((S)-5-amino-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, compound 7 (6.1 g, 11.69 mmol, 89% yield) as an off-white solid. LC-MS: C₂₆H44N₅O₆ (M+H): calc. 522.33, found 522.55 (M+H), 422.35 (M−Boc+H).

### Synthesis of tert-butyl ((S)-5-(2-(2,5-dioxo-2,5-dihydro-1H-pyrrol-1-yl)acetamido)-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate: Compound 9

To a stirred solution of tert-butyl ((S)-5-amino-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, compound 7 (3.0 g, 5.75 mmol) and 2,5-dioxopyrrolidin-1-yl 2-(2,5-dioxo-2,5-dihydro-1H-pyrrol-1-yl)acetate, compound 8 (1.45 g, 5.75 mmol) in DMF (30 mL) was added DIPEA (2.01 mL, 11.50 mmol) at rt and the resulting mixture was stirred for 30 min. The reaction mixture was diluted with diethyl ether, the resulting solid was filtered and washed with n-pentane and dried to provide tert-butyl ((S)-5-(2-(2,5-dioxo-2,5-dihydro-1H-pyrrol-1-yl)acetamido)-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, compound 9 (3.1 g, 4.71 mmol, 82% yield) as an off-white solid. LC-MS: C₃₂H47N₆O₉ (M+H): calc. 659.34, found 659.30 (M+H), 559.20 (M−Boc+H).

### Synthesis of tert-butyl ((S)-5-(2-(2,5-dioxo-2,5-dihydro-1H-pyrrol-1-yl)acetamido)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-6-oxohexyl)carbamate: Compound 11

To a stirred solution of tert-butyl ((S)-5-(2-(2,5-dioxo-2,5-dihydro-1H-pyrrol-1-yl)acetamido)-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, /compound 9 (3.1 g, 4.71 mmol) and bis(4-nitrophenyl) carbonate, compound 10 (7.16 g, 23.53 mmol) in DMF (30 mL) was added DIPEA (4.11 mL, 23.53 mmol) at rt and the reaction mixture was stirred for 16 h at rt. The reaction mixture was diluted with diethyl ether and the resulting solids were filtered and washed with n-pentane and dried in vacuo to provide tert-butyl ((S)-5-(2-(2,5-dioxo-2,5-dihydro-1H-pyrrol-1-yl)acetamido)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-6-oxohexyl)carbamate, compound 11 (2.8 g, 3.40 mmol, 72.2%) as an off-white solid. LC-MS: C₃₉H₅₀N₇O₁₃ (M−Boc+H): calc. 724.29, found 724.10 (M−Boc+H).

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(2,5-dioxo-2,5-dihydro-1H-pyrrol-1-yl)acetamido)-13-isopropyl-2,2,16-trimethyl-4,11,14-trioxo-3-oxa-5,12,15-triazaheptadecan-17-amido)benzyl) ethane-1,2-diylbis(methylcarbamate): Compound 13

To a stirred solution of tert-butyl ((S)-5-(3-(2,5-dioxo-2,5-dihydro-1H-pyrrol-1-yl)-2-oxopropanamido)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-6-oxohexyl)carbamate, compound 11 (1.55 g, 1.82 mmol) and DIPEA (1.45 mL, 8.28 mmol) in DMF (10 mL) was added (S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate, compound 12 (prepared as described in Example 1A, 1.0 g, 1.66 mmol) in DMF (10 mL) at 10° C. and the reaction mixture was stirred for another 1 h at 10° C. The reaction mixture was diluted with diethyl ether and the resulting solids were filtered and washed with n-pentane and dried to afford (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(2,5-dioxo-2,5-dihydro-1H-pyrrol-1-yl)acetamido)-13-isopropyl-2,2,16-trimethyl-4,11,14-trioxo-3-oxa-5,12,15-triazaheptadecan-17-amido)benzyl) ethane-1,2-diylbis(methylcarbamate), compound 13 (1.9 g, 0.63 mmol, 37.9% yield) as a yellow solid. LC-MS: C₆₀H₇₅N₁₀O₁₆ (M+H): calc. 1191.54, found 1191.20 (M+H). HPLC: 39% purity.

### Synthesis of 4-((S)-2-((S)-2-((S)-6-amino-2-(2-(2,5-dioxo-2,5-dihydro-1H-pyrrol-1-yl)acetamido)hexanamido)-3-methylbutanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate): Target A, TFA Salt

To a stirred solution of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(2,5-dioxo-2,5-dihydro-1H-pyrrol-1-yl)acetamido)-13-isopropyl-2,2,16-trimethyl-4,11,14-trioxo-3-oxa-5,12,15-triazaheptadecan-17-amido)benzyl) ethane-1,2-diylbis(methylcarbamate), compound 13 (1.9 g, 1.60 mmol) in DCM (30 mL) was added TFA (6.14 mL, 80 mmol) at rt and the reaction mixture was stirred for 2 h. The reaction mixture was concentrated under reduced pressure. The crude reaction mass was then triturated with EtOAc. The resulting solids were filtered, washed with n-pentane and then dried in vacuo. The obtained crude compound (2.4 g) was purified by HPLC (Mobile phase A: 0.1% TFA in H₂O, Mobile phase B: 100% acetonitrile). Pure fractions were dried by lyophilization for 2 days to obtain 4-((S)-2-((S)-2-((S)-6-amino-2-(2-(2,5-dioxo-2,5-dihydro-1H-pyrrol-1-yl)acetamido)hexanamido)-3-methylbutanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate). Target A, TFA salt (650 mg, 0.581 mmol, 26.4% yield) as a yellow solid.

¹H NMR (DMSO-d₆, 400 MHz): δ 9.61 (s, 1H), 8.14 (d, J=9.2 Hz, 2H), 7.92 (s, 1H), 7.81 (d, J=5.6 Hz, 1H), 7.58-7.50 (m, 6H), 7.35 (s, 1H), 7.26 (d, J=7.6 Hz, 2H), 6.99 (s, 2H), 5.41 (q, J=16.4 Hz, 2H), 5.31 (s, 2H), 5.03 (s, 2H), 4.42-4.39 (m, 1H), 4.35-4.32 (m, 1H), 4.17-4.15 (m, 1H), 4.10 (s, 3H), 3.55 (s, 4H), 3.19-3.13 (m, 2H), 3.05-2.95 (m, 6H), 2.77-2.76 (m, 2H), 2.04-2.02 (m, 1H), 1.92-1.88 (m, 2H), 1.73-1.71 (m, 1H), 1.57-1.55 (m, 3H), 1.33-1.28 (m, 8H), 0.92-0.83 (m, 9H). LC-MS: C₅₅H₆₇N₁₀O₁₄ (M+H): calc. 1091.48, found 1091.25 (M+H). HPLC: 97.50% purity.

### Example 1A: Synthesis of (S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate: Compound 12

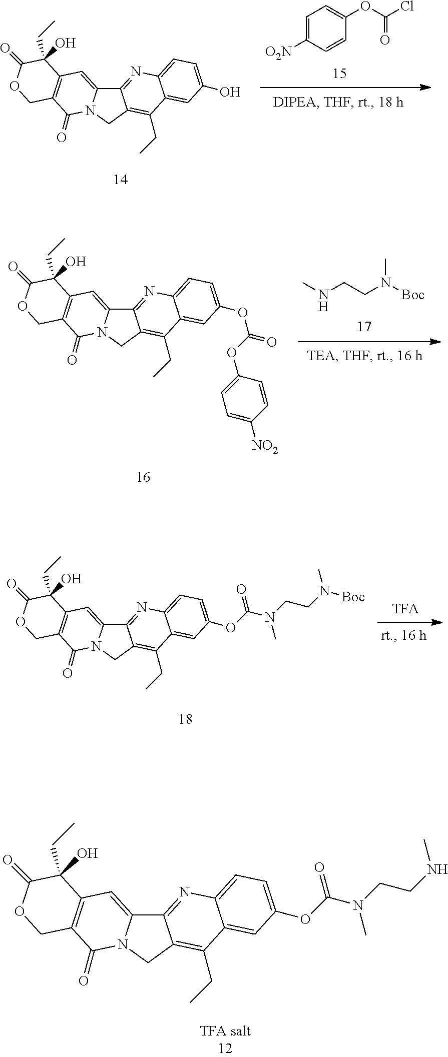 [SMILES: CCc1c2c(nc3ccc(O)cc13)-c1cc3c(c(=O)n1C2)COC(=O)[C@]3(O)CC ; O=C(Cl)Oc1ccc([N+](=O)[O-])cc1 ; CCc1c2c(nc3ccc(OC(=O)Oc4ccc([N+](=O)[O-])cc4)cc13)-c1cc3c(c(=O)n1C2)COC(=O)[C@]3(O)CC ; CNCCN(C)C(=O)OC(C)(C)C ; CCc1c2c(nc3ccc(OC(=O)N(C)CCN(C)C(=O)OC(C)(C)C)cc13)-c1cc3c(c(=O)n1C2)COC(=O)[C@]3(O)CC ; CCc1c2c(nc3ccc(OC(=O)N(C)CCNC)cc13)-c1cc3c(c(=O)n1C2)COC(=O)[C@]3(O)CC]

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-nitrophenyl) carbonate: Compound 16

To a stirred solution of (S)-4,11-diethyl-4,9-dihydroxy-1,12-dihydro-14H-pyrano[3′,4′:6,7]indolizino[1,2-b]quinoline-3,14(4H)-dione, compound 14 (3.0 g, 7.65 mmol) and bis(4-nitrophenyl) carbonate, compound 15 (4.65 g, 15.29 mmol) in THF (60 mL) was added DIPEA (4.01 mL, 22.94 mmol) and the resulting mixture was stirred at rt for 16 h. The reaction mixture was diluted with diethyl ether and the resulting solid was filtered and washed with n-pentane and dried in vacuo to provide (S)-4,11-diethyl-4-hydroxy-3,14-dioxo-3,4,12,14-tetrahydro-1H-pyrano[3′,4′:6,7]indolizino[1,2-b]quinolin-9-yl (4-nitrophenyl) carbonate, compound 16 (4.0 g, 5.81 mmol, 76% yield) as an off-white solid. LC-MS: C₂₉H₂₄N₃O₉ (M+H): calc. 558.15, found 558.05 (M+H). HPLC: 81% purity.

### Synthesis of (S)-tert-butyl (4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate): Compound 18

To a stirred solution of (S)-4,11-diethyl-4-hydroxy-3,14-dioxo-3,4,12,14-tetrahydro-1H-pyrano[3′,4′:6,7]indolizino[1,2-b]quinolin-9-yl (4-nitrophenyl) carbonate, compound 16 (4.0 g, 7.17 mmol) and tert-butyl methyl(2-(methylamino)ethyl)carbamate, compound 17 (2.70 g, 14.35 mmol) in THF (100 mL) was added DIPEA (3.76 mL, 21.52 mmol) at rt and the resulting mixture was stirred at rt for 16 h. The reaction mixture was diluted with diethyl ether and the resulting solids were filtered, washed with n-pentane and dried in vacuo to afford S)-tert-butyl (4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate), compound 18 (4.0 g, 5.60 mmol, 78% yield) as an off-white solid. LC-MS: C₃₂H₃₉N₄O₈ (M+H): calc. 607.28, found 607.15 (M+H). HPLC: 85% purity.

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate: Compound 12

To a stirred solution of (S)-tert-butyl (4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate), compound 18 (2 g, 3.30 mmol) in DCM was added TFA (5.08 mL, 65.9 mmol) at 0° C. and the resulting mixture was stirred at rt for 16 h. The solvent was removed by purging with N₂ gas. The crude reaction mass was then triturated with diethyl ether and the obtained solids were washed with n-pentane and dried in vacuo to provide (S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate, compound 12 (2.0 g, 2.96 mmol, 90% yield) as a yellow solid. LC-MS: C₂₇H₃₁N₄O₆ (M+H): calc. 507.22, found 507.70 (M+H). HPLC: 89% purity.

### Example 2: Synthesis of Target C, TFA Salt

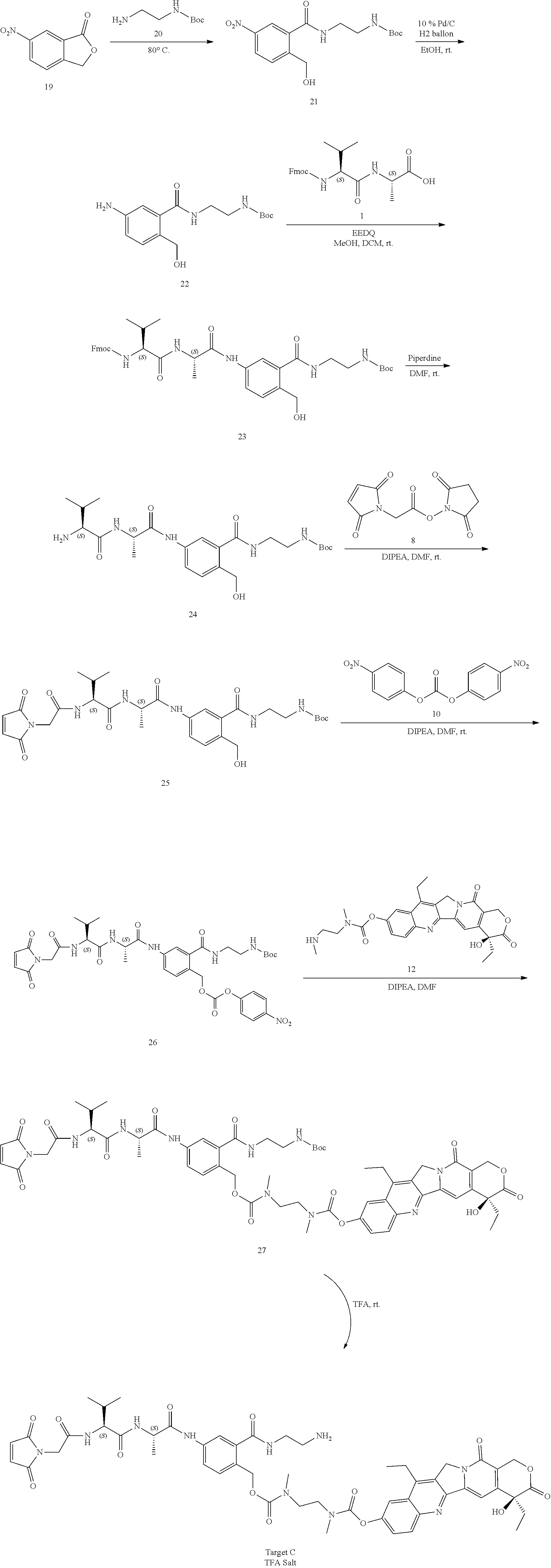

### Synthesis of tert-butyl (2-(2-(hydroxymethyl)-5-nitrobenzamido)ethyl)carbamate: Compound 21

6-nitroisobenzofuran-1(3H)-one, compound 19 (5.0 g, 27.9 mmol) and tert-butyl (2-aminoethyl)carbamate, compound 20 (4.92 g, 30.7 mmol) were mixed, heated at 90° C. for 2 h. The reaction was monitored by TLC. The reaction mixture was diluted with diethyl ether (50 mL) to provide tert-butyl (2-(2-(hydroxymethyl)-5-nitrobenzamido)ethyl)carbamate, compound 21 (6.5 g, 19.15 mmol, 68.6% yield) as a white solid. LC-MS: C₁₅H₂₂N₃O₆ (M+H): calc. 340.15, found 340.05 (M+H).

### Synthesis of tert-butyl (2-(5-amino-2-(hydroxymethyl)benzamido)ethyl)carbamate: Compound 22

To a stirred solution of tert-butyl (2-(2-(hydroxymethyl)-5-nitrobenzamido)ethyl)carbamate, compound 21 (6.5 g, 19.15 mmol) dissolved in ethanol (50 mL) and THF (50 mL) was added 10% palladium on carbon (1.02 g, 0.96 mmol). The reaction mixture was stirred at rt for 16 h under a hydrogen balloon (50 psi). Reaction progress was monitored by TLC. The reaction mixture was filtered through a celite pad. The collected filtrate was evaporated under reduced pressure to provide tert-butyl (2-(5-amino-2-(hydroxymethyl)benzamido)ethyl)carbamate, compound 22 (5.8 g, 18.75 mmol, 98% yield) as a white solid. LC-MS: C₁₅H₂₄N₃O₄ (M-OH): calc. 292.16, found 291.95 (M-OH).

### Synthesis of (9H-fluoren-9-yl)methyl ((S)-1-(((S)-1-((3-((2-((tert-butoxycarbonyl)amino)ethyl)carbamoyl)-4-(hydroxymethyl)phenyl)amino)-1-oxopropan-2-yl)amino)-3-methyl-1-oxobutan-2-yl)carbamate: Compound 23

To a stirred solution of (((9H-fluoren-9-yl)methoxy)carbonyl)-L-valyl-L-alanine, compound 1 (6.0 g, 14.62 mmol) and tert-butyl (2-(5-amino-2-(hydroxymethyl)benzamido)ethyl)carbamate, compound 22 (5.43 g, 17.54 mmol) in DCM (60 mL)/MeOH (20 mL) was added ethyl 2-ethoxyquinoline-1(2H)-carboxylate (7.23 g, 29.2 mmol). The reaction mixture was stirred at rt for 16 h. The reaction mixture was diluted with diethyl ether (100 mL). The obtained solid was filtered and dried under reduced pressure to provide (9H-fluoren-9-yl)methyl ((S)-1-(((S)-1-((3-((2-((tert-butoxycarbonyl)amino)ethyl)carbamoyl)-4-(hydroxymethyl)phenyl)amino)-1-oxopropan-2-yl)amino)-3-methyl-1-oxobutan-2-yl)carbamate, compound 23 (8.0 g, 11.40 mmol, 78% yield) as a white solid. LC-MS: C₃₈H₄₈N₅O₈ (M-OH): calc. 684.34, found 684.15 (M-OH).

### Synthesis of tert-butyl (2-(5-((S)-2-((S)-2-amino-3-methylbutanamido)propanamido)-2-(hydroxymethyl)benzamido)ethyl)carbamate: Compound 24

To a stirred solution of (9H-fluoren-9-yl)methyl ((S)-1-(((S)-1-((3-((2-((tert-butoxycarbonyl)amino)ethyl)carbamoyl)-4-(hydroxymethyl)phenyl)amino)-1-oxopropan-2-yl)amino)-3-methyl-1-oxobutan-2-yl)carbamate, compound 23 (8.0 g, 11.40 mmol) in DMF (60 mL) was added piperidine (3.39 mL, 34.2 mmol). The resulting mixture was stirred at rt for 30 min. The solvent was then evaporated under reduced pressure. The crude residue was washed with hexane (2×50 mL), diethyl ether (2×50 mL) then dried under vacuum to afford tert-butyl (2-(5-((S)-2-((S)-2-amino-3-methylbutanamido)propanamido)-2-(hydroxymethyl)benzamido)ethyl)carbamate, compound 24 (4.0 g, 8.34 mmol, 73.2% yield) as white solid. LC-MS: C₂₃H₃₈N₅O₆ (M+H): calc. 480.28, found 480.35 (M+H).

### Synthesis of tert-butyl (2-(5-((S)-2-((S)-2-(2-(2,5-dioxo-2,5-dihydro-1H-pyrrol-1-yl)acetamido)-3-methylbutanamido)propanamido)-2-(hydroxymethyl)benzamido)ethyl)carbamate: Compound 25

To a stirred solution of tert-butyl (2-(5-((S)-2-((S)-2-amino-3-methylbutanamido)propanamido)-2-(hydroxymethyl)benzamido)ethyl)carbamate, compound 24 (4.0 g, 8.34 mmol) in DMF (50 mL) was added 2,5-dioxopyrrolidin-1-yl 2-(2,5-dioxo-2,5-dihydro-1H-pyrrol-1-yl)acetate, compound 8 (2.1 g, 8.34 mmol) and DIPEA (1.46 mL, 8.34 mmol) at rt and the resulting mixture was stirred for 1 h. The reaction mixture was diluted with diethyl ether (200 mL) and then filtered. The obtained solid was washed with diethyl ether (3×) and dried under reduced pressure to provide tert-butyl (2-(5-((S)-2-((S)-2-(2-(2,5-dioxo-2,5-dihydro-1H-pyrrol-1-yl)acetamido)-3-methylbutanamido)propanamido)-2-(hydroxymethyl)benzamido)ethyl)carbamate, compound 25 (3.0 g, 4.86 mmol, 58.3% yield) as a white solid. LC-MS: C₂₉H₄₁N₆O₉ (M+H): calc. 617.29, found 617.10 (M+H).

### Synthesis of tert-butyl (2-(5-((S)-2-((S)-2-(2-(2,5-dioxo-2,5-dihydro-1H-pyrrol-1-yl)acetamido)-3-methylbutanamido)propanamido)-2-((((4-nitrophenoxy)carbonyl)oxy)methyl)benzamido)ethyl)carbamate: Compound 26

To a stirred solution of tert-butyl (2-(5-((S)-2-((S)-2-(2-(2,5-dioxo-2,5-dihydro-1H-pyrrol-1-yl)acetamido)-3-methylbutanamido)propanamido)-2-(hydroxymethyl)benzamido)ethyl)carbamate, compound 25 (3.00 g, 4.86 mmol) in DMF (50 mL) was added bis(4-nitrophenyl) carbonate, compound 10 (7.40 g, 24.32 mmol) and DIPEA (4.25 mL, 24.32 mmol) and the resulting mixture was stirred at rt for 1 h. The reaction mixture was diluted with diethyl ether (250 mL), the obtained solid was filtered and dried under vacuum to provide tert-butyl (2-(5-((S)-2-((S)-2-(2-(2,5-dioxo-2,5-dihydro-1H-pyrrol-1-yl)acetamido)-3-methylbutanamido)propanamido)-2-((((4-nitrophenoxy)carbonyl)oxy)methyl)benzamido)ethyl)carbamate, compound 26 (2.8 g, 2.90 mmol, 81% yield) as an off-white solid. LC-MS: C₃₆H₄₄N₇O₁₃ (M−Boc+H): calc. 682.25, found 681.95 (M−Boc+H).

### Synthesis of 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate): Compound 27

To a stirred solution of tert-butyl (2-(5-((S)-2-((S)-2-(2-(2,5-dioxo-2,5-dihydro-1H-pyrrol-1-yl)acetamido)-3-methylbutanamido)propanamido)-2-((((4-nitrophenoxy)carbonyl)oxy)methyl)benzamido)ethyl)carbamate, compound 26 (363 mg, 0.46 mmol) and (S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate 12 (200 mg, 0.33 mmol) in DMF (8 mL) was added DIPEA (0.29 mL, 1.66 mmol) and the resulting mixture was stilled at rt for 1 h. The reaction mixture was evaporated under pressure. The crude compound (0.8 g) was purified by prep HPLC (Mobile phase A: 0.1% TFA in H₂O, Mobile phase B: 100% acetonitrile). The pure fractions were dried by lyophilization for 2 days to obtained 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate), compound 27 (100 mg, 0.074 mmol, 10.68% yield) as a yellow solid. LC-MS: C₅₇H₆₉N₁₀O₁₆ (M+H): calc. 1149.45, found 1149.05 (M+H). HPLC: 85.43% purity.

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((S)-2-((S)-2-(2-(2,5-dioxo-2,5-dihydro-1H-pyrrol-1-yl)acetamido)-3-methylbutanamido)propanamido)-2-((2-((2,2,2-trifluoroacetyl)-14-azaneyl)ethyl)carbamoyl)benzyl) ethane-1,2-diylbis(methylcarbamate): Target C, TFA Salt

2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate), compound 27 (250 mg, 0.218 mmol) in DCM (5 mL) was added TFA (0.34 mL, 4.35 mmol) at 0° C. and the reaction mixture was stirred at rt for 3 h. The reaction mixture was concentrated under reduced pressure. The crude reaction mass was then triturated with diethyl ether. The resulting solid was filtered, washed with n-pentane and dried in vacuo. The crude compound (250 mg) was purified by HPLC (Mobile phase A: 0.1% TFA in H₂O, Mobile phase B: 100% acetonitrile). Pure fractions were dried by lyophilization for 2 days to obtained (S)-4,11-diethyl-4-hydroxy-3,14-dioxo-3,4,12,14-tetrahydro-1H-pyrano[3′,4′:6,7]indolizino[1,2-b]quinolin-9-yl (4-((S)-2-((S)-2-(2-(2,5-dioxo-2,5-dihydro-1H-pyrrol-1-yl)acetamido)-3-methylbutanamido)propanamido)-2-((2-((2,2,2-trifluoroacetyl)-14-azaneyl)ethyl)carbamoyl)benzyl) ethane-1,2-diylbis(methylcarbamate) Target C, TFA salt (110 mg, 0.094 mmol, 43.1%) as a yellow solid.

¹H NMR (DMSO, 400 MHz): δ 9.72 (s, 1H), 8.25 (t, J=5.2 Hz, 1H), 8.15 (d, J=9.2 Hz, 1H), 7.97-7.88 (m, 3H), 7.81 (s, 1H), 7.71 (brs, 2H), 7.58 (dd, J=9.2 Hz, 1H), 7.50 (d, J=7.6 Hz, 1H), 7.38-7.34 (m, 2H), 6.98 (s, 2H), 5.41 (q, J=16.4 Hz, 2H), 5.31 (s, 2H), 5.21 (s, 2H), 4.42 (t, J=7.2 Hz, 1H), 4.20-4.14 (m, 3H), 3.54-3.49 (m, 4H), 3.19-3.15 (m, 3H), 3.00-2.95 (m, 8H), 2.04-1.87 (m, 3H), 1.33-1.29 (m, 6H), 1.25 (s, 2H), 0.92-0.85 (m, 9H). LC-MS: C₅₂H₆₁N₁₀O₁₄ (M+H): calc. 1049.44, found 1049.20 (M+H). HPLC: 97.91% purity.

### Example 3: Synthesis of Target E, TFA Salt

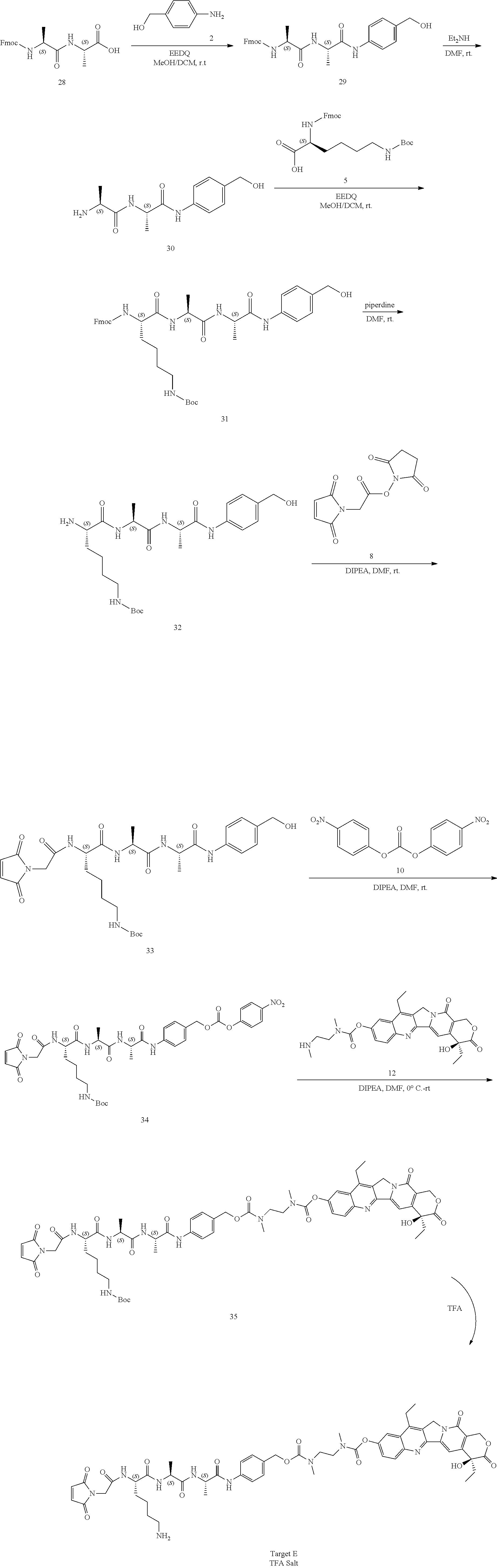

### Synthesis of (9H-fluoren-9-yl)methyl ((S)-1-(((S)-1-((4-(hydroxymethyl)phenyl)amino)-1-oxopropan-2-yl)amino)-1-oxopropan-2-yl)carbamate: Compound 29

To a stirred solution of (((9H-fluoren-9-yl)methoxy)carbonyl)-L-alanyl-L-alanine 1 (3.0 g, 7.84 mmol), compound 28 and (4-aminophenyl)methanol, compound 2 (1.45 g, 11.77 mmol) in DCM (50 mL) and MeOH (10 mL), was added EEDQ (3.49 g, 14.12 mmol). The reaction mixture was stirred at rt for 3 h. The mixture was concentrated under reduced pressure. The crude reaction mixture was then diluted with diethyl ether (50 mL) and stirred for 10 minutes at rt. The solid residue was filtered under vacuum, washed with diethyl ether (3×50 mL) and dried to provide (9H-fluoren-9-yl)methyl ((S)-1-(((S)-1-((4-(hydroxymethyl)phenyl)amino)-1-oxopropan-2-yl)amino)-1-oxopropan-2-yl)carbamate, compound 29 (3.62 g, 7.38 mmol, 94% yield) as a white solid. LC-MS: C₂₈H₃₀N₃O₅ (M+H): calc. 488.22, found 488.10 (M+H).

### Synthesis of (S)-2-amino-N—((S)-1-((4-(hydroxymethyl)phenyl)amino)-1-oxopropan-2-yl)propenamide: Compound 30

To a stirred solution of (9H-fluoren-9-yl)methyl ((S)-1-(((S)-1-((4-(hydroxymethyl)phenyl)amino)-1-oxopropan-2-yl)amino)-1-oxopropan-2-yl)carbamate, compound 29 (2.5 g, 5.13 mmol) in DMF (50 mL), was added diethylamine (10.18 mL, 97 mmol). The reaction mixture was stirred at rt for 16 h. The solvent was removed under vacuum and triturated with EtOAc (100 mL). The resulting solid was filtered and dried under vacuum to provide (S)-2-amino-N—((S)-1-((4-(hydroxymethyl)phenyl)amino)-1-oxopropan-2-yl)propenamide, compound 30 (1.04 g, 3.85 mmol, 98% yield) as a white solid. LC-MS: C₁₃H₂₀N₃O₃ (M+H): calc. 266.15, found 266.30 (M+H).

### Synthesis of (9H-fluoren-9-yl)methyl tert-butyl ((S)-6-(((S)-1-(((S)-1-((4-(hydroxymethyl)phenyl)amino)-1-oxopropan-2-yl)amino)-1-oxopropan-2-yl)amino)-6-oxohexane-1,5-diyl)dicarbamate: Compound 31

To a stirred solution of (S)-2-amino-N—((S)-1-((4-(hydroxymethyl)phenyl)amino)-1-oxopropan-2-yl), compound 30 (2.88 g, 10.86 mmol) and N2-(((9H-fluoren-9-yl)methoxy)carbonyl)-N6-(tert-butoxycarbonyl)-L-lysine, compound 5 (7.63 g, 16.28 mmol) in DCM (75 mL) and MeOH (15 mL), was added EEDQ (4.83 g, 19.54 mmol) at rt. The reaction mixture was allowed to stirred at rt for 16 h. The reaction mixture was concentrated under reduced pressure and triturated with diethyl ether (100 mL). The resulting solid was washed with diethyl ether (3×50 mL) and dried under vacuum to provide (9H-fluoren-9-yl)methyl tert-butyl ((S)-6-(((S)-1-(((S)-1-((4-(hydroxymethyl)phenyl)amino)-1-oxopropan-2-yl)amino)-1-oxopropan-2-yl)amino)-6-oxohexane-1,5-diyl)dicarbamate, compound 31 (7.4 g, 9.57 mmol, 88% yield) as a white solid. LC-MS: C₃₉H₅₀N₅O₈(M−Boc+H): calc. 616.31, found 616.25 (M−Boc+H).

### Synthesis of tert-butyl ((S)-5-amino-6-(((S)-1-(((S)-1-((4-(hydroxymethyl)phenyl)amino)-1-oxopropan-2-yl)amino)-1-oxopropan-2-yl)amino)-6-oxohexyl)carbamate: Compound 32

To a stirred solution of (9H-fluoren-9-yl)methyl tert-butyl ((S)-6-(((S)-1-(((S)-1-((4-(hydroxymethyl)phenyl)amino)-1-oxopropan-2-yl)amino)-1-oxopropan-2-yl)amino)-6-oxohexane-1,5-diyl)dicarbamate, compound 31 (7.4 g, 10.34 mmol) in DMF (50 mL) was added diethylamine (20.52 mL, 196 mmol) at rt. The reaction mixture was stirred at rt for 16 h. The solvent was evaporated under rotary evaporator and triturated with EtOAc (100 mL). The resulting solid was filtered, washed with diethyl ether (100 mL) and dried under vacuum to provide tert-butyl ((S)-5-amino-6-(((S)-1-(((S)-1-((4-(hydroxymethyl)phenyl)amino)-1-oxopropan-2-yl)amino)-1-oxopropan-2-yl)amino)-6-oxohexyl)carbamate, compound 32 (4.9 g, 9.23 mmol, 89% yield) as a white solid. LC-MS: C₂₄H₄₀N₅O₆ (M+H): calc. 494.30, found 494.55 (M+H).

### Synthesis of tert-butyl ((S)-5-(2-(2,5-dioxo-2,5-dihydro-1H-pyrrol-1-yl)acetamido)-6-(((S)-1-(((S)-1-((4-(hydroxymethyl)phenyl)amino)-1-oxopropan-2-yl)amino)-1-oxopropan-2-yl)amino)-6-oxohexyl)carbamate: Compound 33

To a stirred solution of tert-butyl ((S)-5-amino-6-(((S)-1-(((S)-1-((4-(hydroxymethyl)phenyl)amino)-1-oxopropan-2-yl)amino)-1-oxopropan-2-yl)amino)-6-oxohexyl)carbamate, compound 32 (4.6 g, 9.32 mmol) in DMF (50 mL) was added DIPEA (3.26 mL, 18.64 mmol) followed by the addition of 2,5-dioxopyrrolidin-1-yl 2-(2,5-dioxo-2,5-dihydro-1H-pyrrol-1-yl)acetate, compound 8 (2.35 g, 9.32 mmol) at rt. The reaction mixture was stirred at rt for 16 h. The solvent was evaporated under rotary evaporator and triturated with EtOAc (50 mL). The resulting solid was filtered, washed with diethyl ether (100 mL) and dried under vacuum to provide tert-butyl ((S)-5-(2-(2,5-dioxo-2,5-dihydro-1H-pyrrol-1-yl)acetamido)-6-(((S)-1-(((S)-1-((4-(hydroxymethyl)phenyl)amino)-1-oxopropan-2-yl)amino)-1-oxopropan-2-yl)amino)-6-oxohexyl)carbamate, compound 33 (5.7 g, 6.97 mmol, 74.8% yield) as brown solid. LC-MS: C₃₀H₄₃N₆O₉ (M−Boc+H): calc. 531.26, found 531.25 (M−Boc+H).

### Synthesis of tert-butyl ((S)-5-(2-(2,5-dioxo-2,5-dihydro-1H-pyrrol-1-yl)acetamido)-6-(((S)-1-(((S)-1-((4-((((4-nitrophenoxy)carbonyl)oxy)methyl)phenyl)amino)-1-oxopropan-2-yl)amino)-1-oxopropan-2-yl)amino)-6-oxohexyl)carbamate: Compound 34

To a stirred solution of tert-butyl ((S)-5-(2-(2,5-dioxo-2,5-dihydro-1H-pyrrol-1-yl)acetamido)-6-(((S)-1-(((S)-1-((4-(hydroxymethyl)phenyl)amino)-1-oxopropan-2-yl)amino)-1-oxopropan-2-yl)amino)-6-oxohexyl)carbamate, compound 33 (5.7 g, 9.04 mmol) in DMF (60 mL) was added DIPEA (7.89 mL, 45.2 mmol) and followed by addition of bis(4-nitrophenyl) carbonate, compound 10 (13.75 g, 45.2 mmol) at rt. The reaction mixture was stirred at rt for 16 h. The solvent was evaporated under rotary evaporator and triturated with diethyl ether (100 mL). The resulting solid was filtered, washed with diethyl ether (100 mL) and dried under vacuum to provide the crude compound which was purified by combi flash using 7-10% MeOH in DCM to provide tert-butyl ((S)-5-(2-(2,5-dioxo-2,5-dihydro-1H-pyrrol-1-yl)acetamido)-6-(((S)-1-(((S)-1-((4-((((4-nitrophenoxy)carbonyl)oxy)methyl)phenyl)amino)-1-oxopropan-2-yl)amino)-1-oxopropan-2-yl)amino)-6-oxohexyl)carbamate, compound 34 (batch 1: 1.3 g, 1.55 mmol, 17.2% yield, 95% by HPLC; batch 2: 2.3 g, 2.77 mmol, 30.7%, 67% by HPLC) as a brown solid. LC-MS: C₃₇H₄₆N₇O₁₃ (M−Boc+H): calc. 696.26, found 695.95 (M−Boc+H).

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(2,5-dioxo-2,5-dihydro-1H-pyrrol-1-yl)acetamido)-2,2,13,16-tetramethyl-4,11,14-trioxo-3-oxa-5,12,15-triazaheptadecan-17-amido)benzyl) ethane-1,2-diylbis(methylcarbamate): Compound

To a stirred solution of tert-butyl ((S)-5-(2-(2,5-dioxo-2,5-dihydro-1H-pyrrol-1-yl)acetamido)-6-(((S)-1-(((S)-1-((4-((((4-nitrophenoxy)carbonyl)oxy)methyl)phenyl)amino)-1-oxopropan-2-yl)amino)-1-oxopropan-2-yl)amino)-6-oxohexyl)carbamate, compound 34 (791 mg, 0.994 mmol), and (S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate, compound 12 (prepared as described in Example 1A, 600 mg, 0.99 mmol) in DMF (6 mL) was added DIPEA (0.87 mL, 4.97 mmol) and the reaction mixture was stirred at rt for 1 h. The reaction mixture was evaporated under pressure. The crude compound was purified by HPLC (Mobile phase A: 0.1% TFA in H₂O, Mobile phase B: 100% acetonitrile). Pure fractions were dried by lyophilization for 2 days to obtained (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,135,16S)-10-(2-(2,5-dioxo-2,5-dihydro-1H-pyrrol-1-yl)acetamido)-2,2,13,16-tetramethyl-4,11,14-trioxo-3-oxa-5,12,15-triazaheptadecan-17-amido)benzyl) ethane-1,2-diylbis(methylcarbamate), compound 35 (95 mg, 0.078 mmol, 7.86% yield) as a yellow solid. LC-MS: C₅₈H₇₁N₁₀O₁₆ (M+H): calc. 1163.51, found 1163.15 (M+H). HPLC: 95.65% purity.

### Synthesis of 4-((S)-2-((S)-2-((S)-6-amino-2-(2-(2,5-dioxo-2,5-dihydro-1H-pyrrol-1-yl)acetamido)hexanamido)propanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate): Target E, TFA salt

To (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(2,5-dioxo-2,5-dihydro-1H-pyrrol-1-yl)acetamido)-2,2,13,16-tetramethyl-4,11,14-trioxo-3-oxa-5,12,15-triazaheptadecan-17-amido)benzyl) ethane-1,2-diylbis(methylcarbamate), compound 35 (95 mg, 0.08 mmol) in DCM (5 mL) was added TFA (0.38 mL, 4.90 mmol) at 0° C. and the reaction mixture was stirred at rt for 1 h. The reaction mixture was concentrated under reduced pressure. The crude product was then triturated with diethyl ether to provide 4-((S)-2-((S)-2-((S)-6-amino-2-(2-(2,5-dioxo-2,5-dihydro-1H-pyrrol-1-yl)acetamido)hexanamido)propanamido)propanamido)benzyl ((S)-4,11-diethyl-4-hydroxy-3,14-dioxo-3,4,12,14-tetrahydro-1H-pyrano[3′,4′:6,7]indolizino[1,2-b]quinolin-9-yl) ethane-1,2-diylbis(methylcarbamate) Target E, TFA salt (90 mg, 0.08 mmol, 99% yield) as a yellow solid.

¹H NMR (DMSO-d₆, 400 MHz): δ 9.58 (s, 1H), 8.16-8.12 (m, 2H), 7.92 (s, 1H), 7.78 (d, J=6.8 Hz, 1H), 7.68 (d, J=7.2 Hz, 1H), 7.58-7.51 (m, 6H), 7.35 (s, 1H), 7.27 (d, J=8.0 Hz, 2H), 6.99 (s, 2H), 6.30-6.05 (brs, 1H), 5.41 (q, J=16.0 Hz, 2H), 5.31 (s, 2H), 5.03 (s, 1H), 4.42-4.39 (t, J=7.2 Hz, 1H), 4.31-4.25 (m, 2H), 4.11 (s, 2H), 3.55-3.51 (brs, 4H), 3.20-3.15 (m, 4H), 2.95 (s, 3H), 2.78-2.75 (m, 2H), 1.92-1.87 (m, 2H), 1.75-1.65 (m, 1H), 1.56-1.54 (m, 3H), 1.31-1.25 (m, 12H), 0.90 (t, J=7.2 Hz, 3H). LC-MS: C₅₃H₆₃N₁₀O₁₄ (M+H): calc. 1063.45, found 1063.20 (M+H); HPLC: 95.17% purity.

### Example 4: Synthesis of Target H, TFA Salt

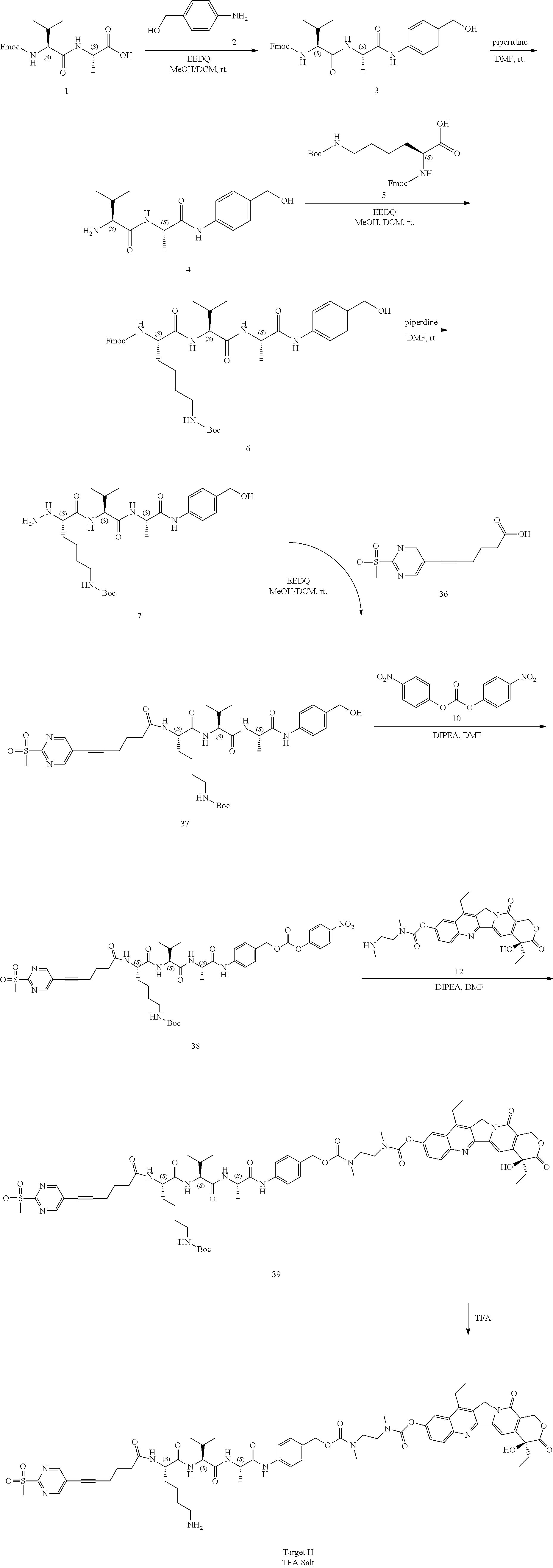

### Synthesis of tert-butyl ((S)-6-(((S)-1-(((S)-1-((4-(hydroxymethyl)phenyl)amino)-1-oxopropan-2-yl)amino)-3-methyl-1-oxobutan-2-yl)amino)-5-(6-(2-(methylsulfonyl)pyrimidin-5-yl)hex-5-ynamido)-6-oxohexyl)carbamate: Compound 37

To a stirred solution of tert-butyl ((S)-5-amino-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate 7 (prepared as described in Example 1, 0.65 g, 1.25 mmol) in DCM (15 mL) and MeOH (3.75 mL) were added 6-(2-(methylsulfonyl)pyrimidin-5-yl)hex-5-ynoic acid, compound 36 (0.84 g, 3.12 mmol) and EEDQ (1.08 g, 4.36 mmol) at rt. The reaction mixture was stirred at rt for 16 h then quenched by the addition of diethyl ether. The resulting solid was filtered and washed with diethyl ether, dried under vacuum to provide tert-butyl ((S)-6-(((S)-1-(((S)-1-((4-(hydroxymethyl)phenyl)amino)-1-oxopropan-2-yl)amino)-3-methyl-1-oxobutan-2-yl)amino)-5-(6-(2-(methylsulfonyl)pyrimidin-5-yl)hex-5-ynamido)-6-oxohexyl)carbamate, compound 37 (800 mg, 0.91 mmol, 73%) as a pale yellow solid. L^{C}-MS: C₃₇H₅₄N₇O₉S (M−Boc+H): calc. 672.30, found 672.20 (M−Boc+H).

### Synthesis of tert-butyl ((S)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-5-(6-(2-(methylsulfonyl)pyrimidin-5-yl)hex-5-ynamido)-6-oxohexyl)carbamate: Compound 38

To a stirred solution of tert-butyl ((S)-6-(((S)-1-(((S)-1-((4-(hydroxymethyl)phenyl)amino)-1-oxopropan-2-yl)amino)-3-methyl-1-oxobutan-2-yl)amino)-5-(6-(2-(methylsulfonyl)pyrimidin-5-yl)hex-5-ynamido)-6-oxohexyl)carbamate, compound 37 (0.8 g, 1.04 mmol) in DMF (10 ml) was added bis(4-nitrophenyl) carbonate 10 (0.95 g, 3.11 mmol), DIPEA (0.54 mL, 3.11 mmol) at 0° C. The reaction mixture was stirred for 3 h at rt, then quenched by the addition of hexane. The resulting solid was filtered and washed with diethyl ether. The resulting solid was dissolved in acetone and again precipitated by the addition of diethyl ether. The solid was washed with diethyl ether (3×) and dried under reduced pressure to provide tert-butyl ((S)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-5-(6-(2-(methylsulfonyl)pyrimidin-5-yl)hex-5-ynamido)-6-oxohexyl)carbamate, compound 38 (1 g, 0.94 mmol, 90%) as a white solid. LC-MS: C₄₄H₅₆N₈O₁₃S (M−Boc): calc. 836.32, found 836.95 (M−Boc).

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-13-isopropyl-2,2,16-trimethyl-10-(6-(2-(methylsulfonyl)pyrimidin-5-yl)hex-5-ynamido)-4,11,14-trioxo-3-oxa-5,12,15-triazaheptadecan-17-amido)benzyl) ethane-1,2-diylbis(methylcarbamate): Compound 39

(S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate 12 (100 mg, 0.17 mmol) in DMF (1 mL) was added to a stirred solution of tert-butyl ((S)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-5-(6-(2-(methylsulfonyl)pyrimidin-5-yl)hex-5-ynamido)-6-oxohexyl)carbamate, compound 38 (155 mg, 0.17 mmol), DIPEA (0.15 mL, 0.83 mmol) in DMF (2 mL) at 0° C. The reaction mixture was stirred at rt for 1 h, then diluted with diethyl ether. The resulting solids were washed with diethyl ether and n-pentane and then dried in vacuum to provide (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-13-isopropyl-2,2,16-trimethyl-10-(6-(2-(methylsulfonyl)pyrimidin-5-yl)hex-5-ynamido)-4,11,14-trioxo-3-oxa-5,12,15-triazaheptadecan-17-amido)benzyl) ethane-1,2-diylbis(methylcarbamate), compound 39 (190 mg, 0.073 mmol, 44%) as a yellow solid. LC-MS: C₆₅H₈₂N₁₁O₁₆S (M−H): calc. 1302.55, found 1302.25 (M−H). This compound was used in the next step without purification.

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-13-isopropyl-2,2,16-trimethyl-10-(6-(2-(methylsulfonyl)pyrimidin-5-yl)hex-5-ynamido)-4,11,14-trioxo-3-oxa-5,12,15-triazaheptadecan-17-amido)benzyl) ethane-1,2-diylbis(methylcarbamate): Target H, TFA Salt

To a stirred solution of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-13-isopropyl-2,2,16-trimethyl-10-(6-(2-(methylsulfonyl)pyrimidin-5-yl)hex-5-ynamido)-4,11,14-trioxo-3-oxa-5,12,15-triazaheptadecan-17-amido)benzyl) ethane-1,2-diylbis(methylcarbamate), compound 39 (190 mg, 0.15 mmol) in DCM (10 mL) was added TFA (0.56 mL, 7.28 mmol) at 0° C. The reaction mixture was stirred at rt for 4 h, then concentrated and washed with diethyl ether to provide the crude compound (330 mg) that was purified by HPLC (Mobile phase A: 0.1% TFA in H₂O, Mobile phase B: 100% acetonitrile). Pure fractions were dried by lyophilization for 2 days to obtain (S)-4,11-diethyl-4-hydroxy-3,14-dioxo-3,4,12,14-tetrahydro-1H-pyrano[3′,4′:6,7]indolizino[1,2-b]quinolin-9-yl (4-((S)-2-((S)-3-methyl-2-((S)-2-(6-(2-(methylsulfonyl)pyrimidin-5-yl)hex-5-ynamido)-6-((2,2,2-trifluoroacetyl)-14-azaneyl)hexanamido)butanamido)propanamido)benzyl) ethane-1,2-diylbis(methylcarbamate) Target H, TFA salt (40 mg, 0.03 mmol, 13%) as a yellow solid.

¹H NMR (DMSO-d₆, 400 MHz): δ 9.97 (s, 1H), 9.11 (s, 2H), 8.20-8.09 (m, 3H), 7.92 (d, J=8.8 Hz, 1H), 7.69-7.51 (m, 7H), 7.33 (s, 2H), 7.27 (d, J=8.0 Hz, 1H), 6.53 (br s, 1H), 5.45 (s, 2H), 5.35 (s, 2H), 5.05 (s, 1H), 5.00 (d, J=8.6 Hz, 1H), 4.37-4.28 (m, 2H), 4.20 (t, 7.6 Hz, 1H), 3.64 (m, 2H), 3.41 (s, 3H), 3.20-3.10 (m, 3H), 2.97-2.95 (m, 2H), 2.92-2.88 (m, 2H), 2.75-2.74 (m, 2H), 2.34-2.32 (m, 3H), 1.87-1.79 (m, 5H), 1.66-1.65 (m, 1H), 1.51-1.49 (m, 4H), 1.29-1.24 (m, 9H), 0.90-0.80 (m, 10H). LC-MS: C₆₀H₇₄N₁₁O₁₄S (M+H): calc. 1204.51, found 1204.20 (M+H). HPLC: 96.10% purity.

### Example 5: Synthesis of Target I

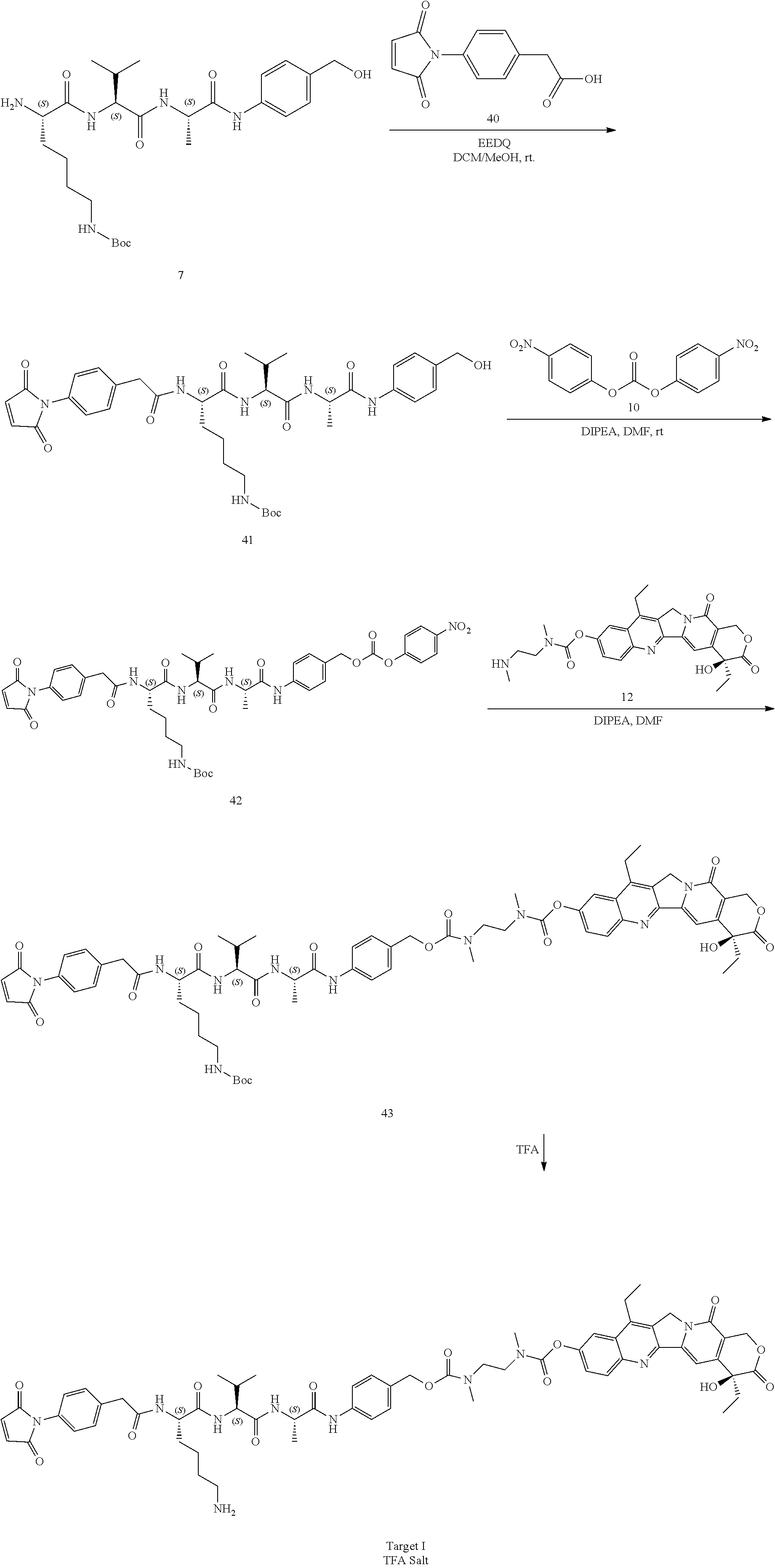

### Synthesis of tert-butyl ((S)-5-(2-(4-(2,5-dioxo-2,5-dihydro-1H-pyrrol-1-yl)phenyl)acetamido)-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate: Compound 41

To a stirred solution of tert-butyl ((S)-5-amino-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, compound 7 (prepared as described in Example 1, 1.0 g, 1.92 mmol) in DCM (30 mL) and MeOH (7.50 mL) was added 2-(4-(2,5-dioxo-2,5-dihydro-1H-pyrrol-1-yl)phenyl)acetic acid, compound 40 (0.54 g, 2.40 mmol), EEDQ (0.95 g, 3.83 mmol) at rt. The reaction mixture was stirred at rt for 16 h, then diluted with diethyl ether. The resulting solid was filtered and washed with diethyl ether (3×), then dried under reduced pressure to provide tert-butyl ((S)-5-(2-(4-(2,5-dioxo-2,5-dihydro-1H-pyrrol-1-yl)phenyl)acetamido)-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, compound 41 (1.4 g, 1.89 mmol, 98%) as a pale yellow solid. LC-MS: C₃₈H₅₁N₆O₉ (M+H): calc. 735.37, found 735.15 (M+H).

### Synthesis of tert-butyl ((S)-5-(2-(4-(2,5-dioxo-2,5-dihydro-1H-pyrrol-1-yl)phenyl)acetamido)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-6-oxohexyl)carbamate: Compound 42

To a stirred solution of tert-butyl ((S)-5-(2-(4-(2,5-dioxo-2,5-dihydro-1H-pyrrol-1-yl)phenyl)acetamido)-6-(((S)-1-(((S)-1-((4-(hydroxymethyl)phenyl)amino)-1-oxopropan-2-yl)amino)-3-methyl-1-oxobutan-2-yl)amino)-6-oxohexyl)carbamate, compound 41 (1.4 g, 1.91 mmol) in DMF (10 ml), was added bis(4-nitrophenyl) carbonate, compound 10 (1.74 g, 5.72 mmol) and DIPEA (1 mL, 5.72 mmol) at 0° C. The reaction mixture was stirred at rt for 3 h, then diluted with hexanes. The resulting solid was filtered and washed with diethyl ether (3×), then dried under reduced pressure to provide tert-butyl ((S)-5-(2-(4-(2,5-dioxo-2,5-dihydro-1H-pyrrol-1-yl)phenyl)acetamido)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-6-oxohexyl)carbamate, compound 42 (1.5 g, 1.53 mmol, 80% yield) as a white solid. LC-MS: C₄₅H₅₄N₇O₁₃ (M−Boc+H): calc. 800.33, found 800.10 (M−Boc+H).

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(4-(2,5-dioxo-2,5-dihydro-1H-pyrrol-1-yl)phenyl)acetamido)-13-isopropyl-2,2,16-trimethyl-4,11,14-trioxo-3-oxa-5,12,15-triazaheptadecan-17-amido)benzyl) ethane-1,2-diylbis(methylcarbamate): Compound 43

(S)-4,11-diethyl-4-hydroxy-3,14-dioxo-3,4,12,14-tetrahydro-1H-pyrano[3′,4′:6,7]indolizino[1,2-b]quinolin-9-yl methyl(2-(methyl(2,2,2-trifluoroacetyl)-14-azaneyl)ethyl)carbamate, compound 12 (prepared as described in Example 1A, 200 mg, 0.33 mmol) in DMF (2 mL) was added to a stirred solution of tert-butyl ((S)-5-(2-(4-(2,5-dioxo-2,5-dihydro-1H-pyrrol-1-yl)phenyl)acetamido)-6-(((S)-3-methyl-1-(((S)-1-((4-((((4-nitrophenoxy)carbonyl)oxy)methyl)phenyl)amino)-1-oxopropan-2-yl)amino)-1-oxobutan-2-yl)amino)-6-oxohexyl)carbamate, compound 42 (328 mg, 0.36 mmol), and DIPEA (0.29 mL, 1.66 mmol) in DMF (3 mL) at 0° C. The reaction mixture was stirred at rt for 0.5 h, then quenched by the addition of diethyl ether. The resulting solid was filtered and washed with diethyl ether (2×), then washed with acetonitrile (ACN) and dried under vacuum to provide crude (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(4-(2,5-dioxo-2,5-dihydro-1H-pyrrol-1-yl)phenyl)acetamido)-13-isopropyl-2,2,16-trimethyl-4,11,14-trioxo-3-oxa-5,12,15-triazaheptadecan-17-amido)benzyl) ethane-1,2-diylbis(methylcarbamate) compound 43 (400 mg, 0.18 mmol, 55%) as a white solid. LC-MS: C₆₆H₇₉N₁₀O₁₆ (M+H): calc. 1267.57, found 1267.20 (M+H).

### Synthesis of (S)-4,11-diethyl-4-hydroxy-3,14-dioxo-3,4,12,14-tetrahydro-1H-pyrano[3′,4′:6,7]indolizino[1,2-b]quinolin-9-yl (4-((S)-2-((S)-2-((S)-2-(2-(4-(2,5-dioxo-2,5-dihydro-1H-pyrrol-1-yl)phenyl)acetamido)-6-((2,2,2-trifluoroacetyl)-14-azaneyl)hexanamido)-3-methylbutanamido)propanamido)benzyl) ethane-1,2-diylbis(methylcarbamate): Target I, TFA Salt

To a stirred solution of (S)-4,11-diethyl-4-hydroxy-3,14-dioxo-3,4,12,14-tetrahydro-1H-pyrano[3′,4′:6,7]indolizino[1,2-b]quinolin-9-yl (4-((10S,13S,16S)-10-(2-(4-(2,5-dioxo-2,5-dihydro-1H-pyrrol-1-yl)phenyl)acetamido)-13-isopropyl-2,2,16-trimethyl-4,11,14-trioxo-3-oxa-5,12,15-triazaheptadecan-17-amido)benzyl) ethane-1,2-diylbis(methylcarbamate), compound 43 (0.4 g, 0.32 mmol) in DCM (10 mL) was added TFA (1.22 mL, 15.78 mmol) at 0° C. The reaction mixture was stirred at rt for 16 h. The reaction mixture was evaporated to dryness, then was washed with diethyl ether and dried under vacuum to afford the crude compound (530 mg) that was purified by HPLC (Mobile phase A: 0.1% TFA in H₂O, Mobile phase B: 100% acetonitrile). Pure fractions were dried by lyophilization for 2 days to obtain (S)-4,11-diethyl-4-hydroxy-3,14-dioxo-3,4,12,14-tetrahydro-1H-pyrano[3′,4′:6,7]indolizino[1,2-b]quinolin-9-yl (4-((S)-2-((S)-2-((S)-2-(2-(4-(2,5-dioxo-2,5-dihydro-1H-pyrrol-1-yl)phenyl)acetamido)-6-((2,2,2-trifluoroacetyl)-14-azaneyl)hexanamido)-3-methylbutanamido)propanamido)benzyl) ethane-1,2-diylbis(methylcarbamate), Target I, TFA salt (30 mg, 0.026 mmol, 8%) as a yellow solid.

¹H NMR (DMSO-d₆, 400 MHz): δ 9.97 (s, 1H), 8.35 (d, J=8.0 Hz, 1H), 8.18-8.11 (m, 2H), 7.91 (d, J=2.4 Hz, 1H), 7.50 (d, 1H), 7.60-7.54 (m, 6H), 7.36-7.22 (m, 7H), 7.18 (s, 2H), 6.53 (s, 1H), 5.44 (s, 2H), 5.34 (s, 2H), 5.05-4.99 (m, 2H), 4.37-4.32 (m, 2H), 4.21-4.18 (m, 1H), 3.64-3.49 (m, 7H), 3.18-3.11 (m, 1H), 2.97-2.88 (m, 6H), 2.72-2.67 (m, 2H), 1.96-1.86 (m, 3H), 1.68-1.55 (m, 4H), 1.33-1.24 (m, 8H), 0.88 (t, J=7.2 Hz, 3H), 0.81 (dd, J=7.2 Hz, 6H). LC-MS: C₆₁H₆₉N₁₀O₁₄ (M−H): calc. 1165.50, found 1165.50 (M−H). HPLC: 97.21% purity.

### Example 6: Synthesis of Target M, TFA Salt

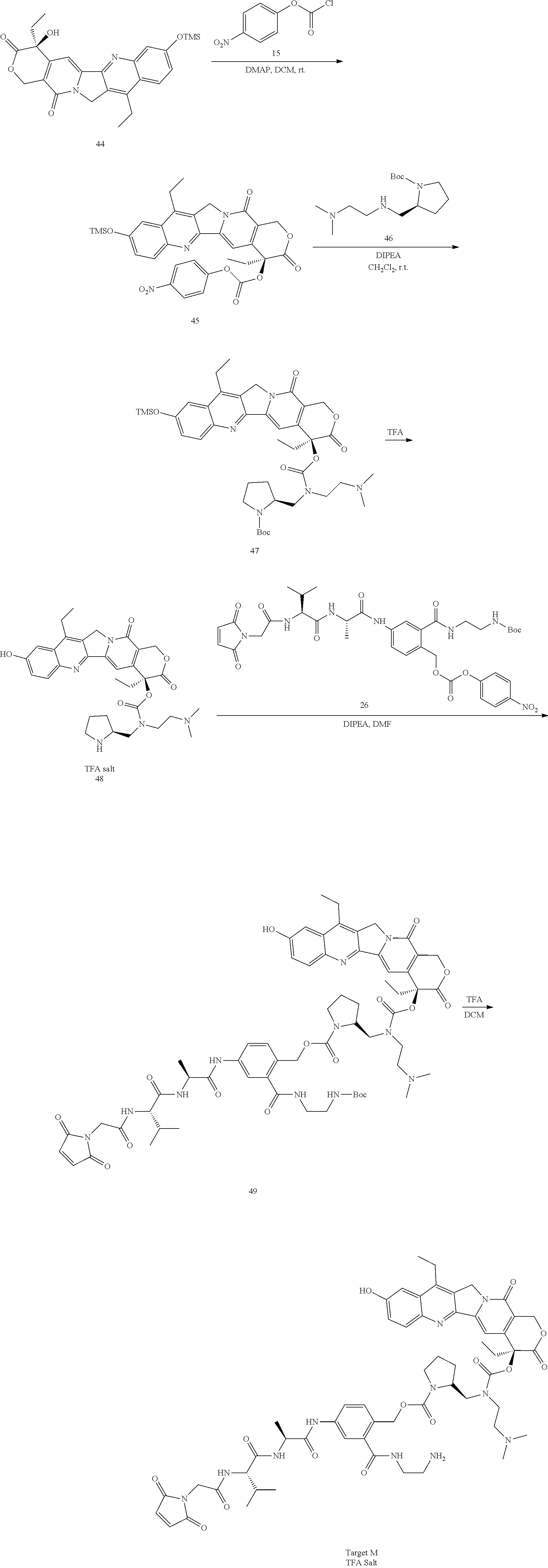

### Synthesis of (S)-4,11-diethyl-3,14-dioxo-9-((trimethylsilyl)oxy)-3,4,12,14-tetrahydro-1H-pyrano[3′,4′:6,7]indolizino[1,2-b]quinolin-4-yl (4-nitrophenyl) carbonate: Compound 45

To a stirred solution of (S)-4,11-diethyl-4-hydroxy-8-((trimethylsilyl)oxy)-1,12-dihydro-14H-pyrano[3′,4′:6,7]indolizino[1,2-b]quinoline-3,14(4H)-dione, compound 44 (100 mg, 0.22 mmol) and 4-nitrophenyl chloroformate, compound 15 (156 mg, 0.78 mmol) in DCM (15 mL) at 0° C. was added DMAP (147 mg, 1.21 mmol). The resulting solution was stirred at rt for 2 h. The progress of the reaction was monitored by TLC. After the reaction was complete, the mixture was diluted with DCM (30 mL) and washed with 0.1 N HCl (10 mL) and brine (20 mL). The organic layer was dried over Na₂SO₄, then concentrated under reduced pressure. The crude compound was purified by combi flash using MeOH in DCM to provide (S)-4,11-diethyl-3,14-dioxo-9-((trimethylsilyl)oxy)-3,4,12,14-tetrahydro-1H-pyrano[3′,4′:6,7]indolizino[1,2-b]quinolin-4-yl (4-nitrophenyl) carbonate, compound 45 (100 mg, 0.15 mmol, 69% yield) as a white solid. LC-MS: C₃₂H₃₁N₃O₉Si (M-TMS): calc. 556.14, found 556.10 (M-TMS).

### Synthesis of (tert-butyl (S)-2-((((((S)-4,11-diethyl-3,14-dioxo-9-((trimethylsilyl)oxy)-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate: Compound 47

To a stirred solution of (S)-4,11-diethyl-3,14-dioxo-9-((trimethylsilyl)oxy)-3,4,12,14-tetrahydro-1H-pyrano[3′,4′:6,7]indolizino[1,2-b]quinolin-4-yl (4-nitrophenyl) carbonate, compound 45 (650 mg, 1.03 mmol) in DCM (15 mL) was added tert-butyl (S)-2-(((2-(dimethylamino)ethyl)amino)methyl)pyrrolidine-1-carboxylate, compound 46 (840 mg, 3.10 mmol) under nitrogen atmosphere followed by the addition of DIPEA (0.72 mL, 4.13 mmol). The reaction mixture was stirred at rt for 2 h. The progress of the reaction was monitored by TLC. After the reaction was complete, the reaction mixture was concentrated under reduced pressure and co-distilled with DCM to provide (tert-butyl (S)-2-((((((S)-4,11-diethyl-3,14-dioxo-9-((trimethylsilyl)oxy)-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 47 (1.0 g, 1.13 mmol, quantitative yield) as a yellow-colored liquid. LC-MS: C₄₀H₅₅N₅O₈Si (M-TMS): calc. 688.33, found 688.20 (M-TMS).

### Synthesis of (S)-4,11-diethyl-9-hydroxy-3,14-dioxo-3,4,12,14-tetrahydro-1H-pyrano[3′,4′:6,7]indolizino[1,2-b]quinolin-4-yl (2-(dimethylamino)ethyl)(((S)-pyrrolidin-2-yl)methyl)carbamate: Compound 48

To a stirred solution of tert-butyl (S)-2-((((((S)-4,11-diethyl-3,14-dioxo-9-((trimethylsilyl)oxy)-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 47 (950 mg, 1.25 mmol) in DCM (25 mL) at 0° C. was added TFA (4.80 mL, 62.3 mmol). The reaction mixture was stirred at rt for 16 h, then concentrated under reduced pressure and co-distilled with DCM (2×). The crude compound was purified by HPLC (Mobile phase A: 0.1% TFA in H₂O, Mobile phase B: 100% acetonitrile). Pure fractions were dried by lyophilization for 2 days to obtained (S)-4,11-diethyl-9-hydroxy-3,14-dioxo-3,4,12,14-tetrahydro-1H-pyrano[3′,4′:6,7]indolizino[1,2-b]quinolin-4-yl (2-(dimethylamino)ethyl)(((S)-pyrrolidin-2-yl)methyl)carbamate, compound 48 (350 mg, 0.59 mmol, 47%) as a yellow solid. LC-MS: C₃₂H₄₀N₅O₆ (M−Boc+H): calc. 590.30, found 590.50 (M−Boc+H).

### Synthesis of 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl (R)-2-((((((S)-4,11-diethyl-9-hydroxy-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate: Compound 49

To a stirred solution of (S)-4,11-diethyl-9-hydroxy-3,14-dioxo-3,4,12,14-tetrahydro-1H-pyrano[3′,4′:6,7]indolizino[1,2-b]quinolin-4-yl (2-(dimethylamino)ethyl)(((S)-pyrrolidin-2-yl)methyl)carbamate, compound 48 (250 mg, 0.42 mmol) and tert-butyl (2-(5-((S)-2-((S)-2-(2-(2,5-dioxo-2,5-dihydro-1H-pyrrol-1-yl)acetamido)-3-methylbutanamido)propanamido)-2-((((4-nitrophenoxy)carbonyl)oxy)methyl)benzamido)ethyl)carbamate, compound 26 (365 mg, 0.47 mmol) in DMF (5 mL) at rt was added DIPEA (0.44 mL, 2.54 mmol). The reaction mixture was stirred at rt for 4 h. The progress of the reaction was monitored by TLC. After the reaction was complete, the reaction mixture was concentrated under reduced pressure. The residue was triturated with diethyl ether and dried under vacuum to provide a yellow solid which was purified by HPLC (Mobile phase A: 0.1% TFA in H₂O, Mobile phase B: 100% acetonitrile). Pure fractions were dried by lyophilization for 2 days to obtained 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl (R)-2-((((((S)-4,11-diethyl-9-hydroxy-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 49 (150 mg, 1.22 mmol, 29% yield) as a yellow solid. LC-MS: C₆₂H₇₇N₁₁O₁₆ (M+): calc. 1231.56, found 1231.35 (M), 1130.40 (M−Boc).

### Synthesis of 4-((S)-2-((S)-2-(2-(2,5-dioxo-2,5-dihydro-1H-pyrrol-1-yl)acetamido)-3-methylbutanamido)propanamido)-2-((2-((2,2,2-trifluoroacetyl)-14-azaneyl)ethyl)carbamoyl)benzyl (R)-2-((((((S)-4,11-diethyl-9-hydroxy-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate: Target M, TFA Salt

To a stirred solution of 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl (R)-2-((((((S)-4,11-diethyl-9-hydroxy-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 49 (175 mg, 0.142 mmol) in DCM (15 mL) was added TFA (1.09 mL, 14.20 mmol) at 0° C. The reaction mixture was stirred at rt for 2 h, then concentrated under reduced pressure and triturated with diethyl ether to provide the crude product which was purified by HPLC (Mobile phase A: 0.1% TFA in H₂O, Mobile phase B: 100% acetonitrile). Pure fractions were dried by lyophilization for 2 days to provide 4-((S)-2-((S)-2-(2-(2,5-dioxo-2,5-dihydro-1H-pyrrol-1-yl)acetamido)-3-methylbutanamido)propanamido)-2-((2-((2,2,2-trifluoroacetyl)-14-azaneyl)ethyl)carbamoyl)benzyl (R)-2-((((((S)-4,11-diethyl-9-hydroxy-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, Target M, TFA salt (80 mg, 0.065 mmol, 46% yield) as a yellow solid.

¹H NMR (DMSO-d₆, 400 MHz): δ 10.40 (s, 1H), 10.14 (s, 1H), 9.60 (s, 1H), 8.55-8.51 (m, 1H), 8.34-8.20 (m, 2H), 7.99 (t, J=9.2 Hz, 1H), 7.84-7.81 (m, 4H), 7.61-7.52 (m, 1H), 7.47-7.34 (m, 3H), 7.11-7.08 (m, 2H), 5.47 (s, 2H), 5.31 (s, 2H), 5.24-5.14 (s, 2H), 4.41-4.33 (m, 1H), 4.21-3.98 (m, 5H), 3.73-3.66 (m, 2H), 3.47-3.43 (m, 6H), 3.20-3.10 (m, 2H), 3.10-3.08 (m, 2H), 2.95-2.94 (m, 4H), 2.74-2.55 (m, 4H), 2.20-2.17 (m, 2H), 2.04-1.95 (m, 3H), 1.31-1.24 (m, 6H), 0.92-0.76 (m, 9H). LC-MS: C₅₇H₇₀N₁₁O₁₄ (M+H): calc. 1032.51, found 1032.40 (M+H). HPLC: 99.74% purity.

### Example 7: Synthesis of Target N, TFA Salt

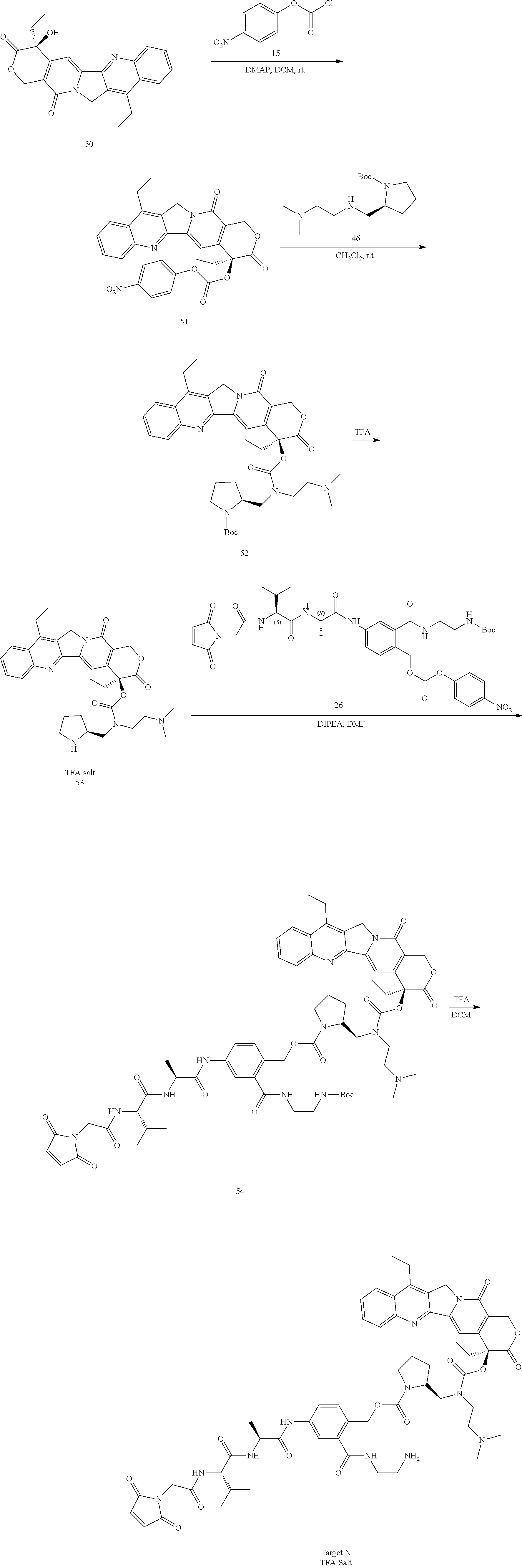

### Synthesis of (S)-4,11-diethyl-3,14-dioxo-3,4,12,14-tetrahydro-1H-pyrano[3′,4′:6,7]indolizino[1,2-b]quinolin-4-yl (4-nitrophenyl) carbonate: Compound 51

To a stirred solution of (S)-4,11-diethyl-4-hydroxy-1,12-dihydro-14H-pyrano[3′,4′:6,7]indolizino[1,2-b]quinoline-3,14(4H)-dione, compound 50 (0.5 g, 1.33 mmol) and 4-nitrophenyl chloroformate, compound 15 (1.07 g, 5.31 mmol) in THF (60 mL) was added DMAP (0.81 g, 6.64 mmol). The resulting solution was stirred at rt for 2 h. The progress of the reaction was monitored by TLC. After the reaction was complete, the mixture was diluted with diethyl ether (50 mL). The obtained solids were filtered and washed with n-pentane, then dissolved in DCM (100 mL) and washed with 1N HCl solution (1×50 mL). The organic layer was dried over Na₂SO₄, then concentrated under reduced pressure to provide (S)-4,11-diethyl-3,14-dioxo-3,4,12,14-tetrahydro-1H-pyrano[3′,4′:6,7]indolizino[1,2-b]quinolin-4-yl (4-nitrophenyl) carbonate, compound 51 (0.5 g, 69.5% yield) as an off white solid. LC-MS: C₂₉H₂₃N₃O₈ (M+H): calc. 542.16, found 542.00 (M+H).

### Synthesis of tert-butyl (S)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate: Compound 52

To a stirred solution of (S)-4,11-diethyl-3,14-dioxo-3,4,12,14-tetrahydro-1H-pyrano[3′,4′:6,7]indolizino[1,2-b]quinolin-4-yl (4-nitrophenyl) carbonate, compound 51 (400 mg, 0.74 mmol) in DCM (20 mL) was added compound tert-butyl (S)-2-(((2-(dimethylamino)ethyl)amino)methyl)pyrrolidine-1-carboxylate, compound 46 (401 mg, 1.48 mmol) under nitrogen atmosphere followed by DIPEA (0.39 mL, 2.22 mmol). The reaction was stirred at rt. for 2 h. The progress of the reaction was monitored by TLC. After the reaction was complete, the reaction mixture was concentrated under reduced pressure and co-distilled with DCM to provide tert-butyl (S)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 52 (700 mg, 0.77 mmol, quantitate yield) as a yellow liquid. LC-MS: C₃₇H₄₇N₅O₇ (M+H): calc. 674.36, found 674.90 (M+H).

### Synthesis of (S)-4,11-diethyl-3,14-dioxo-3,4,12,14-tetrahydro-1H-pyrano[3′,4′:6,7]indolizino[1,2-b]quinolin-4-yl (2-(dimethylamino)ethyl)(((2S)-1-(2,2,2-trifluoroacetyl)-114-pyrrolidin-2-yl)methyl)carbamate: Compound 53

To a stirred solution of tert-butyl (S)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 52 (700 mg, 1.039 mmol) in DCM (10 mL) at 0° C. was added TFA (0.79 mL, 10.39 mmol). The reaction mixture was stirred at rt. for 2 h, then diluted with diethyl ether. The resulting solid was filtered, washed with n-pentane and dried in vacuo to obtained (S)-4,11-diethyl-3,14-dioxo-3,4,12,14-tetrahydro-1H-pyrano[3′,4′:6,7]indolizino[1,2-b]quinolin-4-yl (2-(dimethylamino)ethyl)(((2S)-1-(2,2,2-trifluoroacetyl)-114-pyrrolidin-2-yl)methyl)carbamate, compound 53 (700 mg, 96% yield) as a yellow solid. LC-MS: C₃₂H₃₉N₅O₅ (M+H): calc. 574.30, found 574.50 (M+H).

### Synthesis of 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl (R)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate: Compound 54

To a stirred solution of (S)-4,11-diethyl-3,14-dioxo-3,4,12,14-tetrahydro-1H-pyrano[3′,4′:6,7]indolizino[1,2-b]quinolin-4-yl (2-(dimethylamino)ethyl)(((2S)-1-(2,2,2-trifluoroacetyl)-114-pyrrolidin-2-yl)methyl)carbamate, compound 53 (200 mg, 0.30 mmol) and tert-butyl (2-(5-((S)-2-((S)-2-(2-(2,5-dioxo-2,5-dihydro-1H-pyrrol-1-yl)acetamido)-3-methylbutanamido)propanamido)-2-((((4-nitrophenoxy)carbonyl)oxy)methyl)benzamido)ethyl)carbamate, compound 26 (256 mg, 0.33 mmol) in DMF (10 mL) was added DIPEA (0.44 mL, 2.54 mmol) 0° C. The reaction mixture was stirred at rt for 1 h. After the reaction was complete, the reaction mixture was concentrated under reduced pressure. The residue was triturated with diethyl ether and dried under vacuum to provide a yellow solid which was purified by HPLC (Mobile phase A: 0.1% TFA in H₂O, Mobile phase B: 100% acetonitrile). Pure fractions were dried by lyophilization for 2 days to obtain 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl (R)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 54 (200 mg, 31% yield) as a yellow solid. LC-MS: C₆₂H₇₇N₁₁O₁₅ (M+H): calc. 1216.57, found 1216.25 (M+H).

### Synthesis of 4-((S)-2-((S)-2-(2-(2,5-dioxo-2,5-dihydro-1H-pyrrol-1-yl)acetamido)-3-methylbutanamido)propanamido)-2-((2-((2,2,2-trifluoroacetyl)-14-azaneyl)ethyl)carbamoyl)benzyl (R)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate: Target N, TFA salt

To a stirred solution of To a stirred solution of 2-((2-((tert-butoxycarbonyl)amino)ethyl)carbamoyl)-4-((S)-2-((S)-2-(2-(2,5-dioxo-2,5-dihydro-1H-pyrrol-1-yl)acetamido)-3-methylbutanamido)propanamido)benzyl (R)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, compound 54 (250 mg, 0.21 mmol) in DCM (5 mL) was added TFA (0.32 mL, 4.11 mmol) at 0° C. The reaction mixture was stirred at rt. for 3 h, then concentrated under reduced pressure and triturated with diethyl ether to provide the crude product which was purified by preparative HPLC (Mobile phase A: 0.1% TFA in H₂O, Mobile phase B: 100% acetonitrile). Pure fractions were dried by lyophilization for 2 days to obtain 4-((S)-2-((S)-2-(2-(2,5-dioxo-2,5-dihydro-1H-pyrrol-1-yl)acetamido)-3-methylbutanamido)propanamido)-2-((2-((2,2,2-trifluoroacetyl)-14-azaneyl)ethyl)carbamoyl)benzyl (R)-2-((((((S)-4,11-diethyl-3,14-dioxo-3,4,12,14-tetrahydro-1H-pyrano[3′,4′:6,7]indolizino[1,2-b]quinolin-4-yl)oxy)carbonyl)(2-(dimethylamino)ethyl)amino)methyl)pyrrolidine-1-carboxylate, Target N, TFA salt (95 mg, 36.19% yield) as a yellow solid.

¹H NMR (DMSO-d₆, 400 MHz, VT): δ 9.72 (s, 1H), 8.28-8.26 (m, 2H), 8.10 (d, J=8.8 Hz, 1H), 7.97-7.94 (m, 1H), 7.89-7.87 (m, 1H), 7.84-7.78 (m, 2H), 7.74-7.70 (m, 2H), 7.53-7.51 (m, 1H), 7.38-7.36 (m, 1H), 7.11 (brs, 1H), 6.98-6.97 (m, 2H), 5.45 (s, 2H), 5.33 (s, 2H), 5.23 (brs, 2H), 4.44-4.40 (m, 1H), 4.19-4.14 (m, 3H), 3.50-3.46 (m, 5H), 3.27-3.23 (m, 5H), 3.00-2.97 (m, 6H), 2.50 (s, 6H), 2.22-2.16 (m, 3H), 2.02-1.99 (m, 3H), 1.36-1.32 (m, 6H), 0.94 (t, J=7.6 Hz, 3H), 0.87 (t, J=7.6 Hz, 6H). LC-MS: C₅₇H₆₉N₁₁O₁₃ (M−H): calc. 1114.50, found 1114.40 (M−H), 558.90 (M/2+H). HPLC: 97.25% purity.

### Example 8: Synthesis of Target DF, TFA Salt

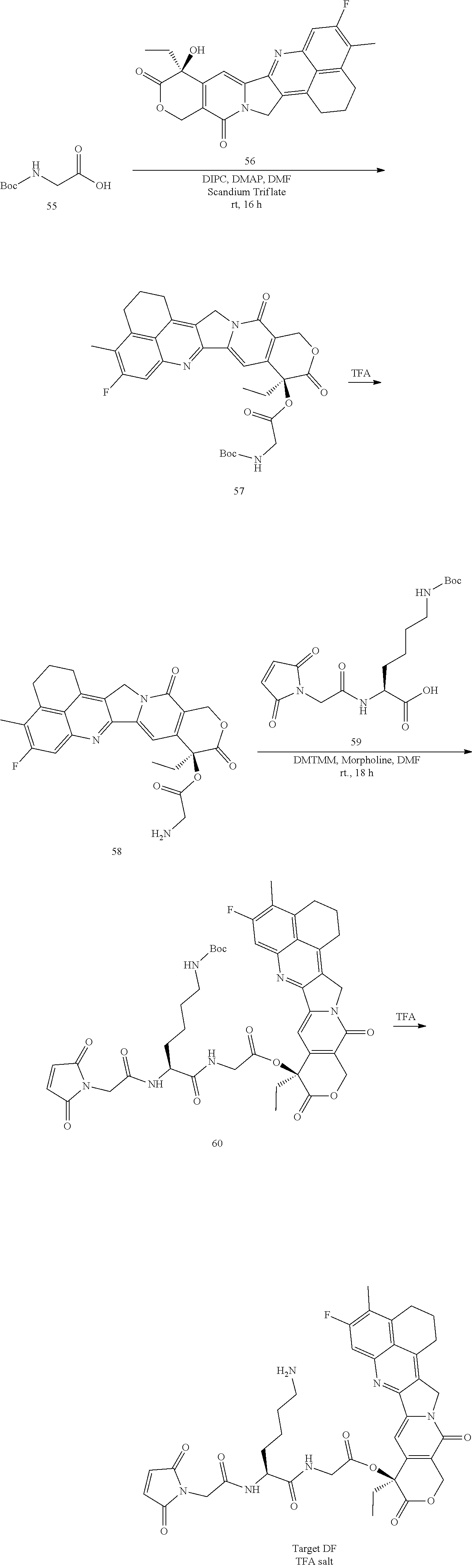

### Synthesis of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl (tert-butoxycarbonyl)glycinate: Compound 57

To a stirred solution of (S)-9-ethyl-5-fluoro-9-hydroxy-4-methyl-1,2,3,9,12,15-hexahydro-10H,13H-benzo[de]pyrano[3′,4′:6,7]indolizino[1, 2-b]quinoline-10, 13-di one, compound 56 (200 mg, 0.48 mmol) and (tert-butoxycarbonyl)glycine, compound 55 (417 mg, 2.38 mmol) in DCM (10 mL) at 0° C., were added scandium trifluoromethanesulfonate (234 mg, 0.48 mmol), N,N′-Diisopropylcarbodiimide (0.371 ml, 2.38 mmol) and DMAP (291 mg, 2.38 mmol). The reaction mixture was stirred at 26° C. for 16 h. Then the reaction mixture was diluted with DCM and washed with 1N HCL. The organic layer was dried over Na₂SO₄, then concentrated under reduced pressure. The crude compound was purified by combi flash using 0-80% EtOAc in Hexane to provide (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl (tert-butoxycarbonyl)glycinate, compound 57 (200 mg, 61.900 yield) as a white solid. LC-MS: C₃₁H₃₂FN₃O₇: calc 578.60 (M+H), found 578.30 (M+H).

### Synthesis of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl (tert-butoxycarbonyl)glycinate: Compound 58

To a stirred solution of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl (tert-butoxycarbonyl)glycinate, compound 57 (200 mg, 0.35 mmol) in DCM (5 mL) at 0° C. was added TFA (0.03 ml, 0.35 mmol). The reaction mixture was stirred at 26° C. for 2 h, then concentrated under reduced pressure. The resulting residue was washed with diethyl ether and dried to afford (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 2-((2,2,2-trifluoroacetyl)-14-azaneyl)acetate, compound 58 (200 mg, 100% yield) as a yellow solid. LC-MS: C₂₆H₂₄FN₃O₅: calc 478.17 (M+H), found 478.10. (M+H).

### Synthesis of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N6-(tert-butoxycarbonyl)-N2-(2-(2,5-dioxo-2,5-dihydro-1H-pyrrol-1-yl)acetyl)-L-lysylglycinate: Compound 60

To a stirred solution of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl 2-((2,2,2-trifluoroacetyl)-14-azaneyl)acetate, compound 58 (200 mg, 0.35 mmol) and N6-(tert-butoxycarbonyl)-N2-(2-(2,5-dioxo-2,5-dihydro-1H-pyrrol-1-yl)acetyl)-L-lysine, compound 59 (267 mg, 0.70 mmol) in DMF (5 mL) at 0° C. was added 4-(4,6-dimethoxy-1,3,5-triazin-2-yl)-4-methylmorpholin-4-ium chloride (193 mg, 0.70 mmol), morpholine (0.15 ml, 1.74 mmol). The reaction mixture was stirred at 26° C. for 2 h, then concentrated under reduced pressure. The crude compound was purified by combi flash using 0-80% EtOAc in hexane to provide (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N6-(tert-butoxycarbonyl)-N2-(2-(2,5-dioxo-2,5-dihydro-1H-pyrrol-1-yl)acetyl)-L-lysylglycinate, compound 60 (190 mg, 0.14 mmol, 40.6%) as a yellow solid. LC-MS: C₄₃H₄₇FN₆O₁₁: calc 843.17 (M+H), found 843.20. (M+H).

### Synthesis of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl ((S)-2-(2-(2,5-dioxo-2,5-dihydro-1H-pyrrol-1-yl)acetamido)-6-((2,2,2-trifluoroacetyl)-14-azaneyl)hexanoyl)glycinate: Target DF, TFA Salt

To a stirred solution of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl N6-(tert-butoxycarbonyl)-N2-(2-(2,5-dioxo-2,5-dihydro-1H-pyrrol-1-yl)acetyl)-L-lysylglycinate, compound 60 (190 mg, 0.23 mmol) in DCM (10 mL) at 0° C., added TFA (0.02 ml, 0.23 mmol). The reaction mixture was stirred at 26° C. for 1 h, then concentrated under reduced pressure. The crude product was purified by preparative HPLC (Mobile phase A: 0.1% TFA in H₂O, Mobile phase B: 100% acetonitrile). Pure fractions were dried by lyophilization for 2 days to provide (S)-9-ethyl-5-fluoro-4-methyl-10,13-dioxo-2,3,9,10,13,15-hexahydro-1H,12H-benzo[de]pyrano[3′,4′:6,7]indolizino [1,2-b]quinolin-9-yl ((S)-2-(2-(2,5-dioxo-2,5-dihydro-1H-pyrrol-1-yl) acetamido)-6-((2,2,2-trifluoroacetyl)-14-azaneyl)hexanoyl) glycinate, Target DF, TFA salt (90 mg, 47.83% yield) as a yellow solid

¹H NMR (DMSO-d₆, 400 MHz): δ 8.56 (t, J=6.0 Hz, 1H), 8.41 (d, J=8.4 Hz, 1H), 7.71 (d, J=11.2 Hz, 1H), 7.59 (brs, 3H, NH₃+), 7.10 (s, 1H), 7.07 (s, 2H), 5.50 (s, 2H), 5.26 (s, 2H), 4.28-4.27 (m, 1H), 4.16-4.01 (m, 4H), 3.16-3.09 (m, 4H), 2.71-2.67 (m, 2H), 2.37 (s, 3H), 2.15-2.08 (m, 4H), 1.69-1.67 (m, 1H), 1.50-1.46 (m, 3H), 1.30-1.25 (m, 2H), 0.91 (t, J=7.6 Hz, 3H). LC-MS: C₃₈H₃₉FN₆O₉: calc 743.28 (M+H), found 743.20. (M+H). HPLC: 96.31%.

### Example 9: Synthesis of Target DG, TFA Salt

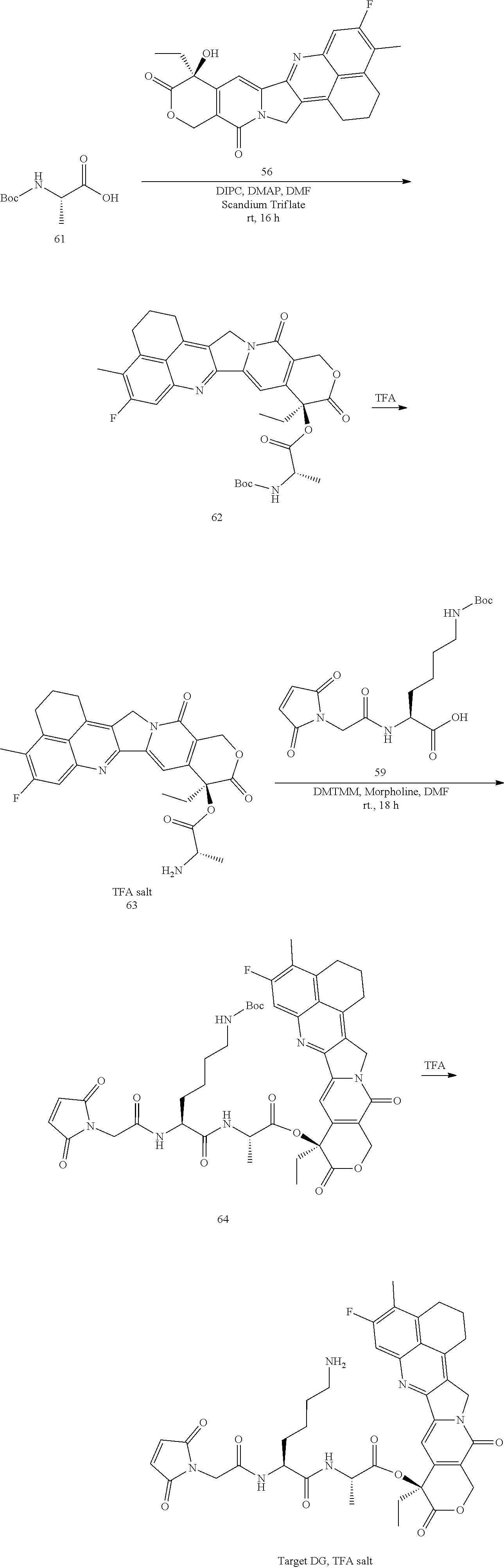

### Synthesis of (S)-9-ethyl-5-fluoro-4-methyl-10,13-dioxo-2,3,9,10,13,15-hexahydro-1H,12H-benzo[de]pyrano[3′,4′:6,7]indolizino[1,2-b]quinolin-9-yl ((S)-2-(2-(2,5-dioxo-2,5-dihydro-1H-pyrrol-1-yl)acetamido)-6-((2,2,2-trifluoroacetyl)-14-azaneyl)hexanoyl)-L-alaninate: Target DG, TFA Salt

The title compound was prepared as described in Example 8 except (tert-butoxycarbonyl)-L-alanine, compound 61 was used instead of (tert-butoxycarbonyl)glycine, compound 55.

¹H NMR (DMSO-d₆, 400 MHz): δ 8.55 (d, J=7.6 Hz, 1H), 8.46 (d, J=8.4 Hz, 1H), 7.69 (d, J=10.4 Hz, 1H), 7.60 (brs, 3H, NH3+), 7.10 (s, 2H), 7.02 (s, 1H), 5.50 (s, 2H), 5.27 (s, 2H), 4.53 (t, J=7.2 Hz, 1H), 4.36-4.35 (m, 1H), 4.16-4.06 (m, 2H), 3.15-3.09 (m, 4H), 2.73-2.67 (m, 2H), 2.38 (s, 3H), 2.15-2.08 (m, 4H), 1.76-1.73 (m, 1H), 1.51-1.48 (m, 3H), 1.44 (d, J=7.2 Hz, 3H), 1.35-1.33 (m, 2H), 0.91 (t, J=7.2 Hz, 3H). LC-MS: C₃₉H₄₁FN₆O₉: calc 757.29 (M+H), found 757.40. (M+H). HPLC: 96.22% purity.

### Example 10: Synthesis of di-tert-butyl 3,3′-((2-(2 azidoethoxy)acetyl)azanediyl)dipropionate: Target U

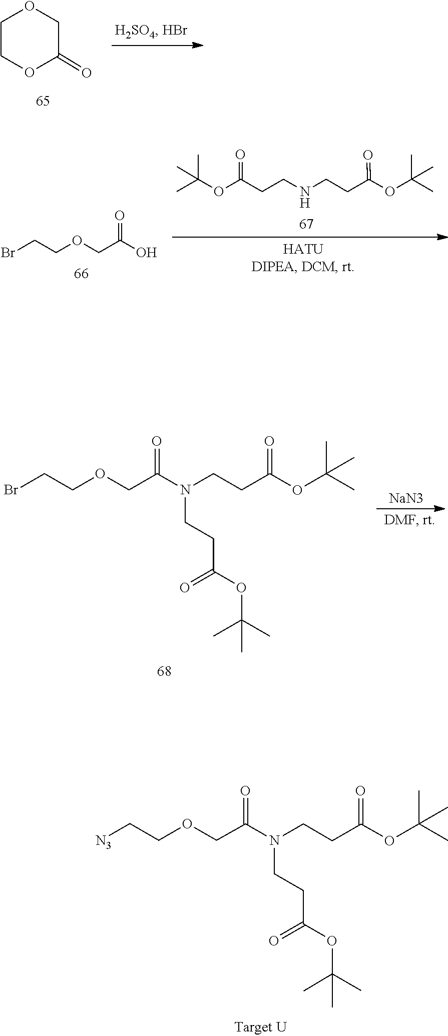 [SMILES: O=C1COCCO1 ; CC(C)(C)OC(=O)CCNCCC(=O)OC(C)(C)C ; O=C(O)COCCBr ; CC(C)(C)OC(=O)CCN(CCC(=O)OC(C)(C)C)C(=O)COCCBr ; CC(C)(C)OC(=O)CCN(CCC(=O)OC(C)(C)C)C(=O)COCCN=[N+]=[N-] ; "NaN3"]

### Synthesis of 2-(2-bromoethoxy)acetic acid: Compound 66

To the stirred solution of HBr (47% aq., 18.62 mL, 343 mmol) was added H₂SO₄ (5.22 mL, 98 mmol) cautiously at 0° C., then the mixture was stirred for 10 min at 0° C., then 1,4-dioxan-2-one compound, 65 (5.0 g, 49.0 mmol) was added and resulting mixture was stirred at rt for 1 h, then heated to 100° C. for 2.5 h, and then stirred at rt for 1 h. The reaction mixture was poured into a beaker with ice water and extracted with EtOAc. The organic layers were dried over sodium sulphate, filtered and the filtrate was concentrated in a rotary evaporator to provide 2-(2-bromoethoxy)acetic acid, compound 66 (4.5 g, 50.2% yield) as a white solid. ¹H NMR (CDCl₃, 400 MHz): δ 4.23 (s, 2H), 3.92 (t, J=6.0 Hz, 2H), 3.52 (t, J=6.0 Hz, 2H).

### Synthesis of di-tert-butyl 3,3′-((2-(2-bromoethoxy)acetyl)azanediyl)dipropionate: Compound 68

To a stirred solution of 2-(2-bromoethoxy)acetic acid, compound 66 (4.5 g, 24.59 mmol) in DCM (60 mL) was added DMAP (0.15 g, 1.230 mmol), EDC (5.66 g, 29.5 mmol) at 0° C., and then di-tert-butyl 3,3′-azanediyldipropionate, compound 67 (7.39 g, 27.0 mmol) was added and the reaction mixture was stirred at rt for 16 h. Then 1N HCl (100 mL) was added to the reaction mixture and extracted with DCM (200 mL). The organic layers were dried over anhydrous sodium sulphate, filtered and concentrated under reduced pressure to provide the crude compound. The crude compound was purified by combi flash (20-30% EtOAc in n-hexane eluant) to provide di-tert-butyl 3,3′-((2-(2-bromoethoxy)acetyl)azanediyl)dipropionate, compound 68 (6.0 g, 55.7% yield) as a colorless oily liquid. ESI-MS: C₁₈H₃₃BrNO₆ (M+H): calc. 438.14, found 438.15 (M+H).

### Synthesis of di-tert-butyl 3,3′-((2-(2-azidoethoxy)acetyl)azanediyl)dipropionate: Target U

To a stirred solution of di-tert-butyl 3,3′-((2-(2-bromoethoxy)acetyl)azanediyl)dipropionate, compound 68 (3.0 g, 6.84 mmol) in DMF (20 mL) at 0° C., was added sodium azide (1.335 g, 20.53 mmol). The reaction mixture was stirred at rt for 16 h. The progress of the reaction was monitored by TLC. The reaction mixture was diluted with water (100 mL) and extracted with diethyl ether (2×100 mL) and then washed with water (50 mL) and brine (50 mL). The organic layers were dried over anhydrous Na₂SO₄ and concentrated under reduced pressure to provide the crude product as a colorless oily liquid. The crude compound was purified by combi flash (30-40% EtOAc in n-hexane as eluant) to provide di-tert-butyl 3,3′-((2-(2-azidoethoxy)acetyl)azanediyl)dipropionate, Target U (1.7 g, 4.19 mmol, 61.2% yield) as a colorless liquid.

¹H NMR (CDCl₃, 400 MHz): δ 4.27 (s, 2H), 3.73-3.70 (m, 2H), 3.61-3.53 (m, 4H), 3.46-3.44 (m, 2H), 2.56-2.51 (m, 4H), 1.449 (s, 9H), 1.442 (s, 9H). ESI-MS: C₁₈H₃₃N₄₀₆ (M+H): calc. 401.24, found 401.30 (M+H). HPLC: 98.64%. purity

### Example 11: Synthesis of 3-(2-(2-azidoethoxy)-N-(3-oxo-3-(((2S,3R,4R,5R)-2,3,4,5,6 pentahydroxyhexyl)amino)propyl)acetamido)-N-((2S,3R,4R,5R)-2,3,4,5,6-pentahydroxyhexyl)propenamide: Target K

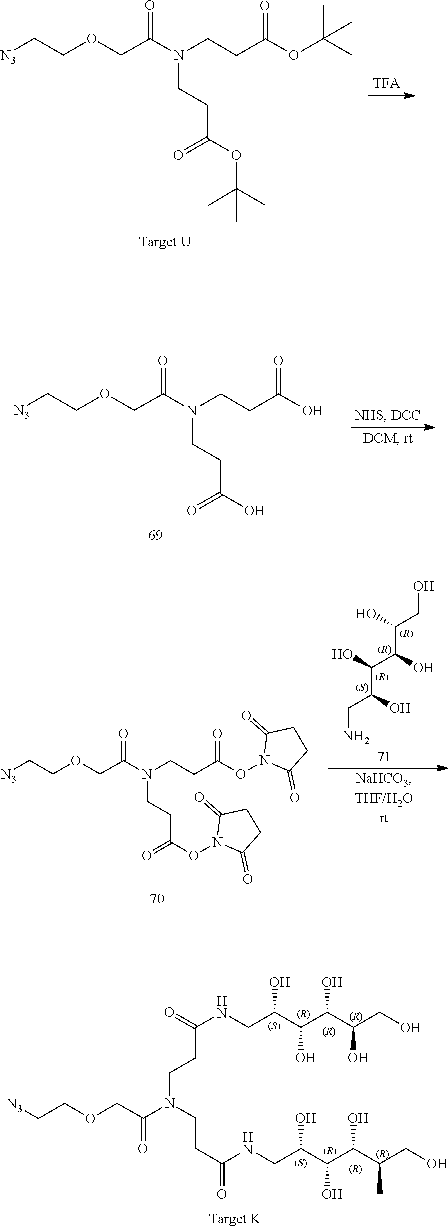

### Synthesis of 3,3′-((2-(2-azidoethoxy)acetyl)azanediyl)dipropionic acid, Compound 54

To a stirred solution of di-tert-butyl 3,3′-((2-(2-azidoethoxy)acetyl)azanediyl)dipropionate, Target U (prepared as described in Example 10, 4.5 g, 11.24 mmol) in DCM (50 mL) at 0° C. was added TFA (8.66 mL, 112 mmol). The reaction mixture was stirred at rt for 4 hr, then concentrated under reduced pressure and triturated with diethyl ether to provide 3,3′-((2-(2-azidoethoxy)acetyl)azanediyl)dipropionic acid, compound 69 (2.5 g, 8.67 mmol, 77% yield) as a brown viscous liquid. ESI-MS: C₁₀H₁₇N₄O₆ (M+H): calc. 289.11, found 289.10 (M+H).

### Synthesis of bis(2,5-dioxopyrrolidin-1-yl) 3,3′-((2-(2-azidoethoxy)acetyl)azanediyl)dipropionate: Compound 70

To a stirred solution of 3,3′-((2-(2-azidoethoxy)acetyl)azanediyl)dipropionic acid, compound 69 (2.5 g, 8.67 mmol) and NHS (2.5 g, 21.68 mmol) in DCM (60 mL) at rt was added a solution of DCC (4.47 g, 21.68 mmol) in DCM (20 mL). The reaction mixture was stirred at rt for 16 h, then filtered and washed with ice cold DCM (10 mL). The filtrate was concentrated under reduced pressure to provide bis(2,5-dioxopyrrolidin-1-yl) 3,3′-((2-(2-azidoethoxy)acetyl)azanediyl)dipropionate, compound 70 (1.5 g, 2.425 mmol, 28.0%) as a white gummy solid. ESI-MS: C10H16N4O6 (M+H): calc. 289.11, found 289.1 (M+H).

### Synthesis of 3-(2-(2-azidoethoxy)-N-(3-oxo-3-(((2S,3R,4R,5R)-2,3,4,5,6-pentahydroxyhexyl)amino)propyl)acetamido)-N-((2S,3R,4R,5R)-2,3,4,5,6-pentahydroxyhexyl)propenamide: Target K

To a stirred solution of bis(2,5-dioxopyrrolidin-1-yl) 3,3′-((2-(2-azidoethoxy)acetyl)azanediyl)dipropionate, compound 70 (1.50 g, 3.11 mmol) and (2R,3R,4R,5S)-6-aminohexane-1,2,3,4,5-pentaol, compound 71 (1.69 g, 9.33 mmol) in THF (20 mL) was added sodium bicarbonate (1.57 g, 18.66 mmol) in H₂O (20 mL) at rt. The reaction mixture was stirred at rt for 16 h, then concentrated under reduced pressure to provide an off-white gummy liquid. The crude compound was purified by HPLC (Mobile phase A: 0.1% TFA in H₂O, Mobile phase B: 100% acetonitrile). Pure fractions were dried by lyophilization for 2 days to provide 3-(2-(2-azidoethoxy)-N-(3-oxo-3-(((2S,3R,4R,5R)-2,3,4,5,6 pentahydroxyhexyl)amino)propyl)acetamido)-N-((2S,3R,4R,5R)-2,3,4,5,6-pentahydroxyhexyl)propenamide, Target K (530 mg, 27.7% yield) as a colorless sticky liquid.

¹H NMR (DMSO-d₆, 400 MHz): δ 7.93 (t, J=5.2 Hz, 1H), 7.83 (d, J=5.2 Hz, 1H), 4.75 (d, J=4.8 Hz, 2H), 4.47 (t, J=4.4 Hz, 2H), 4.37 (d, J=6.0 Hz, 2H), 4.33 (t, J=5.6 Hz, 2H), 4.26-4.24 (m, 4H), 3.62-3.55 (m, 8H), 3.49-3.34 (m, 14H), 3.27-3.26 (m, 2H), 3.05-2.98 (m, 2H), 2.39 (t, J=7.2 Hz, 1H), 2.32 (t, J=7.2 Hz, 1H). LC-MS: C₂₂H₄₃N₆O₁₄ (M+H): calc. 615.28, found 615.35 (M+H). HPLC: 99% purity.

### Example 12: Synthetic Scheme for Target AA

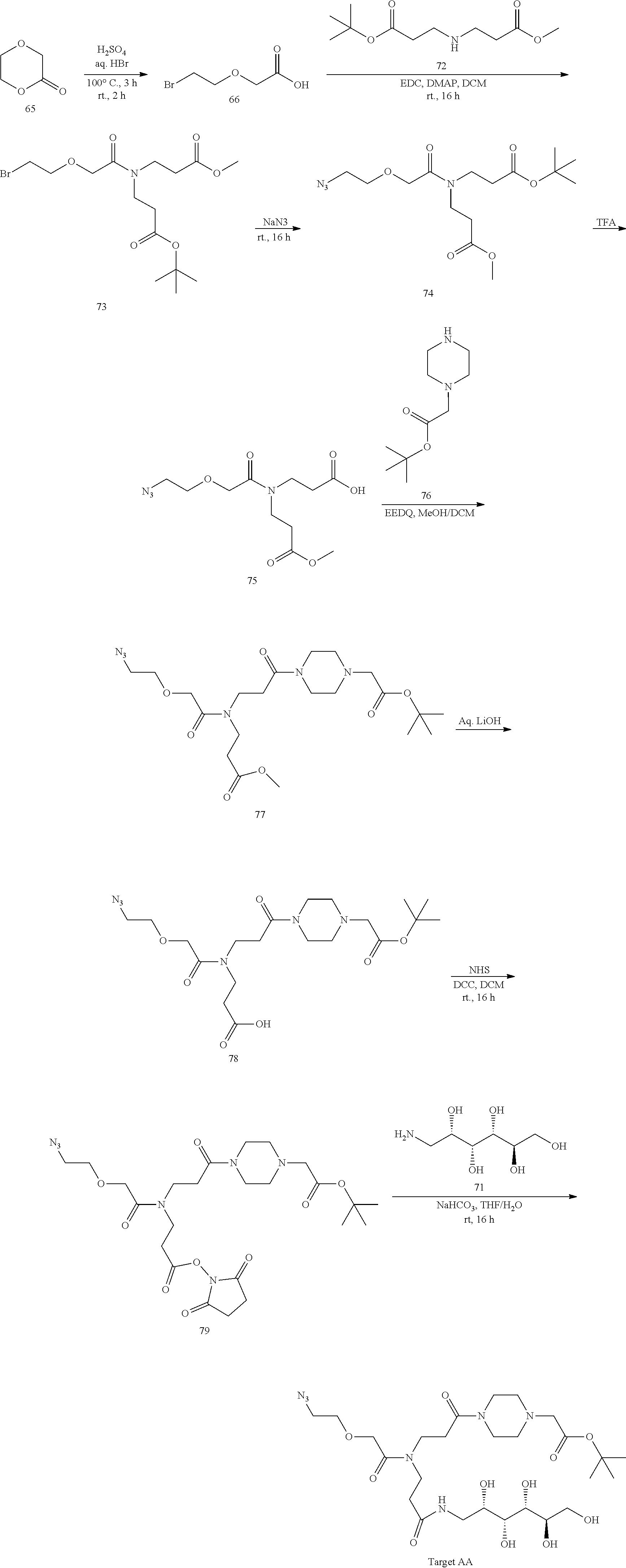

### Synthesis of tert-butyl 3-(2-(2-bromoethoxy)-N-(3-methoxy-3-oxopropyl)acetamido)propanoate: Compound 73

The title compound was prepared as described in Example 10 except di-tert-butyl 3,3′-azanediyldipropionate, compound 72 was used instead of di-tert-butyl 3,3′-azanediyldipropionate, compound 67, to provide crude tert-butyl 3-(2-(2-bromoethoxy)-N-(3-methoxy-3-oxopropyl)acetamido)propanoate, compound 73 (9.2 g, 49.4% yield) as a colorless oily liquid. LCMS: Ci₅H₂₆BrNO₆ (M+H): calc. 397.29, found 397.85 (M+H).

### Synthesis of tert-butyl 3-(2-(2-azidoethoxy)-N-(3-methoxy-3-oxopropyl)acetamido)propanoate: Compound 74

To a stirred solution of tert-butyl 3-(2-(2-bromoethoxy)-N-(3-methoxy-3-oxopropyl)acetamido)propanoate, compound 73 (9.2 g, 23.22 mmol) in DMF (20 mL) at 0° C., was added sodium azide (4.53 g, 69.6 mmol). the reaction mixture was stirred at rt. for 16 h. The progress of the reaction was monitored by TLC. The reaction mixture was diluted with water (100 mL), extracted with diethyl ether (2×100 mL) and washed with water (50 mL) and brine (50 mL). The organic layers were dried over anhydrous Na₂SO₄ and concentrated under reduced pressure to provide tert-butyl 3-(2-(2-azidoethoxy)-N-(3-methoxy-3-oxopropyl)acetamido)propanoate, compound 74 (6.6 g, 79% yield) as a colorless liquid. LCMS: C₁₅H₂₆N₄O₆ (M+H): calc. 359.19, found 359.20 (M+H).

### Synthesis of 3-(2-(2-azidoethoxy)-N-(3-methoxy-3-oxopropyl)acetamido)propanoic acid: Compound 75

To a stirred solution of di-tert-butyl 3,3′-((2-(2-azidoethoxy)acetyl)azanediyl)dipropionate, compound 74 (4.1 g, 11.44 mmol) in DCM (50 mL) at 0° C. was added TFA (8.81 mL, 114 mmol). The reaction mixture was stirred at rt. for 4 hr, then concentrated under reduced pressure and triturated with diethyl ether to provide 3-(2-(2-azidoethoxy)-N-(3-methoxy-3-oxopropyl)acetamido)propanoic acid, compound 75 (3.0 g, 87% yield) as a yellow liquid. ESI-MS: C₁₁H₁₈N₄O₆ (M+H): calc. 303.13, found 303.40 (M+H).

### Synthesis of methyl 3-(2-(2-azidoethoxy)-N-(3-(4-(2-(tert-butoxy)-2-oxoethyl)piperazin-1-yl)-3-oxopropyl)acetamido)propanoate: Compound 77

To a stirred solution of 3-(2-(2-azidoethoxy)-N-(3-methoxy-3-oxopropyl)acetamido)propanoic acid, compound 75 (3.0 g, 9.92 mmol) and tert-butyl 2-(piperazin-1-yl)acetate, compound 76 (2.98 g, 14.89 mmol) in DCM (50 ml) and MeOH (10 ml) at rt., was added ethyl 2-ethoxyquinoline-1(2H)-carboxylate (4.91 g, 19.85 mmol). The reaction mixture stirred at rt for 16 h. The progress of the reaction was monitored by TLC. After the reaction was complete, the reaction mixture was concentrated under reduced pressure to obtain the crude material which was purified by combi-flash using 4-6% MeOH in DCM to provide methyl 3-(2-(2-azidoethoxy)-N-(3-(4-(2-(tert-butoxy)-2-oxoethyl)piperazin-1-yl)-3-oxopropyl)acetamido)propanoate, compound 77 (2.35 g, 48.9% yield) as a colorless sticky liquid. LC-MS: C₂₁H₃₆N₆O₇ (M+H): calc. 485.55, found 485.65 (M+H).

### Synthesis of 3-(2-(2-azidoethoxy)-N-(3-(4-(2-(tert-butoxy)-2-oxoethyl)piperazin-1-yl)-3 oxopropyl)acetamido)propanoic acid: Compound 78

To a stirred solution of methyl 3-(2-(2-azidoethoxy)-N-(3-(4-(2-(tert-butoxy)-2-oxoethyl)piperazin-1-yl)-3-oxopropyl)acetamido)propanoate, compound 77 (2.00 g, 4.13 mmol) in THF (30 mL) was added LiOH (0.20 g, 8.26 mmol) in H₂O (3 mL) at 0° C. The reaction mixture was stirred at rt for 4 h. The progress of the reaction was monitored by TLC. The reaction mixture was concentrated under reduced pressure to get crude material which was purified by combi-flash using 6-15% MeOH in DCM to provide 3-(2-(2-azidoethoxy)-N-(3-(4-(2-(tert-butoxy)-2-oxoethyl)piperazin-1-yl)-3-oxopropyl)acetamido)propanoic acid, compound 78 (1.5 g, 76% yield) as a colorless sticky liquid. LC-MS: C₂₀H₃₄N₆O . . . (M+H): calc. 471.53, found 471.55 (M+H).

### Synthesis of 2,5-dioxopyrrolidin-1-yl 3-(2-(2-azidoethoxy)-N-(3-(4-(2-(tert-butoxy)-2-oxoethyl)piperazin-1-yl)-3-oxopropyl)acetamido)propanoate: Compound 79

To a stirred solution of 3-(2-(2-azidoethoxy)-N-(3-(4-(2-(tert-butoxy)-2-oxoethyl)piperazin-1-yl)-3-oxopropyl)acetamido)propanoic acid, compound 78 (1.5 g, 3.19 mmol) and 1-hydroxypyrrolidine-2,5-dione (0.44 g, 3.83 mmol) in DCM (5 mL) at rt, was added a solution of DCC (0.92 g, 4.46 mmol) in DCM (2 mL). The reaction mixture was stirred rt for 16 h. Then the reaction mixture was filtered and washed with ice cold DCM (5 mL). The filtrate was concentrated under reduced pressure to provide 2,5-dioxopyrrolidin-1-yl 3-(2-(2-azidoethoxy)-N-(3-(4-(2-(tert-butoxy)-2-oxoethyl)piperazin-1-yl)-3-oxopropyl)acetamido)propanoate, compound 79 (1.7 g, 47.9% yield) as a white gummy liquid. LC-MS: C₂₄H₃₇N₇O₉ (M+H): calc. 568.60, found 568.60 (M+H).

### Synthesis of tert-butyl 2-(4-(3-(2-(2-azidoethoxy)-N-(3-oxo-3-(((2S,3R,4R,5R)-2,3,4,5,6 pentahydroxyhexyl)amino)propyl)acetamido)propanoyl)piperazin-1-yl)acetate: Target AA

To a stirred solution of 2,5-dioxopyrrolidin-1-yl 3-(2-(2-azidoethoxy)-N-(3-(4-(2-(tertbutoxy)-2-oxoethyl)piperazin-1-yl)-3-oxopropyl)acetamido)propanoate, compound 79 (1.5 g, 3.64 mmol) and (2R,3R,4R,5S)-6-aminohexane-1,2,3,4,5-pentaol, compound 71 (0.48 g, 2.64 mmol) in THF (20 mL) was added sodium bicarbonate (0.22 g, 2.64 mmol) in H₂O (20 mL) at rt. The reaction mixture was stirred at rt. for 16 h then concentrated under reduced pressure to provide an off-white gummy liquid which was purified by prep HPLC (Mobile phase A: 0.1% TFA in H₂O, Mobile phase B: acetonitrile (100%)). The pure fractions were dried by lyophilization for 2 days to obtain tert-butyl 2-(4-(3-(2-(2-azidoethoxy)-N-(3-oxo-3-(((2S,3R,4R,5R)-2,3,4,5,6pentahydroxyhexyl)amino)propyl)acetamido)propanoyl)piperazin-1-yl)acetate, Target AA (350 mg, 0.53 mmol, 20%) as a colorless sticky liquid.

¹H NMR (DMSO-d₆, 400 MHz): δ 7.94 (t, J=5.6 Hz, 1H), 7.86 (t, J=5.6 Hz, 1H), 4.25 (s, 4H), 3.64-3.56 (m, 8H), 3.45-3.37 (m, 12H), 3.32-3.20 (m, 3H), 3.05-3.00 (m, 2H), 2.68-2.67 (m, 2H), 2.57-2.55 (m, 2H), 2.40 (t, J=6.8 Hz, 2H), 2.34-2.31 (m, 1H), 1.43 (s, 9H). LC-MS: C₂₆H₄₇N₇O₁₁ (M+H): calc. 634.70, found 634.30 (M+H). HPLC: 95.16% purity.

### Example 13: Synthesis of Target AC

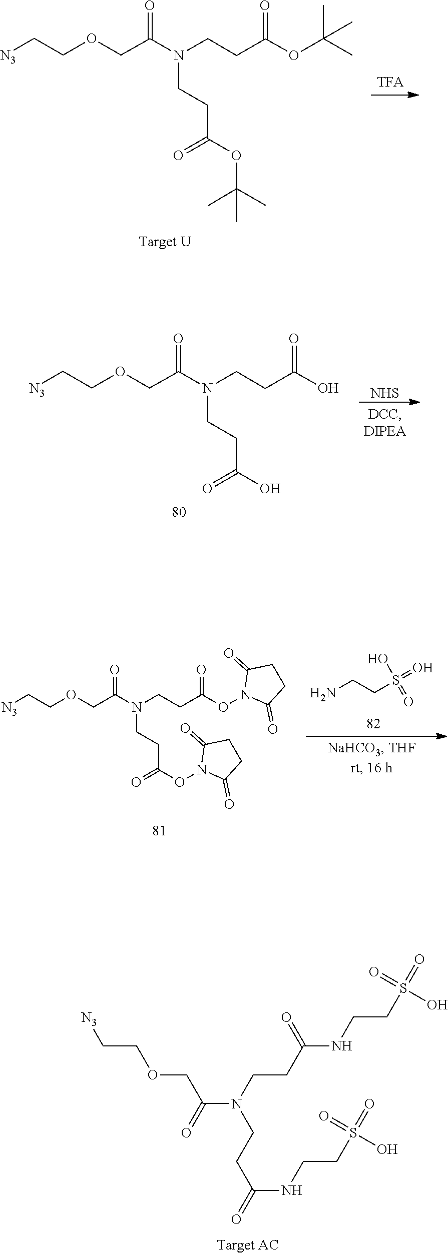

### Synthesis of 3,3′-((2-(2-azidoethoxy)acetyl)azanediyl)dipropionic acid: Compound 80

To a stirred solution of di-tert-butyl 3,3′-((2-(2-azidoethoxy)acetyl)azanediyl)dipropionate, Target U (prepared as described in Example 12, 4.5 g, 11.24 mmol) in DCM (50 mL) at 0° C. was added TFA (8.66 mL, 112 mmol). The reaction mixture was stirred at rt. for 4 hr, then concentrated under reduced pressure and triturated with diethyl ether to provide 3,3′-((2-(2-azidoethoxy)acetyl)azanediyl)dipropionic acid, compound 80 (2.5 g, 77% yield) as a brown viscous liquid. ESI-MS: C₁₀H₁₆N₄O₆ (M+H): calc. 289.11, found 289.10 (M+H).

### Synthesis of bis(2,5-dioxopyrrolidin-1-yl) 3,3′-((2-(2-azidoethoxy)acetyl)azanediyl)dipropionate: Compound 81

To a stirred solution of 3,3′-((2-(2-azidoethoxy)acetyl)azanediyl)dipropionic acid, compound 80 (2.5 g, 8.67 mmol) and 1-hydroxypyrrolidine-2,5-dione (2.5 g, 21.68 mmol) in DCM (60 mL) at rt. was added a solution of DCC (4.47 g, 21.68 mmol) in DCM (20 mL). The reaction mixture was stirred at rt for 16 h, then filtered and washed with ice cold DCM (10 mL). The filtrate was concentrated under reduced pressure to afford bis(2,5-dioxopyrrolidin-1-yl) 3,3′-((2-(2-azidoethoxy)acetyl)azanediyl)dipropionate, compound 81 (1.5 g, 28.0% yield) as a white gummy solid: C₁₈H₂₂N₆O₁₀ (M+H): calc. 483.15, found 483.20 (M+H).

### Synthesis of 2,2′-((3,3′-((2-(2-azidoethoxy)acetyl)azanediyl)bis(propanoyl))bis(azanediyl))bis(ethane-1-sulfonic acid): Target AC

To a stirred solution of bis(2,5-dioxopyrrolidin-1-yl) 3,3′-((2-(2-azidoethoxy)acetyl)azanediyl)dipropionate 81 (0.8 g, 1.66 mmol) and, 2-aminoethane-1-sulfonic acid, compound 82 (0.42 g, 3.32 mmol) in THF (5 mL) was added sodium bicarbonate (0.70 g, 8.29 mmol) in H₂O (5 mL) at rt. Then the reaction mixture was allowed to stirred at rt. for 16 h. Reaction mixture was concentrated under reduced pressure to provide an off white gummy liquid. The obtained crude compound was purified by prep HPLC (Mobile phase A: 0.1% TFA in H₂O, Mobile phase B: acetonitrile (100%)). Pure fractions were dried by lyophilization for 2 days to obtain 2,2′-((3,3′-((2-(2-azidoethoxy)acetyl)azanediyl)bis(propanoyl))bis(azanediyl))bis(ethane-1-sulfonic acid) Target AC (100 mg, 10% yield) as a colorless sticky liquid.

¹H NMR (D₂O, 400 MHz): δ 4.26 (s, 2H), 3.62-3.59 (m, 2H), 3.49-3.39 (m, 10H), 2.94 (t, J=6.8 Hz, 4H), 2.44 (t, J=6.8 Hz, 2H), 2.39 (t, J=6.8 Hz, 2H). LC-MS: C₁₄H₂₆N₆O₁₀S₂ (M+H): calc. 503.12, found 502.95 (M+H). HPLC: 84.05% purity.

### Example 14: Synthesis of Target AD

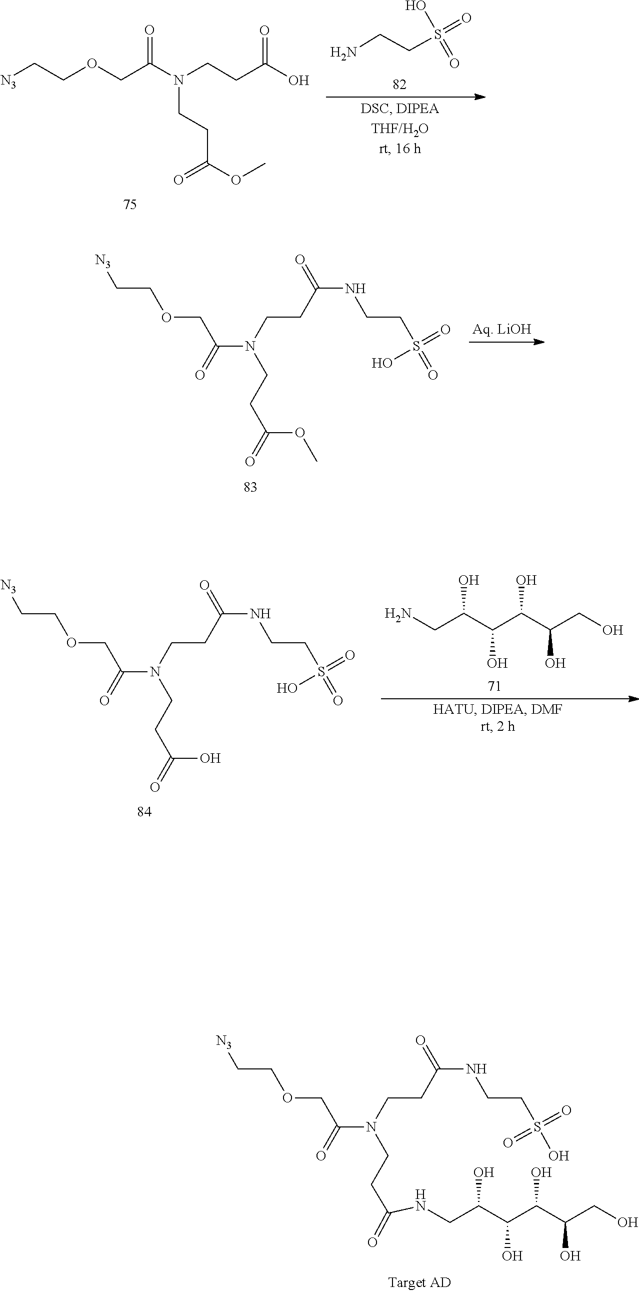 [SMILES: COC(=O)CCN(CCC(=O)O)C(=O)COCCN=[N+]=[N-] ; NCCS(=O)(=O)O ; COC(=O)CCN(CCC(=O)NCCS(=O)(=O)O)C(=O)COCCN=[N+]=[N-] ; [N-]=[N+]=NCCOCC(=O)N(CCC(=O)O)CCC(=O)NCCS(=O)(=O)O ; NC[C@H](O)[C@@H](O)[C@H](O)[C@H](O)CO ; [N-]=[N+]=NCCOCC(=O)N(CCC(=O)NCCS(=O)(=O)O)CCC(=O)NC[C@H](O)[C@@H](O)[C@H](O)[C@H](O)CO]

### Synthesis of 2-(3-(2-(2-azidoethoxy)-N-(3-methoxy-3-oxopropyl)acetamido)propanamido)ethane-1-sulfonic acid: Compound 83

To a stirred solution of 3-(2-(2-azidoethoxy)-N-(3-methoxy-3-oxopropyl)acetamido)propanoic acid, compound 75 (prepared as described in Example 12, 3.0 g, 9.92 mmol) and bis(2,5-dioxopyrrolidin-1-yl) carbonate (3.81 g, 14.89 mmol) in THF (30 mL) was added DIPEA (5.20 mL, 29.8 mmol) at rt. The reaction mixture was then stirred at rt for 3 h, followed by the addition of a solution of 2-aminoethane-1-sulfonic acid, compound 82 (2.48 g, 19.85 mmol) in water (15 mL). The reaction mixture and stirred at rt for 16 h, then concentrated under reduced pressure to provide the crude compound which was purified by prep HPLC (Mobile phase A: 0.1% TFA in H₂O, Mobile phase B: Acetonitrile (100%)). Pure fractions were dried by lyophilization for 2 days to obtain 2-(3-(2-(2-azidoethoxy)-N-(3-methoxy-3-oxopropyl)acetamido)propanamido)ethane-1-sulfonic acid, compound 83 (1.5 g, 25% yield) as a yellow solid. LC-MS: C₁₃H₂₃N₅O_{8S} (M+H): calc. 410.13, found 410.10 (M+H).

### Synthesis of 3-(2-(2-azidoethoxy)-N-(3-oxo-3-((2-sulfoethyl)amino)propyl)acetamido)propanoic acid: Compound 84

To a stirred solution of 2-(3-(2-(2-azidoethoxy)-N-(3-methoxy-3-oxopropyl)acetamido)propanamido)ethane-1-sulfonic acid, compound 83 (1.5 g, 3.66 mmol) in THF (15 mL) and water (7.50 mL) mixture was added LiOH mono hydrate (0.31 g, 7.33 mmol) at rt and the reaction mixture was stirred at rt for 3 h. The reaction mixture was then acidified by the addition of 1N HCl solution (pH~2) and then concentrated under reduced pressure to provide 3-(2-(2-azidoethoxy)-N-(3-oxo-3-((2-sulfoethyl)amino)propyl)acetamido)propanoic acid, compound 84 (1.5 g, 100% yield) as a solid. LC-MS: C₁₂H₂₁N₅O₈S (M+H): calc. 396.11, found 395.95 (M+H).

### Synthesis of 2-(3-(2-(2-azidoethoxy)-N-(3-oxo-3-(((2S,3R,4R,5R)-2,3,4,5,6-pentahydroxyhexyl)amino)propyl)acetamido)propanamido)ethane-1-sulfonic acid: Target AD

To a stirred solution of 3-(2-(2-azidoethoxy)-N-(3-oxo-3-((2-sulfoethyl)amino)propyl)acetamido)propanoic acid, compound 84 (2.0 g, 5.06 mmol) and DIPEA (2.65 mL, 15.18 mmol) in DMF (30 mL) was added HATU (3.85 g, 10.12 mmol) at rt and the reaction mixture was stirred at rt for 5 min, followed by the addition of (2R,3R,4R,5S)-6-aminohexane-1,2,3,4,5-pentaol, compound 71 (0.92 g, 5.06 mmol). The reaction mixture was stirred for 2 h at rt, then washed with ethyl acetate and concentrated under reduced pressure. The crude compound was purified by prep HPLC (Mobile phase A: 10 mM ammonium acetate in H₂O, Mobile phase B: Acetonitrile (100%)). Pure fractions were dried by lyophilization for 2 days to obtain 2-(3-(2-(2-azidoethoxy)-N-(3-oxo-3-(((2S,3R,4R,5R)-2,3,4,5,6-pentahydroxyhexyl)amino)propyl)acetamido)propanamido)ethane-1-sulfonic acid, Target AD (150 mg, 5.14% yield) as a colorless sticky liquid.

¹H NMR (D₂O, 400 MHz): δ 4.25 (s, 2H), 3.61-3.44 (m, 5H), 3.44-3.24 (m, 7H), 3.09-3.07 (m, 1H), 2.72-2.70 (m, 2H), 2.41-2.34 (m, 3H), 1.78 (s, 2H), 1.26 (d, J=6.4 Hz, 3H). LC-MS: C₁₈H₃₄N₆O₁₂S (M+H): calc. 559.20, found 559.10 (M+H). HPLC: 96.42%.

### Example 15: Synthesis of Target Z

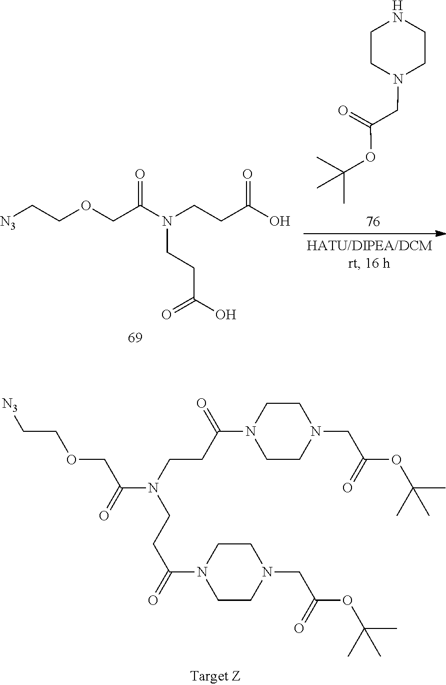 [SMILES: [N-]=[N+]=NCCOCC(=O)N(CCC(=O)O)CCC(=O)O ; CC(C)(C)OC(=O)CN1CCNCC1 ; CC(C)(C)OC(=O)CN1CCN(C(=O)CCN(CCC(=O)N2CCN(CC(=O)OC(C)(C)C)CC2)C(=O)COCCN=[N+]=[N-])CC1]

### Synthesis of di-tert-butyl 2,2′-((3,3′-((2-(2-bromoethoxy)acetyl)azanediyl)bis(propanoyl))bis(piperazine-4,1-diyl))diacetate: Target Z

To a solution of 3,3′-((2-(2-azidoethoxy)acetyl)azanediyl)dipropionic acid, compound 69 (prepared as described in Example 11, 400 mg, 1.39 mmol) in DCM (10 mL) were added DIPEA (1.21 mL, 6.94 mmol) and tert-butyl 2-(piperazin-1-yl)acetate, compound 76 (834 mg, 4.16 mmol) at 0° C., followed by the addition of HATU (1.58 g, 4.16 mmol). The reaction mixture was stirred at rt for 12 h. The progress of the reaction was monitored by TLC. The reaction mixture was diluted with water (100 mL) and then extracted with ethyl acetate (2×100 mL). The organic layers were dried over anhydrous Na₂SO₄ and concentrated under reduced pressure. The obtained crude compound was purified by prep HPLC (Mobile phase A: 0.1% FA in H₂O, Mobile phase B: Acetonitrile (100%)). Pure fractions were dried by lyophilization for 2 days to provide di-tert-butyl 2,2′-((3,3′-((2-(2-azidoethoxy)acetyl)azanediyl)bis(propanoyl))bis(piperazine-4,1-diyl))diacetate, Target Z (350 mg, 34.5% yield) as an off white solid. ESI-MS: C₃₀H₅₂N₈O₈(M+H): calc. 653.20, found 653.39.

¹H NMR (CDCl₃, 400 MHz): δ 4.30 (s, 2H), 3.72 (t, J=4.8 Hz, 2H), 3.67-3.63 (m, 6H), 3.57-3.45 (m, 8H), 3.14 (d, J=4.4 Hz, 4H), 2.59-2.54 (m, 12H), 1.47 (s, 18H). LC-MS: C₃₀H₅₂N₈O₈ (M+H): calc. 653.39, found 653.70 (M+H). HPLC: 96.77% purity,

### Example 16: Synthesis of Target AL

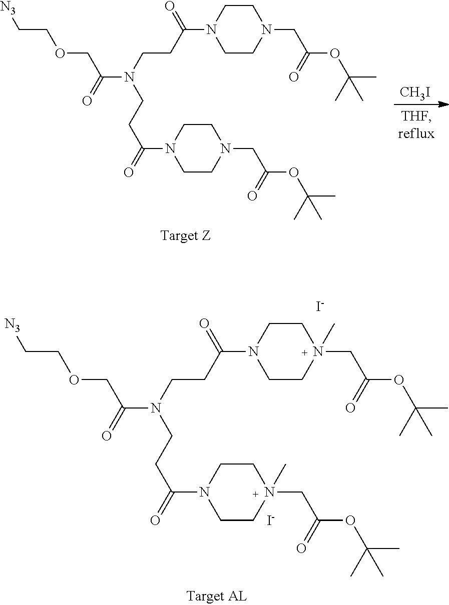

### Synthesis of 4,4′-(3,3′-((2-(2-azidoethoxy)acetyl)azanediyl)bis(propanoyl))bis(1-(2-(tert-butoxy)-2-oxoethyl)-1-methylpiperazin-1-ium): Target AL

To a solution of di-tert-butyl 2,2′-((3,3′-((2-(2-azidoethoxy)acetyl)azanediyl)bis(propanoyl))bis(piperazine-4,1-diyl))diacetate, Target Z (330 mg, 0.51 mmol) in THF (10 mL) was added iodomethane (0.07 ml, 1.11 mmol) at 0° C. and reaction mixture was stirred at 0° C. for 6 h. The progress of the reaction was monitored by TLC. The reaction mixture was concentrated under reduced pressure and the crude compound was purified by trituration with diethyl ether (10 mL) to provide 4,4′-(3,3′-((2-(2-azidoethoxy)acetyl)azanediyl)bis(propanoyl))bis(1-(2-(tert-butoxy)-2-oxoethyl)-1-methylpiperazin-1-ium), Target AL (300 mg, 86.27% yield) as an off-white solid.

¹H NMR (DMSO-d₆, 400 MHz): δ 4.25 (s, 2H), 3.98-3.89 (m, 12H), 3.71-3.62 (m, 12H), 3.51-3.42 (m, 6H), 2.98-2.94 (m, 6H), 2.69 (t, J=6.8 Hz, 2H), 2.40 (s, 1H), 1.46 (s, 18H). LC-MS: C₃₂H₅₈I₂N₈O₈(M−2I+H): calc. 683.44, found 684.10 (M−2I+H). HPLC: 97.38% purity.

### Example 17: Syntheses of POZ Polymer Drug-Linker Moiety: PEOZ 2K-Target A

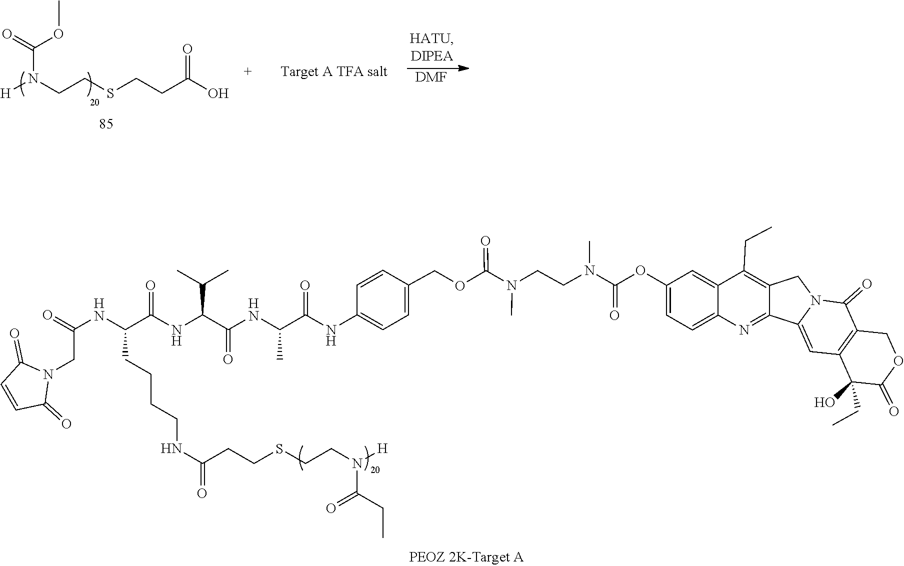

PEOZ acid 2K, compound 85 (77.0 mg, 0.0365 mmol) was dissolved in anhydrous ACN (6 mL) and the solution was evaporated to dryness. To the residual was added anhydrous DMF (6 mL). Under argon atmosphere, HATU (15.1 mg, 0.0398 mmol) was added, followed by addition of DIPEA (23.1 μL, 0.133 mmol). The solution was stirred under argon for ten minutes followed by the addition of Target A TFA salt (prepared as described in Example 1, 40.0 mg, 0.0332 mmol). The reaction was conducted at ambient temperature under argon for one hour. The reaction was monitored by RP-HPLC. After 1 h MeOH (5 mL) was added and the mixture was stirred for 20 min, followed by the addition of acetic acid (7.6 μL, 0.133 mmol). The reaction mixture was evaporated to dryness under vacuum at 40° C. The crude product was purified by flash chromatography (Mobile phase A: 0.1% Acetic acid in water (A), Mobile phase B: 0.1% Acetic acid in ACN). Pure fractions were concentrated by rotary evaporation, and then lyophilized to provide PEOZ 2K-Target A (61 mg, 100% purity) as a white colored solid. The molecular weight was 3175 Da.

### Example 18: Synthesis of PMOZ 2K (Target K)₂ (ZH-82-160)

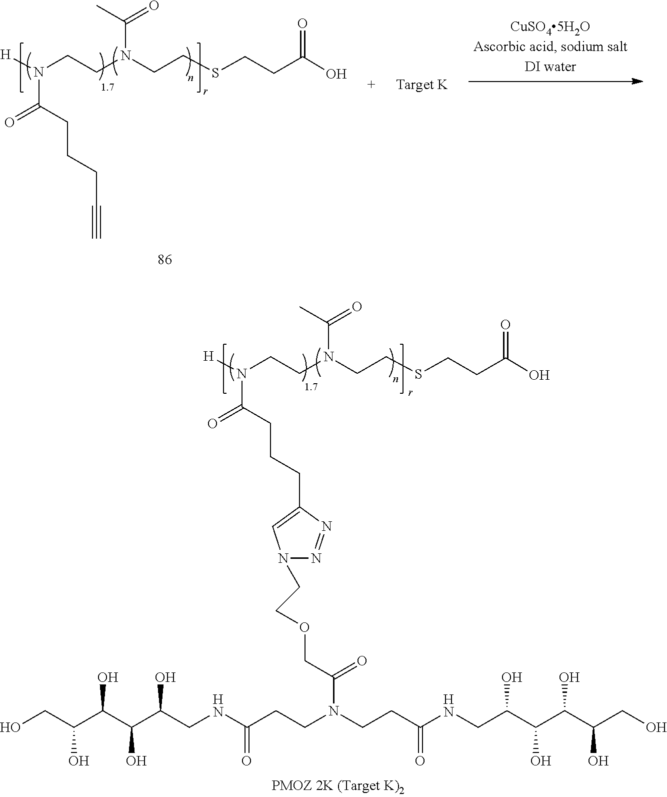

To Target K (1620 mg, 2.636 mmol, prepared as described in Example 11, and PMOZ 2p 2K (2.653 g, 1.277 mmol) was added deionized water (162 mL) to dissolve the mixture. The clear solution was purged with argon for 15 minutes at rt, then Na Ascorbate (287.2 mg, 1.450 mmol) was added followed by the immediate addition of CuSO₄·5H₂O (361.9 mg, 1.450 mmol). The solution was then stirred at ambient temperature. The reaction was monitored by RP-HPLC then purified on Ambersep M4195 media to remove copper ion. The copper-free eluent was concentrated by rotary evaporation, then purified by flash chromatography (Mobile phase A: water, Mobile phase B: ACN). Pure fractions were concentrated by rotary evaporation, followed by lyophilization, to provide PMOZ 2K (Target K)₂ (2.79 g, 100% purity) as a white solid. The molecular weight was 3076 Da.

### Example 19: Synthesis of PMOZ 2K (Target K)₂-Target A−

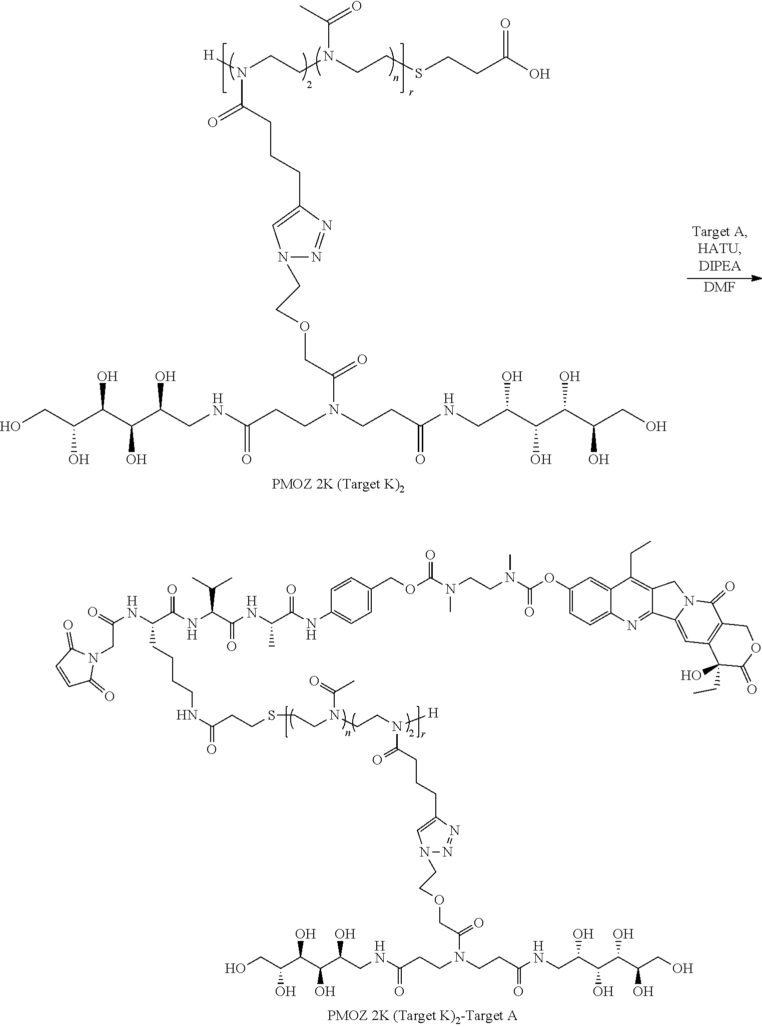

To PMOZ 2K (Target K)₂ (167.2 mg, 0.0548 mmol, prepared as described in Example 18) was added ACN (10 mL), then the mixture was evaporated to dryness and the resulting residue was dissolved in anhydrous DMF (6 mL). Under argon, HATU (22.7 mg, 0.0597 mmol) was added, followed by the addition of DIPEA (34.7 mL, 0.199 mmol). The solution was stirred under argon for 10 min, then Target A (60 mg, 0.0498 mmol, prepared as described in Example 1) was added. The resulting solution was stirred at rt under a slow argon flow for 60 min. The reaction was monitored by RP-HPLC. After 105 minutes MeOH (1 mL) was added, then the mixture, was stirred for 20 min, followed by the addition of. acetic acid (11.4 mL, 0.199 mmol. The reaction mixture, was evaporated by rotary evaporation to dryness at 45° C. The crude product was purified by flash chromatography (Mobile phase A: 0.1% Acetic acid in water, Mobile phase B: 0.1% Acetic acid in ACN). Selected fractions were combined and concentrated to 7 mL. The concentrated solution was then lyophilized to provide PMOZ 2K Target K)₂-Target A (126.3 mg, 99.4% purity) as a white colored solid. The molecular weight was 4104 Da.

### Example 20: Synthesis of PEOZ 2K Target K-Target A

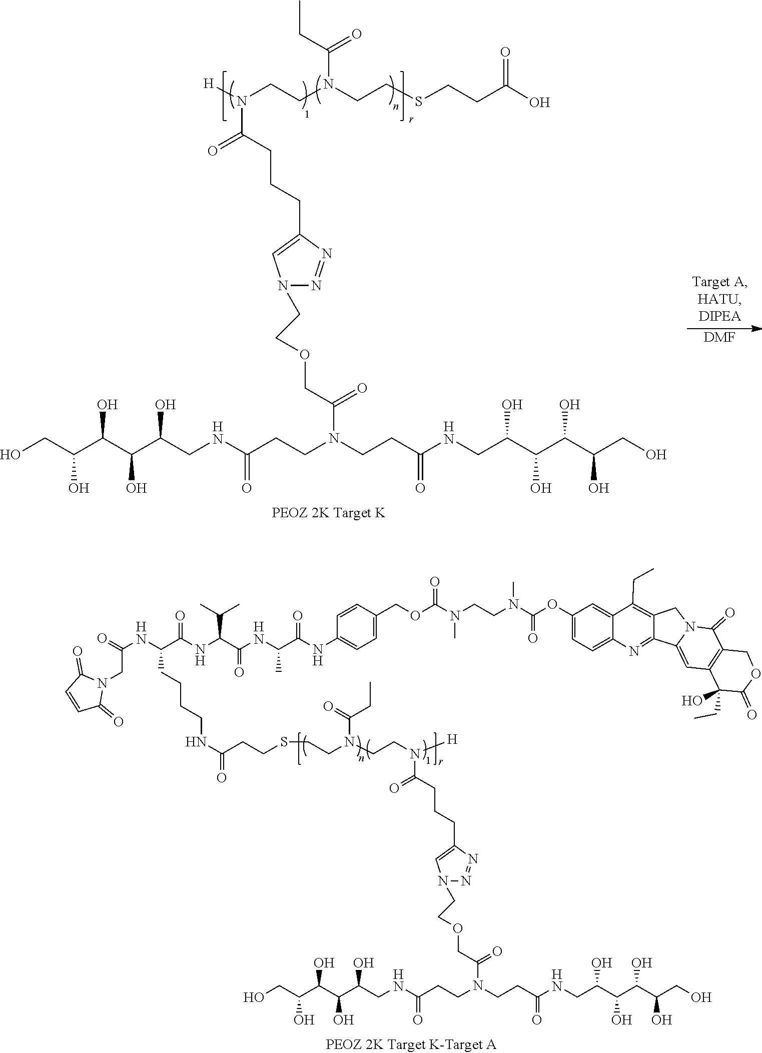

PEOZ 2K Target K-Target A was prepared as described in Example 19 except PEOZ 2K Target K (104.1 mg, 0.0365 mmol) was used to provide PEOZ 2K Target K− Target A (37.6 mg, 100% purity) as a white colored solid. The molecular weight was 3997 Da(MALDI-TOF).

### Example 21: Synthesis of PMOZ 2K (Target K)₂-Target DF

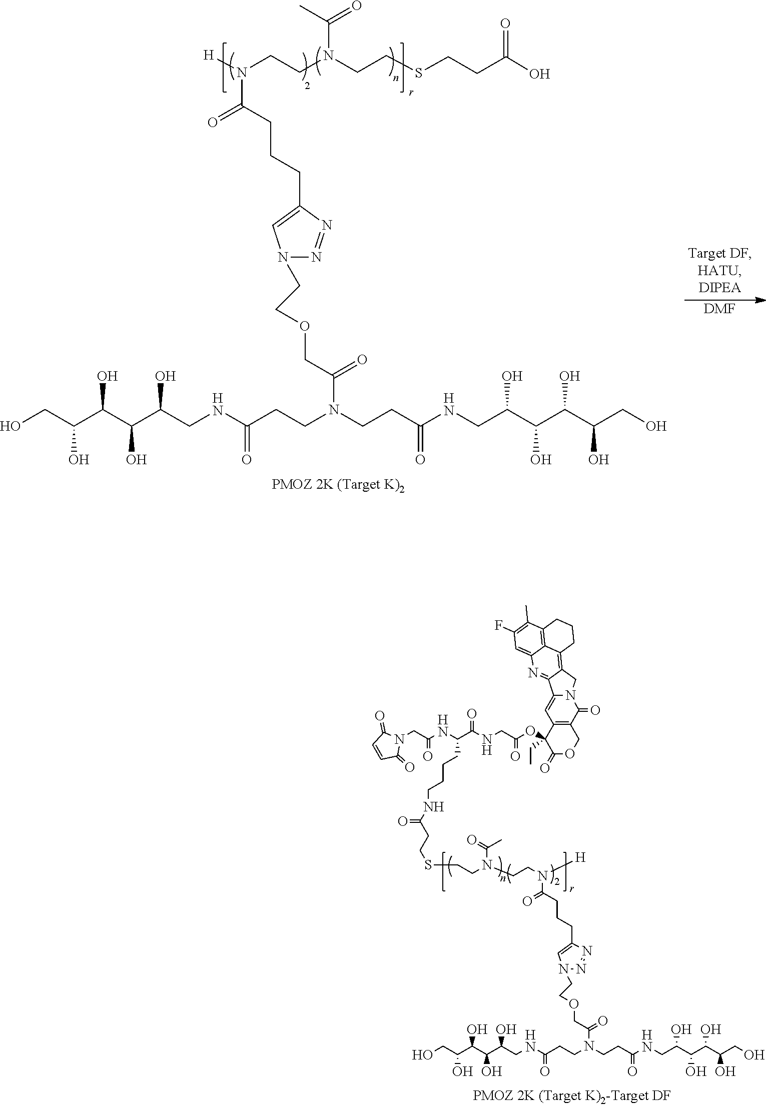

PMOZ 2K (Target K)₂-Target DF was prepared as described in Example 19 except PMOZ 2K (Target K)₂ (631.6 mg, 0.202 mmol prepared as described in Example 18) and Target DF (100 mg, 0.112 mmol, prepared as described in Example 8) were used to provide PMOZ 2K (Target K)₂-Target DF (363.6 mg, 99.4% purity) as an amber-colored solid. The molecular weight was 3671 Da.

### Example 22: Synthesis of PMOZ 2K (Target K)₂-Target DG

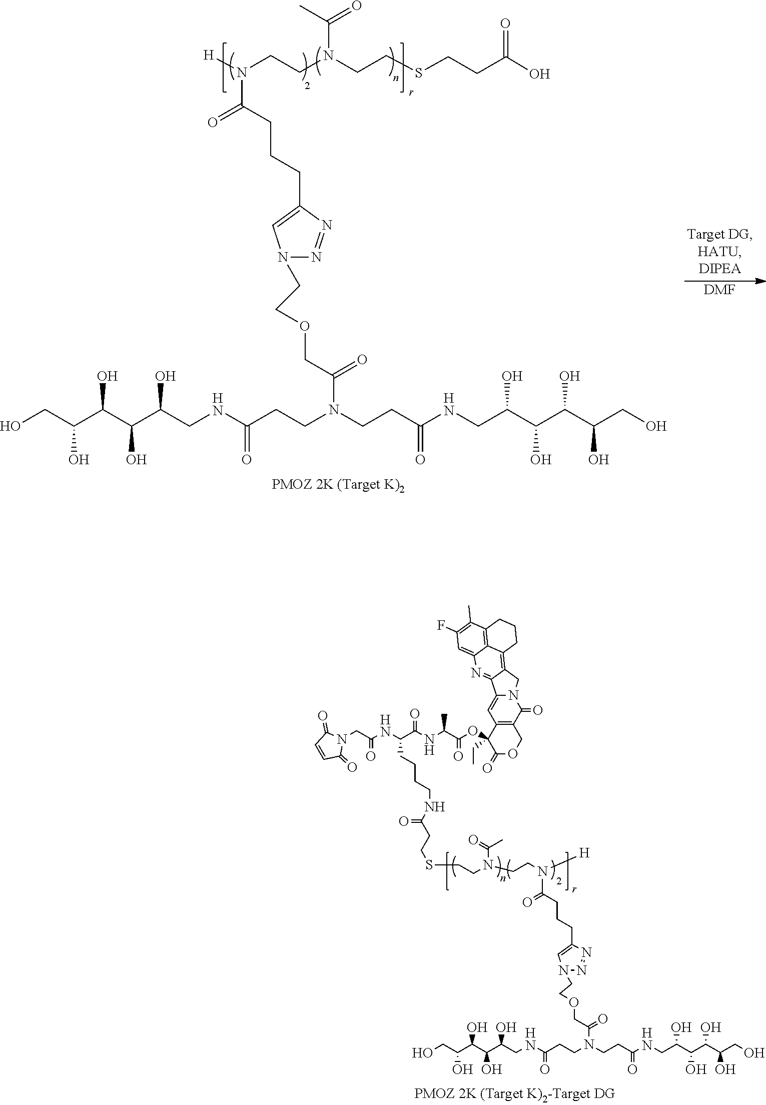

PMOZ 2K (Target K)₂-Target DG was prepared as described in Example 19 except PMOZ 2K(Target K)₂ (344.8 mg, 0.110 mmol, prepared as described in Example 18) and Target DG (50 mg, 0.055 mmol, prepared as

described in Example 9) were used to provide PMOZ 2K (Target K)₂-Target DG (168.7 mg, 1000 purity) as a white colored solid. The molecular weight was 3803 Da.

### Example 23: Syntheses of POZ Polymer Drug-Linker Moieties

Using the procedure described in Example 17, the POZ polymer drug-linker moieties listed in Table 1 were synthesized

TABLE 1

| POZ MW | Drug-linker Moiety | POZ polymer Drug-linker Moiety |
|---|---|---|
| 1K | Target A TFA salt | PEOZ 1K-Target A |
| 2K | Target A TFA salt | PEOZ 2K-Target A |
| 5K | Target A TFA salt | PEOZ 5K-Target A |
| 2K | Target A TFA salt | PEOZ 5K-Target A |
| 2K | Target C TFA salt | PEOZ 2K-Target C |
| 2K | Target M TFA salt | PEOZ 2K-Target M |
| 2K | Target N TFA salt | PEOZ 2K-Target N |
| 2K | Target I TFA salt | PEOZ 2K-Target I |
| 2K | Target H TFA salt | PEOZ 2K-Target H |
| 2K | Target E TFA salt | PEOZ 2K-Target E |
| 2K | Target A TFA salt | PMOZ 2K-(Target K)₂-Target A |
| 2K | Target A TFA salt | PEOZ 2K-Target K-Target A |
| 2K | Target DF. TFA salt | PMOZ 2K-(Target K)-Target DF |
| 2K | Target DG.TFA salt | PMOZ 2K (Target K)₂-Target DG |

### Example 24: General Procedure for the Synthesis of a POZ Polymer Antibody-Drug Conjugate

To a solution of trastuzumab (12.5 mg, 0.083 mol) in 1.25 mL PBS, (pH 7.4) was added an aqueous solution of TCEP (0.83 mol, 0.083 mL of a 10 mM solution). The reaction mixture was then incubated in a thermomixer at 37° C. for 1 h. To the solution of reduced antibody was then added the POZ polymer drug-linker moiety (1.16 μmol, 0.116 mL of a 10 mM solution in ACN). The reaction mixture was then incubated on a tube rotor at rt for 2 h, then desalted using a HiTrap desalting column (5 mL, PBS pH 7.4 eluant). The resulting POZ polymer antibody-drug conjugate was characterized (Table 2)

TABLE 2

| POZ polymer antibody-drug conjugate | DAR |
|---|---|
| Trastuzumab PEOZ 5K-Target A | 8 |
| Trastuzumab PEOZ 2K-Target A | 8 |
| Trastuzumab PEOZ 2K-Target C | 8 |
| Trastuzumab PEOZ 2K-Target M | 8 |
| Trastuzumab PEOZ 2K-Target N | 8 |
| Trastuzumab PEOZ 2K-Target I | 8 |
| Trastuzumab PEOZ 2K-Target H | 8 |
| Trastuzumab PEOZ 2K-Target E | 8 |
| Trastuzumab-PMOZ 2K Target A | 8 |
| Trastuzumab-PMOZ 2K (Target K)₂-Target | 8 |
| DF | |
| Trastuzumab-PMOZ 2K (Target K)₂-Target | 8 |
| DG | |

### Example 25: Characterization of POZ Polymer Antibody-Drug Conjugate

The POZ polymer trastuzumab-drug conjugates were characterized by determining the change in retention time of the antibody drug conjugate relative to the trastuzumab antibody by hydrophobic interaction chromatography. Table 3 summarizes the results for the change in retention time of the POZ polymer trastuzumab-drug conjugates relative to the trastuzumab antibody and the percent aggregation of each antibody drug conjugate. Table 4 summarizes the results for the change in retention time of the POZ azido acetylene click polymer trastuzumab-drug conjugates relative to the unmodified trastuzumab antibody and the percent aggregation of each antibody drug conjugate

TABLE 3

| | | | Aggregation |
|---|---|---|---|
| ADC | DAR | HIC shift | (%) |
| Trastuzumab-PEOZ 5K Target A | 8.2 | 8.1 min | <1 |
| Trastuzumab-PEOZ 2K Target A | 8.2 | 6.4 min | <1 |
| Trastuzumab-PEOZ 2K Target C | 8.2 | 6.1 min | <1 |
| Trastuzumab-PEOZ 2K Target M | 8.2 | 5.8 min | <1 |
| Trastuzumab-PEOZ 2K Target N | 8.2 | 5.8 min | <1 |
| Trastuzumab-PEOZ 2K Target I | 8.2 | 6.6 min | <1 |
| Trastuzumab-PEOZ 2K Target H | 8.2 | 6.5 min | <1 |
| Trastuzumab-PEOZ 2K Target E | 8.2 | 6.7 min | <1 |
| Trastuzumab-PMOZ 2K Target A | 8.2 | 2.2 min | <1 |

TABLE 4

| | No of Pendent | HIC | Aggregation |
|---|---|---|---|
| ADC | Groups | shift | (%) |
| Trastuzumab-PEOZ | None | 6.4 min | <1 |
| 2K Target A | | | |
| Trastuzumab-PEOZ | 1.2 | 3.5 min | <1 |
| 2K Target K- | | | |
| Target A | | | |
| Trastuzumab-PEOZ | 2 | 3.0 min | <1 |
| 2K Target K- | | | |
| Target A | | | |
| Trastuzumab-PEOZ | 4.8 | 2.9 min | <1 |
| 2K Target K- | | | |
| Target A | | | |
| Trastuzumab-PMOZ | None | 2.2 min | <1 |
| 2K Target A | | | |
| Trastuzumab-PMOZ | 1.8 | 1.2 min | <1 |
| 2K Target K- | | | |
| Target A | | | |

The result in Table 3 show that changes in POZ molecular weight and structure (PEOZ vs PMOZ) can impact ADC hydrophilicity. The data also show that in each case, the ADCs do not show any aggregation. The results in Table 4 show that the presence of a pendent group in the POZ azido acetylene click polymer trastuzumab-drug conjugate makes the conjugate more hydrophilic.

### Example 26: Stability of POZ Polymer Antibody-Drug Conjugates in Mouse, Rat, Monkey or Human Plasma

The stability of the POZ polymer antibody-drug conjugates in mouse, rat, monkey and human plasma was determined by LC-MS/MS. The 3 POZ polymer antibody-drug conjugates listed in Table 5 and Enhertu and trastuzuman-val-cit-SN38 as positive controls were incubated with the plasma at 37° C. for 14 days. The amount of antibody-drug conjugate remaining was measured at 0, 1, 2, 3, 4, 6, 8, 23, 32, 56, 79, 96, 120, 144, 168, 192, 220, 264, 288, 312 and 336 h by LC-MS/MS. Table 4 give the results for the amount of antibody-drug conjugate remaining at 14 days.

TABLE 5

| | % ADC Remaining in Plasma after 14 | | | |
|---|---|---|---|---|
| | days (336 h) | | | |
| ADC | Mouse | Rat | Monkey | Human |
| Trastuzumab-PEOZ | 85 | 80 | 96 | 91 |
| 2K Target A | | | | |
| Trastuzumab-PEOZ | 72 | 100 | 92 | 100 |
| 2K Target H | | | | |
| Trastuzumab-PEOZ | 83 | 73 | 86 | 90 |
| 2K Target I | | | | |
| Trastuzumab-Dxd | 40 | 35 | 40 | 35 |
| Trastuzumab- | 41 | 44 | 40 | 35 |
| Val-Cit-SN38 | | | | |

The results show that the POZ polymer antibody-drug conjugates are stable in mouse, rat, monkey and human plasma.

All publications, including, e.g., non-patent literature, patent applications, and patents, cited in this specification are incorporated herein by reference for all purposes. The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### EQUIVALENT

The details of one or more embodiments of the invention are set forth in the accompanying description above. Although any methods and materials similar or equivalent to those described herein can be used in the practice or testing of the present disclosure, the methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the invention to the precise form disclosed, but by the claims appended hereto.

What is claimed is:

1. A polymer antibody drug conjugate of Formula (I), or a pharmaceutically acceptable salt or solvate thereof: 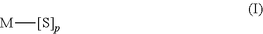
wherein:
M is a recognition moiety; and
S is a scaffold of Formula (II): 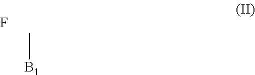
wherein:
F has Formula (III): 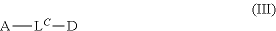
wherein:
A is A¹ after conjugation to M and A² prior to conjugation to M, wherein A¹ is a divalent linker moiety connecting M to L^{C};
L^{C} is a linker moiety connecting A¹ to D; and
D is a drug moiety; and
B¹ is a polymer moiety; and
p is an integer ranging from about 1 to about 8.

2. The conjugate of claim 1, wherein A² is a monovalent linker moiety comprising a functional group capable of forming a covalent bond with a functional group of the recognition moiety.

3. The conjugate of claim 1, wherein the polymer moiety is a water-soluble polymer.

4. The conjugate of claim 3, wherein the water-soluble polymer is a polyoxazoline (POZ) polymer.

5. The conjugate of claim 4, wherein the water-soluble polymer is a co-polymer comprising about 50 to about 99.5 weight percent of a polyoxazoline polymer by weight of the total polymer components.

6. The conjugate of claim 1, wherein the linker moiety L^{C} has the Formula (IV): 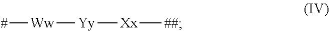
wherein:
each W independently is a peptide unit comprising at least two amino acids;
Y is a self immolative moiety, a non-self-immolative releasable moiety or a non-cleavable moiety;
X is a spacer moiety;
w is an integer from about 0 to about 12;
y is an integer from 0 to 1;
x is an integer from 0 to 1;
# donates attachment to A¹ or A²; and
## donates attachment to D.

7. The conjugate of claim 6, wherein each A¹ independently is a divalent linker moiety connecting the recognition moiety to variable W, if present, or to variable Y, if W is absent; or connecting the recognition moiety to variable X, if both variables W and Y are absent; or connecting the recognition moiety to the drug moiety D, if variables W, Y and X are absent.

8. The conjugate of claim 7, wherein each A¹ independently is: 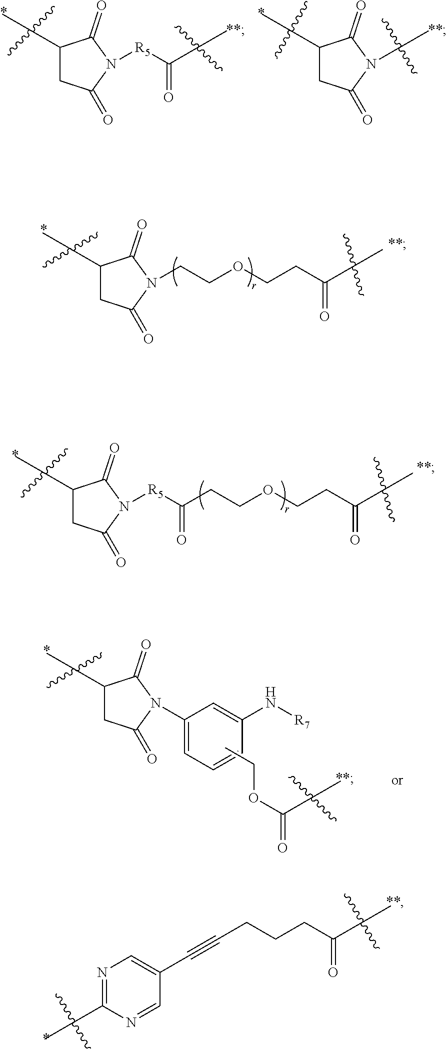
wherein:
R₅ is —O—, —NR₆—, —(C₁-C₁₀)-alkyl-, —(C₁-C₁₀)-alkenyl-, —(C₁-C₁₀)-alkynyl-, —(C₃-C₈)-cycloalkyl-, -aryl-, —O—(C₁-C₈)-alkyl-, —O—(C₁-C₁₀)-alkenyl-, —O—(C₁-C₁₀)-alkynyl-, -aryl-(C₁-C₁₀)-alkyl-C(O), —(C₁-C₁₀)-alkyl-aryl-, —O—C(O)—(CH₂CH₂O)r-(CH₂)₂—, —(CH₂CH₂O)r-, or —(CH₂CH₂O)r-(CH₂)₂—,
R₆ is H, hydroxy, or (C₁-C₄)-alkyl;
R₇ is one or more amino acids;
r is an integer ranging from about 1 to about 12;
* denotes attachment to the recognition moiety;
** denotes attachment to L^{C}; and
*** donates attachment to the polymer moiety B¹.

9. The conjugate of claim 7, wherein each A² independently is: 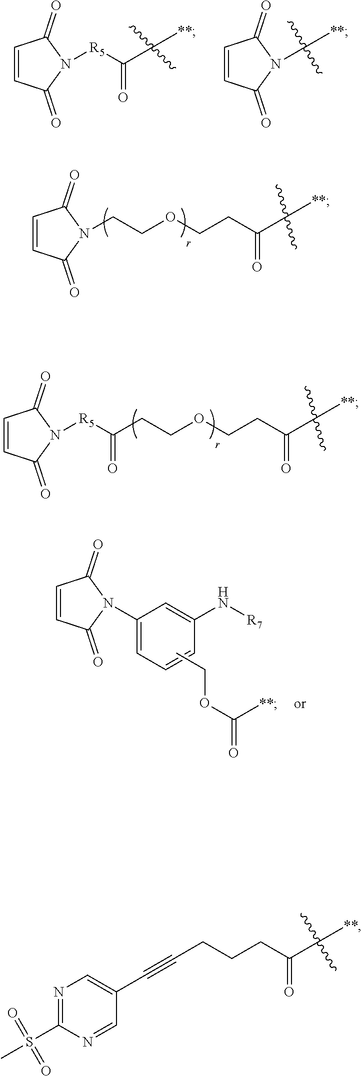
wherein
r is an integer ranging from about 1 to about 12;
** denotes attachment to L^{C}; and
*** donates attachment to the polymer moiety B¹.

10. The conjugate of claim 6, wherein each W independently is a peptide unit connecting variable A¹ or A² to variable Y, if present, or to variable X if Y is absent; or connecting variable A¹ or A² to the drug moiety D if both variables Y and X are absent.

11. The conjugate of claim 1, wherein the drug moiety D is a camptothecin drug or a topoisomerase II inhibitor drug moiety, or a prodrug, solvate, pharmaceutically acceptable salt thereof.

12. The conjugate of claim 11, wherein the camptothecin drug moiety (D) is a compound of Formula (VI), or a prodrug, solvate, pharmaceutically acceptable salt thereof: 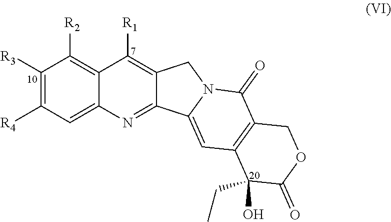
wherein:
R₁ is H, 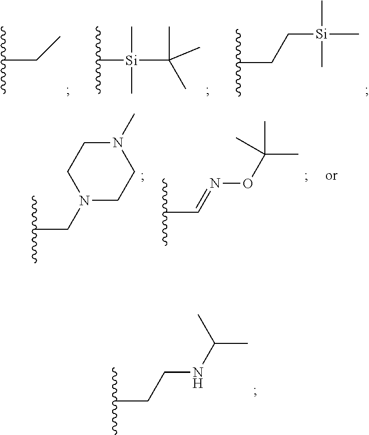
R₂ is H or 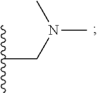 [SMILES: *CN(C)C]
or R₁ and R₂ together are 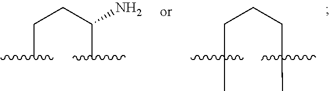 [SMILES: *CC[C@@H](*)N ; *CCC*]
R₃ is H, —OH, or —CH₃; or
R₄ is H or F; or R₃ and R₄ together are 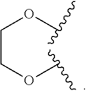
wherein: 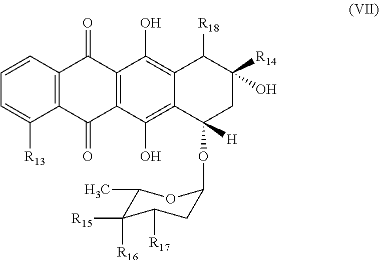
R₁₃ is H, hydroxy or —OCH₃;
R₁₄ is —C₂H₅, —C(O)CH₃, —C(O)CH₂OH, —C(O)CH₂O—C(O)CH(OC₂H₅)₂, C(O)CH₂O—C(O)C₄H₉;
one of R₁₅ and R₁₆ is H; and the other is H, hydroxy or a tetrahydropyran-2-yloxy (OTHP) group;
R₁₇ is —NH₂, —N(CH₃)₂, 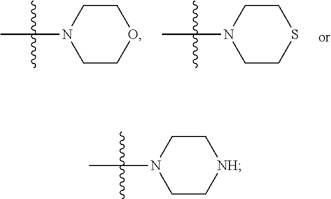 [SMILES: *N1CCOCC1 ; *N1CCSCC1 ; *N1CCNCC1]
and
R₁₈ is H or —COOCH₃.

13. The conjugate of claim 11, wherein the topoisomerase II inhibitor drug moiety is an anthracycline derivative of Formula (VII), or a prodrug, solvate, pharmaceutically acceptable salt thereof: 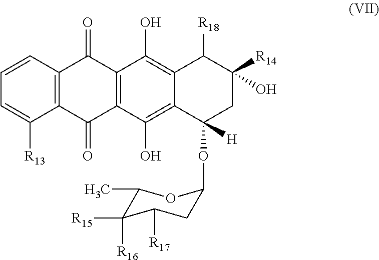
wherein:
R₁₃ is H, hydroxy or —OCH₃;
R₁₄ is —C₂H₅, —C(O)CH₃, —C(O)CH₂OH, —C(O)CH₂O—C(O)CH(OC₂H₅)₂, C(O)CH₂O—C(O)C₄H₉;
one of R₁₅ and R₁₆ is H; and the other is H, hydroxy or a tetrahydropyran-2-yloxy (OTHP) group;
R₁₇ is —NH₂, —N(CH₃)₂, 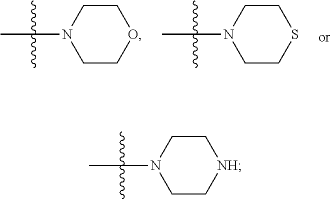 [SMILES: *N1CCOCC1 ; *N1CCSCC1 ; *N1CCNCC1]
and
R₁₈ is H or —COOCH₃.

14. The conjugate of claim 1, wherein B₁ is a polymer of Formula (VIII-A), Formula (VIII-B), or a pharmaceutically acceptable salt thereof, 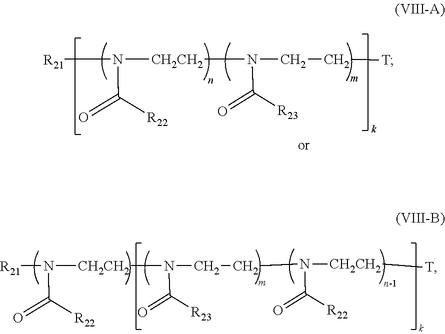
wherein
R₂₁ is an initiating group;
R₂₂ is independently selected for each repeating unit and is a pendent moiety containing an active functional group;
R₂₃ is a non-reactive pendent moiety and is independently selected for each repeating unit;
k indicates that the polymer units m and n are connected to each other in a random order;
n is an integer from 0 to 3;
m is an integer from about 1 to about 50, provided that the sum of n and m is less than or equal to 50; and
Tm is a terminating group.

15. The conjugate of claim 1, wherein B₁ is a polymer of Formula (VIII-J), Formula (VIII-K), Formula (VIII-L), Formula (VIII-M), Formula (VIII-N), Formula (VIII-O), Formula (VIII-P), Formula (VIII-Q) or Formula (VIII-R): 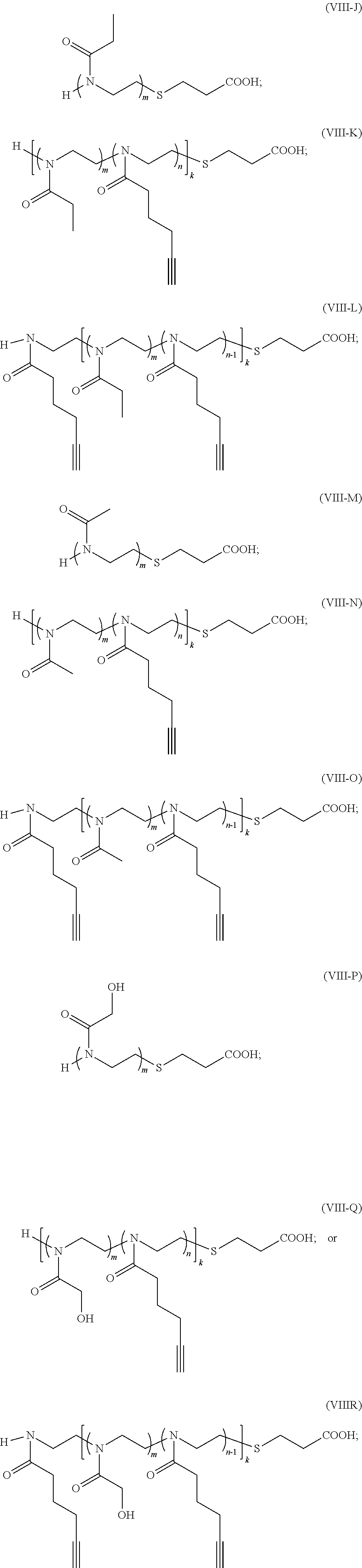
wherein
k indicates that the polymer units m and n and polymer units m and (n−1) are connected to each other in a random order;
m is an integer from about 10 to about 50; and
n is an integer from 1 to 3.

16. A scaffold of Formula (IIₐ), or a pharmaceutically acceptable salt or solvate thereof: 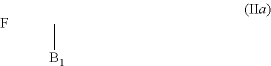
wherein F is a drug linker moiety having Formula (IIIa), or a pharmaceutically acceptable salt or solvate thereof: 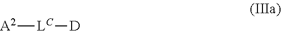
wherein:
A² is a monovalent linker moiety comprising a functional group capable of forming a covalent bond with a functional group of the recognition moiety.
L^{C} is a linker moiety connecting A² to D; and
D is a drug moiety; and
B¹ is a polymer moiety.

17. The scaffold of claim 16, wherein the linker moiety L^{C} has the Formula (IV): 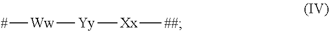
wherein:
each W independently is a peptide unit comprising at least two amino acids;
Y is a self immolative moiety, a non-self-immolative releasable moiety or a non-cleavable moiety;
X is a spacer moiety;
w is an integer from about 0 to about 12;
y is an integer from 0 to 1;
x is an integer from 0 to 1;
# donates attachment to A¹ or A²; and
## donates attachment to D.

18. The scaffold of claim 16, wherein the drug moiety D is a camptothecin drug or a topoisomerase II inhibitor drug moiety, or a prodrug, solvate, pharmaceutically acceptable salt thereof.

19. The scaffold of claim 18, wherein the camptothecin drug moiety (D) is a compound of Formula (VI), or a prodrug, solvate, pharmaceutically acceptable salt thereof: 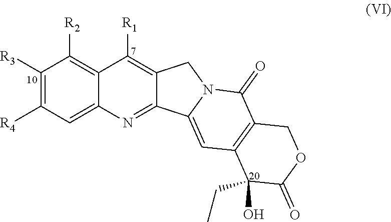
wherein:
R₁ is H, 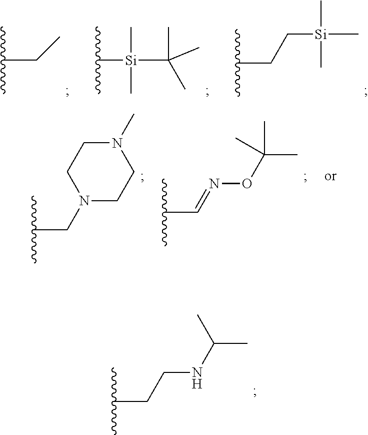
R₂ is H or 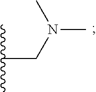 [SMILES: *CN(C)C]
or R₁ and R₂ together are 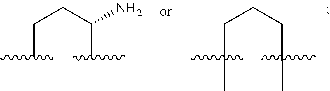 [SMILES: *CC[C@@H](*)N ; *CCC*]
R₃ is H, —OH, or —CH₃; or
R₄ is H or F; or R₃ and R₄ together are 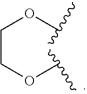
wherein: 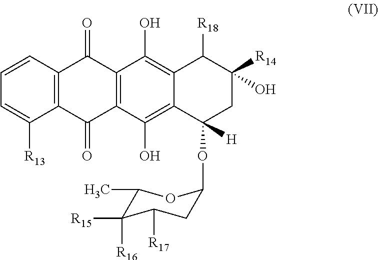
R₁₃ is H, hydroxy or —OCH₃;
R₁₄ is —C₂H₅, —C(O)CH₃, —C(O)CH₂OH, —C(O)CH₂O—C(O)CH(OC₂H₅)₂, C(O)CH₂O—C(O)C₄H₉;
one of R₁₅ and R₁₆ is H; and the other is H, hydroxy or a tetrahydropyran-2-yloxy (OTHP) group;
R₁₇ is —NH₂, —N(CH₃)₂, 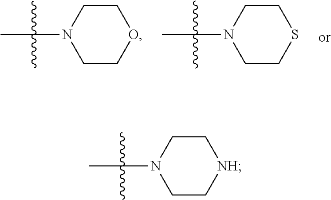 [SMILES: *N1CCOCC1 ; *N1CCSCC1 ; *N1CCNCC1]
and R₁₈ is H or —COOCH₃.

20. The scaffold of claim 18, wherein the topoisomerase II inhibitor drug moiety is an anthracycline derivative of Formula (VII), or a prodrug, solvate, pharmaceutically acceptable salt thereof:
wherein: 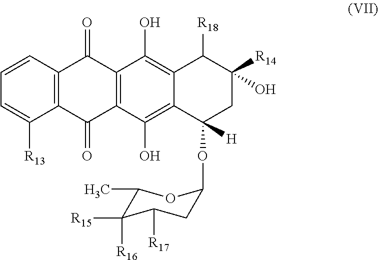
R₁₃ is H, hydroxy or —OCH₃;
R₁₄ is —C₂H₅, —C(O)CH₃, —C(O)CH₂OH, —C(O)CH₂O—C(O)CH(OC₂H₅)₂, C(O)CH₂O—C(O)C₄H₉;
one of R₁₅ and R₁₆ is H; and the other is H, hydroxy or a tetrahydropyran-2-yloxy (OTHP) group;
R₁₇ is —NH₂, —N(CH₃)₂, 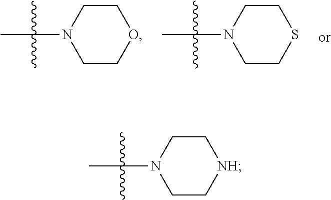 [SMILES: *N1CCOCC1 ; *N1CCSCC1 ; *N1CCNCC1]
and
R₁₈ is H or —COOCH₃.

21. The scaffold of claim 16, wherein the polymer moiety is a polyoxazoline (POZ) polymer.

22. The scaffold of claim 16, wherein the scaffold is: 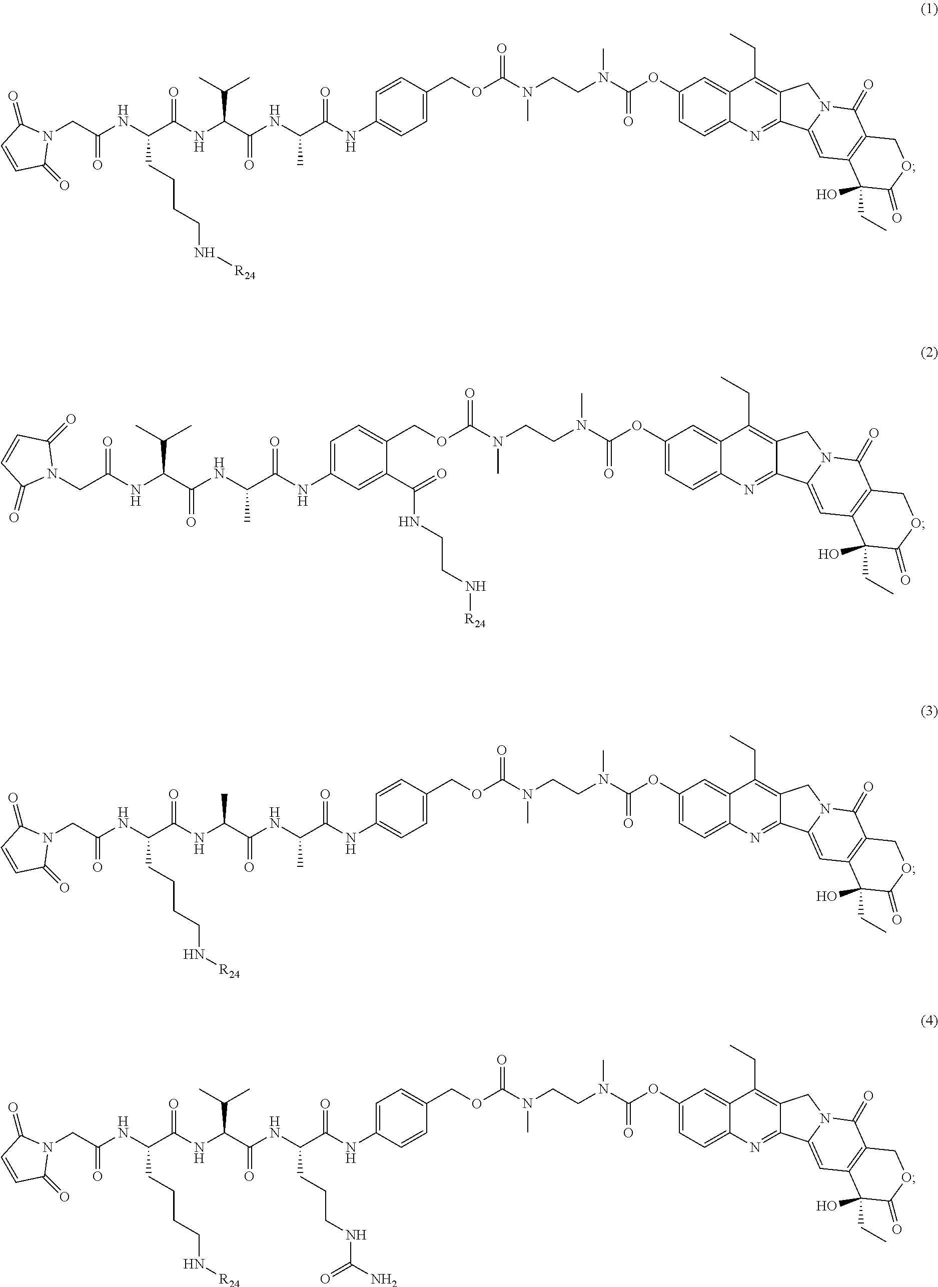 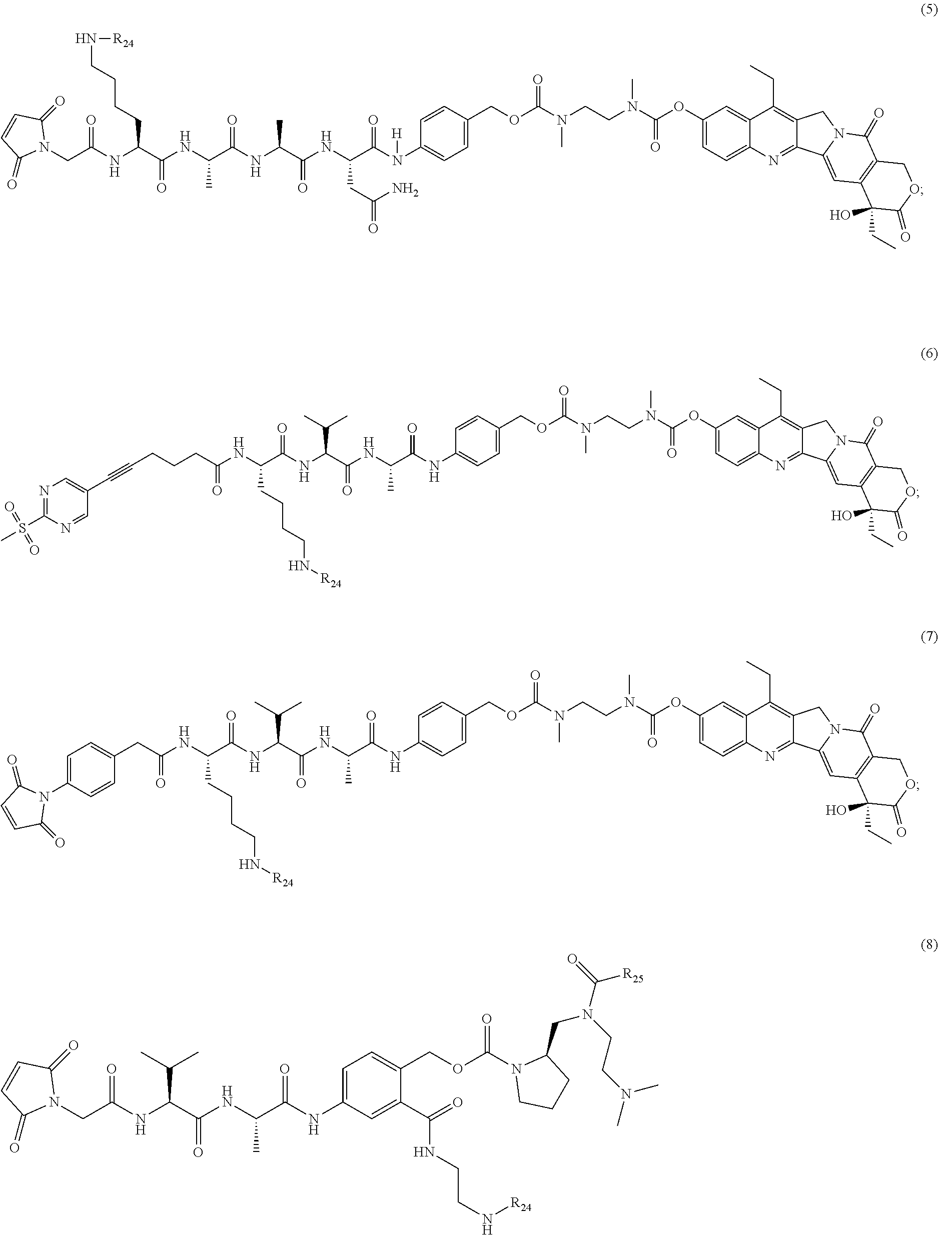 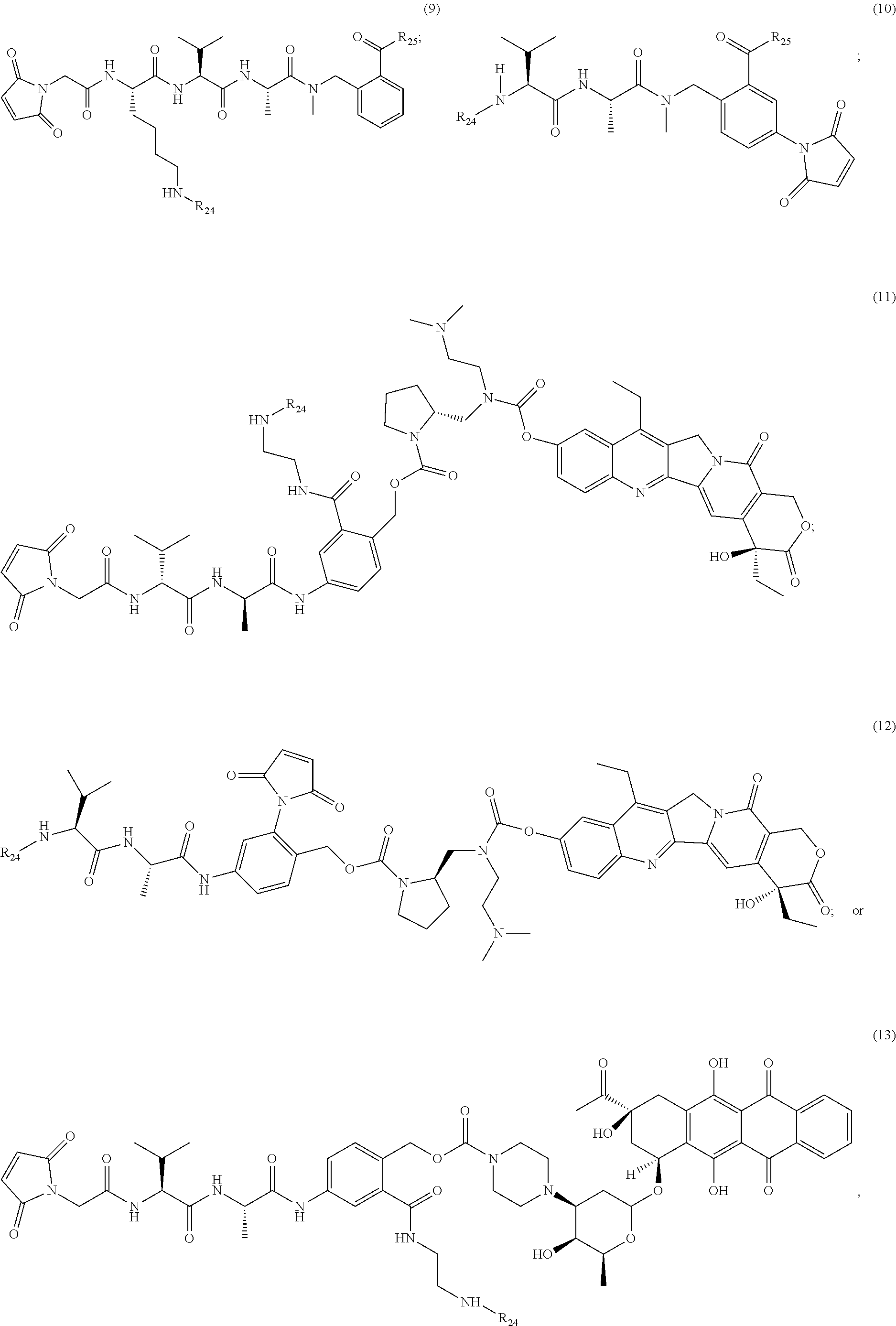
wherein:
R₂₄ is 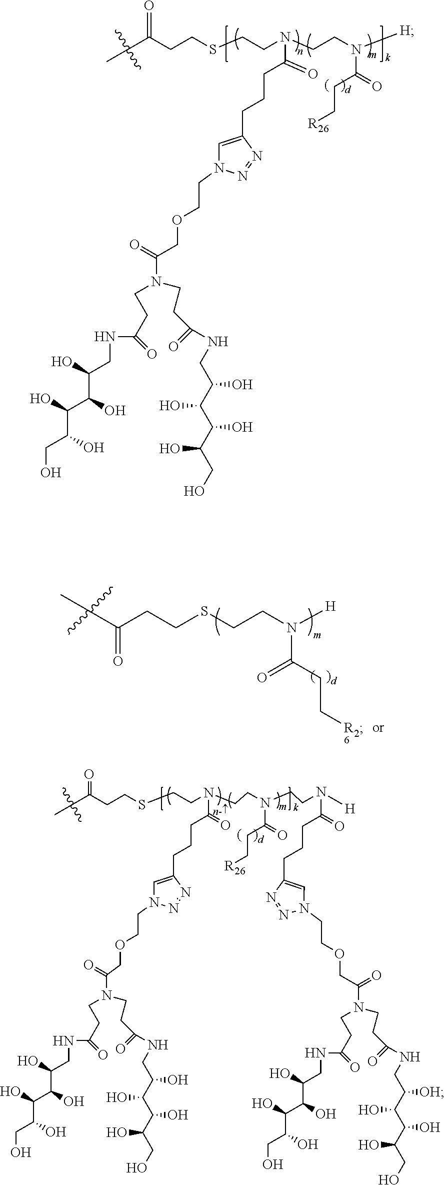
R₂₅ is: 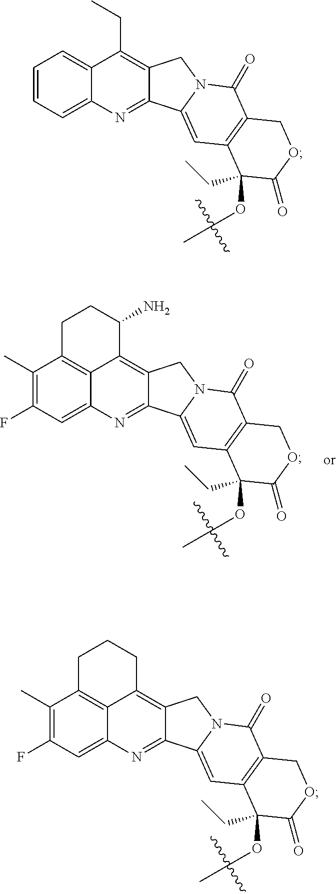 [SMILES: *O[C@]1(CC)C(=O)OCc2c1cc1n(c2=O)Cc2c-1nc1ccccc1c2CC ; *O[C@]1(CC)C(=O)OCc2c1cc1n(c2=O)Cc2c-1nc1cc(F)c(C)c3c1c2[C@@H](N)CC3 ; *O[C@]1(CC)C(=O)OCc2c1cc1n(c2=O)Cc2c-1nc1cc(F)c(C)c3c1c2CCC3]
R₂₆ is H or OH;
d is an integer 0 or 1;
m is an integer from about 10, 20 or 50;
n is an integer ranging from 1 to 3; and
k indicates that the polymer units m and n and polymer units m and (n−1) are connected to each other in a random order.

23. A polymer antibody-drug conjugate having one of the following formulas: 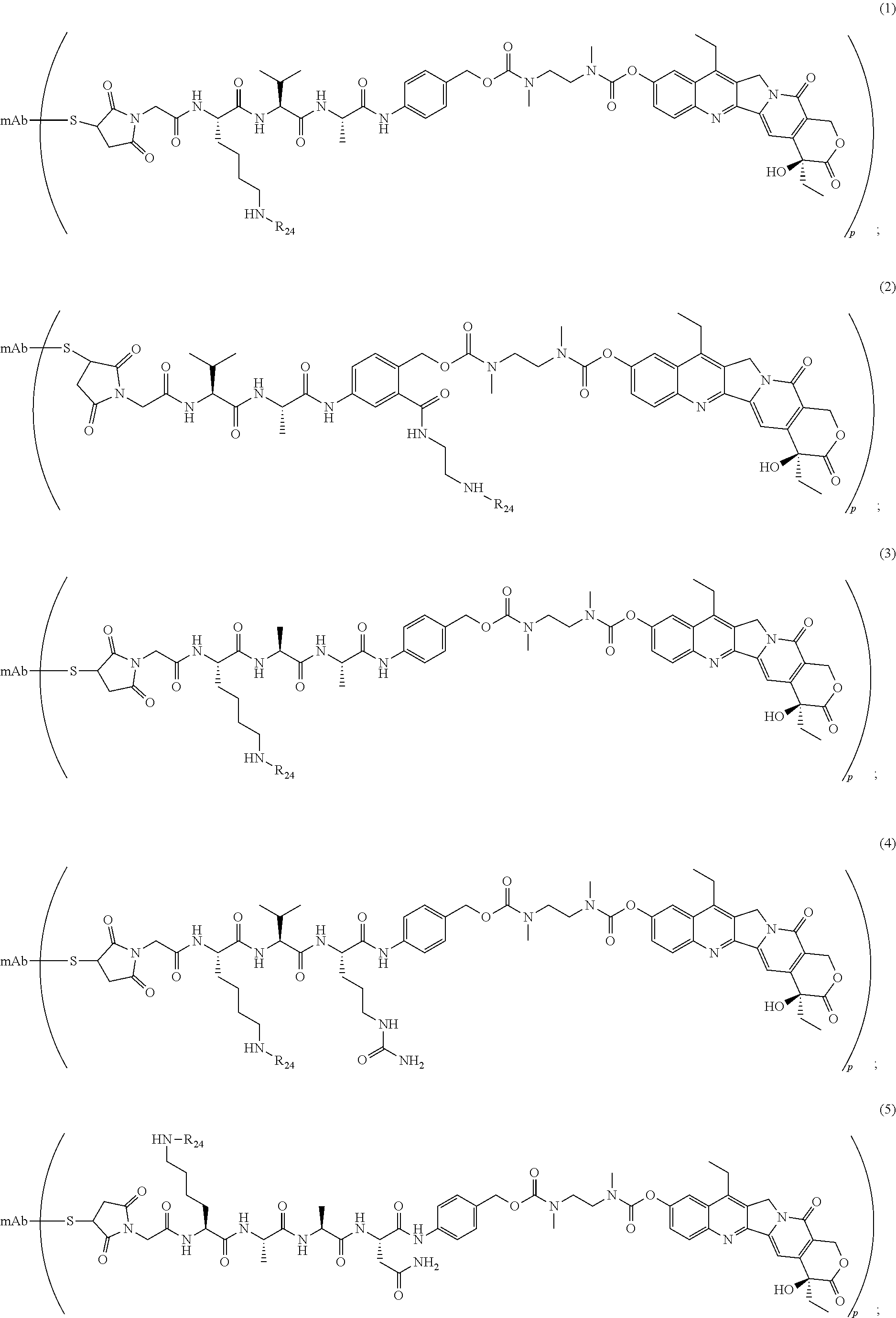 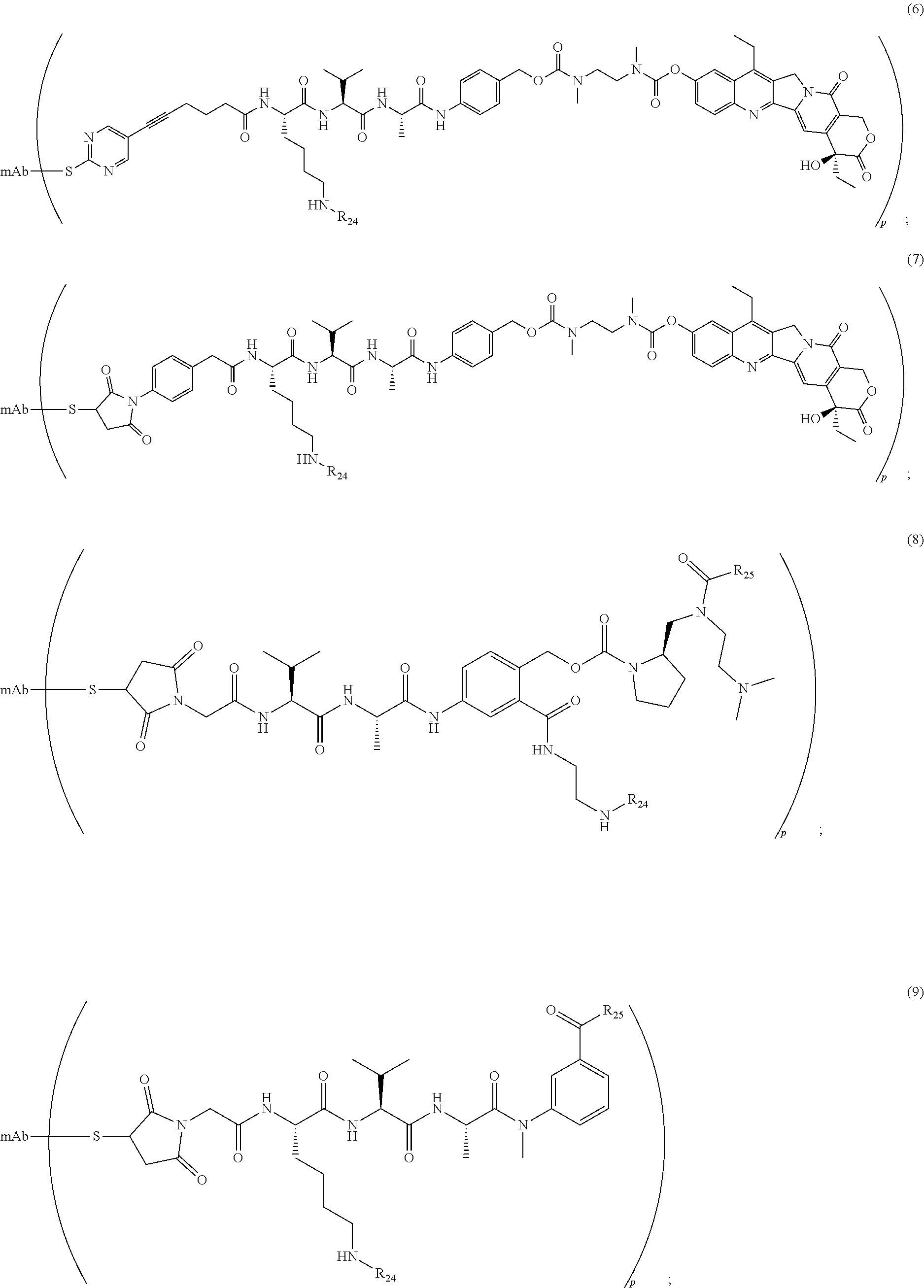 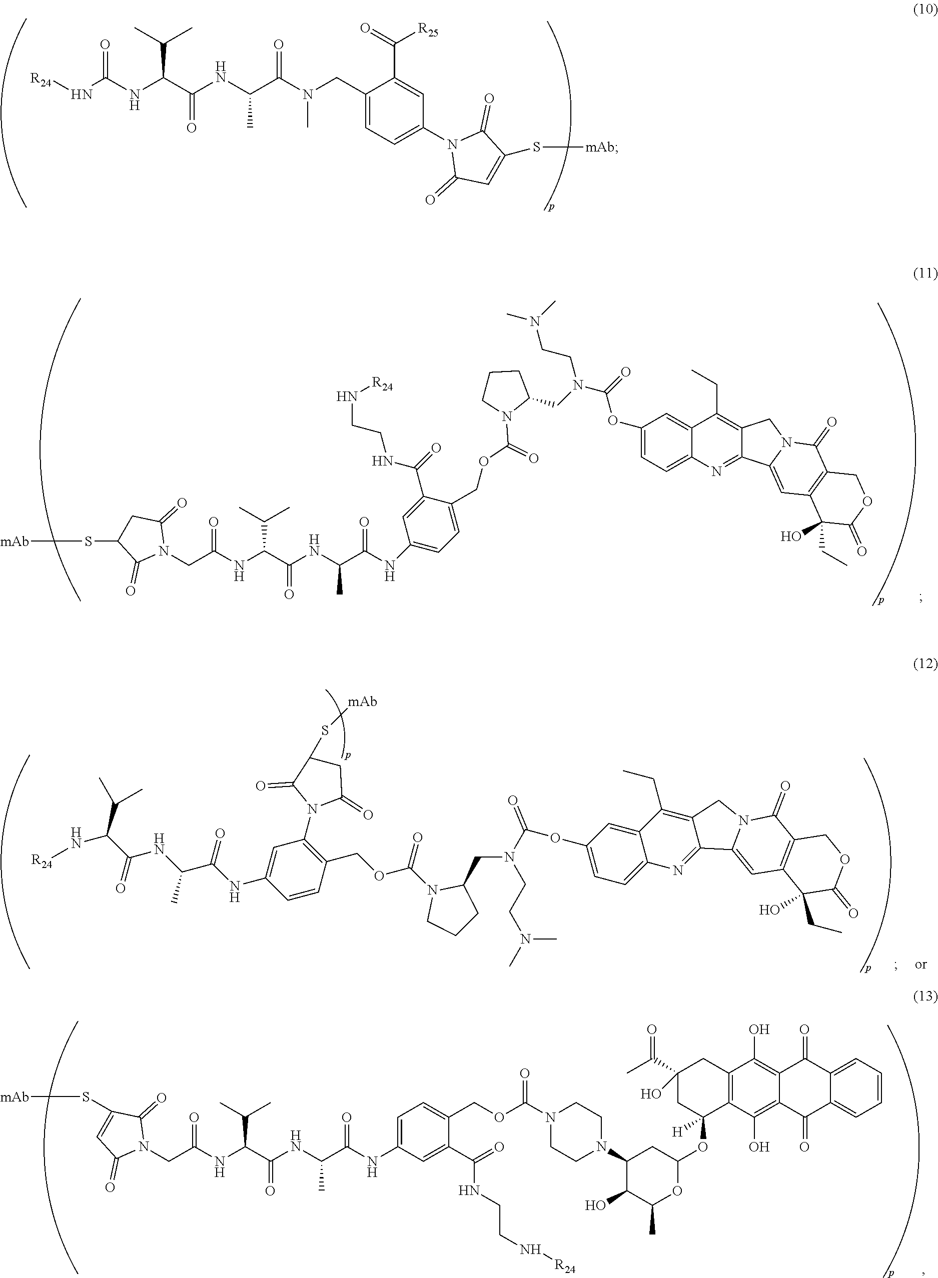
wherein:
R₂₄ is 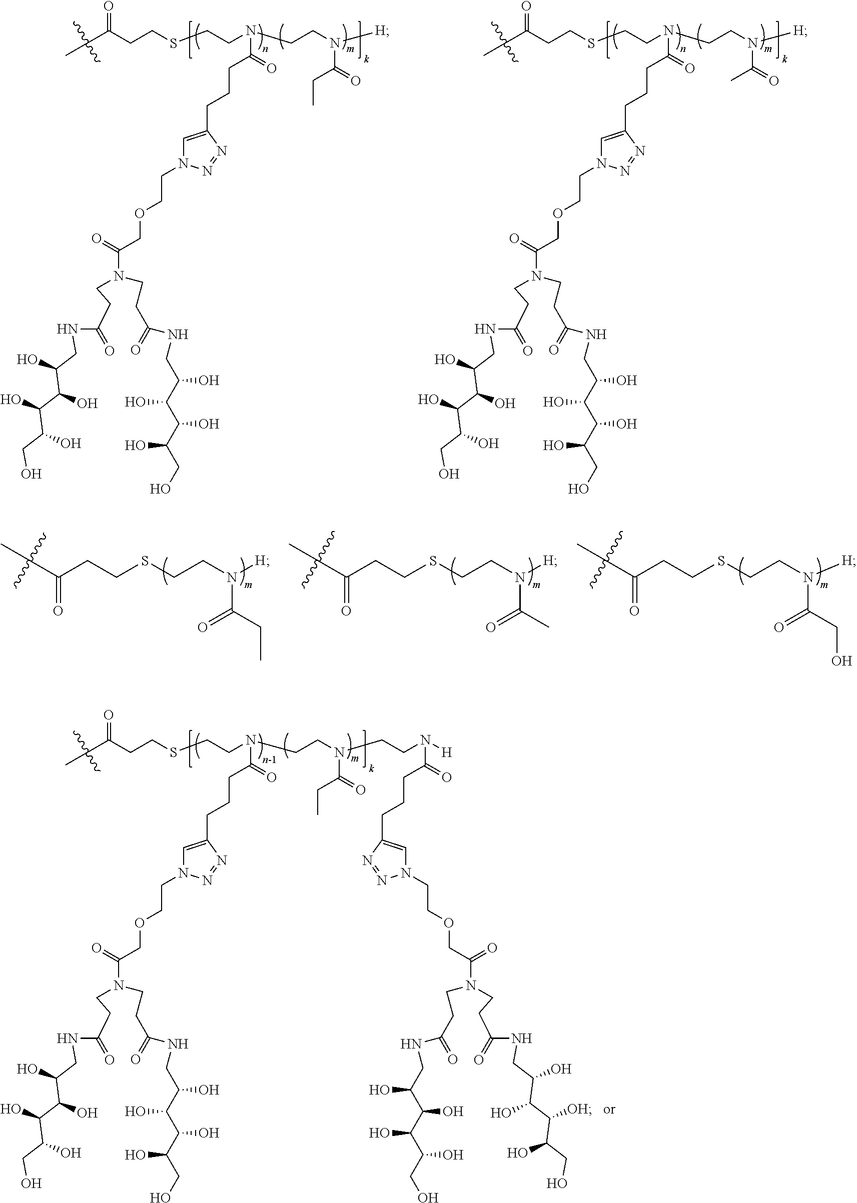 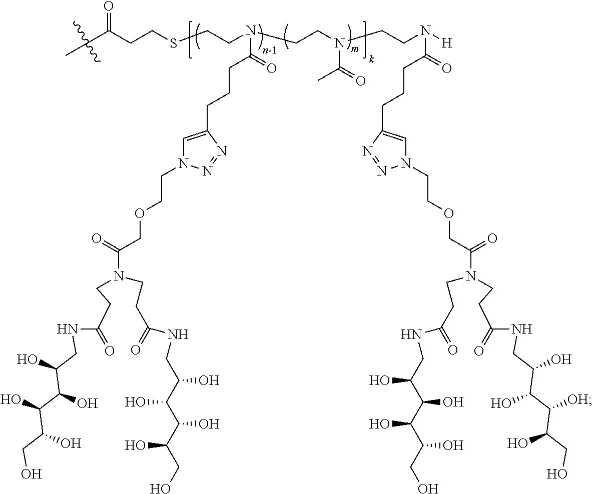
R₂₅ is: 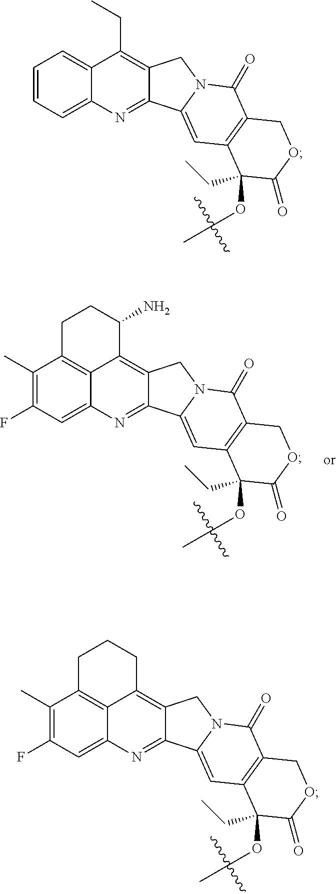 [SMILES: *O[C@]1(CC)C(=O)OCc2c1cc1n(c2=O)Cc2c-1nc1ccccc1c2CC ; *O[C@]1(CC)C(=O)OCc2c1cc1n(c2=O)Cc2c-1nc1cc(F)c(C)c3c1c2[C@@H](N)CC3 ; *O[C@]1(CC)C(=O)OCc2c1cc1n(c2=O)Cc2c-1nc1cc(F)c(C)c3c1c2CCC3]
S is the sulfur atom of an antibody (mAb) cysteine residue;
m is an integer from about 10, 20 or 50;
n ranges from 1 to 3;
k indicates that the polymer units m and n and polymer units m and (n−1) are connected to each other in a random order; and
p is 6 or 8.

24. The polymer antibody drug conjugate of claim 23, wherein R₂₄ is 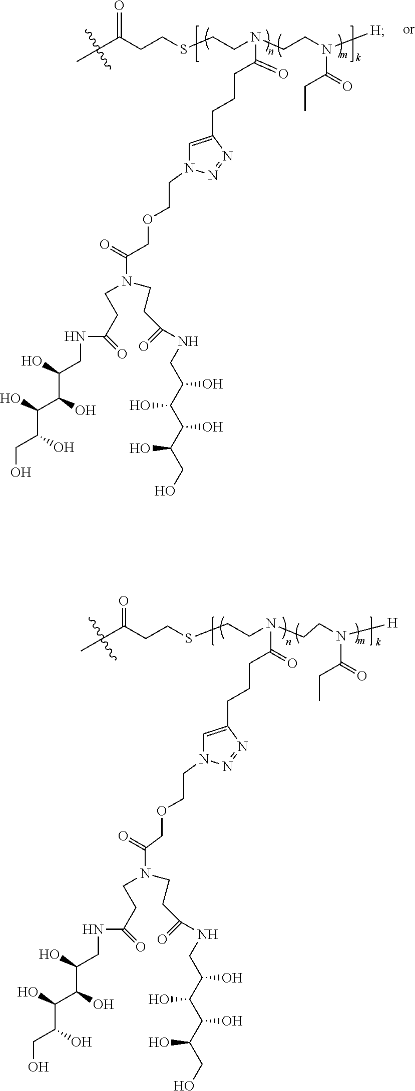
wherein n, m and k are as defined herein.

25. The polymer antibody-drug conjugate of claim 23, wherein R₂₄ is: 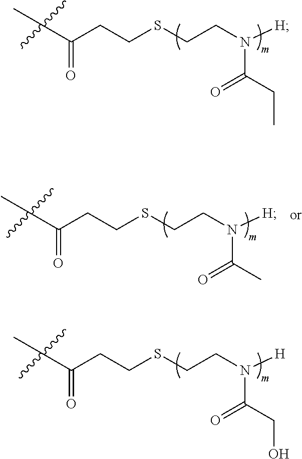
wherein m is as defined herein.
